(19) United States
(12) Patent Application Publication
(10) Pub. No.: US 2011/0027264 A1
(43) Pub. Date: Feb. 3, 2011

(54) GAMMA SECRETASE MODULATORS FOR THE TREATMENT OF ALZHEIMER'S DISEASE

(76) Inventors: Xianhai Huang, Warren, NJ (US); Xianghai Huang, Warren, NJ (US); Anandan Palani, Bridgewater, NJ (US); Robert G. Aslanian, Rockaway, NJ (US); Zhaoning Zhu, Plainsboro, NJ (US); Dmitri A. Pissarnitski, Scotch Plains, NJ (US); Hongmei Li, Warren, NJ (US); William J. Greenlee, Teaneck, NJ (US); Zhiqiang Zhao, Scotch Plains, NJ (US); Robert D. Mazzola, JR., Stewartsville, NJ (US); Jun Qin, Somerset, NJ (US); Pawan Dhondi, Somerset, NJ (US)

Correspondence Address: MERCK;PATENT DEPARTMENT (K-6-1, 1990), 2000 GALLOPING HILL ROAD, KENILWORTH, NJ, 07033-0530, US

(21) Appl. No.: 12/919,574

(22) PCT Filed: Feb. 26, 2009
(86) PCT No.: PCT/US09/35266

§ 371 Date: Oct. 20, 2010

Related U.S. Application Data
(60) Provisional application No. 61/032,595, filed on Feb. 29, 2008.

(51) Int. Cl. A61K 39/395; C07D 401/12; C07D 221/20; A61K 38/16; A61K 31/454; A61K 31/438; A61P 25/28

(52) U.S. Cl. 424/130.1; 546/210; 546/16; 514/17.8; 514/326; 514/278

(57) ABSTRACT

This invention provides novel compounds that are modulators of gamma secretase. The compounds have the formula: Also disclosed are methods of modulating gamma secretase activity and methods of treating Alzheimer's disease using the compounds of formula (I).

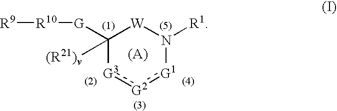

### REFERENCE TO RELATED APPLICATION

This application claims the benefit of U.S. Provisional Application Ser. No. 61/032,595 filed Feb. 29, 2008.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the β amyloid protein is critical for the seeding of amyloid formation: implications for the pathogenesis of Alzheimer's disease, Biochemistry, May 11, 1993, 32(18), p. 4693-4697) and constitute the main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249).

Furthermore, it is known that mutations of APP and presenelin genes, which is are observed in familial Alzheimer's disease, increase production of Aβ40 and Aβ42 (for example, see Gouras G K, et al, Intraneuronal Aβ142 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on β-amyloid accumulation and secretion in neurons and nonneuronal cells, Journal of Biological Chemistry, Dec. 19, 1997, 272(51), p. 32247-32253). Therefore, compounds which reduce production of Aβ40 and Aβ42 are expected to be agents for controlling progress of Alzheimer's disease or for preventing the disease.

These Aβs are produced when APP is cleaved by beta secretase and subsequently cleaved by gamma secretase. In consideration of this, creation of inhibitors of γ-secretase and β-secretase has been attempted for the purpose of reducing production of Aβs. Many of these known secretase inhibitors are peptides or peptidomimetics such as L-685,458. L-685,458, an aspartyl protease transition state mimic, is a potent inhibitor of γ-secretase activity, Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone A G, published May 4, 2006); WO 2004/110350 (Neurogenetics, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

This invention provides novel compounds that are gamma secretase modulators, said novel compounds are of the formula:

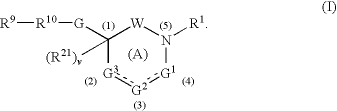

or a pharmaceutically acceptable salt, ester, or solvate thereof, wherein all substituents are defined below.

This invention also provides a compound of formula (I) in pure and isolated form.

This invention also provides a compound of formula (I) selected from the group consisting of: (ID) to (IG), (IM) to (IQ), 1 to 32, 1A to 32A, 1C to 32C, 1E to 32E, B1 to B3, B6, B9 and B10, or a pharmaceutically acceptable salt thereof, or a pharmaceutically acceptable ester thereof, or a solvate thereof.

This invention also provides a compound of formula (I) selected from the group consisting of: A1 to A6, A10, A12 to A107, B4, B5, B7, and B8.

This invention also provides a compound of formula (I) selected from the group consisting of: A1 to A6, A10, A12 to A107, B4, B5, B7, and B8, in pure and isolated form.

This invention also provides a compound of formula (I) selected from the group consisting of: A1 to A6, A10, A12 to A107, B4, B5, B7, and B8, wherein one or more hydrogens are deuterium.

This invention also provides a compound selected from the group consisting of: A7, A8, A9 and A11.

This invention also provides a compound selected from the group consisting of: A7, A8, A9 and A11, in pure and isolated form.

This invention also provides a compound selected from the group consisting of: A7, A8, A9 and A11 wherein one or more hydrogens are deuterium.

This invention also provides compounds of formula (I) wherein from one up to the total number of hydrogens are deuterium.

This invention provides compounds of formula (I) wherein at least one H is deuterium.

This invention provides compounds of formula (I) wherein 1 to 5H are deuterium.

This invention provides compounds of formula (I) wherein one H is deuterium.

This invention provides a pharmaceutical composition comprising an effective amount of one or more (e.g., one) compounds of formula (I), or a pharmaceutically acceptable acceptable salt, ester or solvate thereof, and a pharmaceutically acceptable carrier.

This invention also provides a pharmaceutical composition comprising an effective amount of one or more (e.g., one) compounds of formula (I), or a pharmaceutically acceptable salt, ester or solvate thereof, and an effective amount of one or more (e.g., one) other pharmaceutically active ingredients (e.g., drugs), and a pharmaceutically acceptable carrier.

The compounds of formula (I) can be useful as gamma secretase modulators and can be useful in the treatment and prevention of diseases such as, for example, central nervous system disorders such as Alzheimers disease and Downs Syndrome.

Thus, this invention also provides methods for: (1) method for modulating (including inhibiting, antagonizing and the like) gamma-secretase; (2) treating one or more neurodegenerative diseases; (3) inhibiting the deposition of amyloid protein (e.g., amyloid beta protein) in, on or around neurological tissue (e.g., the brain); (4) Alzheimer's disease; and (5) treating Downs syndrome; wherein each method comprises administering an effective amount of one or more (e.g., one) compounds of formula (I) to a patient in need of such treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula (I) and the administration of an effective amount of one or more (e.g., one) other pharmaceutical active ingredients (e.g., drugs).

This invention also provides methods for: (1) treating mild cognitive impairment; (2) treating glaucoma; (3) treating cerebral amyloid angiopathy; (4) treating stroke; (5) treating dementia; (6) treating microgliosis; (7) treating brain inflammation; and (8) treating olfactory function loss; wherein each method comprises administering an effective amount of one or more (e.g., one) compounds of formula (I) to a patient in need of such treatment.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described below), the combined quantities of the compound of formula (I) and the other pharmaceutically active ingredient being effective to treat the diseases or conditions mentioned in any of the above methods.

This invention also provides any of the above mentioned methods, pharmaceutical compositions or kit wherein the compound of formula (I) is selected from the group consisting of: (ID) to (IG), (IM) to (IQ), 1 to 32, 1A to 32A, 1C to 32C, 1E to 32E, B1 to B3, B6, B9, B10, A1 to A6, A10, A12 to A107, B4, B5, B7, and B8.

This invention also provides any of the above mentioned methods, pharmaceutical compositions or kit wherein the compound of formula (I) is selected from the group consisting of: compounds A1 to A6, A10, A12 to A107, B4, B5, B7, and B8.

This invention also provides any of the above mentioned methods, pharmaceutical compositions or kit wherein a compound selected from the group consisting of A7, A8, A9 and A11 is used instead of a compound of formula (I).

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) and the administration of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form. The combination therapies are also directed to methods comprising the administration of one or more (e.g. one) compounds selected from the group consisting of: A7, A8, A9 and A11, and the administration of one or more (e.g., one) other pharmaceutical active ingredients (e.g., drugs). The compounds selected from the group consisting of: A7, A8, A9 and A11, and the other drugs can be administered separately (i.e., each is in its own separate dosage form), or the compounds selected from the group consisting of: A7, A8, A9 and A11, can be combined with the other drugs in the same dosage form.

### DETAILED DESCRIPTION OF THE INVENTION

This invention provides compounds, useful as gamma secretase modulators, of formula (I):

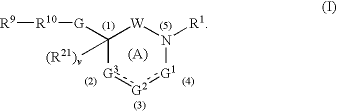

or a pharmaceutically acceptable salt, ester, or solvate thereof, wherein:

the numbers (1), (2), (3), (4), and (5) are reference numbers to identify positions of the Ring (A); G³ is at position (2), G² is at position (3), G¹ is at position (4) and the N is at position (5);

R¹, R⁹, R¹⁰, R²¹, v, G¹, G², G³, and W are each independently selected;

the dotted line ( [symbol] ) represents an optional bond between positions (2) and (3) or positions (3) and (4), that is when the optional bond is present between positions (2) and (3) the optional bond is absent between positions (3) and (4), and when the optional bond is present between positions (3) and (4) the optional bond is absent between positions (2) and (3);

d is 0 or 1 (and those skilled in the art will appreciate that when d is 0 in the —N(R²)_{d}— moiety there is no substituent on the N, thus, the moiety —N(R²)_{d}— is —N═ or —NH— when d is 0, i.e., when d is 0 in a moiety there is the appropriate number of H atoms on the N to fill the required valences);

m is 0 to 6;

n is 1 to 5;

p is 0 to 5;

q is 0, 1 or 2, and each q is independently selected (and those skilled in the art will appreciate that when q is 0 in the moiety —C(R²¹)_{q} this means that there is no R²¹ substituent on the carbon, and the —C(R²¹)_{q} moiety is —CH═ or —CH₂—, i.e., when q is 0 in a moiety there is the appropriate number of H atoms on the carbon to fill the required valences);

r is 1 to 3;

t is 1 or 2

v (for said R²¹ group at position (1)) is 0 or 1, and those skilled in the art will appreciate that when v is 0 there is no R²¹ substituent on the carbon and there is a H to fill the required valence, in one example, v is 0 and there is a H bound to the carbon at position (1), as well as moiety G;

W is selected from the group consisting of: —C(O)—, —S(O)₂—, —S(O)—, and —C(═NR²)—;

G is selected from the group consisting of: a direct bond (i.e., R¹⁰ is bound directly to either G³ or G⁴), —C(O)—, —(C═NR²)—, —(C═C(R⁶)₂)—, —CHR³— (e.g., —CHOH), —C(R⁴)₂—, —CF₂—, —N(R²)— (and in one example, —NH—), —O—, —S—, —S(O)ₜ, —CR⁴(OH)—, —CR⁴(OR⁴)—, —C═C—, alkynyl, —(CH₂)ᵣN(R²)—, —(CHR⁴)ᵣN(R²)—, —(C(R⁴)₂)ᵣN(R²)—, —N(R²)(CH₂)ᵣ—, —N(R²)(CHR⁴)ᵣ—, —N(R²)(C(R⁴)₂)ᵣ—, —(CH₂)ᵣ—O—, —(CHR⁴)ᵣ—O—, —(C(R⁴)₂)ᵣ—O—, —O—(CH₂)ᵣ—, —O—(CHR⁴)ᵣ—, —O—(C(R⁴)₂)ᵣ—, —(CH₂)ᵣ—O—C(O)—, —(CHR⁴)ᵣ—O—C(O)—, —(C(R⁴)₂)ᵣ—O—C(O)—, —C(O)—O—(CH₂)ᵣ—, —C(O)—O—(CHR⁴)ᵣ—, —C(O)—O—(C(R⁴)₂)ᵣ—, —C(O)NR⁵—, —O—C(O)—, —C(O)—O—, —O—C(O)—NR⁵—, —NR⁵C(O)—, —(CH₂)ᵣNR⁵—C(O)—, —(CHR⁴)ᵣNR⁵—C(O)—, —(C(R⁴)₂)ᵣNR⁵—C(O)—, —C(O)NR⁵(CH₂)ᵣ—, —C(O)NR⁵(CHR⁴)ᵣ—, —C(O)NR⁵(C(R⁴)₂)ᵣ—, —NR⁵S(O)ₜ—, —(CH₂)ᵣNR⁵S(O)ₜ—, —(CHR⁴)ᵣNR⁵S(O)ₜ—, —(C(R⁴)₂)ᵣNR⁵S(O)ₜ—, —S(O)ₜNR⁵—, —S(O)ₜNR⁵(CH₂)ᵣ—, —S(O)ₜNR⁵(CHR⁴)ᵣ—, —S(O)ₜNR⁵(C(R⁴)₂)ᵣ—, —NR⁵—C(O)—O—, —NR⁵—C(O)—NR⁵—, —NR⁵—S(O)ₜ—NR⁵—, —NR⁵—C(═NR²)—NR⁵—, —NR⁵—C(═NR²)—O—, —O—C(═NR²)—NR⁵—, —C(R⁴)═N—O—, —O—N═C(R⁴)—, —O—C(R⁴)═N—, —N═C(R⁴)—O—, —(CH₂)₂₋₃— (i.e., 2 to 3 —CH₂— groups), —(C(R⁴)₂)₂₋₃— (i.e., there are 2 to 3 —(C(R⁴)₂ groups), —(CHR⁴)₂₋₃— (i.e., there are 2 to 3 —(CHR⁴)— groups), cycloalkyl (e.g., C₃ to C₁₀ cycloalkyl), and heterocycloalkyl (comprising 1 to 4 heteroatoms independently selected from the group consisting of: —O—, —NR²—, —S—, —S(O)—, and —S(O)₂);

G¹ is selected from the group consisting of: a direct bond (i.e., the N at (5) is bonded directly to G², and Ring A is a five membered ring), —O—, —C(R²¹)_{q}—, —N(R²)_{d}—, —C(O)—, —C(═NR²)—, —S—, —S(O)₂, and —S(O)—; and with the proviso that when the optional double bond between (3) and (4) is present then:
(a) q for the —C(R²¹)_{q}— group is 0 or 1 (and when 0 there is a H on the carbon), and
(b) d for the —N(R²)_{d}— group is 0 (and there is no H on the N due to the double bond between positions (3) and (4)); and
(c) G¹ is not —O—, —C(O)—, —C(═NR²)—, —S—, —S(O)₂, or S(O)—;

G² is selected from the group consisting of: a direct bond (i.e., G¹ is bonded directly to G³, and Ring A is a five membered ring), —O—, —C(R²¹)_{q}—, —N(R²)_{d}—, —C(O)—, —C(═NR²)—, —S—, —S(O)₂, and —S(O)—; and with the proviso that when the optional double bond between (3) and (4) is present then:
(a) q for the —C(R²¹)_{q}— group is 0 or 1 (and when 0 there is a H on the carbon), and
(b) d for the —N(R²)_{d}— group is 0 (and there is no H on the N due to the double bond between positions (3) and (4)); and
(c) G² is not —O—, —C(O)—, —C(═NR²)—, —S—, —S(O)₂, or —S(O)—;

G³ is selected from the group consisting of: —C(R²¹)_{q}— wherein q is 0, 1 or 2, and when the optional bond between G² and G³ is present then q is 0 or 1 (and when q is 0 there is a H on the carbon), and —N(R²)_{d} wherein d is 0 or 1, and d is 0 when the optional bond between G² and G³ is present;

Optionally,
(a) G¹ and G² can be taken together to form a ring, wherein said ring is a 3 to 8 membered (including the atoms common to both rings) cycloalkyl, heterocycloalkyl, heteroaryl, aryl, cycloalkenyl, or heterocycloalkenyl ring (and in one example the ring is a 5 to 6 membered ring), and wherein said ring is optionally substituted with 1 to 5 independently selected R²¹ substituents, and wherein said heterocycloalkyl, heteroaryl, and heterocycloalkenyl rings comprise 1 to 3 heteroatoms independently selected from the group consisting of: —O—, —S—, —S(O)—, —S(O)₂—, and —N(R²)—, or
(b) G² and G³ can be taken together to form a ring, wherein said ring is a 3 to 8 membered (including the atoms common to both rings) cycloalkyl, heterocycloalkyl, heteroaryl, aryl, cycloalkenyl, or heterocycloalkenyl ring (and in one example the ring is a 5 to 6 membered ring), and wherein said ring is optionally substituted with 1 to 5 independently selected R²¹ substituents, and wherein said heterocycloalkyl, heteroaryl, and heterocycloalkenyl rings comprise 1 to 3 heteroatoms independently selected from the group consisting of: —O—, —S—, —S(O)—, —S(O)₂—, and —N(R²)—, or
(c) G and the Ring (A) carbon to which G is bound can be taken together to form a spiro ring (and in one example the ring is a 3 to 5 membered ring including the atoms common to both rings, and in another example the ring is a 3 membered ring including the atoms common to both rings), wherein said ring is a 3 to 8 membered (including the atom common to both rings) cycloalkyl, heterocycloalkyl, cycloalkenyl, or heterocycloalkenyl ring (and in one example the ring is a 3 to 4 membered ring), and wherein said ring is optionally substituted with 1 to 5 independently selected R²¹ substituents, and wherein said heterocycloalkyl, and heterocycloalkenyl rings comprise 1 to 3 heteroatoms independently selected from the group consisting of: —O—, —S—, —S(O)—, —S(O)₂—, and —N(R²)—, or
(d) G and (R²¹), can be taken together to form a spiro ring wherein said ring is a 3 to 8 membered (including the atom common to both rings) cycloalkyl, heterocycloalkyl, cycloalkenyl, or heterocycloalkenyl ring (and in one example the ring is a 3 to 5 membered ring, and in another example the ring is a 3 to 4 membered ring, and in another example the ring is a five membered ring), and wherein said ring is optionally substituted with 1 to 5 independently selected R²¹ substituents, and wherein said heterocycloalkyl, and heterocycloalkenyl rings comprise 1 to 3 heteroatoms independently selected from the group consisting of: —O—, —S—, —S(O)—, —S(O)₂—, and —N(R²)—; and
   when:
(a) G¹ and G² form a ring then:
   (1) G¹ is selected from the group consisting of: (i) C (i.e., G¹ is the moiety —C(R²¹)_{q}— wherein q is 0) and the optional bond between G¹ and G² is present, (ii) —C(R²¹)_{q}— wherein q is 1 and the optional bond between G¹ and G² is absent, (iii) —CH— and the optional bond between G¹ and G² is absent, (iv) N (i.e., G¹ is the moiety —N(R²)_{d}— wherein d is 0) and the optional bond between G¹ and G² is absent, and (v) —C(═N) and the optional bond between G¹ and G² is absent (and those skilled in the art will appreciate that the N of the —C(═N) group is an atom in the ring formed by G¹ and G²); and wherein in one example, G¹ is —C(R²¹)_{q}—, and
   (2) G² is selected from the group consisting of: (i) C (i.e., G² is the moiety —C(R²¹)_{q}— wherein q is 0) and the optional bond between G¹ and G² is present, (ii) C (i.e., G² is the moiety —C(R²¹)_{q}— wherein q is 0) and the optional bond between G² and G³ is present, (iii) —C(R²¹)_{q}— wherein q is 1 and the optional bond between G¹ and G² is absent, and the optional bond between G² and G³ is absent, (iii) —CH— and the optional bond between G¹ and G² is absent, and the optional bond between G² and G³ is absent, and (iv) N (i.e., G² is the moiety —N(R²)_{d}— wherein d is 0) and the optional bond between G¹ and G² is absent, and the optional bond between G² and G³ is absent; and wherein in one example, G² is —C(R²¹)_{q}—;
(b) G² and G³ form a ring then:
   (1) G² is selected from the group consisting of: (i) C (i.e., G² is the moiety —C(R²¹)_{q}— wherein q is 0) and the optional bond between G¹ and G² is present, (ii) C (i.e., G² is the moiety —C(R²¹)_{q}— wherein q is 0) and the optional bond between G² and G³ is present, (iii) —C(R²¹)_{q}— wherein q is 1 and the optional bond between G¹ and G² is absent, and the optional bond between G² and G³ is absent, (iii) —CH— and the optional bond between G¹ and G² is absent, and the optional bond between G² and G³ is absent, and (iv) N (i.e., G² is the moiety —N(R²)_{d}— wherein d is 0) and the optional bond between G¹ and G² is absent, and the optional bond between G² and G³ is absent; and wherein in one example, G² is —C(R²¹)_{q}—, and
   (2) G³ is selected from the group consisting of: (i) C (i.e., G³ is the moiety —C(R²¹)_{q}— wherein q is 0) and the optional bond between G² and G³ is present, (ii) —C(R²¹)_{q}— wherein q is 1 and the optional bond between G² and G³ is absent, (iii) —CH— and the optional bond between G² and G³ is absent, and (iv) N (i.e., G³ is the moiety —N(R²)_{d}— wherein d is 0) and the optional bond between G² and G³ is absent; and wherein in one example, G³ is C; and
(c) G and the Ring (A) carbon to which G is bound form a spiro ring, then v is 0 for the R²¹ moiety at position 1, and there is no H bound to the carbon at position (1);

R¹ is selected from the group consisting of: alkyl, alkenyl, alkynyl, cycloalkyl, cycloalkylalkyl-, heterocyclyl (e.g., heterocycloalkyl), cycloalkenyl, arylalkyl-, alkylaryl-, aryl (e.g., phenyl), heteroaryl (e.g., pyridyl), heterocyclenyl (i.e., heterocycloalkenyl), fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-), fused benzocycloalkylalkyl- (i.e., benzofusedcycloalkylalkyl-), fused benzoheterocycloalkylalkyl- (i.e., benzofusedheterocycloalkylalkyl-), fused heteroarylcycloalkylalkyl- (i.e., heteroarylfusedcycloalkylalkyl-), fused heteroarylheterocycloalkylalkyl- (i.e., heteroarylfusedheterocycloalkylalkyl-), fused cycloalkylarylalkyl- (i.e., cycloalkyfusedlarylalkyl-), fused heterocycloalkylarylalkyl- (i.e., heterocycloalkylfusedarylalkyl-), fused cycloalkylheteroarylalkyl- (i.e., cycloalkylfusedheteroarylalkyl-), fused heterocycloalkylheteroarylalkyl- (i.e., heterocycloalkylfusedheteroarylalkyl-), and wherein each of said: alkyl, alkenyl, alkynyl, cycloalkyl, heterocyclyl, cycloalkenyl, aryl, heteroaryl, heterocyclenyl, fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R¹ groups is optionally substituted with 1-5 independently selected R²¹ groups; or R¹ taken together with the nitrogen to which it is bound, and taken together with G¹ form a 4 to 8 membered ring fused to Ring (A), wherein said fused ring optionally comprises 1 to 3 additional heteroatoms selected from the group consisting of —NR²—, —O—, —S—, —S(O)—, and —S(O)₂, and wherein said fused ring optionally comprises 1 to 3 double bonds, and wherein said fused ring is optionally, substituted with 1 to 6 independently selected R²¹ groups, and wherein G¹ is selected from the group consisting of: (i) C (i.e., G¹ is the moiety-C(R²¹)_{q}— wherein q is 0) and the optional bond between G¹ and G² is present, (ii) —C(R²¹)_{q}— wherein q is 1 and the optional bond between G¹ and G² is absent, (iii) —CH— and the optional bond between G¹ and G² is absent, (iv) N (i.e., G¹ is the moiety —N(R²)_{d}— wherein d is 0) and the optional bond between G¹ and G² is absent, and (v) —C(═N) and the optional bond between G¹ and G² is absent (and those skilled in the art will appreciate that the N of the —C(═N) group is an atom in the ring formed by G¹ and G²), and wherein in one example, G¹ is —C(R²¹)_{q};

R² is selected from the group consisting of: H, —OH, —O-alkyl (i.e., alkoxy), —O-(halo substituted alky) (such as, for example, —O-fluoroalkyl), —NH(R⁴), —N(R⁴)₂, —NH₂, —S(R⁴), —S(O)R⁴, —S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstitued aryl, substitued aryl, unsubstitued heteroaryl, substitued heteroaryl, unsubstituted alkyl, substituted alkyl, unsubstitued arylalkyl-, substitued arylalkyl-, unsubstitued heteroarylalkyl-, substitued heteroarylalkyl-, unsubstitued alkenyl, substituted alkenyl, unsubstituted alkynyl, substituted alkynyl, unsubstitued cycloalkyl, and substituted cycloalkyl, wherein said substitued aryl, heteroaryl, alkyl, arylalkyl-, heteroarylalkyl-, alkenyl, alkynyl and cycloalkyl groups are substituted with 1 to 5 independently selected R²¹ groups;

R³ is selected from the group consisting of: H, —OH, halo, —O-alkyl (i.e., alkoxy), —O-(halo substituted alky) (such as, for example, —O-fluoroalkyl), —NH(R⁴), —N(R⁴)₂, —NH₂, —S(R⁴), —S(O)R⁴, —S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

Each R⁴ is independently selected from the group consisting of: unsubstitued aryl, substitued aryl, unsubstitued heteroaryl, substitued heteroaryl, unsubstituted alkyl, substituted alkyl, unsubstitued arylalkyl-, substitued arylalkyl-, unsubstitued heteroarylalkyl-, substitued heteroarylalkyl-, unsubstitued alkenyl, substituted alkenyl, unsubstituted alkynyl, substituted alkynyl, unsubstitued cycloalkyl, and substituted cycloalkyl, wherein said substitued aryl, heteroaryl, alkyl, arylalkyl-, heteroarylalkyl-, alkenyl, alkynyl and cycloalkyl groups are substituted with 1 to 5 independently selected R²¹ groups;

Each R⁵ is independently selected from the group consisting of: H, unsubstitued alkyl, substituted alkyl, unsubstitued alkenyl, substituted alkenyl, unsubstitued alkynyl, substituted alkynyl, unsubstitued cycloalkyl, substituted cycloalkyl, unsubstituted aryl, substituted aryl, unsubstituted heteroaryl and substituted heteroaryl; wherein said substituted groups are substituted with one or more (e.g., 1 to 5) substituents independently selected from: R²;

each R⁶ is independently selected from the group consisting of aryl, heteroaryl, halo, —CF₃, —CN, —C(O)R²⁴, —C(O)OR²⁴, C(O)N(R²⁴)(R²⁵), —S(O)N(R²⁴)(R²⁵), —OR⁹, —S(O)₂N(R²⁴)(R²⁵), —C(═NOR²⁴)R²⁵, —P(O)(OR²⁴)(OR²⁵), —N(R²⁴)(R²⁵), —N(R²⁴)C(O)R²⁵, —N(R²⁴)S(O)R^{25A}, —N(R²⁴)S(O)₂R^{25A}, —N(R²⁴)S(O)₂N(R²⁵)(R²⁶), —N(R²⁴)S(O)N(R²⁵)(R²⁶), —N(R²⁴)C(O)N(R²⁵)(R²⁶), —N(R²⁴)C(O)OR²⁵, —S(O)R^{24A} and —S(O)₂R^{24A};

R⁹ is selected from the group consisting of: arylalkoxy-, heteroarylalkoxy-, arylalkylamino-, heteroarylalkylamino-, aryl-, arylalkyl-, cycloalkyl-, cycloalkenyl, cycloalkylalkyl-, heteroaryl-, heteroarylalkyl-, heterocyclyl-, heterocyclenyl-, and heterocyclyalkyl-, wherein each of said R⁹ arylalkoxy-, heteroarylalkoxy-, arylalkylamino-, heteroarylalkylamino-, aryl-, arylalkyl-, cycloalkyl-, cycloalkenyl, cycloalkylalkyl-, heteroaryl-, heteroarylalkyl-, heterocyclyl-, heterocyclenyl-, heterocyclyalkyl- and heterocyclyalkyl- is optionally substituted with 1-5 independently selected R²¹ groups;

R¹⁰ is selected from the group consisting of: aryl- (e.g., phenyl), heteroaryl- (e.g., pyridyl), cycloalkyl-, cycloalkenyl, cycloalkylalkyl-, heterocyclyl-, heterocyclenyl-, heterocyclylalkyl-, heterocyclyalkenyl-, fused benzocycloalkyl- (i.e., benzofusedcycloalkyl-), fused benzoheterocycloalkyl- (i.e., benzofusedheterocycloalkyl-), fused heteroarylcycloalkyl- (i.e., heteroarylfusedcycloalkyl-), fused heteroarylheterocycloalkyl- (i.e., heteroarylfusedheterocycloalkyl-), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-), fused heteroarylheteroaryl- (i.e., heteroarylfusedheteroaryl-), fused heteroarylaryl- (i.e., heteroarylfusedaryl-), fused arylheteroaryl- (i.e., arylfusedheteroaryl-), fused arylaryl- (i.e., arylfusedaryl-), fused heterocycloalkenylaryl- (i.e., heterocycloalkenylfusedaryl-), fused heterocycloalkenylheteroaryl- (i.e., heterocycloalkenylfusedheteroaryl-),

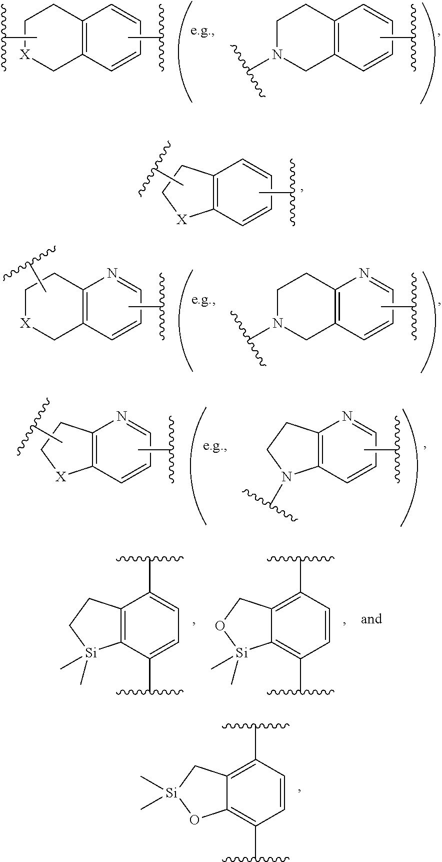

wherein X is selected from the group consisting of: O, —N(R¹⁴)— and —S—; and wherein each of said R¹⁰ moieties is optionally substituted with 1-5 independently selected R²¹ groups; or

R⁹ and R¹⁰ are linked together to form a fused tricyclic ring system wherein R⁹ and R¹⁰ are as defined above and the ring linking R⁹ and R¹⁰ is an alkyl ring, or a heteroalkyl ring, or an aryl ring, or a heteroaryl ring, or an alkenyl ring, or a heteroalkenyl ring (for example, the tricyclic ring system is formed by linking the atoms adjacent to the atoms by which R⁹ and R¹⁰ are bound together);

R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶);

R^{15A} and R^{16A} are independently selected from the group consisting of alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;

Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or

two R¹⁸ moieties on adjacent carbons can be linked together to form a

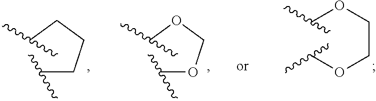

R¹⁹ is selected from the group consisting of: alkyl, cycloalkyl, aryl, arylalkyl and heteroarylalkyl;

R²⁰ is selected from the group consisting of: alkyl, cycloalkyl, aryl, halo substituted aryl, arylalkyl, heteroaryl and heteroarylalkyl;

each R²¹ is independently selected from the group consisting of: alkyl, alkenyl, alkynyl, cycloalkyl, cycloalkylalkyl, cycloalkenyl, heterocycloalkyl, ═O, ═N—R², heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —P(O)(CH₃)₂, —SO(═NR¹⁵)R¹⁶—, —SF₅, —OSF₅, —Si(R^{15A})₃ wherein each R^{15A} is independently selected —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, ═NOR¹⁵, —N₃, —NO₂, —S(O)₂R^{15A}, —O—N═C(R⁴)₂ (wherein each R⁴ is independently selected), and —O—N═C(R⁴)₂ wherein R⁴ is taken together with the carbon atom to which they are bound to form a 5 to 10 membered ring, said ring optionally containing 1 to 3 heteroatoms selected from the group consisting of —O—, —S—, —S(O)—, —S(O)₂—, and —NR²—; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups;

Each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —SF₅, —OSF₅, —Si(R^{15A})₃, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R^{15A} and —S(O)₂R^{15A};

Each R^{24A} and R^{25A} is independently selected from the group consisting of alkyl, cycloalkyl, cycloalkylalkyl, heterocycloalkyl, heterocycloalkylalkyl, aryl, arylalkyl, heteroaryl, heteroarylalkyl, arylcycloalkyl, (R^{27A})ₙ-alkyl, (R^{27A})ₙ-cycloalkyl, (R^{27A})ₙ-cycloalkylalkyl, (R^{27A})ₙ-heterocycloalkyl, (R^{27A})ₙ-heterocycloalkylalkyl, (R^{27A})ₙ-aryl, (R^{27A})ₙ-arylalkyl, (R^{27A})ₙ-heteroaryl and (R^{27A})ₙ-heteroarylalkyl;

Each R²⁴, R²⁵ and R²⁶ is independently selected from the group consisting of H, alkyl, cycloalkyl, cycloalkylalkyl, heterocycloalkyl, heterocycloalkylalkyl, aryl, arylalkyl, heteroaryl, heteroarylalkyl, arylcycloalkyl, (R^{27A})ₙ-alkyl, (R^{27A})ₙ-cycloalkyl, (R^{27A})ₙ-cycloalkylalkyl, (R^{27A})ₙ-heterocycloalkyl, (R^{27A})ₙ-heterocycloalkylalkyl, (R^{27A})ₙ-aryl, (R^{27A})ₙ-arylalkyl, (R^{27A})ₙ-heteroaryl and (R^{27A})ₙ-heteroarylalkyl;

Each R^{27A} is independently selected from the group consisting of alkyl, aryl, arylalkyl, —NO₂, halo, —CF₃, —CN, alkyl-CN, —C(O)R²⁸, —C(O)OH, —C(O)OR²⁸, —C(O)NHR²⁹, —C(O)N(alkyl)₂, —C(O)N(alkyl)(aryl), —C(O)N(alkyl)(heteroaryl), —SR²⁸, —S(O)₂R²⁹, —S(O)NH₂, —S(O)NH(alkyl), —S(O)N(alkyl)(alkyl), —S(O)NH(aryl), —S(O)₂NH₂, —S(O)₂NHR²⁸, —S(O)₂NH(aryl), —S(O)₂NH(heterocycloalkyl), —S(O)₂N(alkyl)₂, —S(O)₂N(alkyl)(aryl), —OH, —OR²⁹, —O-heterocycloalkyl, —O-cycloalkylalkyl, —O-heterocycloalkylalkyl, —NH₂, —NHR²⁹, —N(alkyl)₂, —N(arylalkyl)₂, —N(arylalkyl)(heteroarylalkyl), —NHC(O)R²⁹, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁹, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

R²⁸ is selected from the group consisting of: alkyl, cycloalkyl, arylalkyl and heteroarylalkyl; and

R²⁹ is selected from the group consisting of; alkyl, cycloalkyl, aryl, arylalkyl, heteroaryl or heteroarylalkyl; and

provided that:
(a) Ring A does not have two adjacent —O— atoms in the ring; and
(b) Ring A does not have two adjacent sulfur groups in the ring (i.e., when there is a —S—, —S(O)— or —S(O)₂ group at one position in Ring A, then the adjacent positions in Ring A are not —S—, —S(O)— or —S(O)₂); and
(c) Ring A does not have an —O— atom adjacent to a sulfur group (i.e., Ring A does not have an —O— atom adjacent to a —S—, —S(O)— or —S(O)₂); and
(d) When G¹ is N, then G² is not —O—; and
(e) When G¹ is —O—, then G² is not N; and
(f) When G¹ is N, then G² is not —S—; and
(g) When G¹ is —S—, then G² is not N; and
(h) When G¹ is a direct bond, and G² is —O—, then G³ is not N; and
(i) When G² is a direct bond, and G¹ is —O—, then G³ is not N; and
(j) When G¹ is N, and G³ is N, then G² is not N; and
(k) When G² is N, and G³ is N, then G¹ is not N; and
(l) When G¹ is N, and G² is N, then G³ is not N; and
(m) When W is SO or S(O)₂ then G is not —C(O)—, —(C═NR²)—, —(C═C(R⁶)₂)—, —C(R⁴)₂—, —CF₂—, —CR⁴(OH)—, —CR⁴(OR⁴)—, or —CHR³—; and
(n) When W is —C(O)— then R¹ is not a fused benzocycloalkyl substituted with —NH₂, or a fused benzoheterocycloalkyl substituted with —NH₂, or a fused heteroarylcycloalkyl substituted with —NH₂, or a fused heteroarylheterocycloalkyl substituted with —NH₂; and
(o) When the optional bond between G² and G³ is present (i.e., when the optional bond between position (2) and (3) is present), then v is 1 for the moiety (R²¹)ᵥ (i.e., there is no hydrogen bound to the carbon at position (1)); and
(p) When G is —C(O)—, —(C═NR²)—, —(C═C(R⁶)₂)—, or —C═C—, then v is 1 for the moiety (R²¹)ᵥ (i.e., there is no hydrogen bound to the carbon at position (1)); and
(q) When G¹ is —C(═NR²)—, and G² is a direct bond, and G³ is —N(R²)_{d}—, then G is not —C(O)—, —(C═NR²)—, —(C═C(R⁶)₂)—, —CHR³—, —C(R⁴)₂—, —CR⁴(OH)—, or —CR⁴(OR⁴)—; and
(r) When G² is —C(═NR²)—, and G¹ is direct bond, and G³ is —N(R²)_{d}—, then G is not —C(O)—, —(C═NR²)—, —(C═C(R⁶)₂)—, —CHR³—, —C(R⁴)₂—, —CF₂—, —CR⁴(OH)—, or —CR⁴(OR⁴)—; and
(s) When G¹ is a direct bond, and G² is —C(R²¹)_{q}—, and G³ is —N(R²)_{d}—, and the optional bond between G² and G³ is present, then G is not —C(O)—, —(C═NR²)—, —(C═C(R⁶)₂)—, —CHR³—, —C(R⁴)₂—, —CF₂—, —CR⁴(OH)—, or —CR⁴(OR⁴)—.

The compounds of this invention are useful for treating central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

Thus, for example, the compounds of this invention can be used to treat the following diseases or conditions: Alzheimers disease, mild cognitive impairment (MCI), Downs Syndrome, Glaucoma (Guo et. al., Proc. Natl. Acad. Sci. USA 104, 13444-13449 (2007)), Cerebral amyloid angiopathy, stroke or dementia (Frangione et al., Amyloid: J. Protein folding Disord. 8, suppl. 1, 36-42 (2001), Microgliosis and brain inflammation (M P Lamber, Proc. Natl. Acad. Sci. USA 95, 6448-53 (1998)), and Olfactory function loss (Getchell, et. al. Neurobiology of Aging, 663-673, 24, 2003).

In one embodiment of this invention the compounds are of the formula:

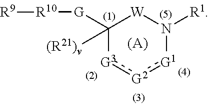

In another embodiment of this invention the compounds are of the formula:

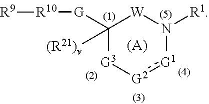

In another embodiment of this invention the compounds are of the formula:

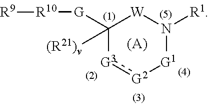

In another embodiment of this invention the compounds are of the formula:

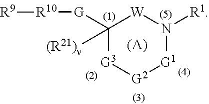

In one embodiment of this invention R¹ is selected from the group consisting of: alkyl, alkenyl, alkynyl, cycloalkyl, cycloalkylalkyl-, heterocyclyl (e.g., heterocycloalkyl), cycloalkenyl, arylalkyl-, alkylaryl-, aryl (e.g., phenyl), heteroaryl (e.g., pyridyl), heterocyclenyl (i.e., heterocycloalkenyl), fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-), fused benzocycloalkylalkyl- (i.e., benzofusedcycloalkylalkyl-), fused benzoheterocycloalkylalkyl- (i.e., benzofusedheterocycloalkylalkyl-), fused heteroarylcycloalkylalkyl- (i.e., heteroarylfusedcycloalkylalkyl-), fused heteroarylheterocycloalkylalkyl- (i.e., heteroarylfusedheterocycloalkylalkyl-), fused cycloalkylarylalkyl- (i.e., cycloalkyfusedlarylalkyl-), fused heterocycloalkylarylalkyl- (i.e., heterocycloalkylfusedarylalkyl-), fused cycloalkylheteroarylalkyl- (i.e., cycloalkylfusedheteroarylalkyl-), fused heterocycloalkylheteroarylalkyl- (i.e., heterocycloalkylfusedheteroarylalkyl-), and wherein each of said: alkyl, alkenyl, alkynyl, cycloalkyl, heterocyclyl, cycloalkenyl, aryl, heteroaryl, heterocyclenyl, fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R¹ groups is optionally substituted with 1-5 independently selected R²¹ groups, provided that provided that no R²¹ group is —NH₂; or R¹ taken together with the nitrogen to which it is bound, and taken together with G¹ form a 4 to 8 membered ring fused to Ring (A), wherein said fused ring optionally comprises 1 to 3 additional heteroatoms selected from the group consisting of —NR²—, —O—, —S—, —S(O)—, and —S(O)₂, and wherein said fused ring optionally comprises 1 to 3 double bonds, and wherein said fused ring is optionally substituted with 1 to 6 independently selected R²¹ groups, and wherein G¹ is selected from the group consisting of: (i) C (i.e., G¹ is the moiety-C(R²¹)_{q}— wherein q is 0) and the optional bond between G¹ and G² is present, (ii) —C(R²¹)_{q}— wherein q is 1 and the optional bond between G¹ and G² is absent, (iii) —CH— and the optional bond between G¹ and G² is absent, (iv) N (i.e., G¹ is the moiety —N(R²)_{d}— wherein d is 0) and the optional bond between G¹ and G² is absent, and (v) —C(═N) and the optional bond between G¹ and G² is absent (and those skilled in the art will appreciate that the N of the —C(═N) group is an atom in the ring formed by G¹ and G²), and wherein in one example, G¹ is —C(R²¹)_{q}.

In one embodiment of this invention R¹ is selected from the group consisting of: alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedheterocycloalkyl), fused heteroarylcycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, -and heterocyclyalkyl-; wherein each of said alkyl-, alkenyl-, alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl and heterocyclyalkyl-R¹ groups is optionally substituted with 1-5 independently selected R²¹ groups.

In another embodiment of this invention is selected from the group consisting of: alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, -and heterocyclyalkyl-; wherein: (a) each of said alkyl-, alkenyl-alkynyl-, aryl-, arylalkyl-, alkylaryl-, cycloalkyl-, cycloalkenyl-, cycloalkylalkyl-, heteroaryl-, heteroarylalkyl-, heterocyclyl-, heterocyclenyl and heterocyclyalkyl-R¹ groups is optionally substituted with 1-5 independently selected R²¹ groups; and (b) each of said fused benzocycloalkyl, fused benzoheterocycloalkyl, fused heteroarylcycloalkyl, and fused heteroarylheterocycloalkyl, R¹ groups is optionally substituted with 1-5 independently selected R²¹ groups, provided that no R²¹ group is an —NH₂ group.

In another embodiment of this invention, R¹ is selected from the group consisting of: alkyl-, alkenyl-, alkynyl-, aryl-, arylalkyl-, alkylaryl-, cycloalkyl-, cycloalkenyl, cycloalkylalkyl-, heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, heteroaryl-, heteroarylalkyl-, heterocyclyl-, heterocyclenyl and heterocyclyalkyl-R¹ groups is optionally substituted with 1-5 independently selected R²¹ groups.

In another embodiment of this invention, R¹ is selected from the group consisting of: fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), and fused heteroarylheterocycloalkyl (i.e., heteroarylfusedheterocycloalkyl); wherein each of said fused benzocycloalkyl, fused benzoheterocycloalkyl, fused heteroarylcycloalkyl, and fused heteroarylheterocycloalkyl R¹ groups is optionally substituted with 1-5 independently selected R²¹ groups, provided that no R²¹ group is —NH₂.

In another embodiment of this invention, R¹ is selected from the group consisting of: fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-), fused benzocycloalkylalkyl- (i.e., benzofusedcycloalkylalkyl-), fused benzoheterocycloalkylalkyl- (i.e., benzofusedheterocycloalkylalkyl-), fused heteroarylcycloalkylalkyl- (i.e., heteroarylfusedcycloalkylalkyl-), fused heteroarylheterocycloalkylalkyl- (i.e., heteroarylfusedheterocycloalkylalkyl-), fused cycloalkylarylalkyl- (i.e., cycloalkyfusedlarylalkyl-), fused heterocycloalkylarylalkyl- (i.e., heterocycloalkylfusedarylalkyl-), fused cycloalkylheteroarylalkyl- (i.e., cycloalkylfusedheteroarylalkyl-), and fused heterocycloalkylheteroarylalkyl- (i.e., heterocycloalkylfusedheteroarylalkyl-).

In another embodiment of this invention, R¹ is selected from the group consisting of: fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-), fused benzocycloalkylalkyl- (i.e., benzofusedcycloalkylalkyl-), fused benzoheterocycloalkylalkyl- (i.e., benzofusedheterocycloalkylalkyl-), fused heteroarylcycloalkylalkyl- (i.e., heteroarylfusedcycloalkylalkyl-), fused heteroarylheterocycloalkylalkyl- (i.e., heteroarylfusedheterocycloalkylalkyl-), fused cycloalkylarylalkyl- (i.e., cycloalkyfusedlarylalkyl-), fused heterocycloalkylarylalkyl- (i.e., heterocycloalkylfusedarylalkyl-), fused cycloalkylheteroarylalkyl- (i.e., cycloalkylfusedheteroarylalkyl-), and fused heterocycloalkylheteroarylalkyl- (i.e., heterocycloalkylfusedheteroarylalkyl-), and wherein each of said: fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R¹ groups is optionally substituted with 1-5 independently selected R²¹ groups, provided that no R²¹ group is —NH₂.

In another embodiment of this invention, R¹ is taken together with the nitrogen to which it is bound, and is taken together with G¹ to form a 4 to 8 membered ring fused to Ring (A), wherein said fused ring optionally comprises 1 to 3 additional heteroatoms selected from the group consisting of —NR²—, —O—, —S—, —S(O)—, and —S(O)₂, and wherein said fused ring optionally comprises 1 to 3 double bonds, and wherein said fused ring is optionally substituted with 1 to 6 independently selected R²¹ groups, and wherein G¹ is selected from the group consisting of: (i) C (i.e., G¹ is the Moiety-C(R²¹)_{q}— wherein q is 0) and the optional bond between G¹ and G² is present, (ii) —C(R²¹)_{q}— wherein q is 1 and the optional bond between G¹ and G² is absent, (iii) —CH— and the optional bond between G¹ and G² is absent, (iv) N (i.e., G¹ is the moiety —N(R²)_{d}— wherein d is 0) and the optional bond between G¹ and G² is absent, and (v) —C(═N) and the optional bond between G¹ and G² is absent (and those skilled in the art will appreciate that the N of the —C(═N) group is an atom in the ring formed by G¹ and G²), and wherein in one example, G¹ is —C(R²¹)_{q}.

In another embodiment of this invention, there are 1 to 5 R²¹ groups present in formula (I), and at least one (e.g., 1 to 2) R²¹ is selected from the group consisting of: —SF₅, —OSF₅ and —Si(R^{15A})₃, wherein each R^{15A} is independently selected.

In another embodiment of this invention, there are 1 to 5 R²¹ groups present in formula (I), and at least one R²¹ is selected from the group consisting of: —SF₅ and —Si(R^{15A})₃, and each R^{15A} is the same or different alkyl group.

In another embodiment of this invention, there are 1 to 5 R²¹ groups present in formula (I), and at least one R²¹ is selected from the group consisting of: —SF₅, —OSF₅ and —Si(CH₃)₃.

In another embodiment of this invention, there are 1 to 5 R²¹ groups present in formula (I), and one of the R²¹ groups is selected from the group consisting of: —SF₅, OSF₅ and —Si(R^{15A})₃.

In another embodiment of this invention, there are 1 to 5 R²¹ groups present in formula (I), and one of the R²¹ groups is selected from the group consisting of: —SF₅, OSF₅ and —Si(R^{15A})₃, and each R^{15A} is the same or different alkyl group.

In another embodiment of this invention, there are 1 to 5 R²¹ groups present in formula (I), and one of the R²¹ groups is selected from the group consisting of: —SF₅, —OSF₅ and —Si(CH₃)₃.

In another embodiment of this invention, there are 2 to 5 R²¹ groups present in formula (I), and two of the R²¹ groups are selected from the group consisting of: —SF₅, OSF₅ and —Si(R^{15A})₃, wherein each R^{15A} is independently selected.

In another embodiment of this invention, there are 2 to 5 R²¹ groups present in formula (I), and two of the R²¹ groups are selected from the group consisting of: —SF₅, OSF₅ and —Si(R^{15A})₃, and each R^{15A} is the same or different alkyl group.

In another embodiment of this invention, there are 2 to 5 R²¹ groups present in formula (I), and two of the R²¹ groups are selected from the group consisting of: —SF₅, —OSF₅ and —Si(CH₃)₃.

In another embodiment of this invention, there are 1 to 5 R²¹ groups present in formula (I), and at least one (e.g., 1 to 2) R²¹ is selected from the group consisting of: —SF₅ and —Si(R^{15A})₃, wherein each R^{15A} is independently selected.

In another embodiment of this invention, there are 1 to 5 R²¹ groups present in formula (I), and at least one R²¹ is selected from the group consisting of: —SF₅ and —Si(R^{15A})₃, and each R^{15A} is the same or different alkyl group.

In another embodiment of this invention, there are 1 to 5 R²¹ groups present in formula (I), and at least one R²¹ is selected from the group consisting of: —SF₅ and —Si(CH₃)₃.

In another embodiment of this invention, there are 1 to 5 R²¹ groups present in formula (I), and one of the R²¹ groups is selected from the group consisting of: —SF₅ and —Si(R^{15A})₃.

In another embodiment of this invention, there are 1 to 5 R²¹ groups present in formula (I), and one of the R²¹ groups is selected from the group consisting of: —SF₅ and —Si(R^{15A})₃, and each R^{15A} is the same or different alkyl group.

In another embodiment of this invention, there are 1 to 5 R²¹ groups present in formula (I), and one of the R²¹ groups is selected from the group consisting of: —SF₅ and —Si(CH₃)₃.

In another embodiment of this invention, there are 2 to 5 R²¹ groups present in formula (I), and two of the R²¹ groups are selected from the group consisting of: —SF₅ and —Si(R^{15A})₃, wherein each R^{15A} is independently selected.

In another embodiment of this invention, there are 2 to 5 R²¹ groups present in formula (I), and two of the R²¹ groups are selected from the group consisting of: —SF₅ and —Si(R^{15A})₃, and each R^{15A} is the same or different alkyl group.

In another embodiment of this invention, there are 2 to 5 R²¹ groups present in formula (I), and two of the R²¹ groups are selected from the group consisting of: —SF₅ and —Si(CH₃)₃.

In another embodiment of this invention, there are 1 to 5 R²¹ groups present in formula (I), and one of the R²¹ groups is —SF₅.

In another embodiment of this invention, there are 2 to 5 R²¹ groups present in formula (I), and two of the R²¹ groups are —SF₅.

In another embodiment of this invention, there are 1 to 5 R²¹ groups present in formula (I), and one of the R²¹ groups is —OSF₅.

In another embodiment of this invention, there are 2 to 5 R²¹ groups present in formula (I), and two of the R²¹ groups are —OSF₅.

In another embodiment of this invention, there are 1 to 5 R²¹ groups present in formula (I), and one of the R²¹ groups is —Si(R^{15A})₃.

In another embodiment of this invention, there are 1 to 5 R²¹ groups present in formula (I), and one of the R²¹ groups is —Si(R^{15A})₃ and each R^{15A} is the same or different alkyl group.

In another embodiment of this invention, there are 1 to 5 R²¹ groups present in formula (I), and one of the R²¹ groups is —Si(CH₃)₃.

In another embodiment of this invention, there are 2 to 5 R²¹ groups present in formula (I), and two of the R²¹ groups are the same or different —Si(R^{15A})₃, wherein each R^{15A} is independently selected.

In another embodiment of this invention, there are 2 to 5 R²¹ groups present in formula (I), and two of the R²¹ groups are the same or different —Si(R^{15A})₃ and each R^{15A} is the same or different alkyl group.

In another embodiment of this invention, there are 2 to 5 R²¹ groups present in formula (I), and two of the R²¹ groups are —Si(CH₃)₃.

In another embodiment of this invention R¹ is substituted with R²¹ groups, and at least one (e.g. 1 to 2) of the R²¹ groups is selected from the group consisting of: —SF₅, —OSF₅ and —Si(R^{15A})₃, wherein each R^{15A} is independently selected.

In another embodiment of this invention R¹ is substituted with R²¹ groups, and at least one (e.g. 1 to 2) of the R²¹ groups is selected from the group consisting of: —SF₅, —OSF₅ and —Si(R^{15A})₃, and each R^{15A} is the same or different alkyl group.

In another embodiment of this invention R¹ is substituted with R²¹ groups, and at least one (e.g. 1 to 2) of the R²¹ groups is selected from the group consisting of: —SF₅, —OSF₅ and —Si(CH₃)₃.

In another embodiment of this invention R¹ is substituted with R²¹ groups, and one R²¹ group is selected from the group consisting of: —SF₅, —OSF₅ and —Si(R^{15A})₃, wherein each R^{15A} is independently selected.

In another embodiment of this invention R¹ is substituted with R²¹ groups, and one R²¹ group is selected from the group consisting of: —SF₅, —OSF₅ and —Si(R^{15A})₃, and each R^{15A} is the same or different alkyl group.

In another embodiment of this invention R¹ is substituted with R²¹ groups, and one R²¹ group is selected from the group consisting of: —SF₅, —OSF₅ and —Si(CH₃)₃.

In another embodiment of this invention R¹ is substituted with R²¹ groups, and two R²¹ groups are selected from the group consisting of: —SF₅, —OSF₅ and —Si(R^{15A})₃, wherein each R^{15A} is independently selected.

In another embodiment of this invention R¹ is substituted with R²¹ groups, and two R²¹ groups are selected from the group consisting of: —SF₅, —OSF₅ and —Si(R^{15A})₃, and each R^{15A} is the same or different alkyl group.

In another embodiment of this invention R¹ is substituted with R²¹ groups, and two R²¹ groups are selected from the group consisting of: —SF₅, —OSF₅ and —Si(CH₃)₃.

In another embodiment of this invention R¹ is substituted with R²¹ groups, and one R²¹ group is —SF₅.

In another embodiment of this invention R¹ is substituted with R²¹ groups, and two R²¹ groups are —SF₅.

In another embodiment of this invention R¹ is substituted with R²¹ groups, and one R²¹ group is —OSF₅.

In another embodiment of this invention R¹ is substituted with R²¹ groups, and two R²¹ groups are —OSF₅.

In another embodiment of this invention R¹ is substituted with R²¹ groups, and one R²¹ group is —Si(R^{15A})₃, wherein each R^{15A} is independently selected.

In another embodiment of this invention R¹ is substituted with R²¹ groups, and one R²¹ group is —Si(R^{15A})₃ and each R^{15A} is the same or different alkyl group.

In another embodiment of this invention R¹ is substituted with R²¹ groups, and one R²¹ group is —Si(CH₃)₃.

In another embodiment of this invention R¹ is substituted with R²¹ groups, and two of the R²¹ groups are the same or different —Si(R^{15A})₃, wherein each R^{15A} is independently selected.

In another embodiment of this invention R¹ is substituted with R²¹ groups, and two of the R²¹ groups are the same or different —Si(R^{15A})₃ group, and each R^{15A} is the same or different alkyl group.

In another embodiment of this invention R¹ is substituted with R²¹ groups, and two of the R²¹ group are —Si(CH₃)₃.

In another embodiment of this invention R¹ is an aryl group substituted with R²¹ groups, and at least one (e.g., 1 to 2) R²¹ group is selected from the group consisting of: —SF₅, —OSF₅ and —Si(R^{15A})₃, wherein each R^{15A} is independently selected.

In another embodiment of this invention R¹ is an aryl group group substituted with R²¹ groups, and at least one (e.g., 1 to 2) R²¹ group is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and at least one (e.g., 1 to 2) R²¹ group is selected from the group consisting of: —SF₅, —OSF₅ and —Si(CH₃)₃.

In another embodiment of this invention R¹ is an aryl group substituted with R²¹ groups, and said aryl moiety is phenyl, and at least one (e.g., 1 to 2) R²¹ group is selected from the group consisting of: —SF₅, —OSF₅ and —Si(R^{15A})₃, wherein each R^{15A} is independently selected.

In another embodiment of this invention R¹ is an aryl group substituted with R²¹ groups, and said aryl moiety is phenyl, and at least one (e.g., 1 to 2) R²¹ group is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and said aryl moiety is phenyl, and at least one (e.g., 1 to 2) R²¹ group is selected from the group consisting of: —SF₅, —OSF₅ and —Si(CH₃)₃.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R^{15A})₃, wherein each R^{15A} is independently selected.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(CH₃)₃.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R^{15A})₃, wherein each R^{15A} is independently selected.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(CH₃)₃.

In another embodiment of this invention R¹ is an aryl group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R^{15A})₃, wherein each R^{15A} is independently selected.

In another embodiment of this invention R¹ is an aryl group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(CH₃)₃.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —SF₅.

In another embodiment of this invention R¹ is an aryl group substituted with R²¹ groups, and said aryl moiety is phenyl, and said phenyl is substituted with at least one (e.g., 1 to 3, or 1 to 2)R²¹ group, and one R¹ group on said phenyl is —OSF₅.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —Si(R^{15A})₃, wherein each R^{15A} is independently selected.

In another embodiment of this invention R¹ is an aryl group group substituted with R²¹ groups, and said aryl moiety is phenyl, and said phenyl is substituted with at least one (e.g., 1 to 3, or 1 to 2) R²¹ group, and one R²¹ group on said phenyl is —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least one (e.g., 1 to 3, or 1 to 2) R²¹ group, and one R²¹ group on said phenyl is —Si(CH₃)₃.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —SF₅.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —OSF₅.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —Si(R^{15A})₃, wherein each R^{15A} is independently selected.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —Si(R^{15A})₃, and each R^{15A} is the same or different alkyl group.

In another embodiment of this invention R¹ is an aryl group substituted with R²¹ groups, and said aryl moiety is phenyl, and said phenyl is substituted with at least two (e.g., 2 to 3) R²¹ groups, and two of the R²¹ groups on said phenyl are —Si(CH₃)₃.

Examples of compounds of formula (I) include but are not limited to:

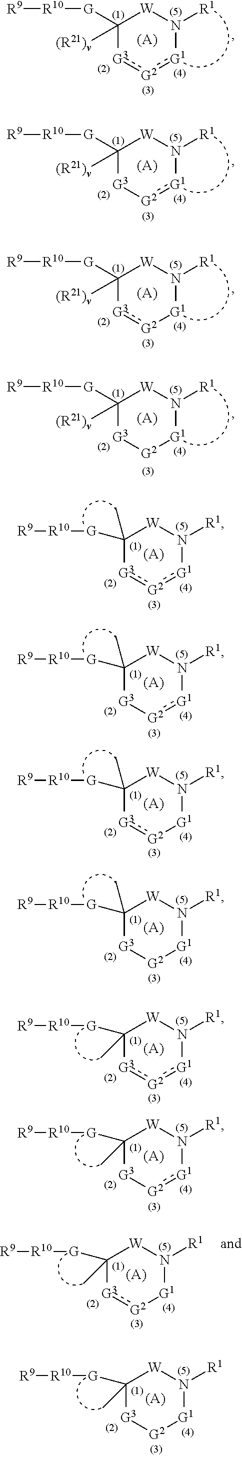

wherein all substituents are as defined for formula (I), and in one example W is —C(O)—.

Examples of compounds of formula (I) include but are not limited to:

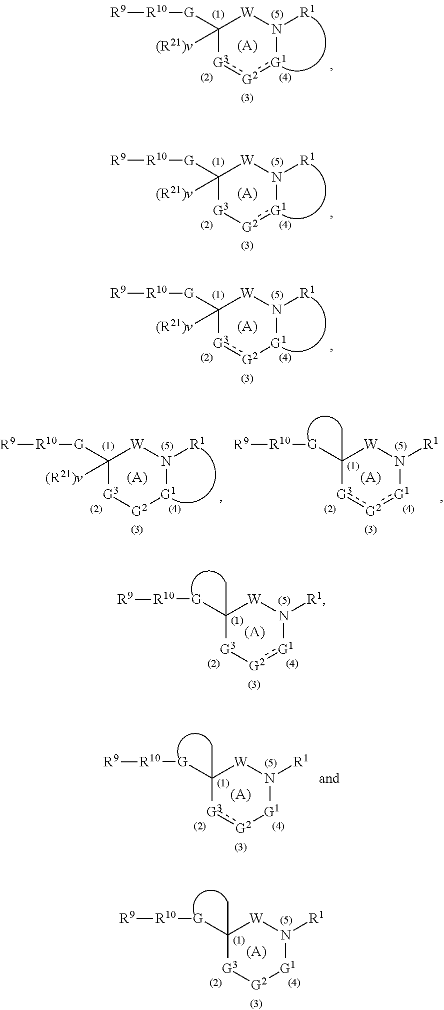

wherein all substituents are as defined for formula (I), and in one example, W is —C(O)—.

Examples of compounds of formula (I) include but are not limited to:

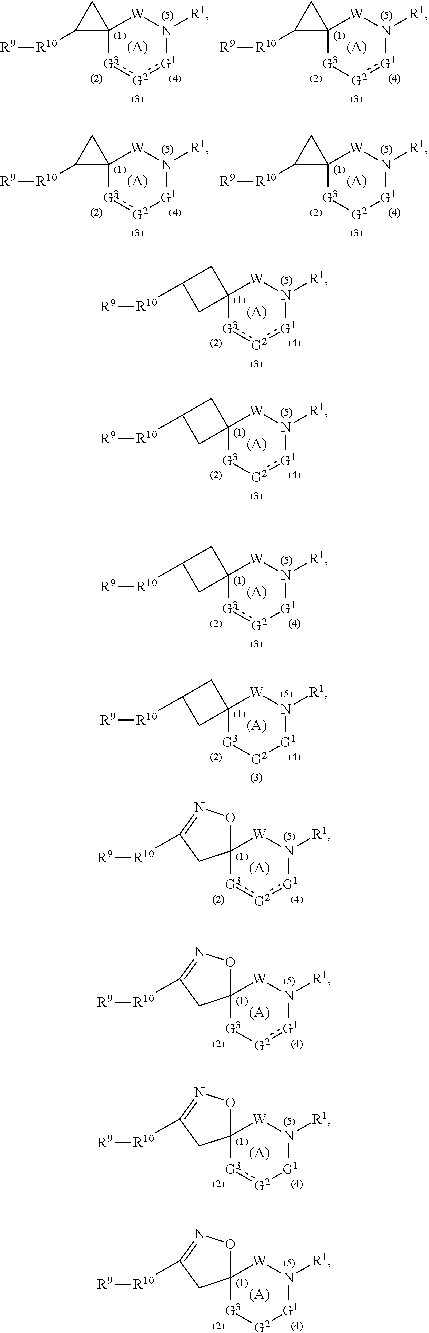

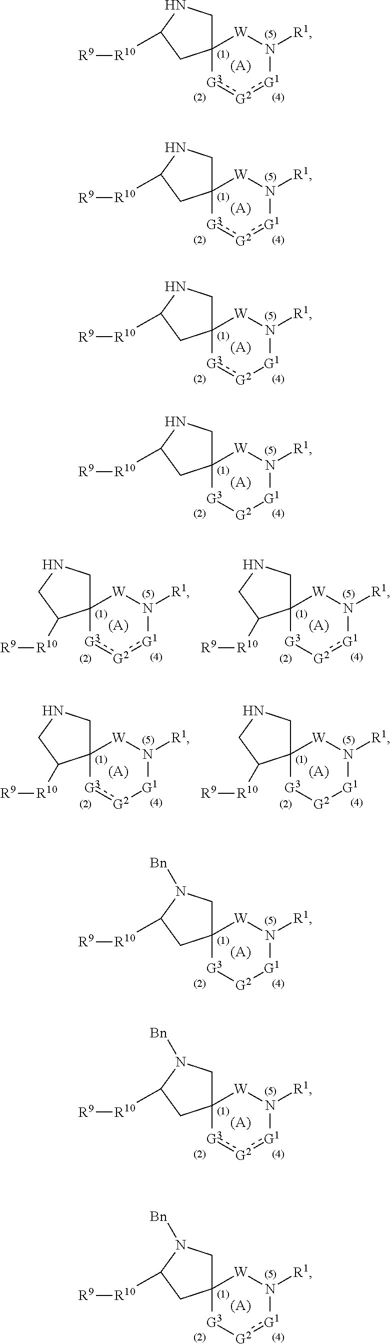

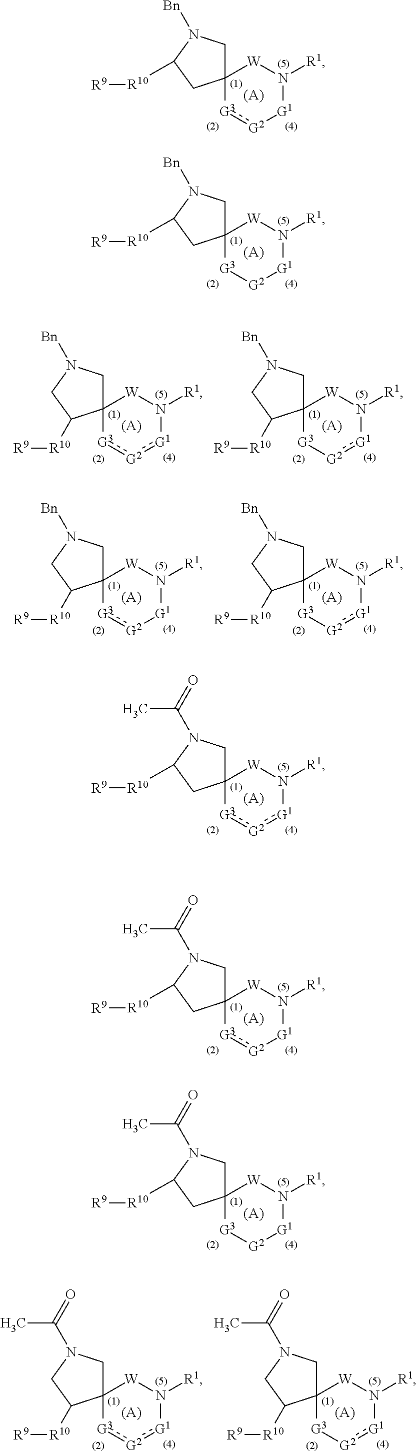

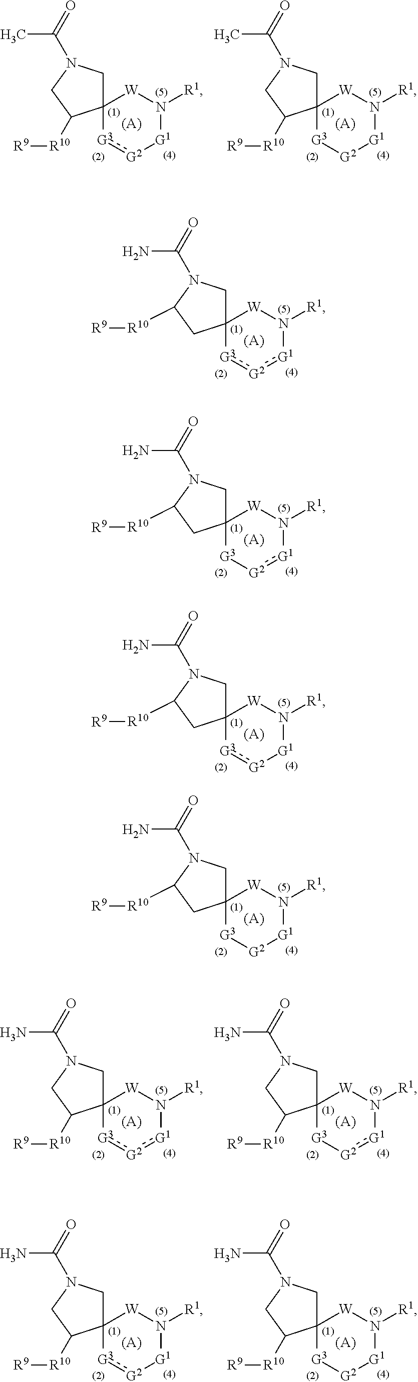

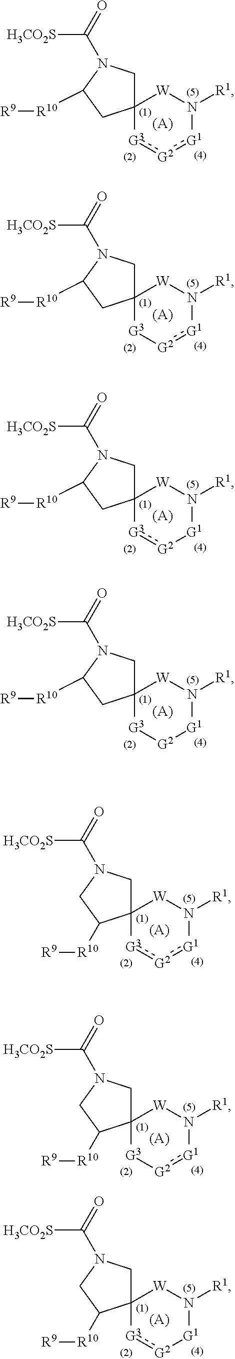

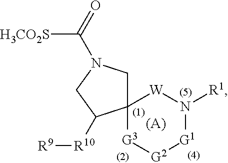

wherein Bn represents benzyl, i.e., —CH₂-phenyl), and wherein all substituents are as defined for formula (I), and in one example, W is —C(O)—.

In another embodiment of this invention R¹ is taken together with the nitrogen to which it is bound, and taken together with G¹ form a 4 to 8 membered ring fused to Ring (A). Thus, one embodiment of this invention is directed to compounds of the formula:

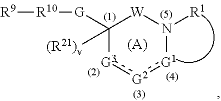

and in one example W is —C(O)—.

Another embodiment of this invention is directed to compounds of the formula:

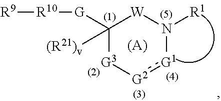

and in one example W is —C(O)—.

Another embodiment of this invention is directed to compounds of the formula:

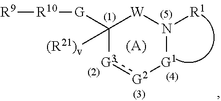

and in one example W is —C(O)—.

Another embodiment of this invention is directed to compounds of the formula:

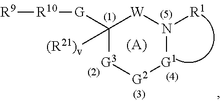

and in one example W is —C(O)—.

In another embodiment of this invention G and the Ring (A) carbon to which G is bound form a spiro ring. Thus, one embodiment of this invention is directed to compounds of the formula:

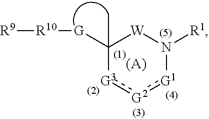

and in one example W is —C(O)—.

Another embodiment of this invention, wherein G and Ring (A) form a spiro ring, is directed to compounds of the formula:

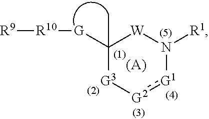

and in one example W is —C(O)—.

Another embodiment of this invention, wherein G and Ring (A) form a spiro ring, is directed to compounds of the formula:

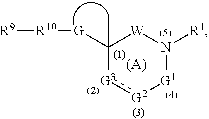

and in one example W is —C(O)—.

Another embodiment of this invention, wherein G and Ring (A) form a spiro ring, is directed to compounds of the formula:

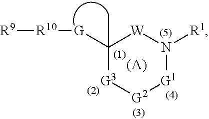

and in one example W is —C(O)—.

In another embodiment of this invention G and (R²¹)ᵥ are taken together to form a spiro ring. Thus, one embodiment of this invention is directed to compounds of the formula:

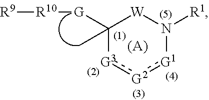

and in one example W is —C(O)—.

Another embodiment of this invention, wherein G and (R²¹)ᵥ are taken together to form a spiro ring, is directed to compounds of the formula:

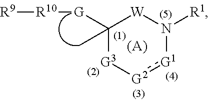

and in one example W is —C(O)—.

Another embodiment of this invention, wherein G and (R²¹)ᵥ are taken together to form a spiro ring, is directed to compounds of the formula:

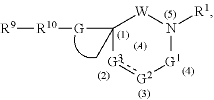

and in one example W is —C(O)—.

Another embodiment of this invention, wherein G and (R²¹)ᵥ are taken together to form a spiro ring, is directed to compounds of the formula:

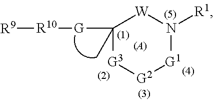

and in one example W is —C(O)—.

Another embodiment of this invention is directed to compounds of the formula:

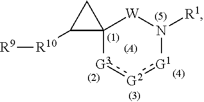

and in one example W is —C(O)—.

Another embodiment of this invention is directed to compounds of the formula:

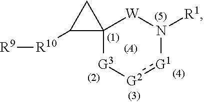

and in one example W is —C(O)—.

Another embodiment of this invention is directed to compounds of the formula:

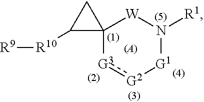

and in one example W is —C(O)—.

Another embodiment of this invention is directed to compounds of the formula:

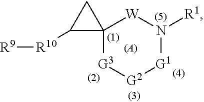

and in one example W is —C(O)—.

Another embodiment of this invention is directed to compounds of the formula:

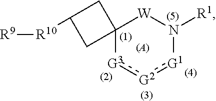

and in one example W is —C(O)—.

Another embodiment of this invention is directed to compounds of the formula:

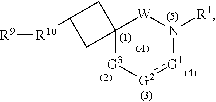

and in one example W is —C(O)—.

Another embodiment of this invention is directed to compounds of the formula:

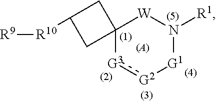

and in one example W is —C(O)—.

Another embodiment of this invention is directed to compounds of the formula:

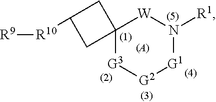

and in one example W is —C(O)—.

Another embodiment of this invention is directed to compounds of the formula:

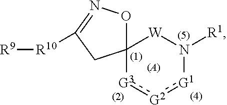

and in one example W is —C(O)—.

Another embodiment of this invention is directed to compounds of the formula:

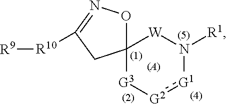

and in one example W is —C(O)—.

Another embodiment of this invention is directed to compounds of the formula:

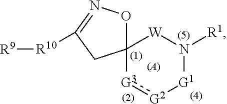

and in one example W is —C(O)—.

Another embodiment of this invention is directed to compounds of the formula:

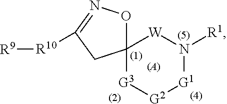

and in one example W is —C(O)—.

Another embodiment of this invention is directed to compounds of the formula:

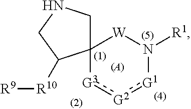

and in one example W is —C(O)—.

Another embodiment of this invention is directed to compounds of the formula:

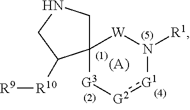

and in one example W is —C(O)—.

Another embodiment of this invention is directed to compounds of the formula:

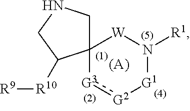

and in one example W is —C(O)—.

Another embodiment of this invention is directed to compounds of the formula:

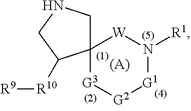

and in one example W is —C(O)—.

Another embodiment of this invention is directed to compounds of the formula:

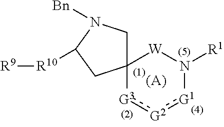

(wherein Bn represents benzyl, i.e., —CH₂-phenyl), and in one example W is —C(O)—.

Another embodiment of this invention is directed to compounds of the formula:

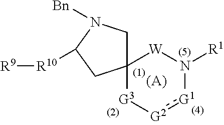

(wherein Bn represents benzyl, i.e., —CH₂-phenyl), and in one example W is —C(O)—.

Another embodiment of this invention is directed to compounds of the formula:

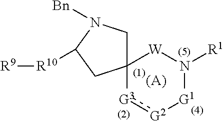

(wherein Bn represents benzyl, i.e., —CH₂-phenyl), and in one example W is —C(O)—.

Another embodiment of this invention is directed to compounds of the formula:

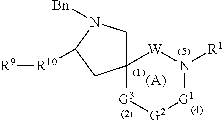

(wherein Bn represents benzyl, i.e., —CH₂-phenyl), and in one example W is —C(O)—.

Another embodiment of this invention is directed to compounds of the formula:

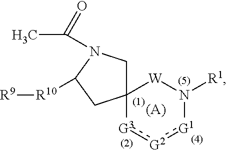

and in one example W is —C(O)—.

Another embodiment of this invention is directed to compounds of the formula:

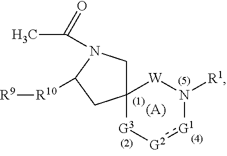

and in one example W is —C(O)—.

Another embodiment of this invention is directed to compounds of the formula:

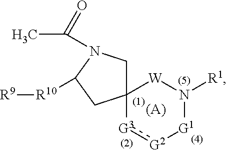

and in one example W is —C(O)—.

Another embodiment of this invention is directed to compounds of the formula:

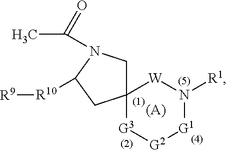

and in one example W is —C(O)—.

Another embodiment of this invention is directed to compounds of the formula:

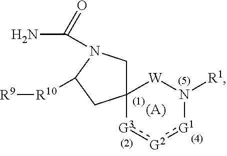

and in one example W is —C(O)—.

Another embodiment of this invention is directed to compounds of the formula:

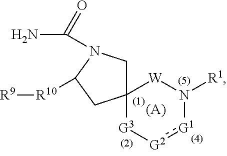

and in one example W is —C(O)—.

Another embodiment of this invention is directed to compounds of the formula:

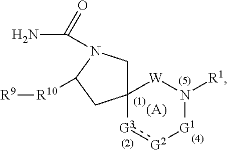

and in one example W is —C(O)—.

Another embodiment of this invention is directed to compounds of the formula:

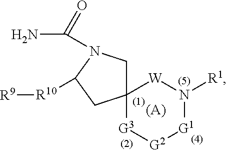

and in one example W is —C(O)—.

Another embodiment of this invention is directed to compounds of the formula:

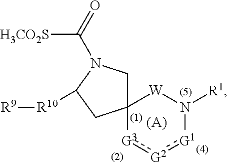

and in one example W is —C(O)—.

Another embodiment of this invention is directed to compounds of the formula:

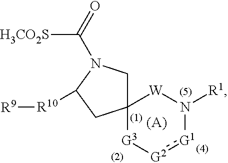

and in one example W is —C(O)—.

Another embodiment of this invention is directed to compounds of the formula:

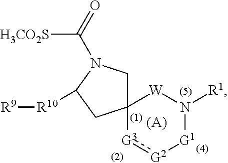

and in one example W is —C(O)—.

Another embodiment of this invention is directed to compounds of the formula:

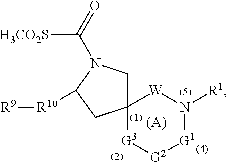

and in one example W is —C(O)—.

In another embodiment of this invention G¹ and G² are taken together to form a ring. Thus, one embodiment one embodiment of this invention is directed to compounds of the formula:

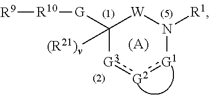

and in one example W is —C(O)—.

Another embodiment of this invention is directed to compounds of the formula:

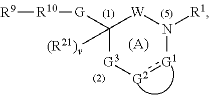

and in one example W is —C(O)—.

Another embodiment of this invention is directed to compounds of the formula:

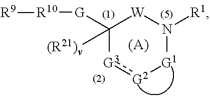

and in one example W is —C(O)—.

Another embodiment of this invention is directed to compounds of the formula:

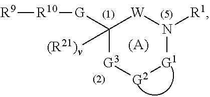

and in one example W is —C(O)—.

In another embodiment of this invention G² and G³ are taken together to form a ring. Thus, one embodiment one embodiment of this invention is directed to compounds of the formula:

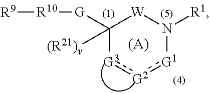

and in one example W is —C(O)—.

Another embodiment of this invention is directed to compounds of the formula:

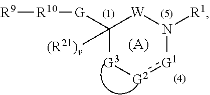

and in one example W is —C(O)—.

Another embodiment of this invention is directed to compounds of the formula:

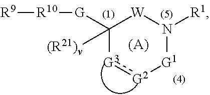

and in one example W is —C(O)—.

Another embodiment of this invention is directed to compounds of the formula:

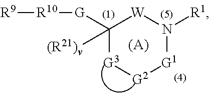

and in one example W is —C(O)—.

In one embodiment of this invention, the cycloalkyl G moiety is a C₃ to C₁₀ cycloalkyl. In one example, said cycloalkyl is selected from the group consisting of: cyclopropyl, cyclobutyl, cyclopentyl and cyclohexyl. In another example of said cycloalkyl G moiety the cycloalkyl ring carbon by which said cycloalkyl moiety is bound to position (1) or (2) is different from the cycloalkyl ring carbon by which said cycloalkyl moiety is bound to moiety R¹⁰. In another example of said cycloalkyl G moiety the cycloalkyl ring is bound to position (1) or (2) and the R¹⁰ moiety by the same cycloalkyl ring carbon.

In another embodiment of this invention, G and the Ring (A) carbon to which G is bound form a spiro ring (e.g., a cyclopropyl or cyclobutyl spiro ring), and v is 0 for the R²¹ moiety at position 1, and there is no H bound to the carbon at position (1).

In one embodiment of this invention, the heterocycloalkyl G moiety comprises 1 to 4 heteroatoms. In one example, said heterocycloalkyl G moiety comprises 1 to 4 heteroatoms. In another example, said heterocycloalkyl G moiety comprises 1 to 3 heteoatoms. In another example, said heterocycloalkyl G moiety comprises 1 to 2 heteroatoms. In another example, said heterocycloalkyl G moiety comprises 1 heteroatom. The heteroatoms in said heterocycloalkyl G moiety are independently selected from the group consisting of —O—, —NR²—, —S—, —S(O)—, and —S(O)₂. In one example, said heterocycloalkyl G moiety is bound to the R¹⁰ moiety and position (1) or (2) by the same heterocycloalkyl ring atom. In another example, said heterocycloalkyl moiety is bound to the R¹⁰ moiety and position (1) or (2) by different heterocycloalkyl ring atoms, and wherein the heterocycloalkyl ring atoms that bind the heterocycloalkyl moiety to R¹⁰ and position (1) or (2) are selected from the group consisting of carbon and nitrogen.

An example of said alkynyl G moiety is:

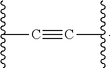

Those skilled in the art will appreciate that the G moiety —(C═NR²)— represents:

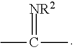

Those skilled in the art will appreciate that the G moiety —(C═C(R⁶)₂)— represents:

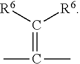

Those skilled in the art will appreciate that when W is —S(O)—, the —S(O)— moiety can be:

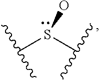

or the —S(O)— moiety can be;

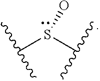

In another embodiment of this invention G is selected from the group consisting of: a direct bond, G is selected from the group consisting of: a direct bond (i.e., R¹⁰ is bound directly to Ring (A) at position (1)), cycloalkyl (e.g., C₃ to C₁₀, and also for example, cyclopropyl, cyclobutyl, cyclopentyl and cyclohexyl, and wherein in one example the cycloalkyl ring carbon by which said cycloalkyl moiety is bound to position (1) is different from the cycloalkyl ring carbon by which said cycloalkyl moiety is bound to moiety R¹⁰, and wherein in another example said cycloalkyl ring is bound to position (1) and the R¹⁰ moiety by the same cycloalkyl ring carbon), heterocycloalkyl (wherein said heterocycloalkyl comprises 1 to 4 heteroatoms, and in one example, 1 to 4 heteroatoms, and in another example 1 to 3 heteoatoms, and in another example 1 to 2 heteroatoms, and in another example 1 heteroatom, and wherein said heteroatoms are selected from the group consisting of —O—, —S—, —S(O)—, and —S(O)₂, and wherein in one example said heterocycloalkyl moiety is bound to the R¹⁰ moiety and position (1) by the same heterocycloalkyl ring atom, and in another example said heterocycloalkyl moiety is bound to the R¹⁰ moiety and position (1) by different heterocycloalkyl ring atoms, and wherein the heterocycloalkyl ring atoms that bind the heterocycloalkyl moiety to R¹⁰ and position (1) are selected from the group consisting of carbon and nitrogen), —C═C—, —CF₂— alkynyl (e.g., —C≡C—), —NH—, —N(R²)— (and in one example, —NH—), —O—, —CR⁴(OH)—, —CR⁴(OR⁴)—, —(CH₂)ᵣN(R²)—, —N(R²)(CH₂)ᵣ—, —(CH₂)₂₋₅—, —(C(R⁴)₂)ᵣ— (wherein each R⁴ is independently selected), —(CHR⁴)₂₋₅— (wherein each R⁴ is independently selected), —S—, —S(O)—, and —S(O)₂.

In another embodiment of this invention v (for the R²¹ group at position (1)) is 0 and there is a H bound to the carbon at position (1) to fill the required valence.

In another embodiment of this invention G¹ and G² are taken together to form a ring.

In another embodiment of this invention G² and G³ are taken together to form a ring.

In another embodiment of this invention no optional ring is formed between G¹ and G², or G² and G³, or G and G³, or G and the Ring (A) carbon to which G is bound (that is there are no optional rings bound to Ring (A) formed by G and the atoms in Ring (A)).

In another embodiment of this invention G is selected from the group consisting of: a direct bond, and —N(R²) (e.g., —NH—).

In another embodiment of this invention G is a cycloalkyl.

In another embodiment of this invention G is a heterocycloalkyl.

In another embodiment of this invention G is —C═C—.

In another embodiment of this invention G is —CF₂—.

In another embodiment of this invention G is alkynyl.

In another embodiment of this invention G is —O—.

In another embodiment of this invention G is —CR⁴(OH)—.

In another embodiment of this invention G is —CR⁴(OR⁴)—.

In another embodiment of this invention G is —(CH₂)ᵣN(R²)—.

In another embodiment of this invention G is —N(R²)(CH₂)ᵣ—.

In another embodiment of this invention G is —(CH₂)₂₋₁₀—.

In another embodiment of this invention G is —(C(R⁴)₂)ᵣ— (wherein each R⁴ is independently selected).

In another embodiment of this invention G is —(CHR⁴)₂₋₁₀— (wherein each R⁴ is independently selected).

In another embodiment of this invention G is —S—.

In another embodiment of this invention G is —S(O)—.

In another embodiment of this invention G is —S(O)₂.

In another embodiment of this invention G¹ is a direct bond.

In another embodiment of this invention G¹ is —O—.

In another embodiment of this invention G¹ is —C(R²¹)_{q}.

In another embodiment of this invention G¹ is —N(R²)_{d}—.

In another embodiment of this invention G¹ is —C(O)—.

In another embodiment of this invention G¹ is —C(═NR²)—.

In another embodiment of this invention G¹ is —S—.

In another embodiment of this invention G¹ is —S(O)₂.

In another embodiment of this invention G¹ is —S(O)—.

In another embodiment of this invention G² is a direct bond.

In another embodiment of this invention G² is —O—.

In another embodiment of this invention G² is —C(R²¹)_{q}.

In another embodiment of this invention G² is —N(R²)_{d}—.

In another embodiment of this invention G² is —C(O)—.

In another embodiment of this invention G² is —C(═NR²)—.

In another embodiment of this invention G² is —S—.

In another embodiment of this invention G² is —S(O)₂.

In another embodiment of this invention G² is —S(O)—.

In another embodiment of this invention R²¹ is selected from the group consisting of: alkyl, —OR¹⁵, —C(O)OR¹⁵, —C(O)NR¹⁵R¹⁶, and alkyl substituted with 1 to 5 independently selected R²² groups (e.g., halo, such as, for example, F, Cl, and Br).

In another embodiment of this invention R²¹ is selected from the group consisting of: alkyl, —OR¹⁵, —C(O)OR¹⁵, —C(O)NR¹⁵R¹⁶, and alkyl substituted with 1 to 5 independently selected R²² groups (e.g., halo, such as, for example, F, Cl, and Br, and wherein in one example the alkyl substituted R²¹ group is —CF₃), wherein R¹⁵ and R¹⁶ are independently selected from the group consisting of: H, alkyl, (R¹⁸)ₙ-arylalkyl- (wherein, for example, n is 1, and R¹⁸ is —OR²⁰, and R²⁰ is alkyl (e.g., methyl), cycloalkyl (e.g., cyclobutyl), and (R¹⁸)ₙ-alkyl (e.g, n is 1, R¹⁸ is —OR²⁰, and R²⁰ is alkyl (e.g., methyl).

In another embodiment of this invention R²¹ is selected from the group consisting of: (a) alkyl, —OR¹⁵ (wherein R¹⁵ is alkyl, e.g., methyl and ethyl), (b) —C(O)OR¹⁵ (wherein R¹⁵ is alkyl, e.g., methyl), (c) —C(O)NR¹⁵R¹⁶ (wherein R¹⁵ and R¹⁶ are independently selected from the group consisting of: H, alkyl, (R¹⁸)ₙ-arylalkyl- (wherein, for example, n is 1, and R¹⁸ is —OR²⁰, and R²⁰ is alkyl (e.g., methyl), cycloalkyl (e.g., cyclobutyl), and (R¹⁸)ₙ-alkyl (e.g, n is 1, R¹⁸ is —OR²⁰, and R²⁰ is alkyl (e.g., methyl), and in one example, only one of R¹⁵ and R¹⁶ is H), and (d) alkyl substituted with 1 to 5 independently selected R²² groups (e.g., halo, such as, for example, F, Cl, and Br, and wherein in one example the alkyl substituted R²¹ group is —CF₃).

Examples of R¹⁰ include, but are not limited to:

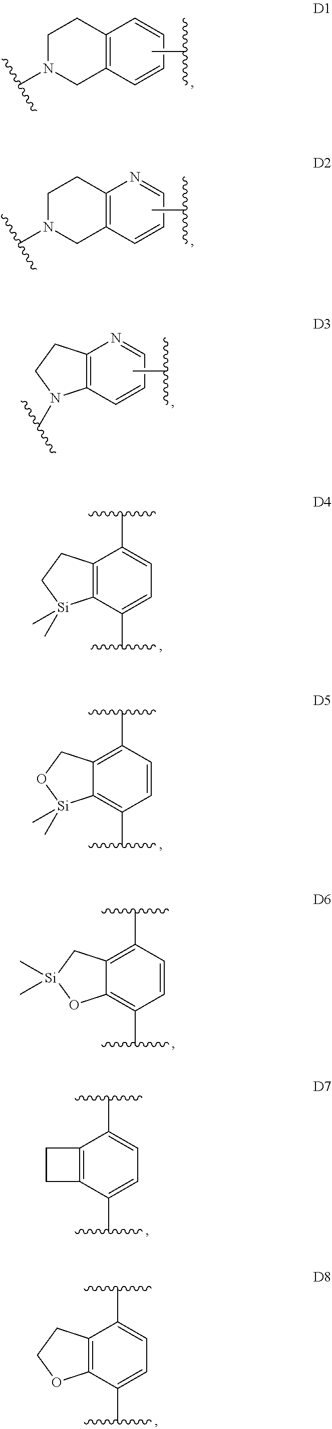

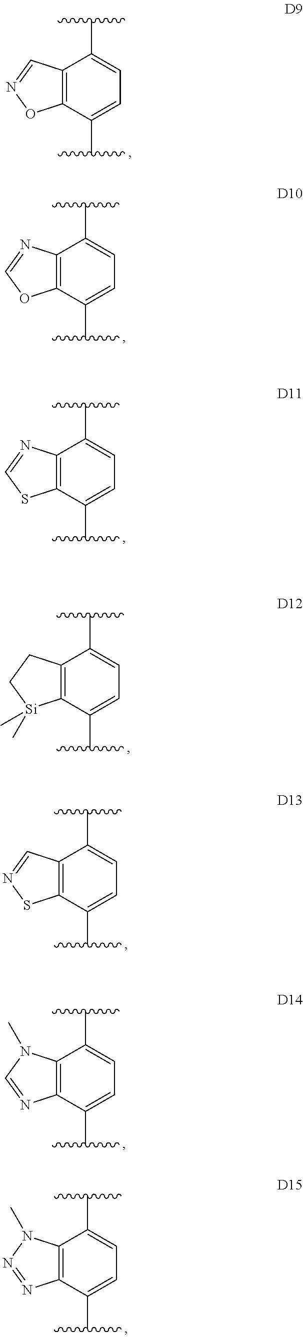

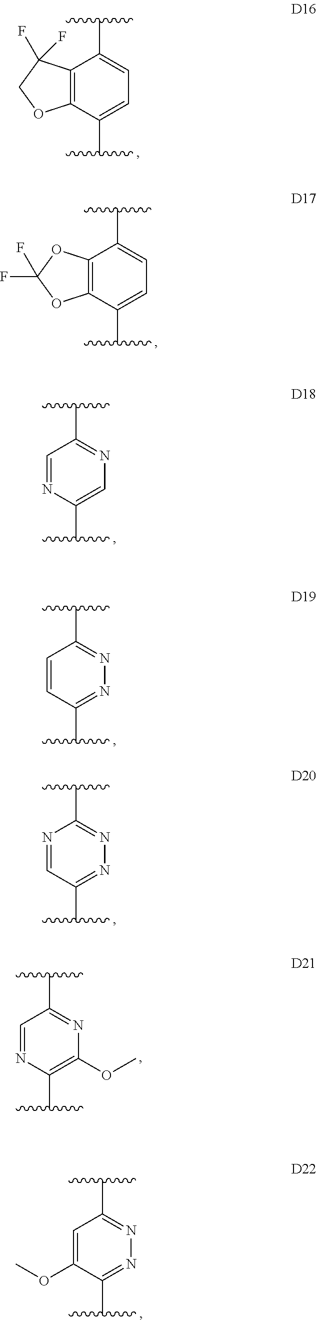

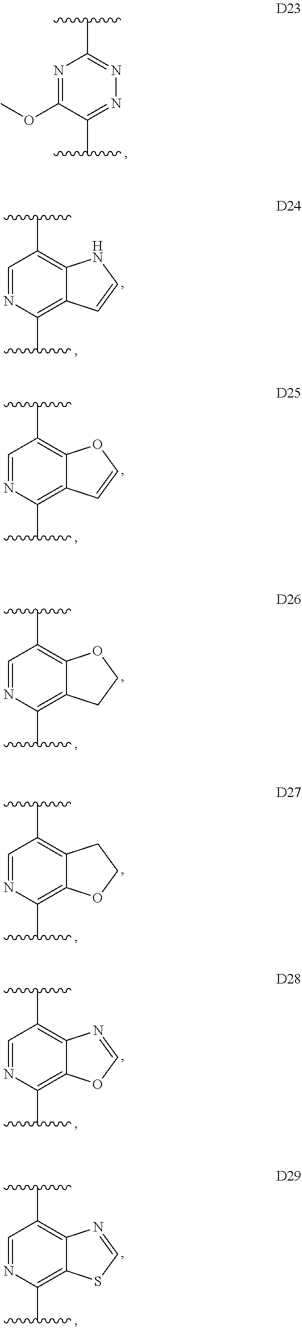

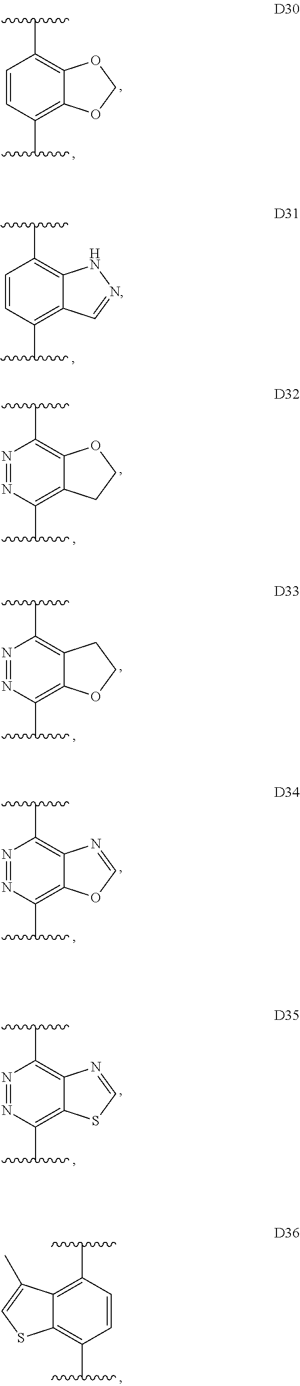

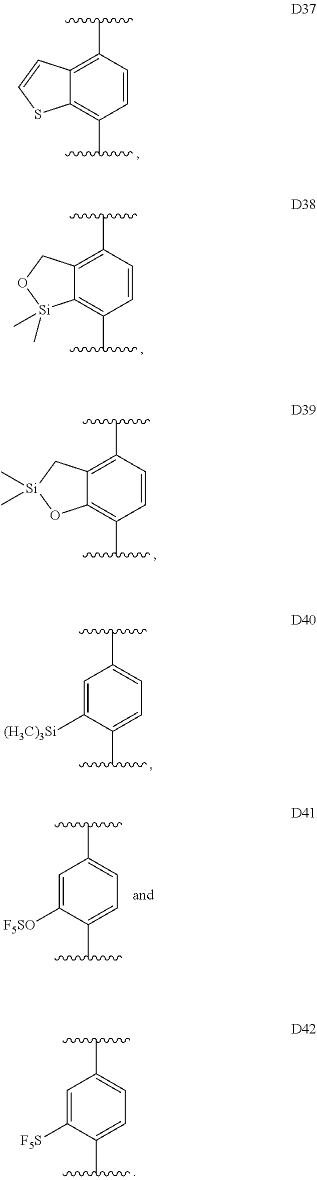

Thus, in one embodiment of this invention, R¹⁰ is selected from the group consisting of the above R¹⁰ groups.

An example of the R¹⁰ group:

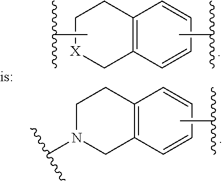

Thus, in one embodiment of this invention, R¹⁰ is the above R¹⁰ group.

An example of the R¹⁰ group:

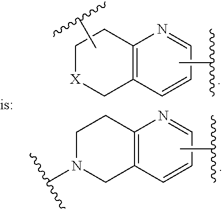

Thus, in one embodiment of this invention, R¹⁰ is the above R¹⁰ group.

An example of the fused cycloalkylaryl-R¹⁰ groups is:

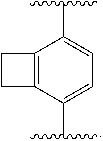

Thus, in one embodiment of this invention, R¹⁰ is the above R¹⁰ groups.

Examples of the fused heterocycloalkylaryl-R¹⁰ groups include:

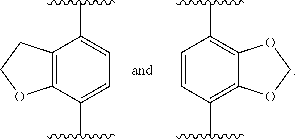

Thus, in one embodiment of this invention, R¹⁰ is selected from the group consisting of the above R¹⁰ groups.

Examples of the substituted fused heterocycloalkylaryl-R¹⁰ groups include:

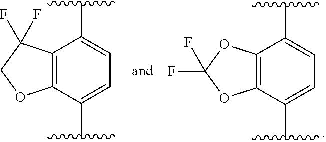

Thus, in one embodiment of this invention, R¹⁰ is selected from the group consisting of the above R¹⁰ groups.

Examples of the fused heterocycloalkenylaryl-R¹⁰ groups include:

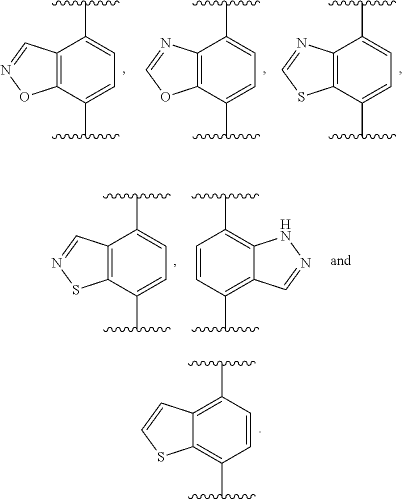

Thus, in one embodiment of this invention, R¹⁰ is selected from the group consisting of the above R¹⁰ groups.

Examples of the substituted fused heterocycloalkenylaryl-R¹⁰ groups include:

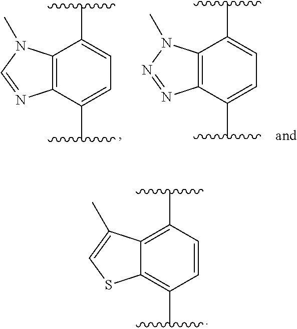

Thus, in one embodiment of this invention, R¹⁰ is selected from the group consisting of the above R¹⁰ groups.

Examples of the heteroaryl-R¹⁰ groups include:

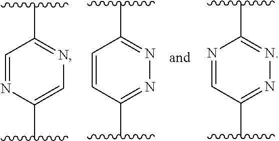

Thus, in one embodiment of this invention, R¹⁰ is selected from the group consisting of the above R¹⁰ groups.

Examples of the substituted heteroaryl-R¹⁰ groups include:

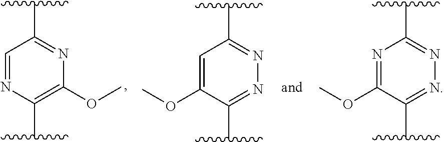

Thus, in one embodiment of this invention, R¹⁰ is selected from the group consisting of the above R¹⁰ groups.

Examples of the substituted fused heterocycloalkenylheteroaryl-R¹⁰ groups include:

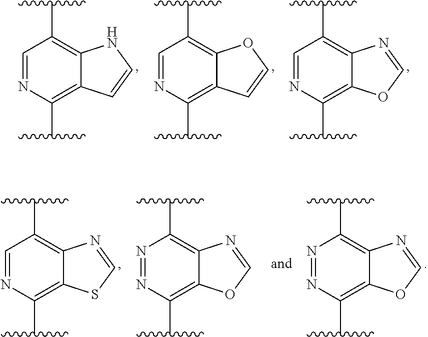

Thus, in one embodiment of this invention, R¹⁰ is selected from the group consisting of the above R¹⁰ groups.

Examples of the fused heterocycloalkylheteroaryl-R¹⁰ groups include:

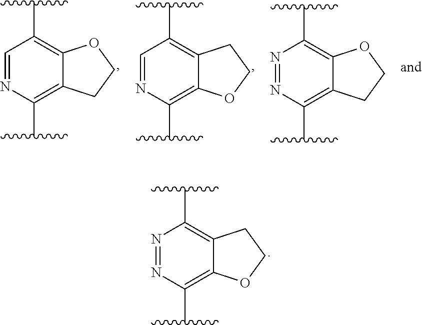

Thus, in one embodiment of this invention, R¹⁰ is selected from the group consisting of the above R¹⁰ groups.

Examples of the substituted aryl-R¹⁰ groups include:

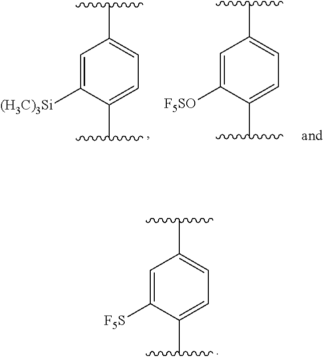

Thus, in one embodiment of this invention, R¹⁰ is selected from the group consisting of the above R¹⁰ groups.

In another embodiment R¹⁰ is D1. In another embodiment R¹⁰ is D2. In another embodiment R¹⁰ is D3. In another embodiment R¹⁰ is D4. In another embodiment R¹⁰ is D5. In another embodiment R¹⁰ is D6. In another embodiment R¹⁰ is D7. In another embodiment R¹⁰ is D8. In another embodiment R¹⁰ is D9. In another embodiment R¹⁰ is D10. In another embodiment R¹⁰ is D11. In another embodiment R¹⁰ is D12. In another embodiment R¹⁰ is D13. In another embodiment R¹⁰ is D14. In another embodiment R¹⁰ is D15. In another embodiment R¹⁰ is D16. In another embodiment R¹⁰ is D17. In another embodiment R¹⁰ is D18. In another embodiment R¹⁰ is D19. In another embodiment R¹⁰ is D20. In another embodiment R¹⁰ is D21. In another embodiment R¹⁰ is D22. In another embodiment R¹⁰ is D23. In another embodiment R¹⁰ is D24. In another embodiment R¹⁰ is D25. In another embodiment R¹⁰ is D26. In another embodiment R¹⁰ is D27. In another embodiment R¹⁰ is D28. In another embodiment R¹⁰ is D29. In another embodiment R¹⁰ is D30. In another embodiment R¹⁰ is D31. In another embodiment R¹⁰ is D32. In another embodiment R¹⁰ is D33. In another embodiment R¹⁰ is D34. In another embodiment R¹⁰ is D35. In another embodiment R¹⁰ is D36. In another embodiment R¹⁰ is D37. In another embodiment R¹⁰ is D38. In another embodiment R¹⁰ is D39. In another embodiment R¹⁰ is D40. In another embodiment R¹⁰ is D41. In another embodiment R¹⁰ is D42.

In another embodiment of this invention R¹⁰ is aryl.

In another embodiment of this invention R¹⁰ is aryl and said aryl is phenyl.

In another embodiment of this invention R¹⁰ is aryl substituted with one or more R²¹ groups.

In another embodiment of this invention R¹⁰ is aryl substituted with one or more R²¹ groups, and said aryl is phenyl, i.e., said R¹⁰ group is phenyl substituted with one or more R²¹ groups.

In another embodiment of this invention R¹⁰ is phenyl substituted with one or more R²¹ groups, and each R²¹ group is the same or different —OR¹⁵ group.

In another embodiment of this invention R¹⁰ is phenyl substituted with one or more R²¹ groups, and each R²¹ group is the same or different —OR¹⁵ group, and said R¹⁵ is alkyl, and each alkyl is independently selected.

In another embodiment of this invention R¹⁰ is phenyl substituted with one R²¹ group, and said R²¹ group is —OR¹⁵, and said R¹⁵ is alkyl.

In another embodiment of this invention R¹⁰ is phenyl substituted with one R²¹ group, and said R²¹ group is —OR¹⁵, and said R¹⁵ is alkyl, and said alkyl is methyl.

In another embodiment of this invention R¹⁰ is heteroaryl.

In another embodiment of this invention R¹⁰ is heteroaryl substituted with one or more R²¹ groups.

In another embodiment of this invention R⁹ is heteroaryl.

In another embodiment of this invention R⁹ is heteroaryl substituted with one or more R²¹ groups.

In another embodiment of this invention R⁹ is heteroaryl substituted with one or more R²¹ groups, and said R²¹ groups are the same or different alkyl.

In another embodiment of this invention R⁹ is heteroaryl substituted with one R²¹ group, and said R²¹ is alkyl.

In another embodiment of this invention R⁹ is heteroaryl substituted with one R²¹ group, and said R²¹ is alkyl, and said alkyl is methyl.

In another embodiment of this invention R⁹ is and said heteroaryl is imidazoyl.

In another embodiment of this invention R⁹ is imidazolyl substituted with one or more R²¹ groups.

In another embodiment of this invention R⁹ is imidazolyl substituted with one or more R²¹ groups, and said R²¹ groups are the same or different alkyl.

In another embodiment of this invention R⁹ is imidazolyl substituted with one R²¹ group, and said R²¹ is alkyl.

In another embodiment of this invention R⁹ is imidazolyl substituted with one R²¹ group, and said R²¹ is alkyl, and said alkyl is methyl.

In another embodiment of this invention R¹⁰ is selected from the group consisting of aryl and aryl substituted with one or more R²¹ groups, and said R⁹ group is selected from the group consisting of heteroaryl and heteroaryl substituted with one or more R²¹ groups, wherein each R²¹ is independently selected.

In another embodiment of this invention R¹⁰ is phenyl substituted with one or more R²¹ groups, and said R⁹ is imidazolyl substituted with one or more R²¹ groups, wherein each R²¹ is independently selected.

In another embodiment of this invention R¹⁰ is phenyl substituted with one R²¹ group, and said R⁹ is imidazolyl substituted with one R²¹ group, wherein each R²¹ is independently selected.

In another embodiment of this invention R¹⁰ is phenyl substituted with one or more independently selected —OR¹⁵ groups, and said R⁹ is imidazolyl substituted with one or more independently selected alkyl groups.

In another embodiment of this invention R¹⁰ is phenyl substituted with one or more independently selected —OR¹⁵ groups, and said R⁹ is imidazolyl substituted with one or more independently selected alkyl groups, and each R¹⁵ is the same or different alkyl group.

In another embodiment of this invention R¹⁰ is phenyl substituted with one —OR¹⁵ group, and said R⁹ is imidazolyl substituted with one alkyl group.

In another embodiment of this invention R¹⁰ is phenyl substituted with one —OR¹⁵ group, and said R⁹ is imidazolyl substituted with one alkyl group, and R¹⁵ is alkyl, and wherein the R¹⁵ alkyl group, and the alkyl group on said imidazolyl are independently selected.

In another embodiment of this invention R¹⁰ is phenyl substituted with one —OR¹⁵ group, and said R⁹ is imidazolyl substituted with one methyl group, and R¹⁵ is methyl, and wherein the R¹⁵ alkyl group, and the alkyl group on said imidazolyl are independently selected.

In another embodiment of this invention the R⁹-R¹⁰— moiety is:

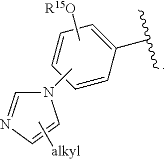

In another embodiment of this invention the R⁹-R¹⁰— moiety is:

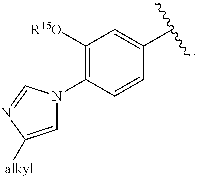

In another embodiment of this invention the R⁹-R¹⁰— moiety is:

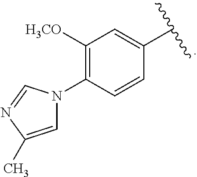

In another embodiment of this invention the R⁹-R¹⁰— moiety is:

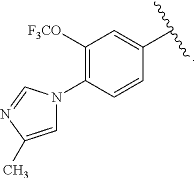

In another embodiment of this invention the R⁹-R¹⁰— moiety is:

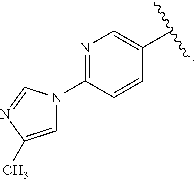

In another embodiment of this invention the R⁹-R¹⁰— moiety is:

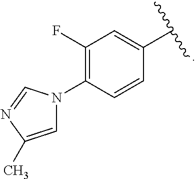

Examples of moieties formed when R¹⁰ and R⁹ are linked together to form a fused tricyclic ring system include, but are not limited to:

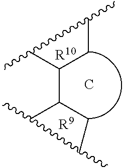

wherein R¹⁰ and R⁹ are as defined for formula (I), and Ring C is the ring linking R¹⁰ and R⁹, that is Ring C is an alkyl ring, or a heteroalkyl ring, or an aryl ring, or a heteroaryl ring, or an alkenyl ring, or a heteroalkenyl ring.

Examples of moieties formed when R¹⁰ and R⁹ are linked together to form a fused tricyclic ring system include, but are not limited to:

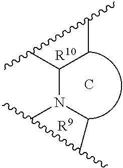

wherein R¹⁰ and R⁹ are as defined for formula (I), and Ring C is the ring linking R¹⁰ and R⁹, that is Ring C is a heteroalkyl ring, or a heteroaryl ring, or a heteroalkenyl ring.

In one example, the fused tricyclic ring system formed when R¹⁰ and R⁹ are linked together is

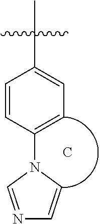

wherein Ring C is a heteroalkyl ring, or a heteroaryl ring, or a heteroalkenyl ring, thus, for example, the tricyclic ring system is formed by linking the atoms adjacent to the atoms by which R¹⁰ and R⁹ are bound together), and wherein said fused tricyclic ring system is optionally substituted with 1 to 5 independently selected R²¹ groups.

Other examples of moieties formed when R¹⁰ and R⁹ are linked together to form a fused tricyclic ring system include, but are not limited to:

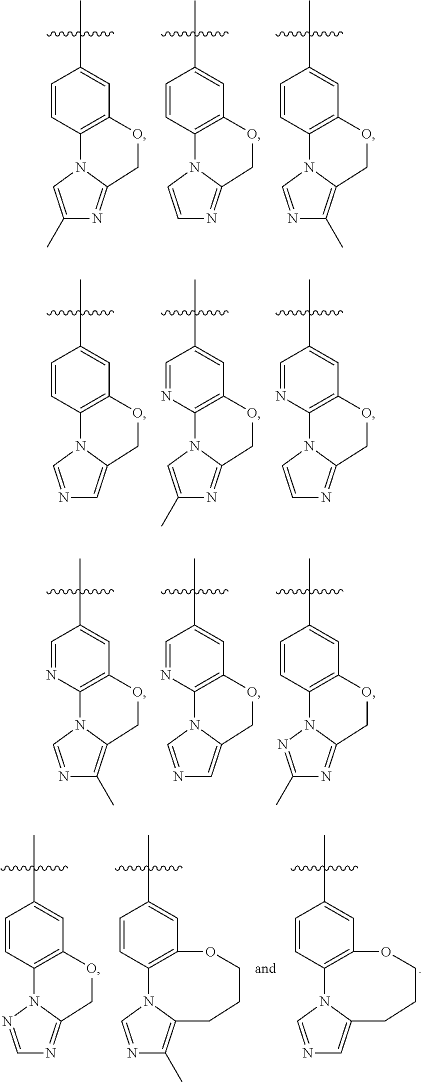

In another embodiment of this invention R¹ is an alkyl group substituted with one or more independently selected R²¹ groups.

In another embodiment of this invention R¹ is:

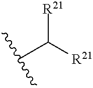

wherein each R²¹ is independently selected, and each R²¹ is independently unsubstituted or substituted with one or more independently selected R²² groups.

In another embodiment of this invention R¹ is:

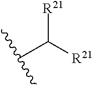

wherein one R²¹ is an unsubstituted or substituted alkyl group.

In another embodiment of this invention R¹ is:

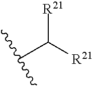

wherein one R²¹ is an unsubstituted alkyl group.

In another embodiment of this invention R¹ is:

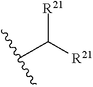

wherein one R²¹ is a substituted alkyl group.

In another embodiment of this invention R¹ is:

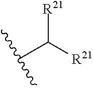

wherein one R²¹ is an unsubstituted or substituted alkyl group, and the other R²¹ is an unsubstituted or substituted aryl (e.g., phenyl) group.

In another embodiment of this invention R¹ is:

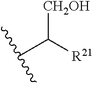

and R²¹ is unsubstituted or substituted with one or more independently selected R²² groups.

In another embodiment of this invention R¹ is:

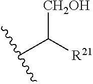

and R²¹ is unsubstituted aryl (e.g., phenyl) or aryl (e.g., phenyl) substituted with one or more independently selected R²² groups.

In another embodiment of this invention R¹ is an alkyl group substituted with one R²¹ group.

In another embodiment of this invention R¹ is an alkyl group substituted with one R²¹ group, and said R²¹ group is substituted with one or more independently selected R²² groups.

In another embodiment of this invention R¹ is:

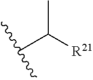

wherein R²¹ is unsubstituted or substituted with one or more independently selected R²² groups.

In another embodiment of this invention R¹ is an alkyl group substituted with one R²¹ group, and said R²¹ group is an aryl group.

In another embodiment of this invention R¹ is an alkyl group substituted with one R²¹ group, and said R²¹ group is an aryl group, said aryl is phenyl.

In another embodiment of this invention R¹ is an ethyl group substituted with one R²¹ group, and said R²¹ group is an aryl group, said aryl is phenyl.

In another embodiment of this invention R¹ is a methyl group substituted with one R²¹ group, and said R²¹ group is an aryl group, said aryl is phenyl.

In another embodiment of this invention R¹ is an alkyl group substituted with one R²¹ group, and said R²¹ group is an aryl group, and said aryl group is substituted with one or more R²² groups.

In another embodiment of this invention R¹ is an alkyl group substituted with one R²¹ group, and said R²¹ group is an aryl group, and said aryl group is substituted with one or more R²² groups, and each R²² group is independently selected from the group consisting of: —SF₅, —OSF₅, —Si(R^{15A})₃.

In another embodiment of this invention R¹ is an alkyl group substituted with one R²¹ group, and said R²¹ group is an aryl group, and said aryl group is substituted with one or two R²² groups, and each R²² group is independently selected from the group consisting of: —SF₅, —OSF₅, —Si(R^{15A})₃.

In another embodiment of this invention R¹ is an alkyl group substituted with one R²¹ group, and said R²¹ group is an aryl group, and said aryl group is substituted with one R²² group, and said R²² group is selected from the group consisting of: —SF₅, —OSF₅, —Si(R^{15A})₃.

In another embodiment of this invention R¹ is an alkyl group substituted with one R²¹ group, and said R²¹ group is an aryl group, and said aryl group is substituted with one or more R²² groups, and each R²² group is the same or different halo.

In another embodiment of this invention R¹ is an alkyl group substituted with one R²¹ group, and said R²¹ group is an aryl group, and said aryl group is substituted with 1 to 3 R²² groups, and each R²² group is the same or different halo.

In another embodiment of this invention R¹ is an alkyl group substituted with one R²¹ group, and said R²¹ group is an aryl group, and said aryl group is substituted with one or two R²² halo groups, and each R²² group is the same or different halo.

In another embodiment of this invention R¹ is an alkyl group substituted with one R²¹ group, and said R²¹ group is an aryl group, and said aryl group is substituted with one or two R²²F groups.

In another embodiment of this invention R¹ is an alkyl group substituted with one R²¹ group, and said R²¹ group is phenyl, and said phenyl is substituted with one or more R²² groups.

In another embodiment of this invention R¹ is an alkyl group substituted with one R²¹ group, and said R²¹ group is phenyl, and said phenyl is substituted with one or more R²² groups, and each R²² group is independently selected from the group consisting of: —SF₅, —OSF₅, —Si(R^{15A})₃.

In another embodiment of this invention R¹ is an alkyl group substituted with one R²¹ group, and said R²¹ group is phenyl, and said phenyl is substituted with one or two R²² groups, and each R²² group is independently selected from the group consisting of: —SF₅, —OSF₅, —Si(R^{15A})₃.

In another embodiment of this invention R¹ is an alkyl group substituted with one R²¹ group, and said R²¹ group is phenyl, and said phenyl is substituted with one R²² group, and said R²² group is selected from the group consisting of: —SF₅, —OSF₅, —Si(R^{15A})₃.

In another embodiment of this invention R¹ is an alkyl group substituted with one R²¹ group, and said R²¹ group is phenyl, and said phenyl is substituted with one or more R²² groups, and each R²² group is the same or different halo.

In another embodiment of this invention R¹ is an alkyl group substituted with one R²¹ group, and said R²¹ group is phenyl, and said phenyl is substituted with one or two R²² halo groups, and each R²² group is the same or different halo.

In another embodiment of this invention R¹ is an alkyl group substituted with one R²¹ group, and said R²¹ group is phenyl, and said phenyl is substituted with one or two R²²F groups.

In another embodiment of this invention R¹ is an ethyl group substituted with one R²¹ group, and said R²¹ group is an aryl group, and said aryl group is substituted with one or more R²² groups.

In another embodiment of this invention R¹ is a methyl group substituted with one R²¹ group, and said R²¹ group is an aryl group, and said aryl group is substituted with one or more R²² groups.

In another embodiment of this invention R¹ is an ethyl group substituted with one R²¹ group, and said R²¹ group is phenyl, and said phenyl is substituted with one or more R²² groups.

In another embodiment of this invention R¹ is an ethyl group substituted with one R²¹ group, and said R²¹ group is phenyl, and said phenyl is substituted with one or more R²² groups, and each R²² group is independently selected from the group consisting of: —SF₅, —OSF₅, —Si(R^{15A})₃.

In another embodiment of this invention R¹ is a methyl group substituted with one R²¹ group, and said R²¹ group is phenyl, and said phenyl is substituted with one or more R²² groups.

In another embodiment of this invention R¹ is an methyl group substituted with one R²¹ group, and said R²¹ group is phenyl, and said phenyl is substituted with one or more R²² groups, and each R²² group is independently selected from the group consisting of: —SF₅, —OSF₅, —Si(R^{15A})₃.

In another embodiment of this invention R¹ is an ethyl group substituted with one R²¹ group, and said R²¹ group is phenyl, and said phenyl is substituted with one or two R²² halo groups, and each R²² group is the same or different halo.

In another embodiment of this invention R¹ is a methyl group substituted with one R²¹ group, and said R²¹ group is phenyl, and said phenyl is substituted with one or two R²² halo groups, and each R²² group is the same or different halo.

In another embodiment of this invention R¹ is an ethyl group substituted with one R²¹ group, and said R²¹ group is phenyl, and said phenyl is substituted with one or two R²²F groups.

In another embodiment of this invention R¹ is a methyl group substituted with one R²¹ group, and said R²¹ group is phenyl, and said phenyl is substituted with one or two R²²F groups.

In another embodiment of this invention R¹ is an ethyl group substituted with one R²¹ group, and said R²¹ group is phenyl, and said phenyl is substituted with one R²² halo group.

In another embodiment of this invention R¹ is a methyl group substituted with one R²¹ group, and said R²¹ group is phenyl, and said phenyl is substituted with one R²² halo group.

In another embodiment of this invention R¹ is an ethyl group substituted with one R²¹ group, and said R²¹ group is phenyl, and said phenyl is substituted with one R²²F group.

In another embodiment of this invention R¹ is a methyl group substituted with one R²¹ group, and said R²¹ group is phenyl, and said phenyl is substituted with one R²²F group.

In another embodiment R¹ is selected from the group consisting of:

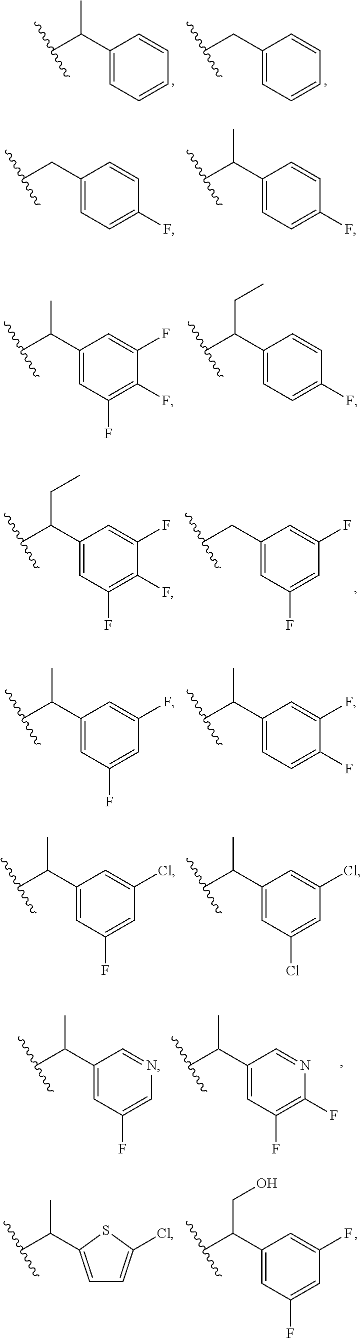

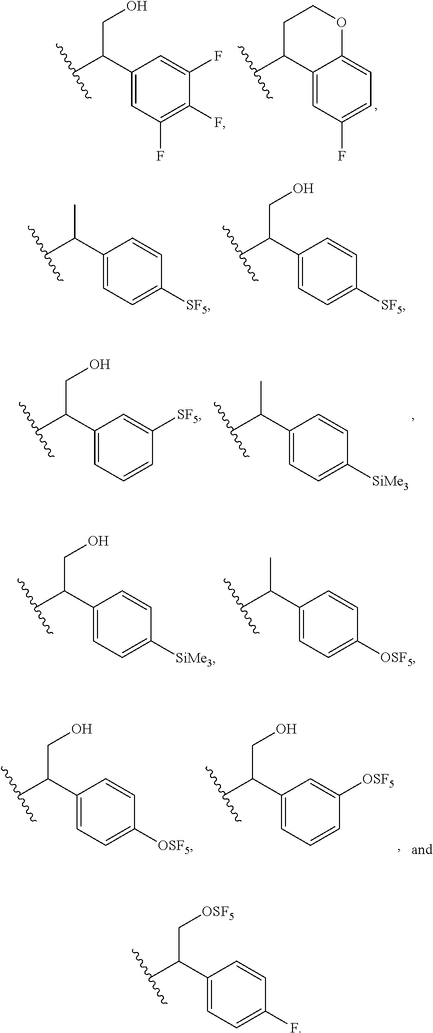

In another embodiment of this invention R¹ is selected from the group consisting of:

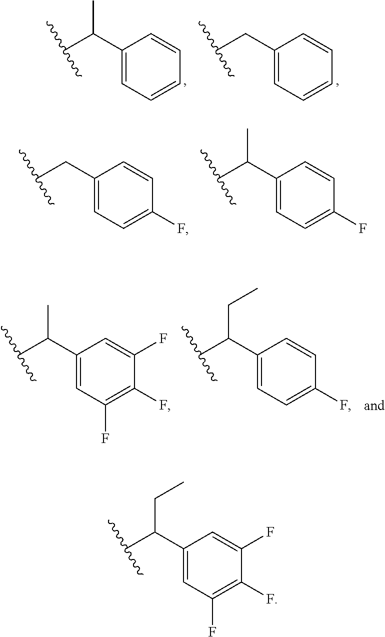

In another embodiment of this invention R¹⁰ is selected from the group consisting of aryl and aryl substituted with one or more R²¹ groups, and said R⁹ group is selected from the group consisting of heteroaryl and heteroaryl substituted with one or more R²¹ groups, and wherein each R²¹ is independently selected.

In another embodiment of this invention: (a) R¹ is an alkyl group substituted with one R²¹ group, or (b) R¹ is an alkyl group substituted with one R²¹ group, and said R²¹ group is substituted with one or more independently selected R²² groups, and (c) R¹⁰ is selected from the group consisting of aryl and aryl substituted with one or more independently selected R²¹ groups, and (d) R⁹ is selected from the group consisting of heteroaryl and heteroaryl substituted with one or more independently selected R²¹ groups.

In another embodiment of this invention: (a) R¹ is an alkyl group substituted with one phenyl group, or (b) R¹ is an alkyl group substituted with one phenyl group, and said phenyl group is substituted with one or more independently selected R²² groups, and (c) R¹⁰ is selected from the group consisting of phenyl and phenyl substituted with one or more independently selected R²¹ groups, and (d) R⁹ is selected from the group consisting of imidazolyl and imidazolyl substituted with one or more independently selected R²¹ groups.

In another embodiment of this invention: (a) R¹ is a methyl or ethyl group substituted with one phenyl, or (b) R¹ is an methyl or alkyl group substituted with one phenyl, and said phenyl is substituted with one or more independently selected halos, and (c) R¹⁰ is selected from the group consisting of phenyl and phenyl substituted with one or more independently selected —OR¹⁵ groups, and (d) R⁹ is selected from the group consisting of imidazolyl and imidazolyl substituted with one or more independently selected alkyl groups groups.

In another embodiment of this invention: (a) R¹ is a methyl or ethyl group substituted with one phenyl, or (b) R¹ is an methyl or alkyl group substituted with one phenyl, and said phenyl is substituted with one or two independently selected halos, and (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups groups.

In another embodiment of this invention: (a) R¹ is a methyl or ethyl group substituted with one phenyl, or (b) R¹ is an methyl or alkyl group substituted with one phenyl, and said phenyl is substituted with one or two F, and (c) R¹⁰ is selected from the group consisting of phenyl and phenyl substituted with one or two independently selected —OR¹⁵ groups, wherein R¹⁵ is methyl, and (d) R⁹ is selected from the group consisting of imidazolyl and imidazolyl substituted with one or two independently selected methyl groups groups.

In another embodiment of this invention: (a) R¹ is a methyl or ethyl group substituted with one phenyl, or (b) R¹ is an methyl or alkyl group substituted with one phenyl, and said phenyl is substituted with one or two F, and (c) R¹⁰ is phenyl substituted with one —OR¹⁵ group, wherein R¹⁵ is methyl, and (d) R⁹ is selected from the group consisting of imidazolyl and imidazolyl substituted with one methyl group.

In another embodiment of this invention R¹ is selected from the group consisting of:

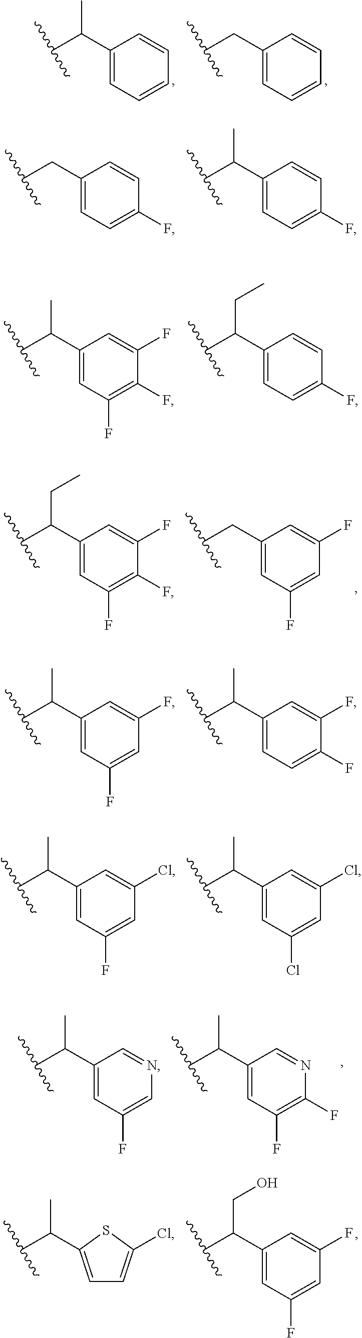

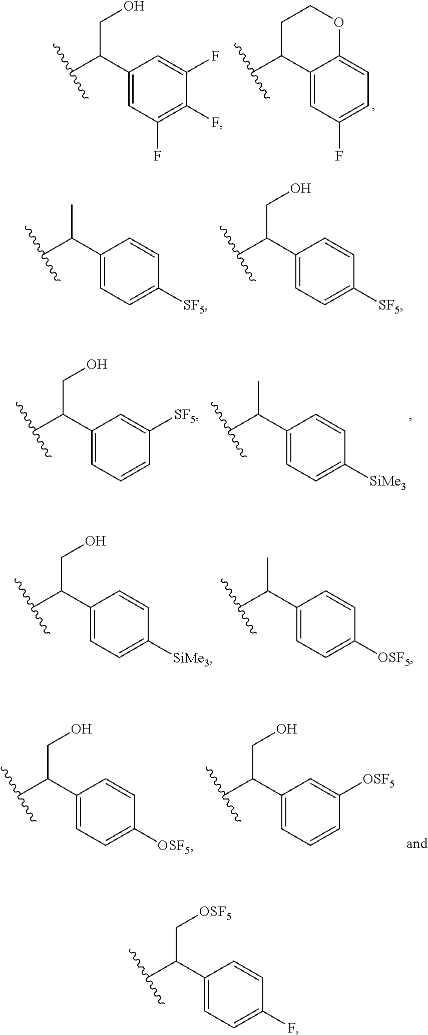

and
wherein the R⁹-R¹⁰— moiety is:

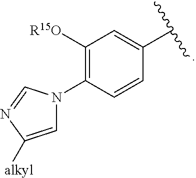

In another embodiment of this invention R¹ is selected from the group consisting of:

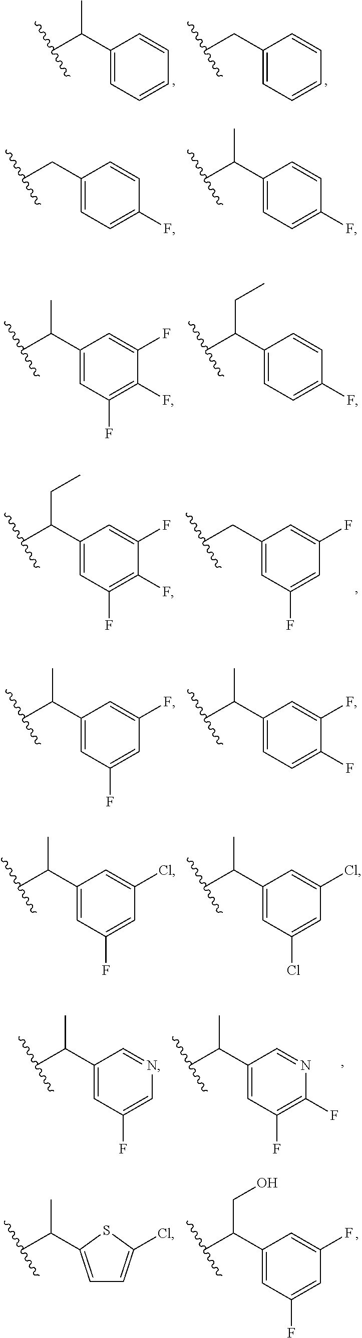

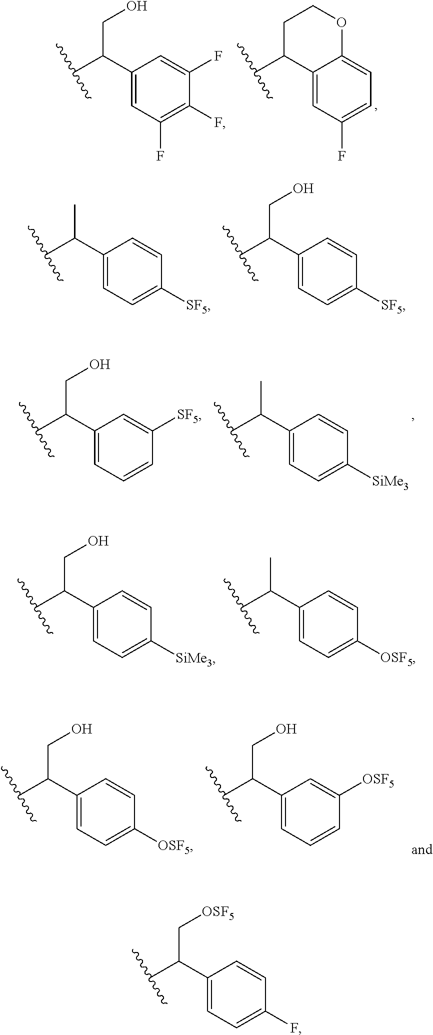

and
wherein the R⁹-R¹⁰— moiety is:

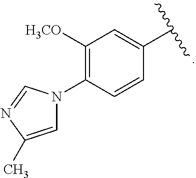

In another embodiment of this invention R¹ is selected from the group consisting of:

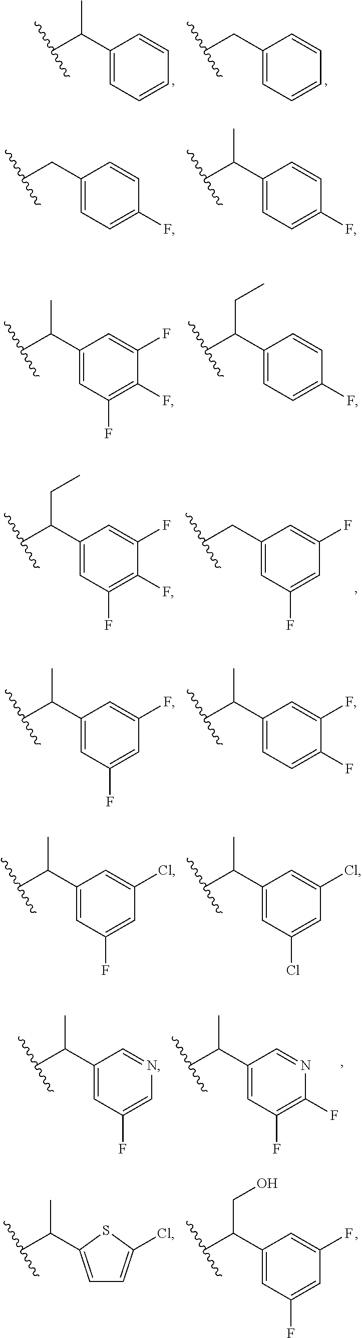

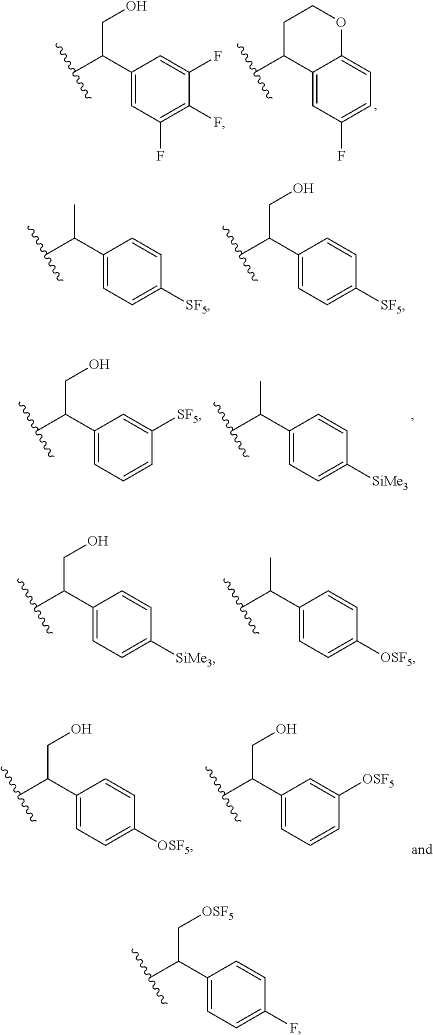

and
wherein the R⁹-R¹⁰— moiety is:

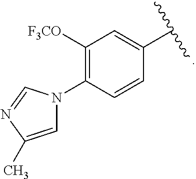

In another embodiment of this invention R¹ is selected from the group consisting of:

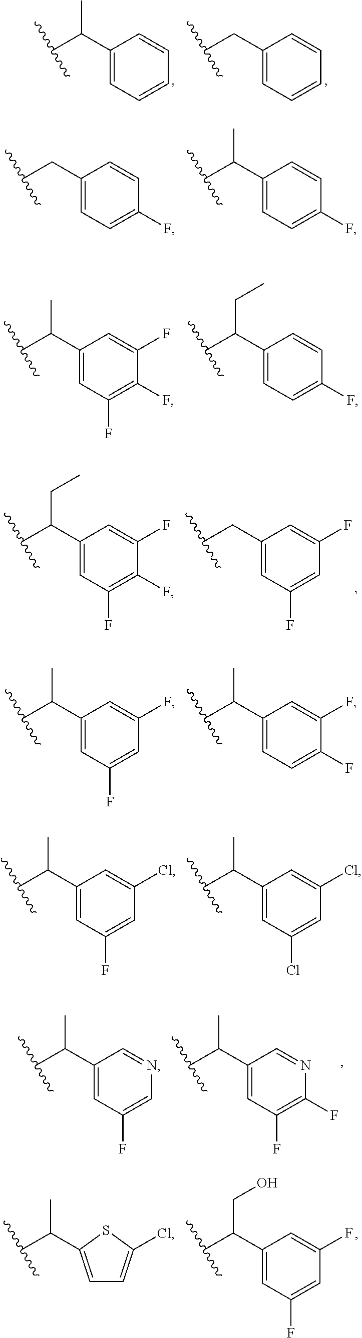

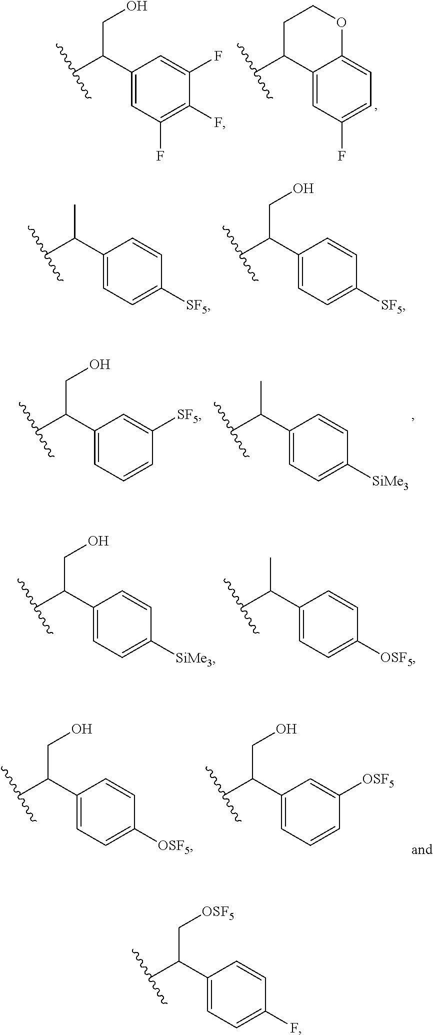

and
wherein the R⁹-R¹⁰— moiety is:

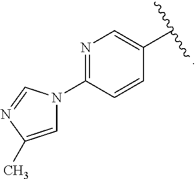

In another embodiment of this invention R¹ is selected from the group consisting of:

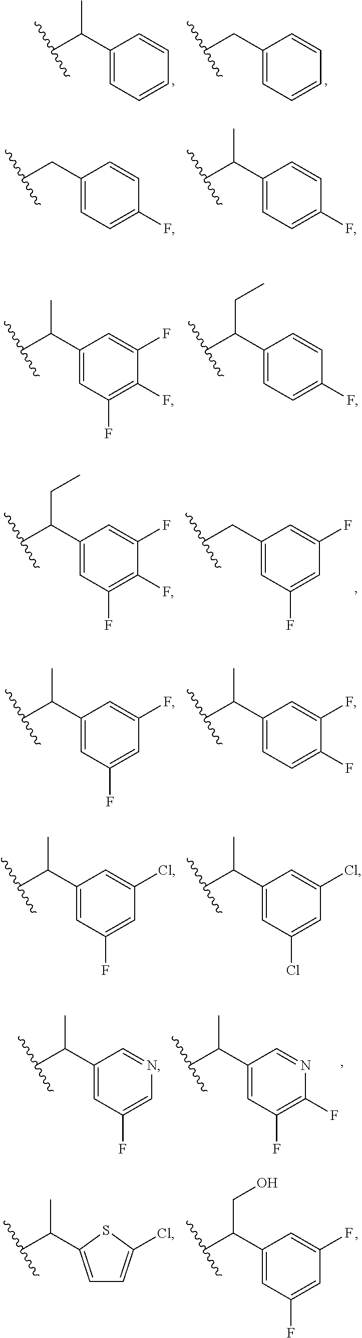

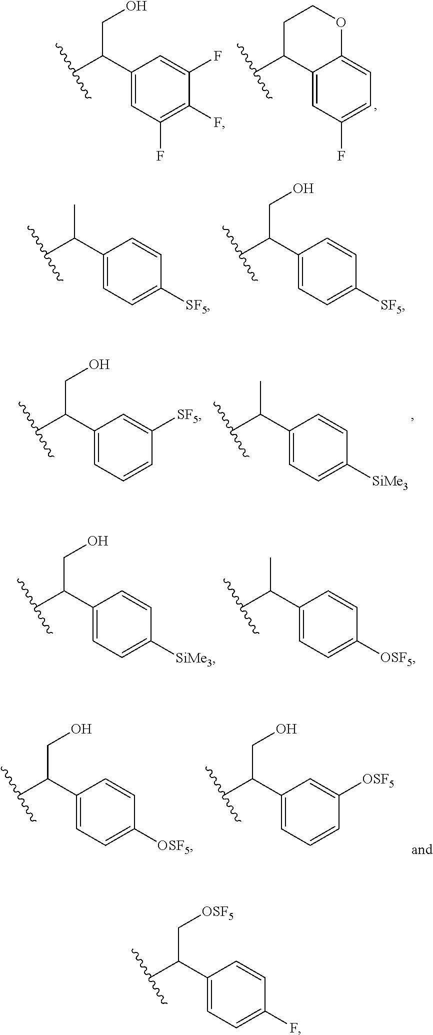

and
wherein the R⁹-R¹⁰— moiety is:

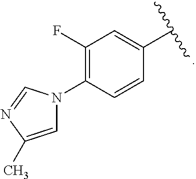

In another embodiment of this invention R¹ is selected from the group consisting of:

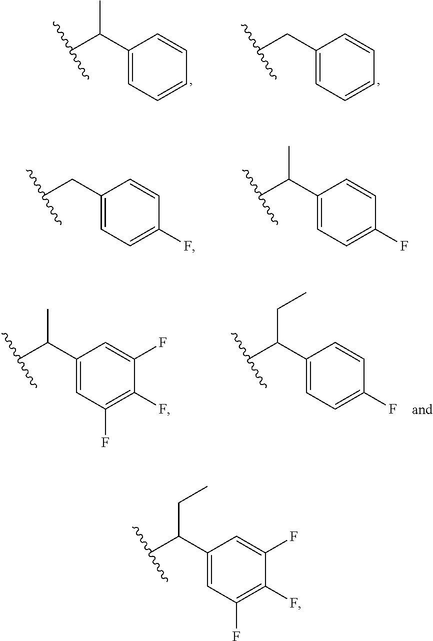

and
wherein the R⁹-R¹⁰— moiety is:

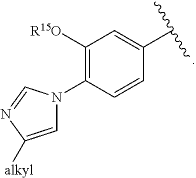

In another embodiment of this invention R¹ is selected from the group consisting of:

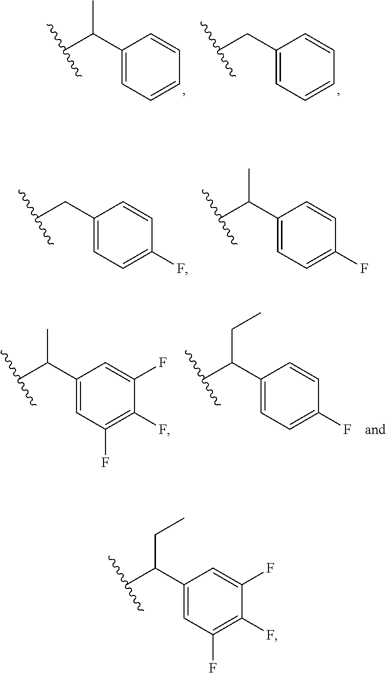

and
wherein the R⁹-R¹⁰— moiety is:

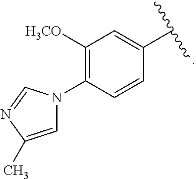

In another embodiment of this invention R¹ is selected from the group consisting of:

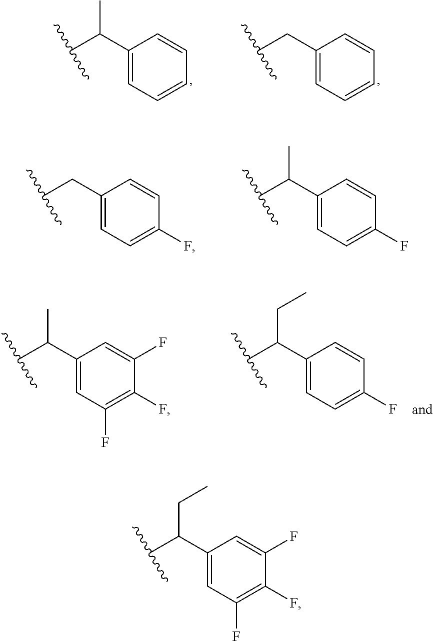

and
wherein the R⁹-R¹⁰— moiety is:

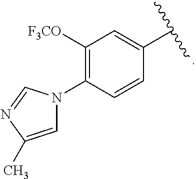

In another embodiment of this invention R¹ is selected from the group consisting of:

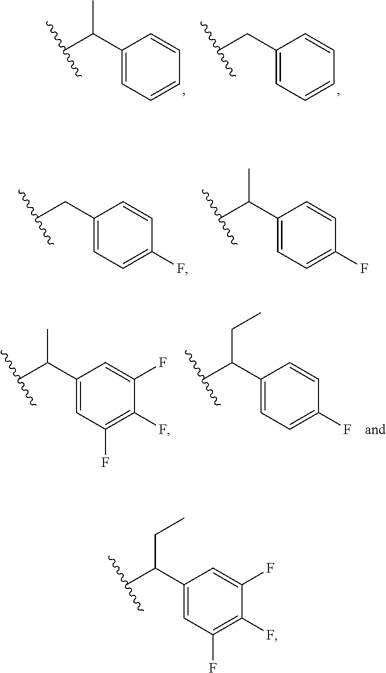

and
wherein the R⁹-R¹⁰— moiety is:

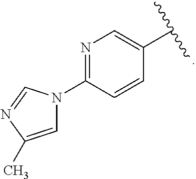

In another embodiment of this invention R¹ is selected from the group consisting of:

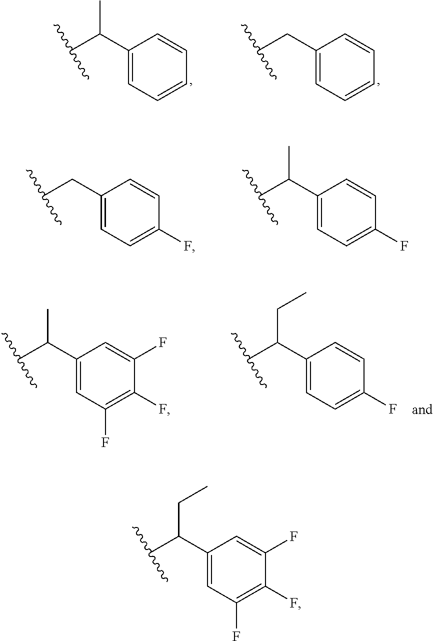

and
wherein the R⁹-R¹⁰— moiety is:

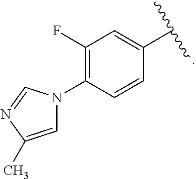

In another embodiment of this invention R¹ is selected from the group consisting of:

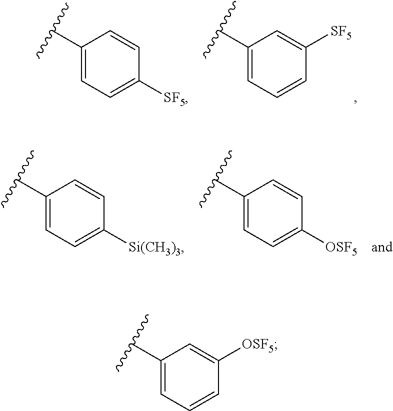

and
the R⁹-R¹⁰— moiety is selected from the group consisting of:

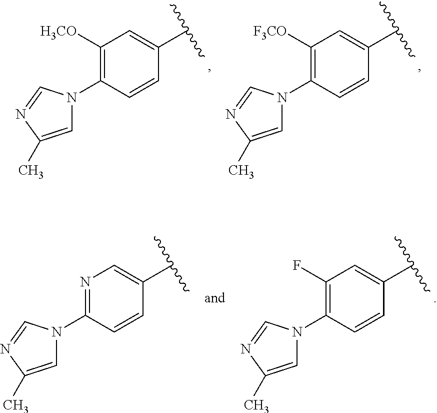

In another embodiment of this invention R⁷ is selected from the group consisting of:

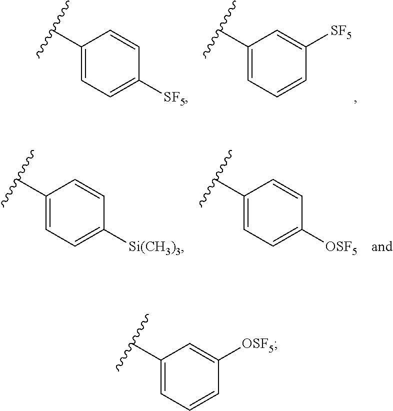

and
the R⁹-R¹⁹— moiety is:

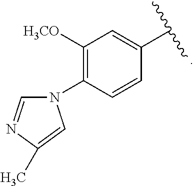

In another embodiment of this invention W is —C(O)—.

In another embodiment of this invention W is —S(O)—.

In another embodiment of this invention W is —S(O)₂—.

In another embodiment of this invention W is —C(═NR¹⁴)—.

In another embodiment of this invention G is —NH—.

In another embodiment of this invention G is a direct bond.

In another embodiment of this invention: (a) R¹ is a methyl or ethyl group substituted with one phenyl, or (b) R¹ is an methyl or alkyl group substituted with one phenyl, and said phenyl is substituted with one or two independently selected halos, and (c) R¹⁰ is selected from the group consisting of phenyl and phenyl substituted with one or two independently selected —OR¹⁵ groups, wherein R¹⁵ is alkyl, and (d) R⁹ is selected from the group consisting of imidazolyl and imidazolyl substituted with one or two independently selected alkyl groups groups, and (e) G is selected from the group consisting of —NH—, and a direct bond.

In another embodiment of this invention: (a) R¹ is a methyl or ethyl group substituted with one phenyl, or (b) R¹ is an methyl or alkyl group substituted with one phenyl, and said phenyl is substituted with one or two F, and (c) R¹⁰ is phenyl substituted with one —OR¹⁵ group, wherein R¹⁵ is methyl, and (d) R⁹ is selected from the group consisting of imidazolyl and imidazolyl substituted with one methyl group, and (e) G is selected from the group consisting of —NH—, and a direct bond.

In another embodiment of this invention R¹ is selected from the group consisting of:

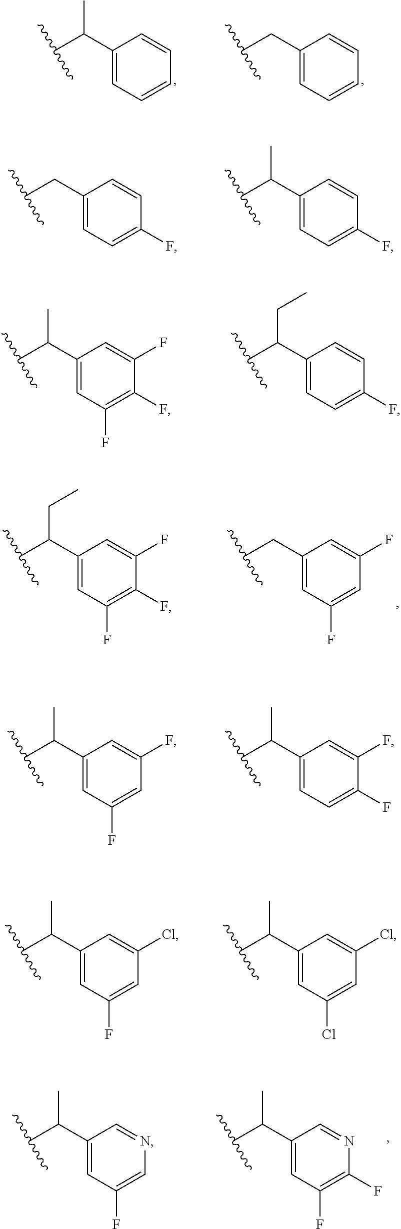

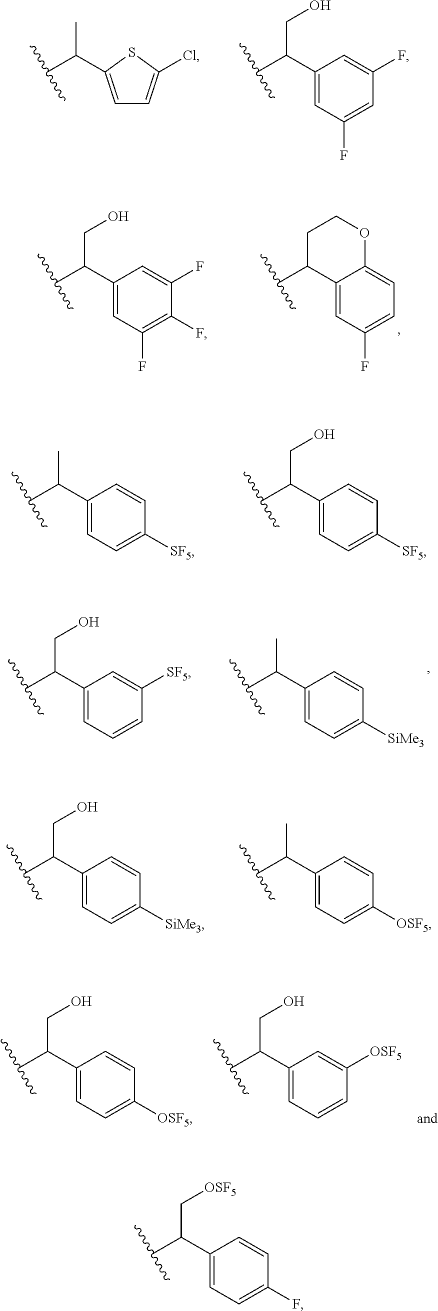

and
wherein the R⁹-R¹⁰— moiety is:

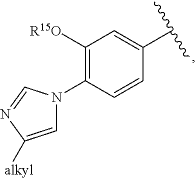

and
G is selected from the group consisting of —NH—, and a direct bond.

In another embodiment of this invention R¹ is selected from the group consisting of:

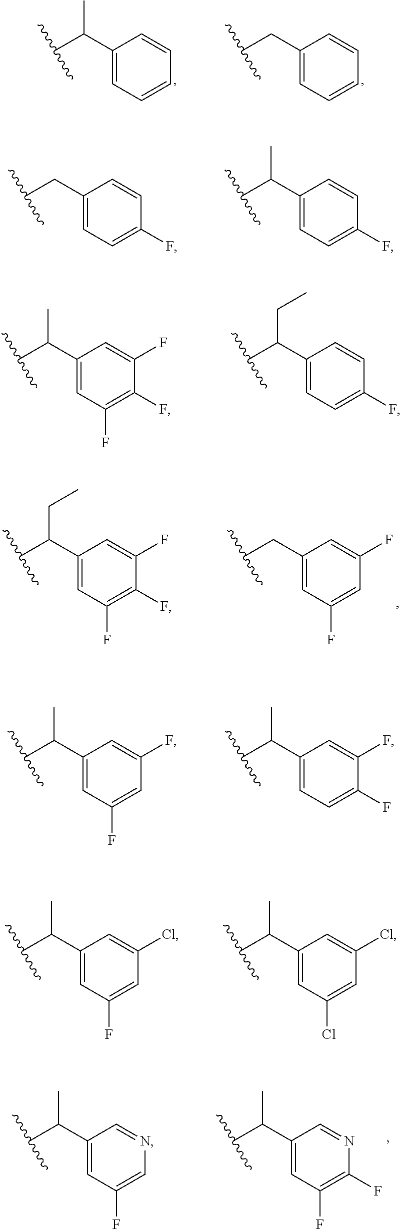

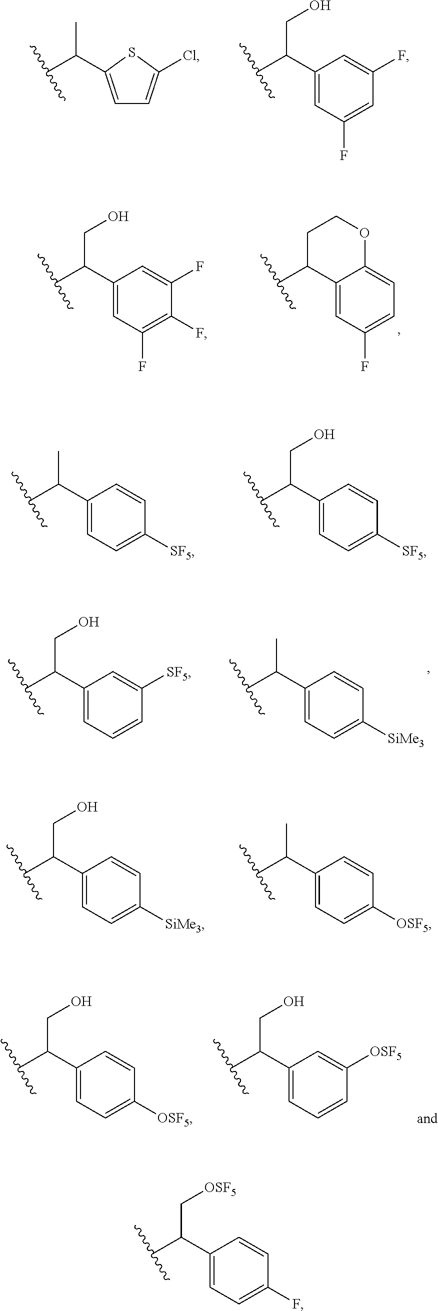

and
wherein the R⁹-R¹⁰— moiety is:

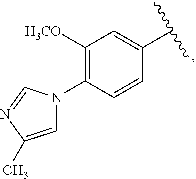

and
G is selected from the group consisting of —NH—, and a direct bond.

In another embodiment of this invention R¹ is selected from the group consisting of:

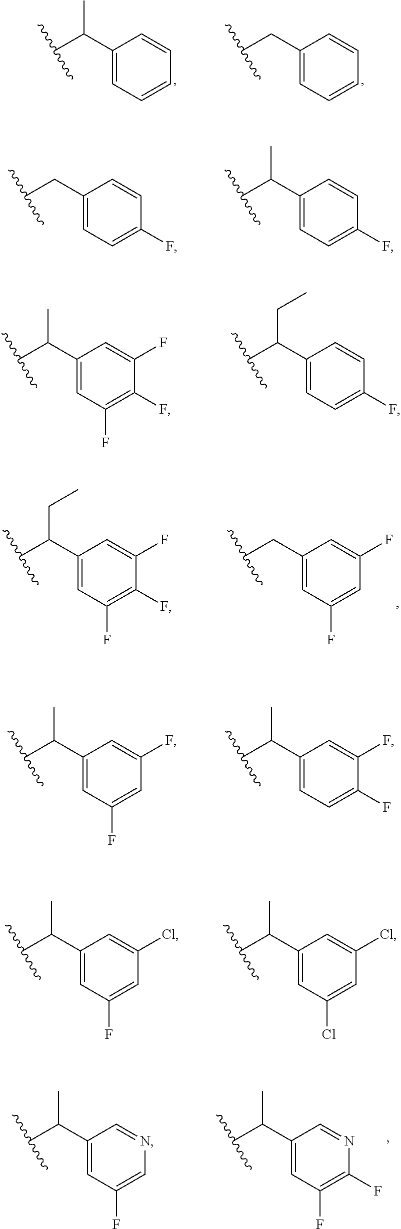

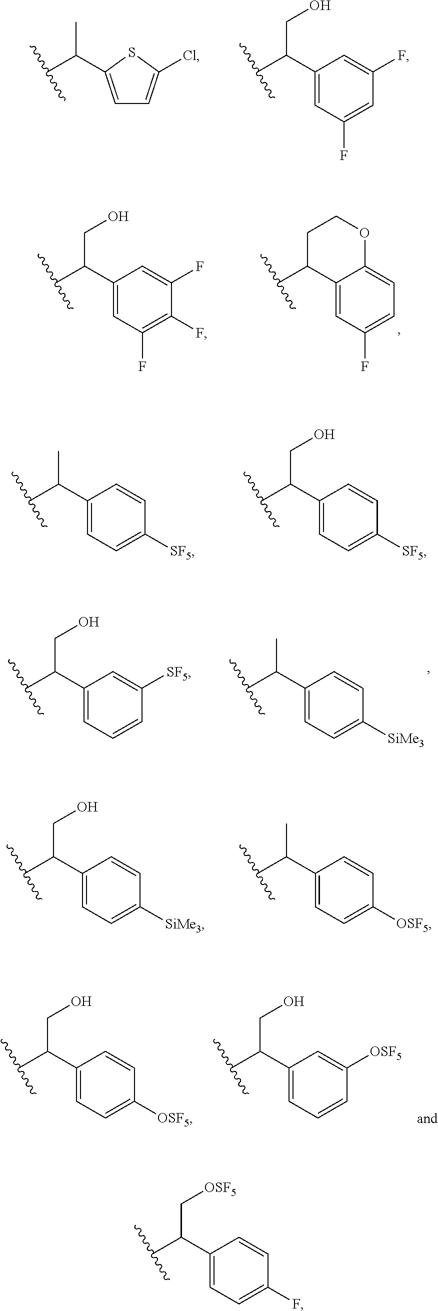

and
wherein the R⁹-R¹⁰— moiety is:

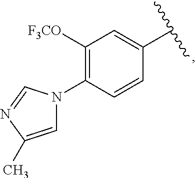

and
G is selected from the group consisting of —NH—, and a direct bond.

In another embodiment of this invention R¹ is selected from the group consisting of:

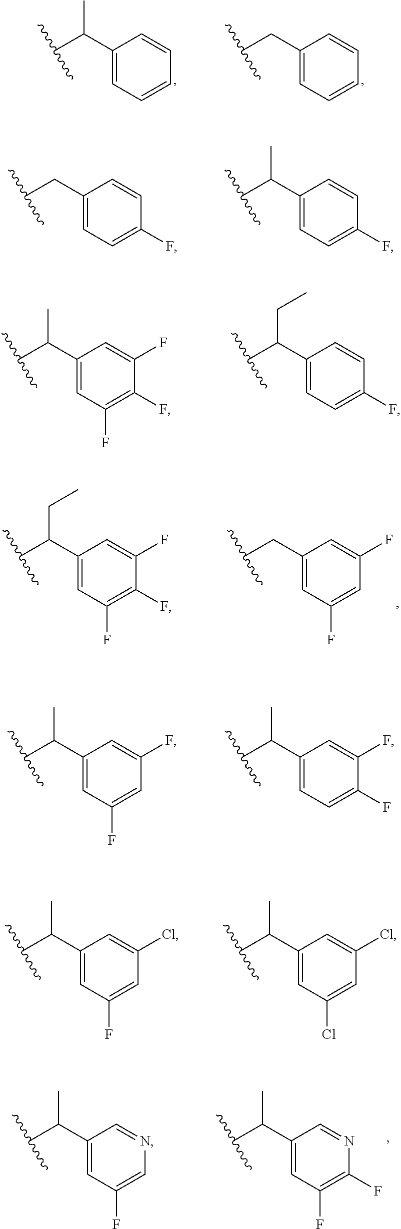

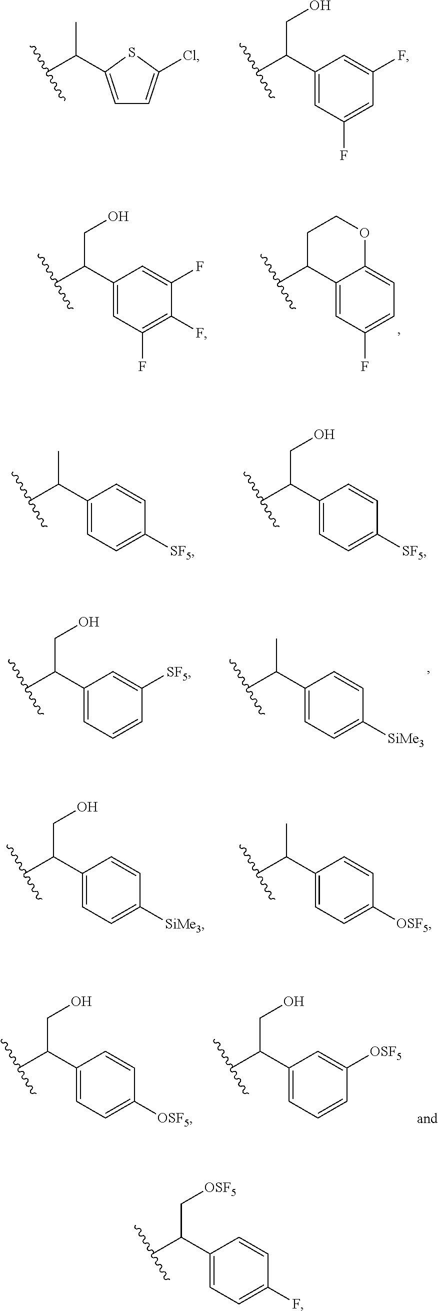

and
wherein the R⁹-R¹⁰— moiety is:

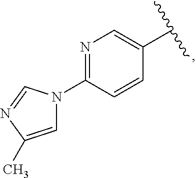

and
G is selected from the group consisting of —NH—, and a direct bond.

In another embodiment of this invention R¹ is selected from the group consisting of:

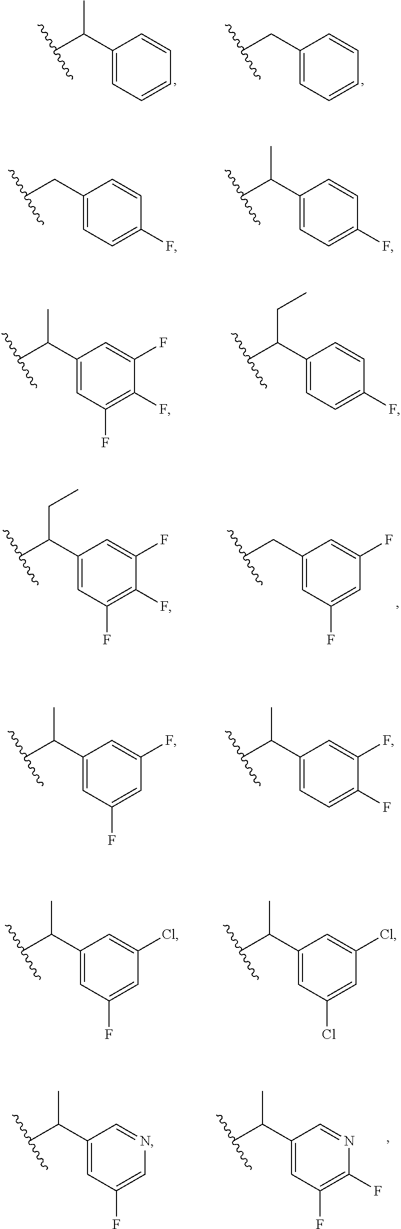

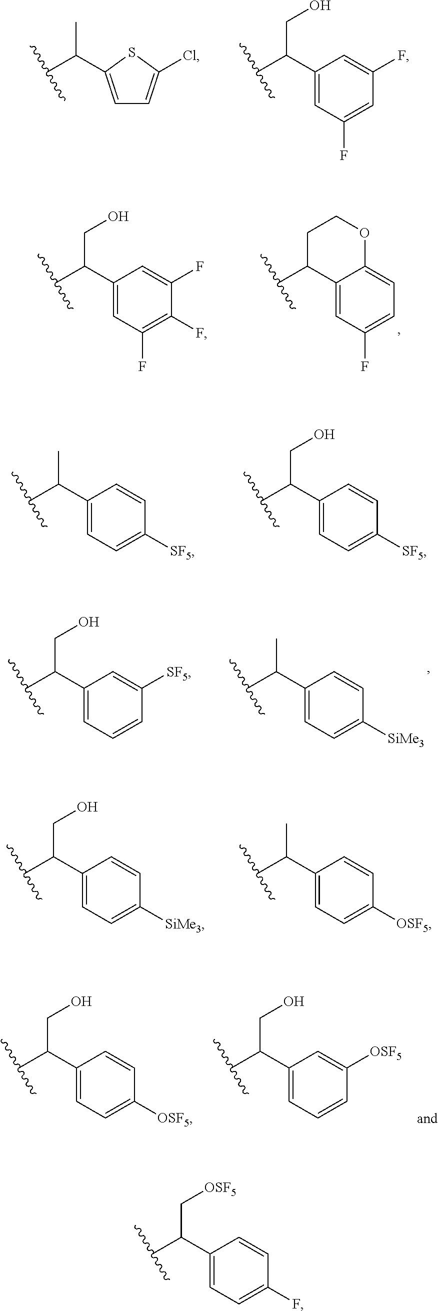

and
wherein the R⁹-R¹⁰— moiety is:

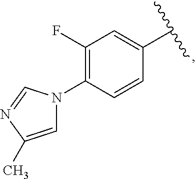

and
G is selected from the group consisting of —NH—, and a direct bond.

In another embodiment of this invention R¹ is selected from the group consisting of:

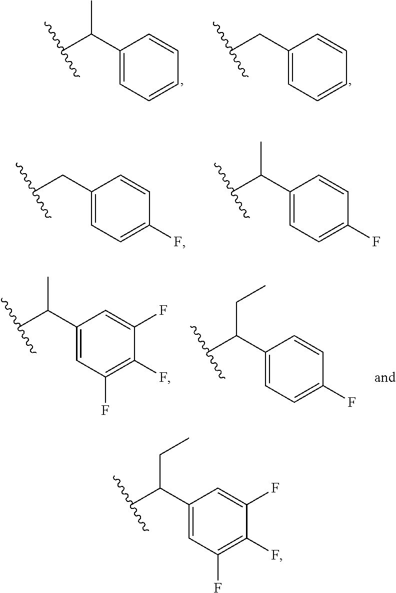

and
wherein the R⁹-R¹⁰— moiety is:

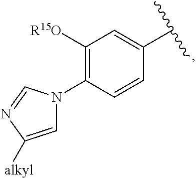

and
G is selected from the group consisting of —NH—, and a direct bond.

In another embodiment of this invention R¹ is selected from the group consisting of:

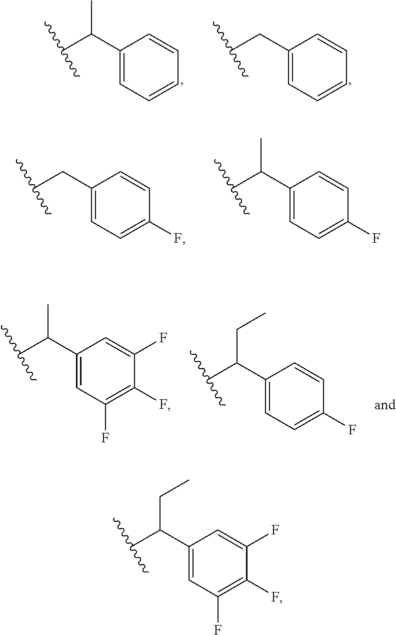

and
wherein the R⁹-R¹⁰— moiety is:

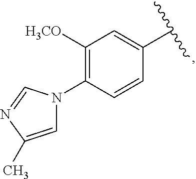

and
G is selected from the group consisting of —NH—, and a direct bond.

In another embodiment of this invention R¹ is selected from the group consisting of:

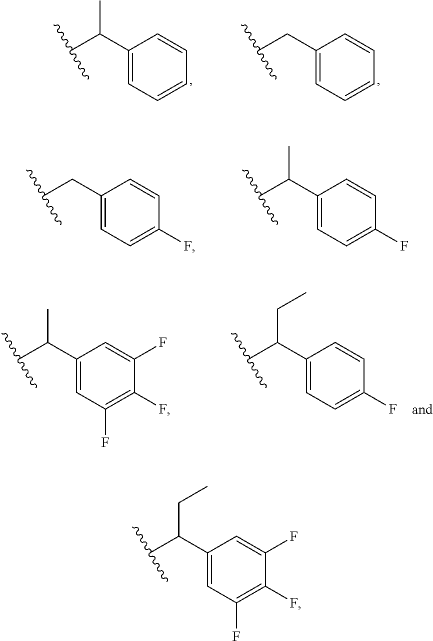

and
wherein the R⁹-R¹⁰— moiety is:

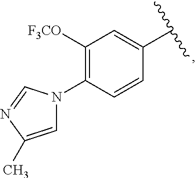

and
G is selected from the group consisting of —NH—, and a direct bond.

In another embodiment of this invention is selected from the group consisting of:

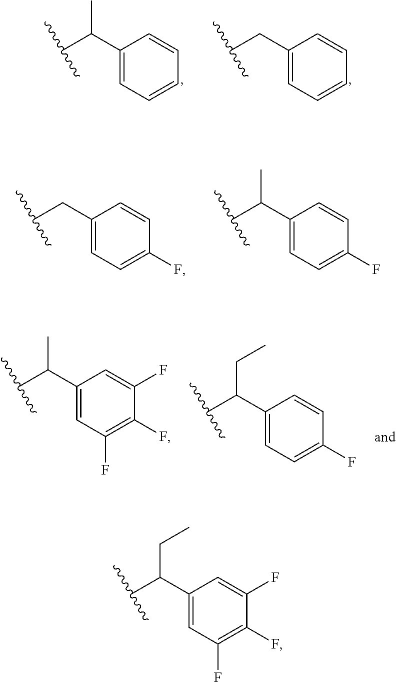

and
wherein the R⁹-R¹⁰— moiety is:

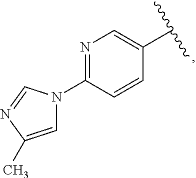

and
G is selected from the group consisting of —NH—, and a direct bond.

In another embodiment of this invention R¹ is selected from the group consisting of:

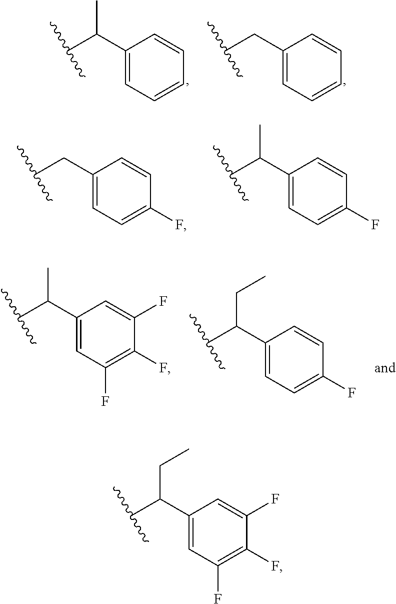

and
wherein the R⁹-R¹⁰— moiety is:

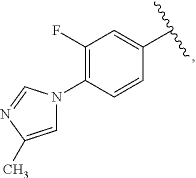

and
G is selected from the group consisting of —NH—, and a direct bond.

In another embodiment of this invention: (a) R¹ is a methyl or ethyl group substituted with one phenyl, or (b) R¹ is an methyl or alkyl group substituted with one phenyl, and said phenyl is substituted with one or two F, and (c) R¹⁰ is phenyl substituted with one —OR¹⁵ group, wherein R¹⁵ is methyl, and (d) R⁹ is selected from the group consisting of imidazolyl and imidazolyl substituted with one methyl group, and (e) G is selected from the group consisting of —NH—, and a direct bond, and (f) W is —C(O)—.

In another embodiment of this invention R¹ is selected from the group consisting of:

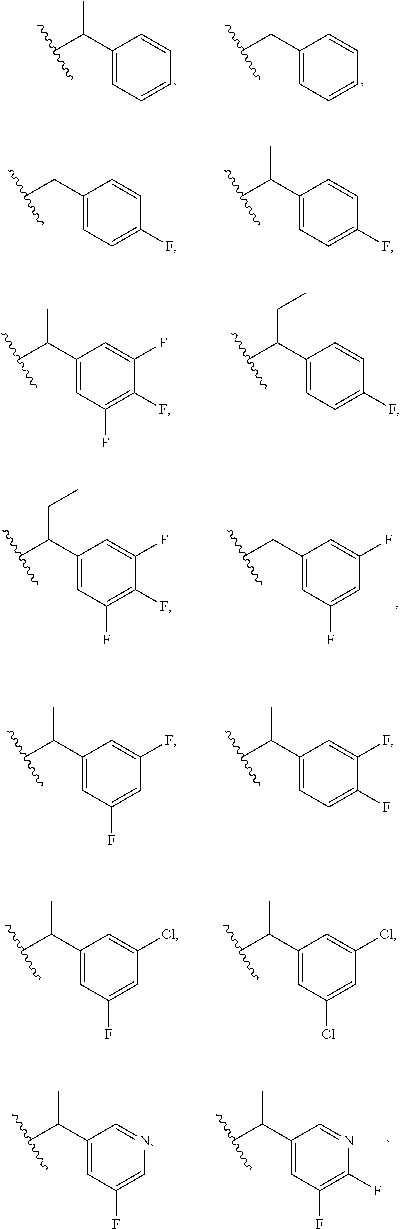

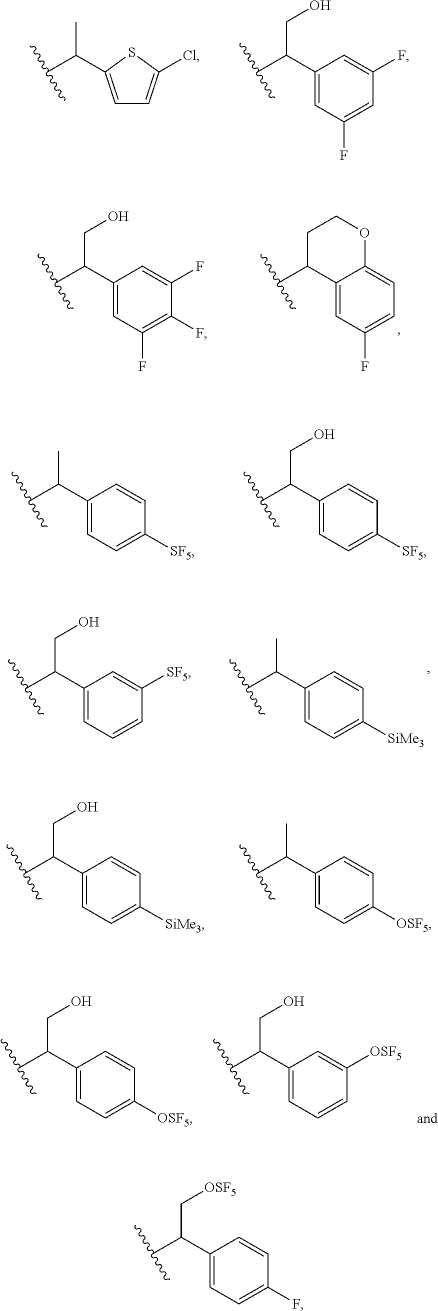

and
wherein the R⁹-R¹⁰— moiety is:

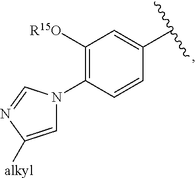

and
G is selected from the group consisting of —NH—, and a direct bond, and W is —C(O)—.

In another embodiment of this invention R¹ is selected from the group consisting of:

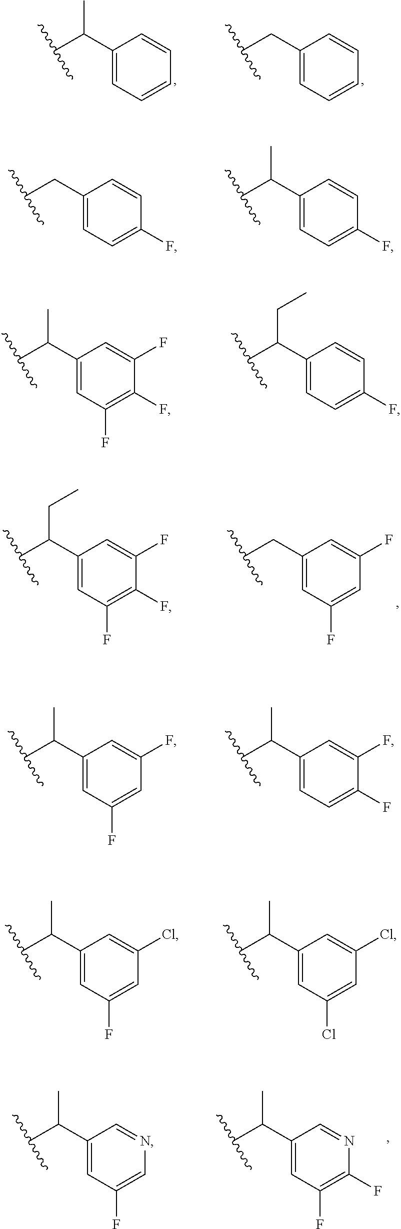

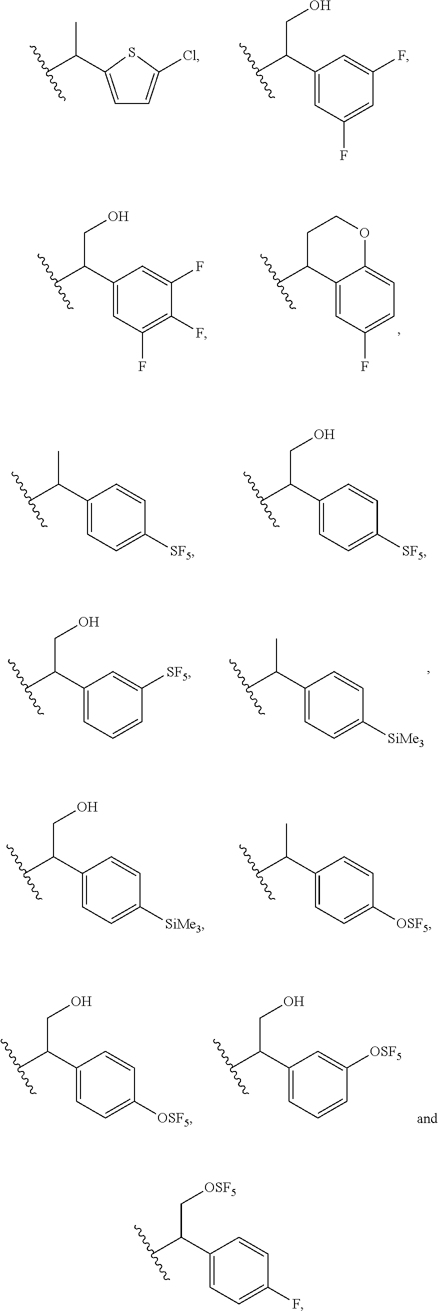

and
wherein the R⁹-R¹⁰— moiety is:

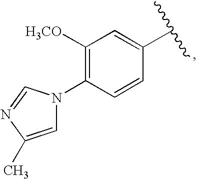

and
G is selected from the group consisting of —NH—, and a direct bond, and W is —C(O)—.

In another embodiment of this invention R¹ is selected from the group consisting of:

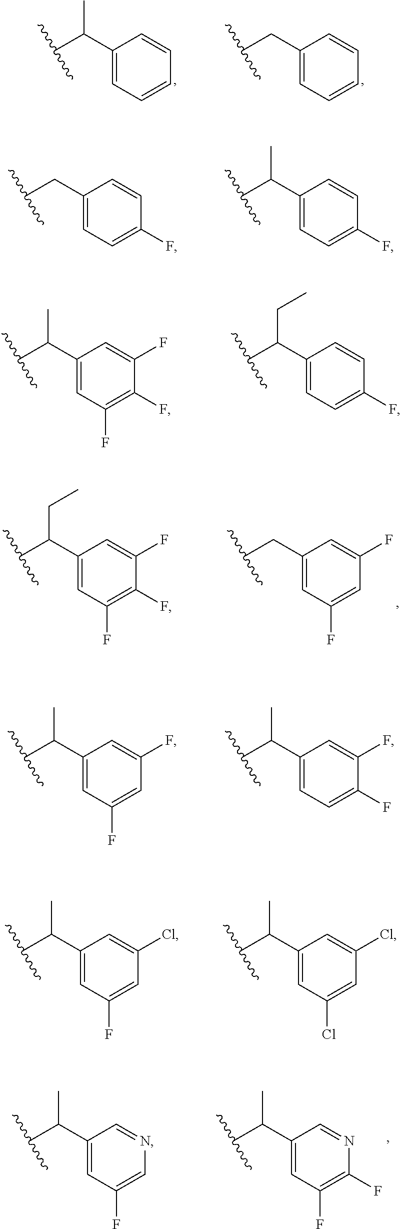

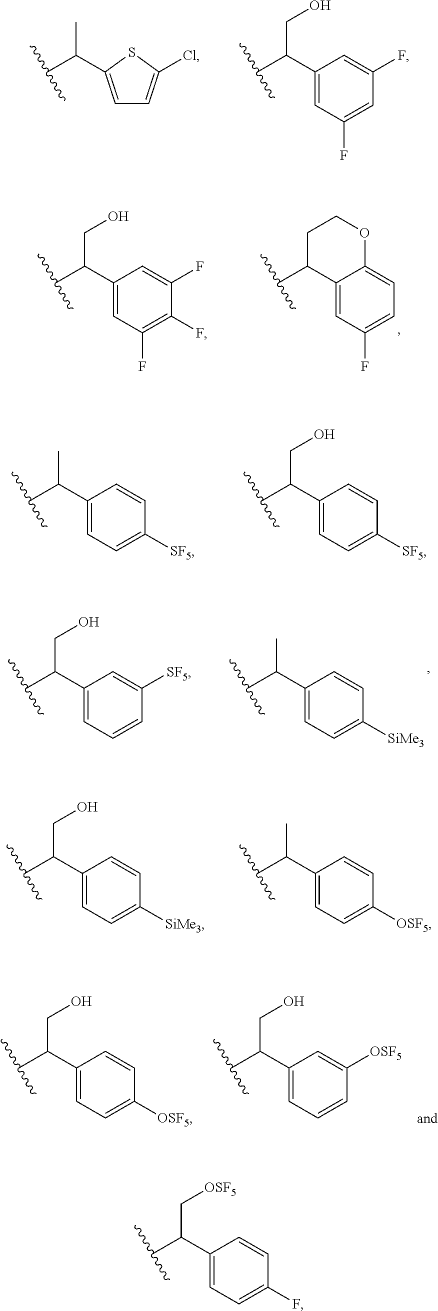

and
wherein the R⁹-R¹⁰— moiety is:

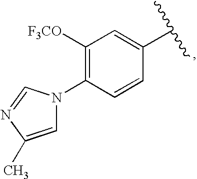

and
G is selected from the group consisting of —NH—, and a direct bond, and W is —C(O)—.

In another embodiment of this invention R¹ is selected from the group consisting of:

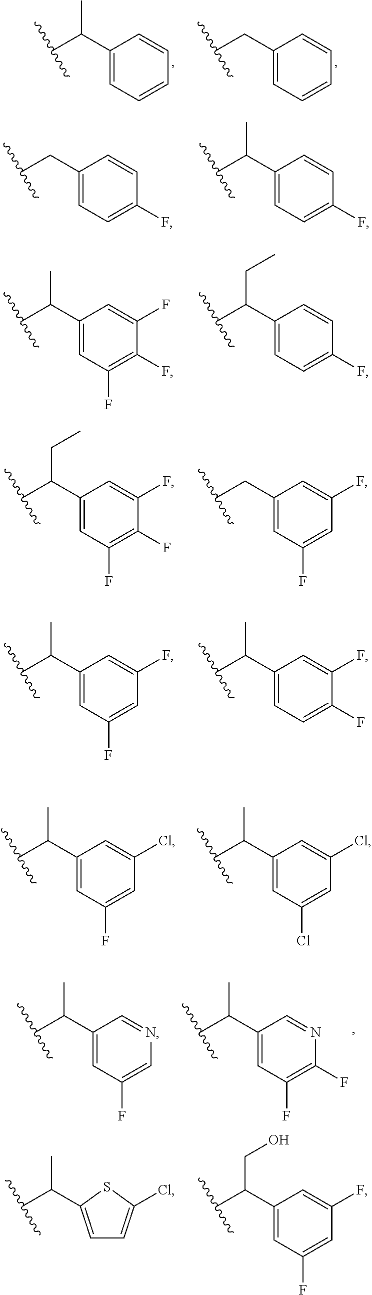

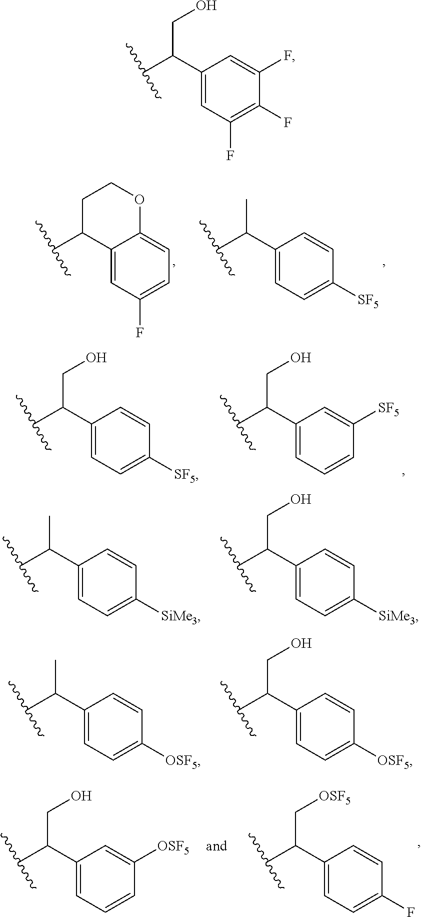

and
wherein the R⁹-R¹⁰— moiety is:

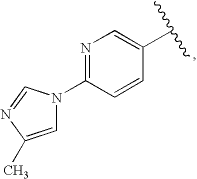

and
G is selected from the group consisting of —NH—, and a direct bond, and W is —C(O)—.

In another embodiment of this invention R¹ is selected from the group consisting of:

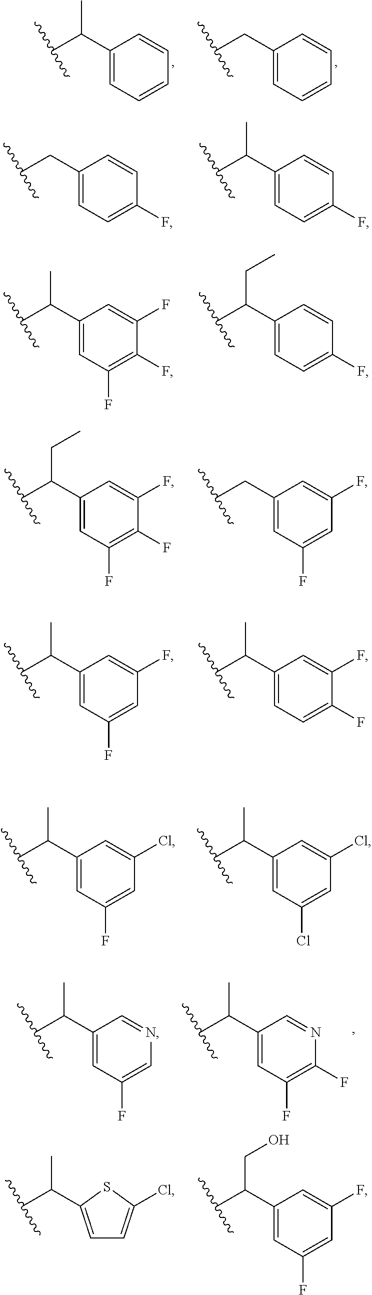

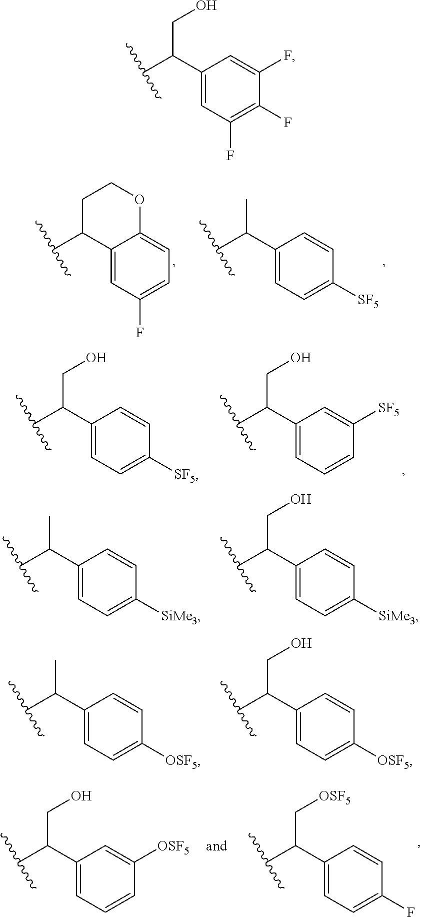

and
wherein the R⁹-R¹⁰— moiety is:

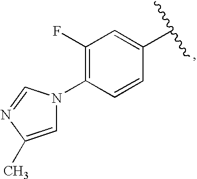

and
G is selected from the group consisting of —NH—, and a direct bond, and W is —C(O)—.

In another embodiment of this invention R¹ is selected from the group consisting of:

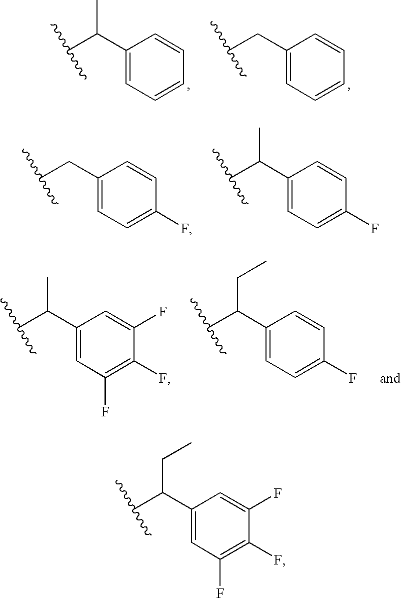

and
wherein the R⁹-R¹⁰— moiety is:

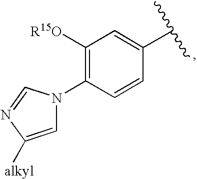

and
G is selected from the group consisting of —NH—, and a direct bond, and W is —C(O)—.

In another embodiment of this invention R¹ is selected from the group consisting of:

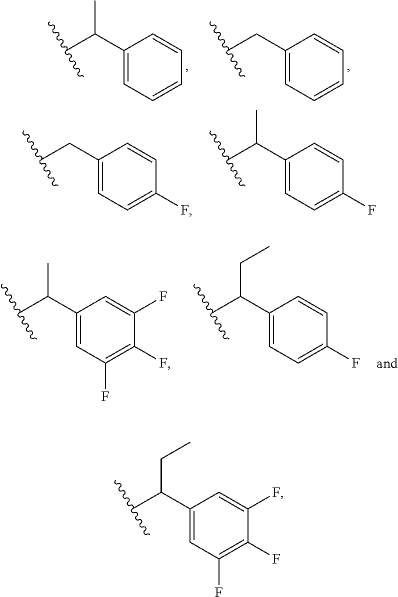

and
wherein the R⁹-R¹⁰— moiety is:

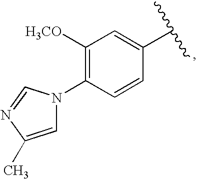

and
G is selected from the group consisting of —NH—, and a direct bond, and W is —C(O)—.

In another embodiment of this invention R¹ is selected from the group consisting of:

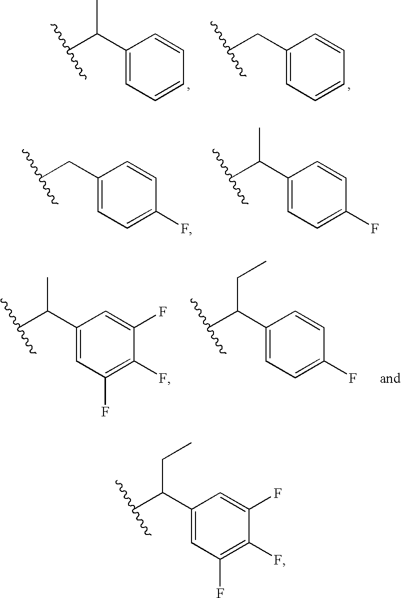

and
wherein the R⁹-R¹⁰— moiety is:

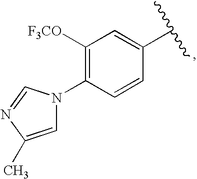

and
G is selected from the group consisting of —NH—, and a direct bond, and W is —C(O)—.

In another embodiment of this invention R¹ is selected from the group consisting of:

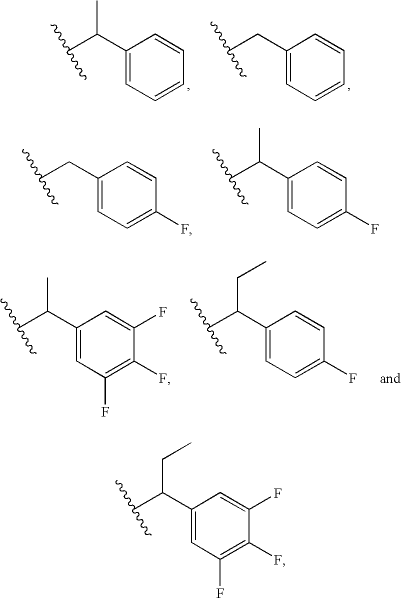

and
wherein the R⁹-R¹⁰— moiety is:

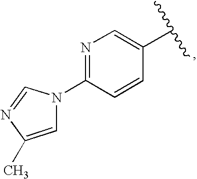

and
G is selected from the group consisting of —NH—, and a direct bond, and W is —C(O)—.

In another embodiment of this invention R¹ is selected from the group consisting of:

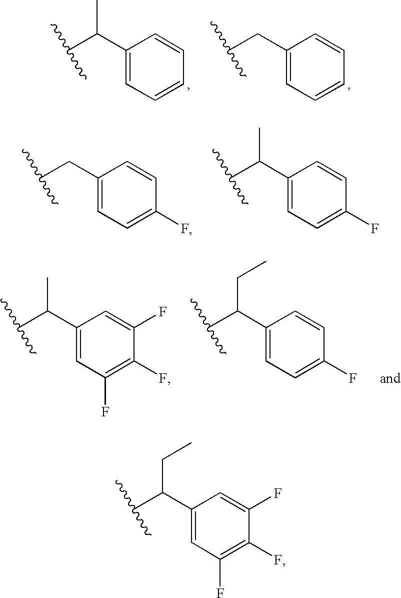

and
wherein the R⁹-R¹⁰— moiety is:

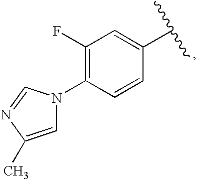

and
G is selected from the group consisting of —NH—, and a direct bond, and W is —C(O)—.

In another embodiment of this invention: (a) R¹ is a methyl or ethyl group substituted with one phenyl, or (b) R¹ is an methyl or alkyl group substituted with one phenyl, and said phenyl is substituted with one or two F, and (c) R¹⁰ is phenyl substituted with one —OR¹⁵ group, wherein R¹⁵ is methyl, and (d) R⁹ is selected from the group consisting of imidazolyl and imidazolyl substituted with one methyl group, and (e) G is selected from the group consisting of —NH—, and a direct bond, and (f) W is —S(O)—.

In another embodiment of this invention R¹ is selected from the group consisting of:

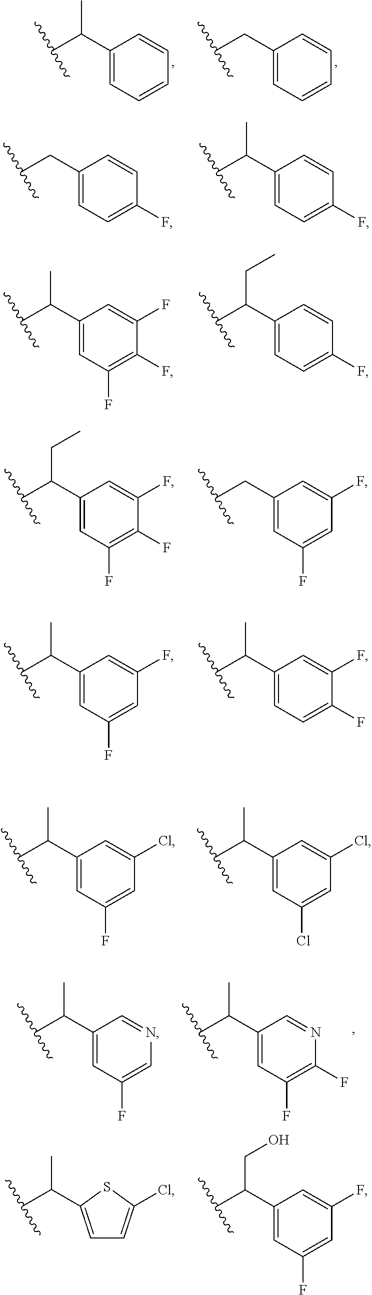

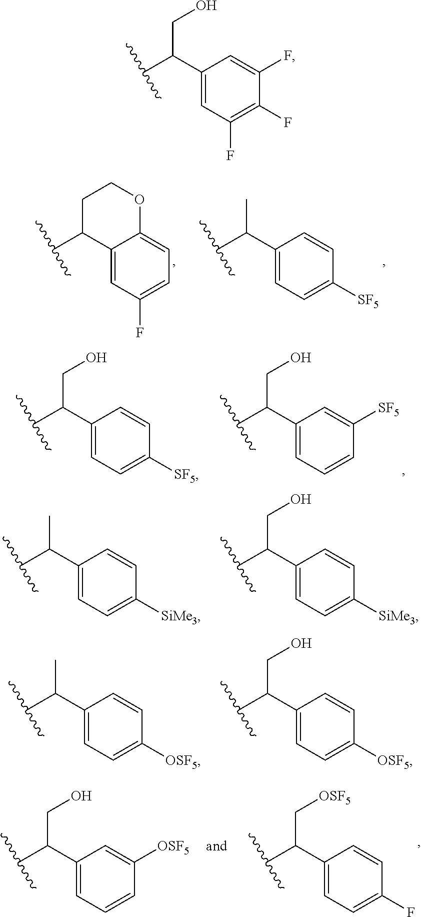

and
wherein the R⁹-R¹⁰— moiety is:

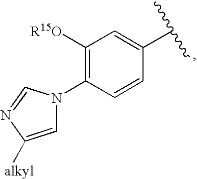

and
G is selected from the group consisting of —NH—, and a direct bond, and W is —S(O)—.

In another embodiment of this invention R¹ is selected from the group consisting of:

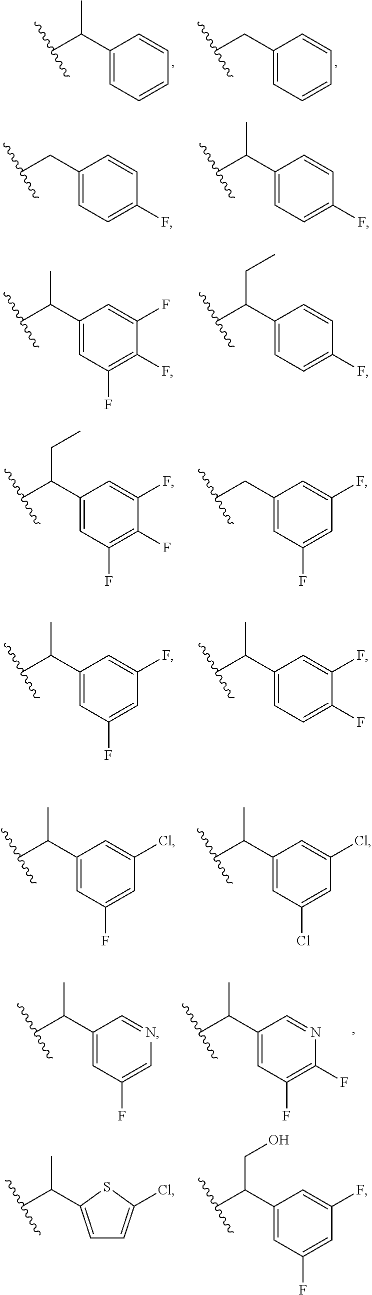

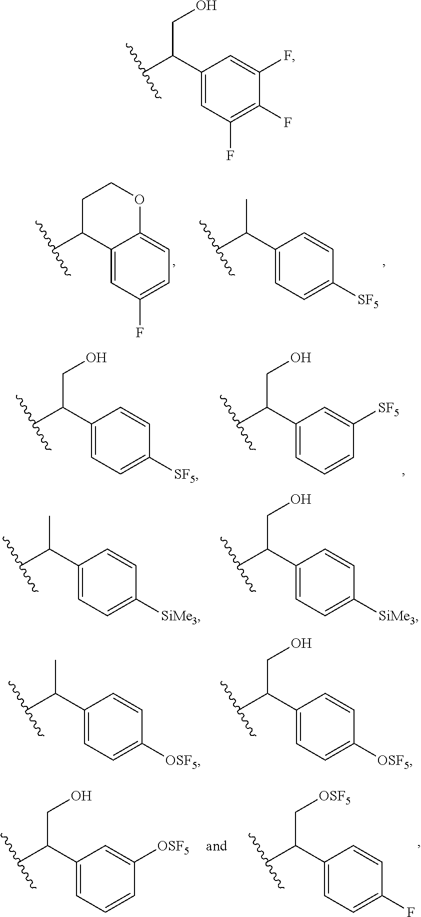

and
wherein the R⁹-R¹⁰— moiety is:

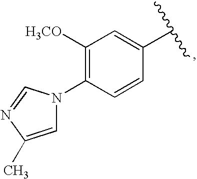

and
G is selected from the group consisting of —NH—, and a direct bond, and W is —S(O)—.

In another embodiment of this invention R¹ is selected from the group consisting of:

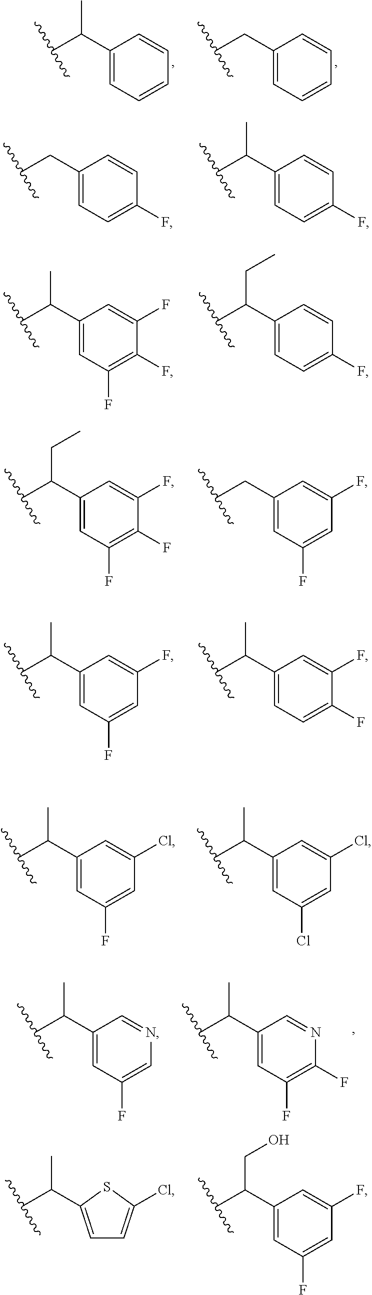

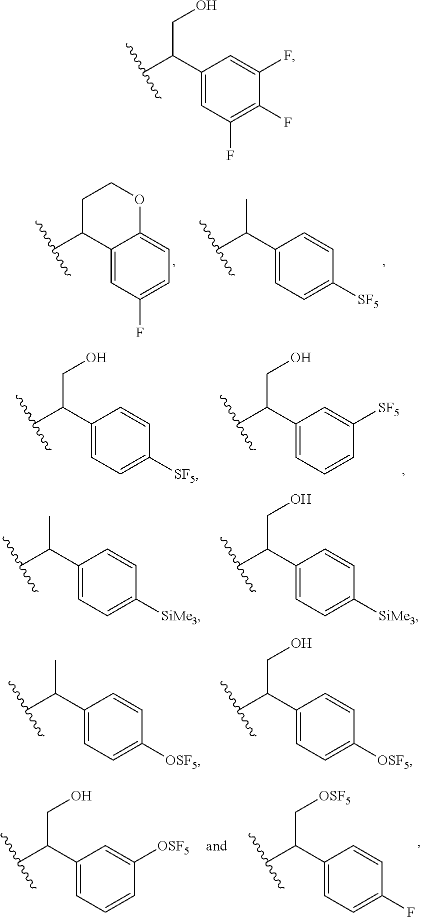

and
wherein the R⁹-R¹⁰— moiety is:

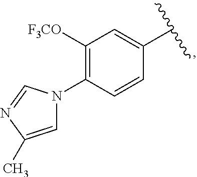

and
G is selected from the group consisting of —NH—, and a direct bond, and W is —S(O)—.

In another embodiment of this invention R¹ is selected from the group consisting of:

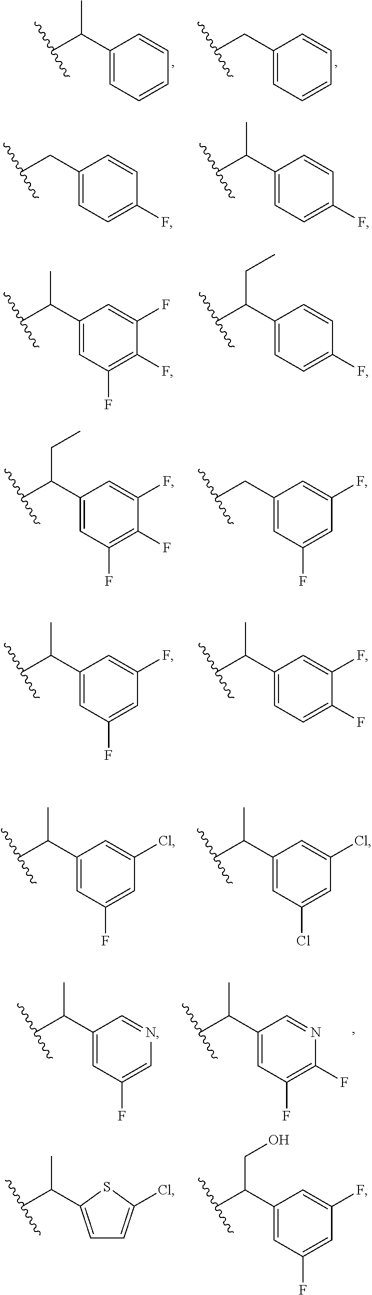

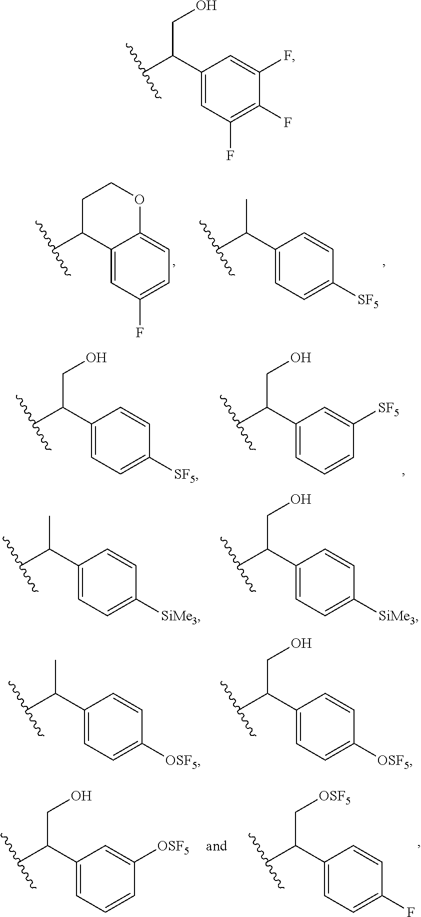

and
wherein the R⁹-R¹⁰— moiety is:

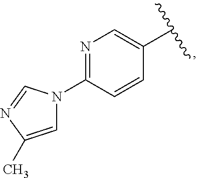

and
G is selected from the group consisting of —NH—, and a direct bond, and W is —S(O)—.

In another embodiment of this invention R¹ is selected from the group consisting of:

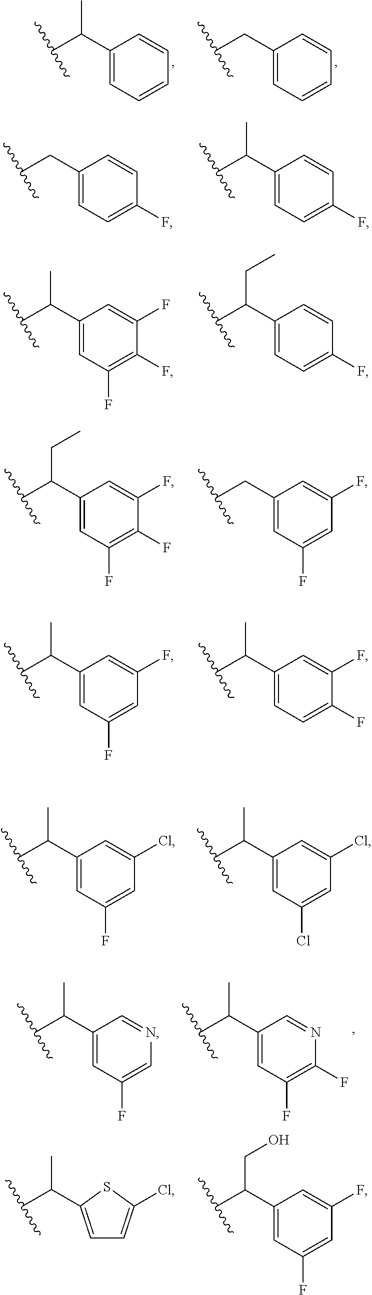

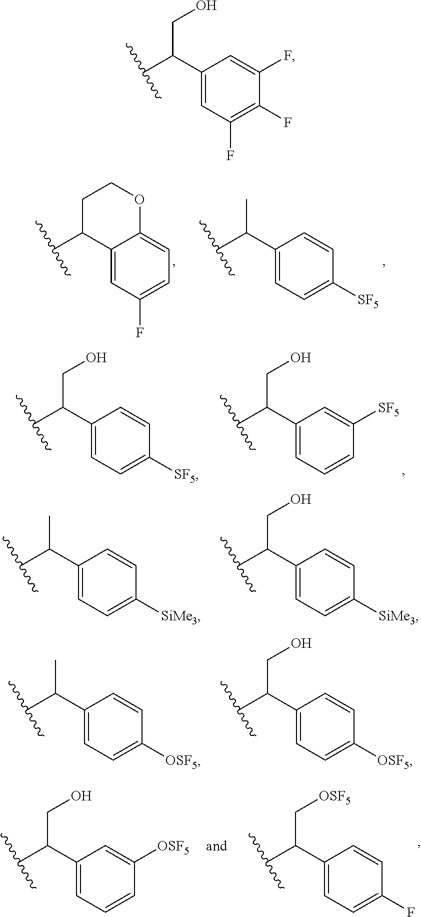

and
wherein the R⁹-R¹⁰— moiety is:

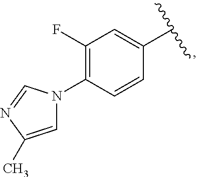

and
G is selected from the group consisting of —NH—, and a direct bond, and W is —S(O)—.

In another embodiment of this invention R¹ is selected from the group consisting of:

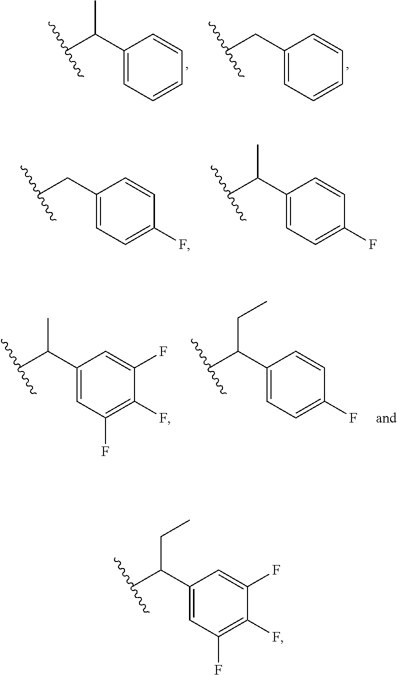

and
wherein the R⁹-R¹⁰— moiety is:

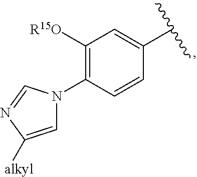

and
G is selected from the group consisting of —NH—, and a direct bond, and W is —S(O)—.

In another embodiment of this invention R¹ is selected from the group consisting of:

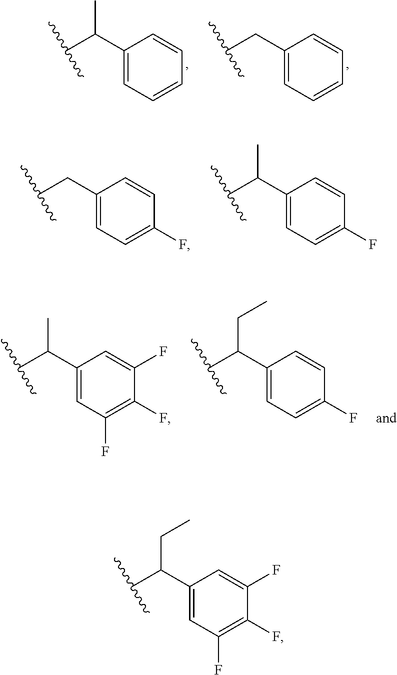

and
wherein the R⁹-R¹⁰— moiety is:

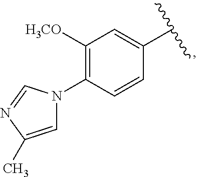

and
G is selected from the group consisting of —NH—, and a direct bond, and W is —S(O)—.

In another embodiment of this invention R¹ is selected from the group consisting of:

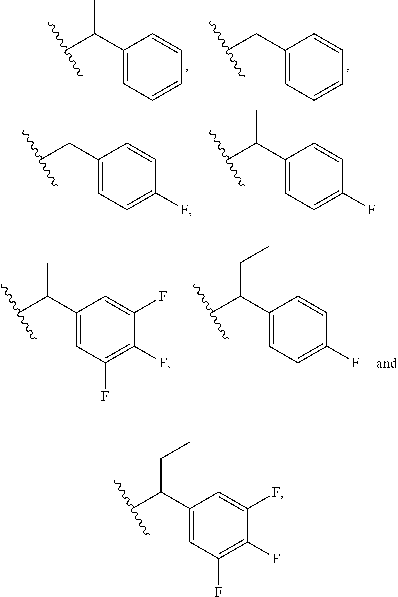

and
wherein the R⁹-R¹⁰— moiety is:

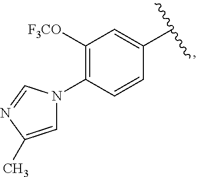

and
G is selected from the group consisting of —NH—, and a direct bond, and W is —S(O)—.

In another embodiment of this invention R¹ is selected from the group consisting of:

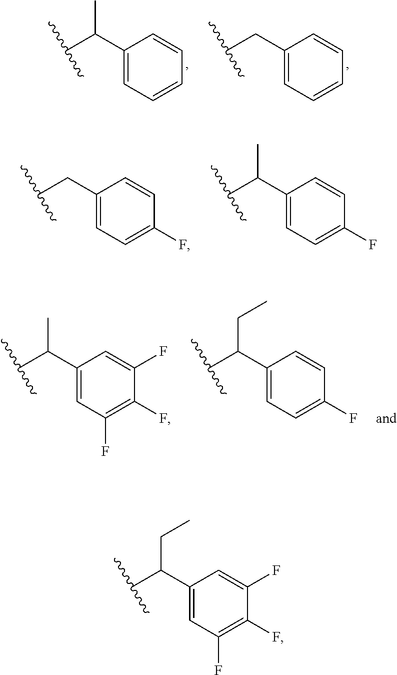

and
wherein the R⁹-R¹⁰— moiety is:

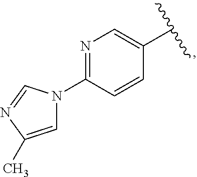

and
G is selected from the group consisting of —NH—, and a direct bond, and W is —S(O)—.

In another embodiment of this invention R¹ is selected from the group consisting of:

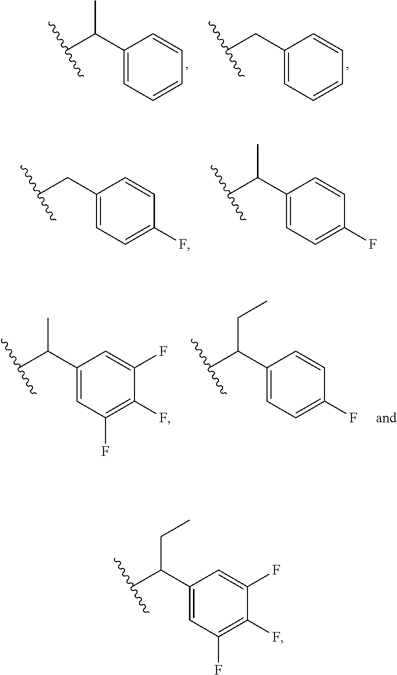

and
wherein the R⁹-R¹⁰— moiety is:

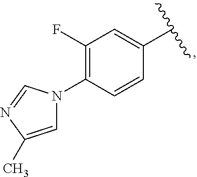

and
G is selected from the group consisting of —NH—, and a direct bond, and W is —S(O)—.

In another embodiment of this invention: (a) R¹ is a methyl or ethyl group substituted with one phenyl, or (b) R¹ is an methyl or alkyl group substituted with one phenyl, and said phenyl is substituted with one or two F, and (c) R¹⁰ is phenyl substituted with one —OR¹⁵ group, wherein R¹⁵ is methyl, and (d) R⁹ is selected from the group consisting of imidazolyl and imidazolyl substituted with one methyl group, and (e) G is selected from the group consisting of —NH—, and a direct bond, and (f) W is —S(O)₂—.

In another embodiment of this invention R¹ is selected from the group consisting of:

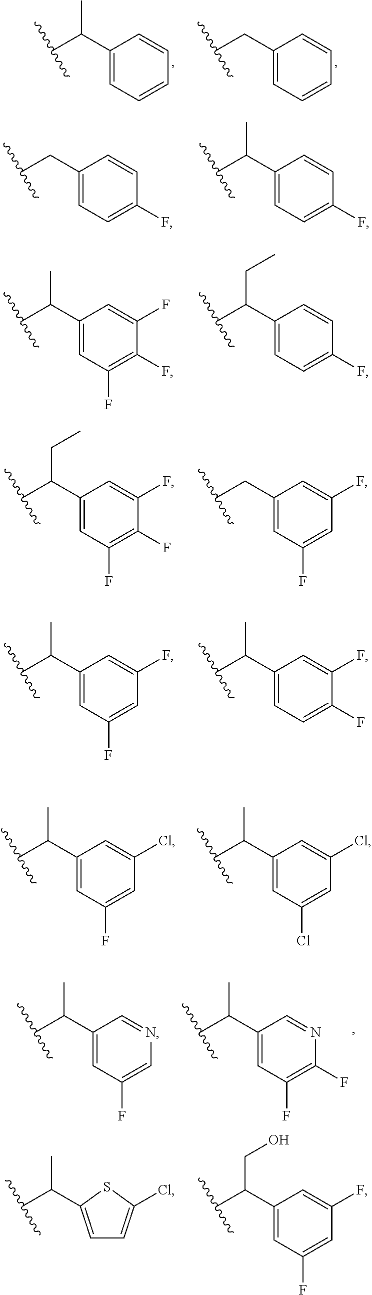

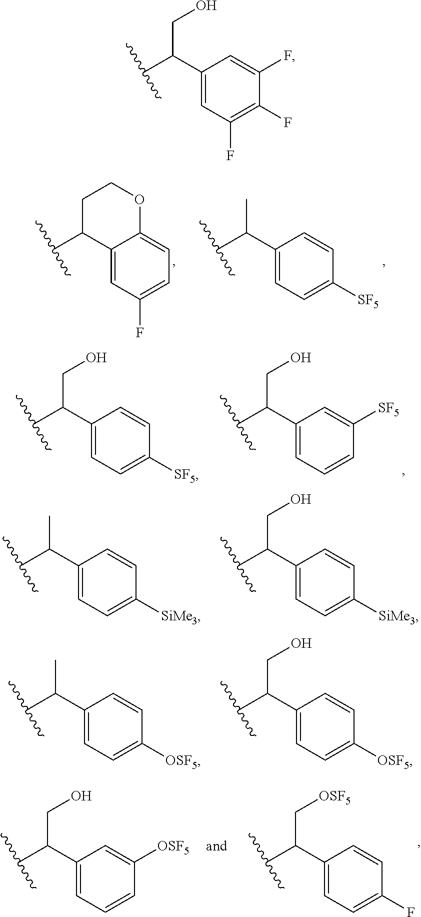

and
wherein the R⁹-R¹⁰— moiety is:

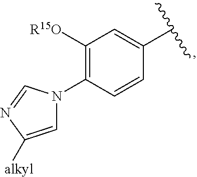

and
G is selected from the group consisting of —NH—, and a direct bond, and W is —S(O)₂—.

In another embodiment of this invention R¹ is selected from the group consisting of:

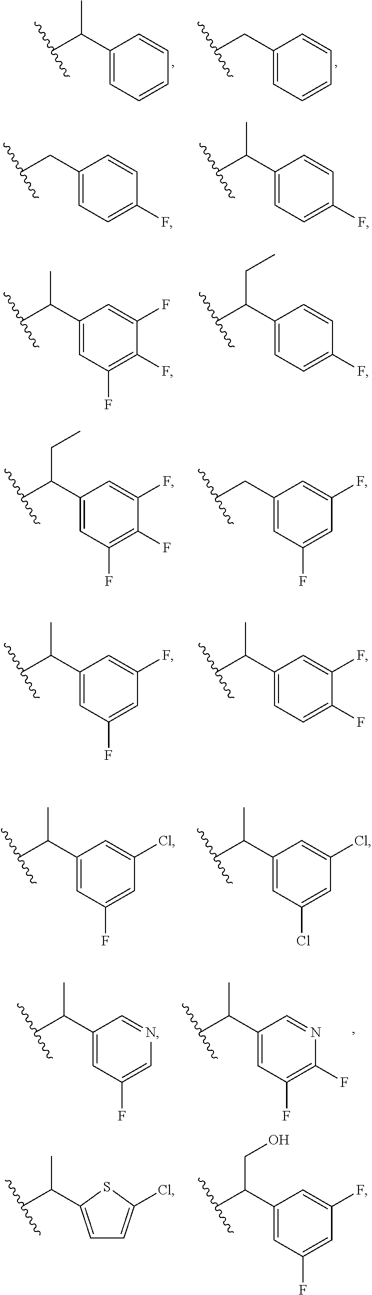

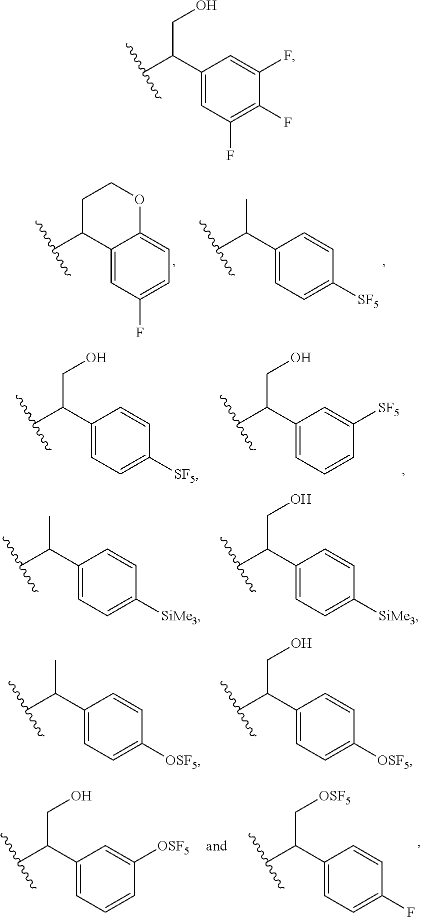

and
wherein the R⁹-R¹⁰— moiety is:

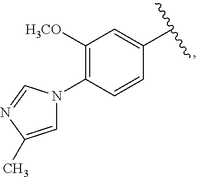

and
G is selected from the group consisting of —NH—, and a direct bond, and W is —S(O)₂—.

In another embodiment of this invention R¹ is selected from the group consisting of:

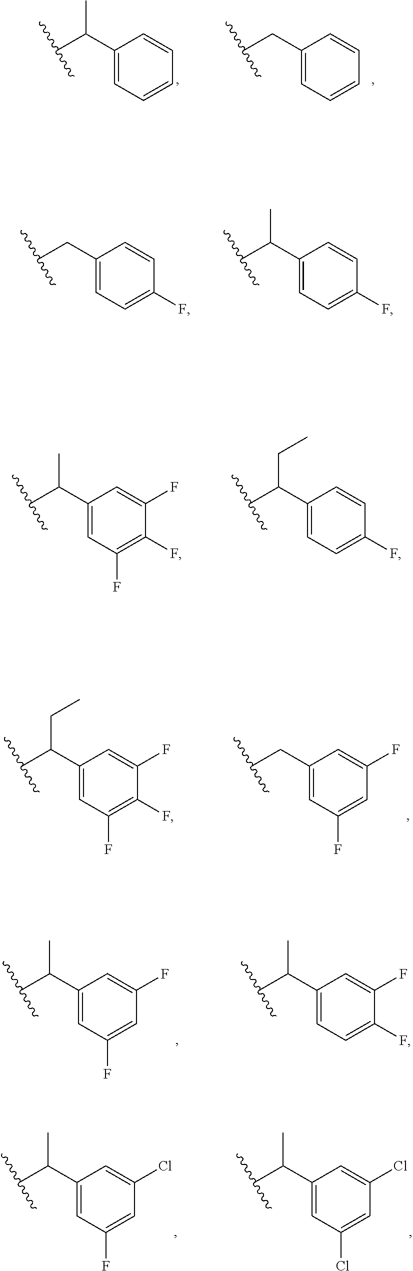

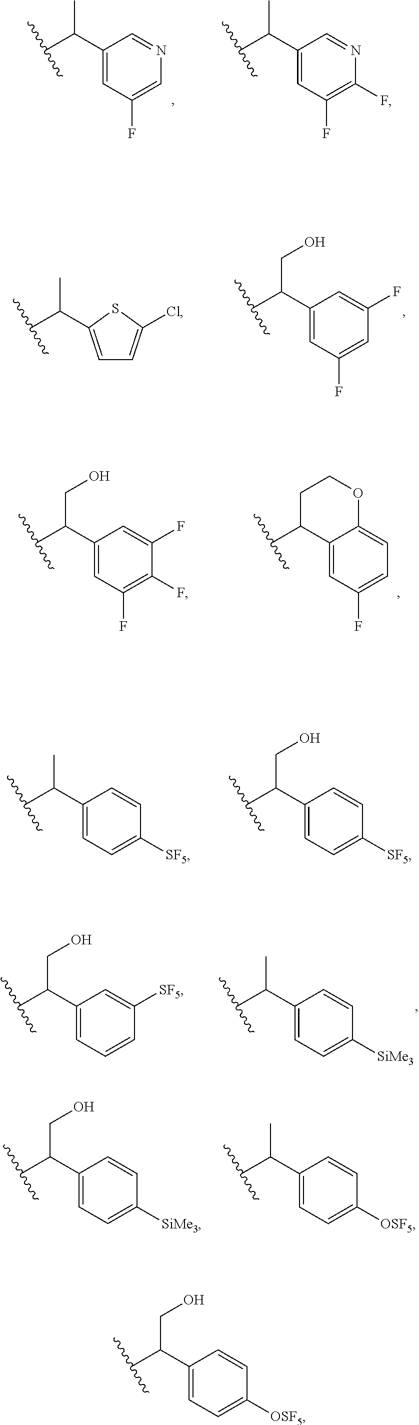

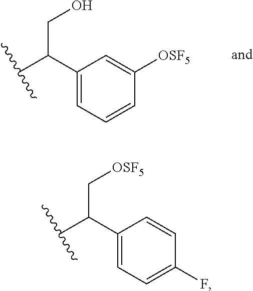

and
wherein the R⁹-R¹⁰— moiety is:

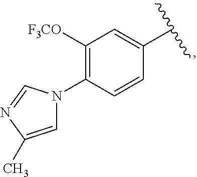

and
G is selected from the group consisting of —NH—, and a direct bond, and W is —S(O)₂—.

In another embodiment of this invention R¹ is selected from the group consisting of:

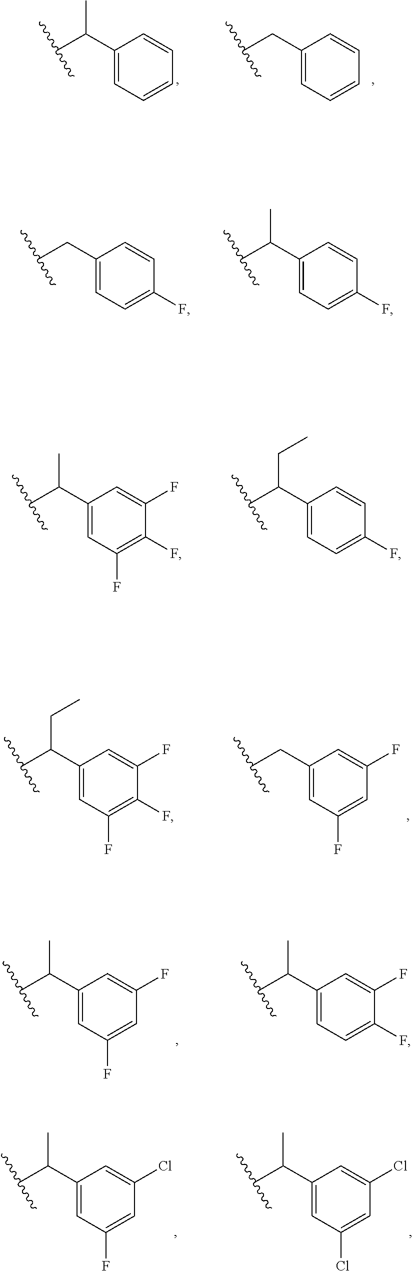

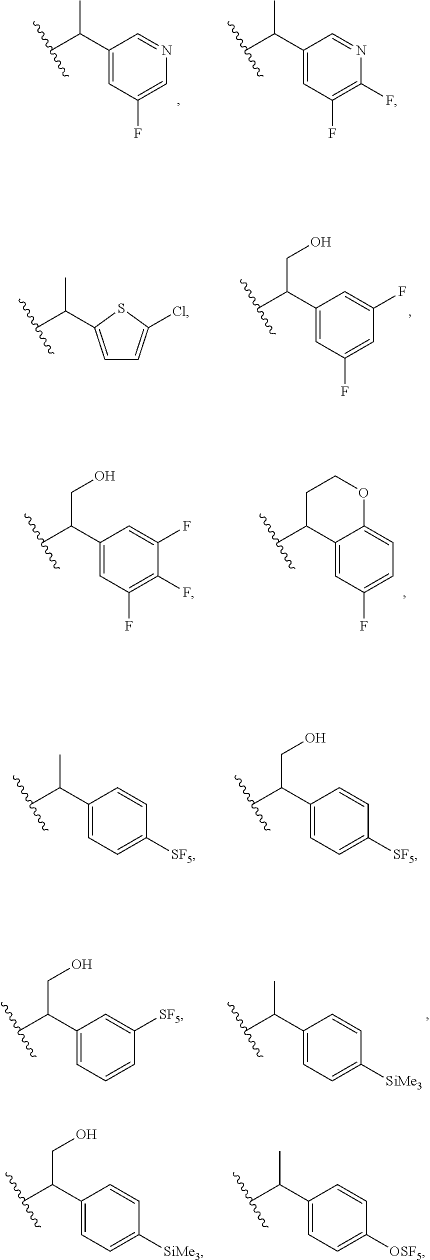

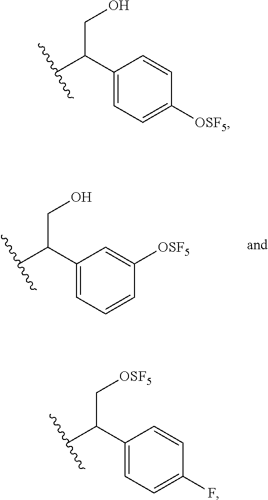

and
wherein the R⁹-R¹⁰— moiety is:

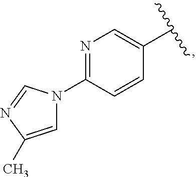

and
G is selected from the group consisting of —NH—, and a direct bond, and W is —S(O)₂—.

In another embodiment of this invention R¹ is selected from the group consisting of:

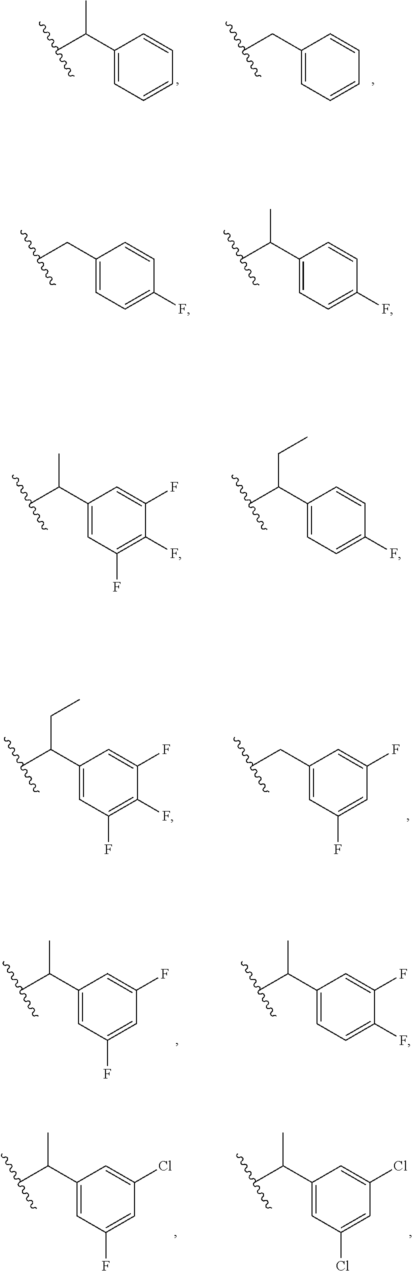

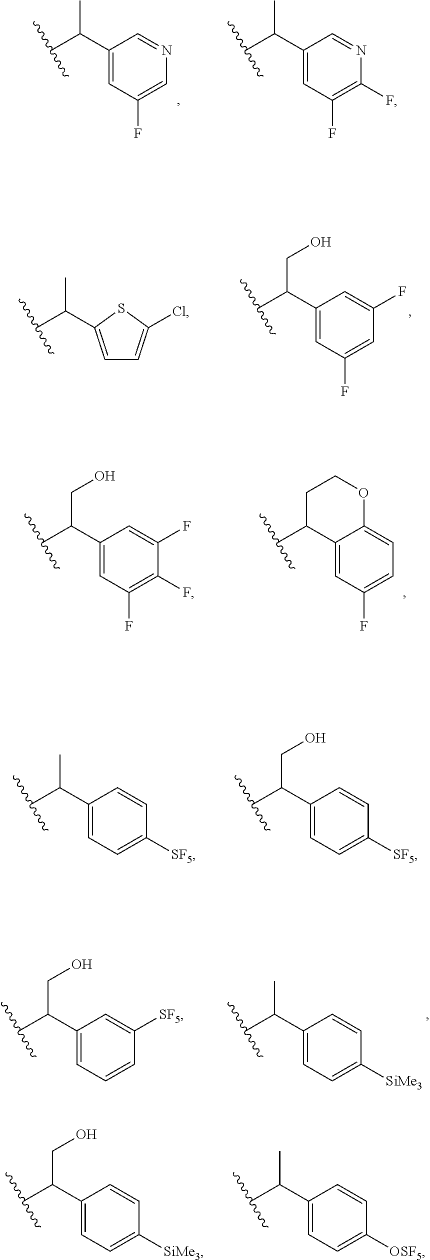

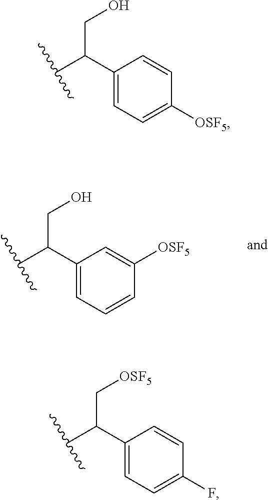

and
wherein the R⁹-R¹⁰— moiety is:

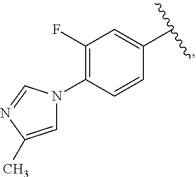

and
G is selected from the group consisting of —NH—, and a direct bond, and W is —S(O)₂—.

In another embodiment of this invention R¹ is selected from the group consisting of:

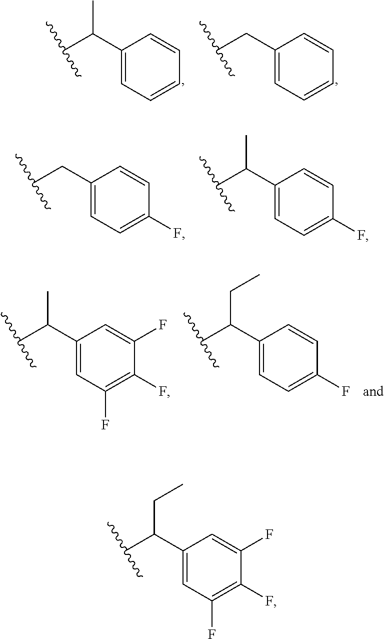

and
wherein the R⁹-R¹⁰— moiety is:

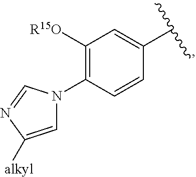

and
G is selected from the group consisting of —NH—, and a direct bond, and W is —S(O)₂—.

In another embodiment of this invention R¹ is selected from the group consisting of:

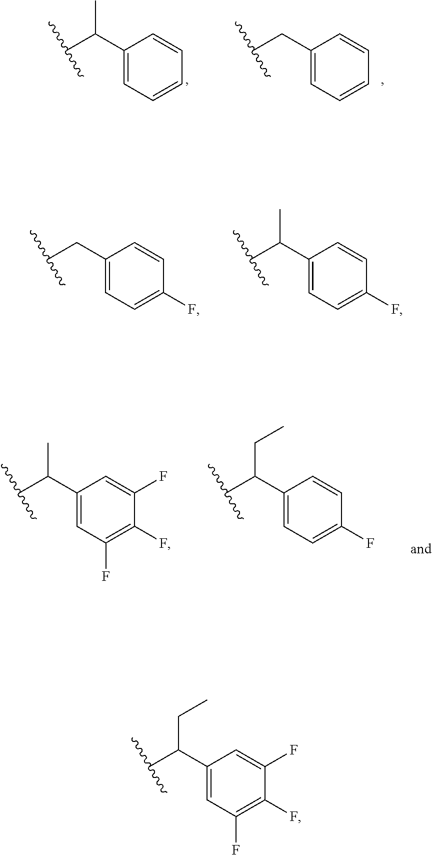

and
wherein the R⁹-R¹⁰— moiety is:

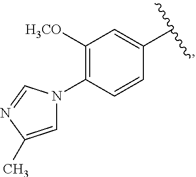

and
G is selected from the group consisting of —NH—, and a direct bond, and W is —S(O)₂—.

In another embodiment of this invention R¹ is selected from the group consisting of:

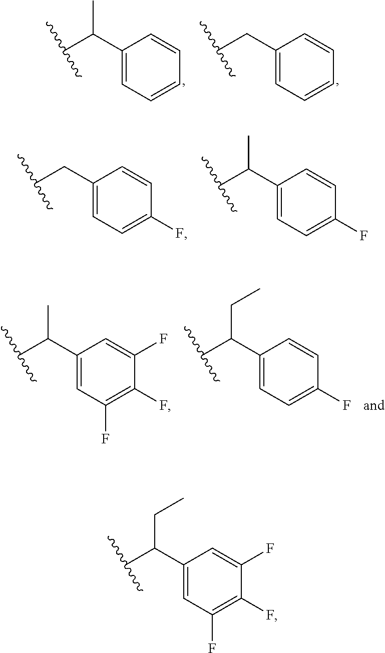

and
wherein the R⁹-R¹⁰— moiety is:

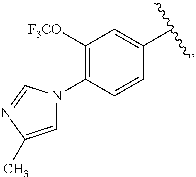

and
G is selected from the group consisting of —NH—, and a direct bond, and W is —S(O)₂—.

In another embodiment of this invention R¹ is selected from the group consisting of:

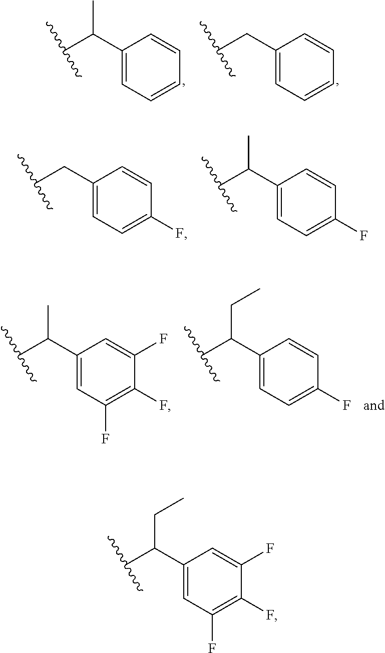

and
wherein the R⁹-R¹⁰— moiety is:

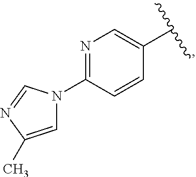

and
G is selected from the group consisting of —NH—, and a direct bond, and W is —S(O)₂—.

In another embodiment of this invention R¹ is selected from the group consisting of:

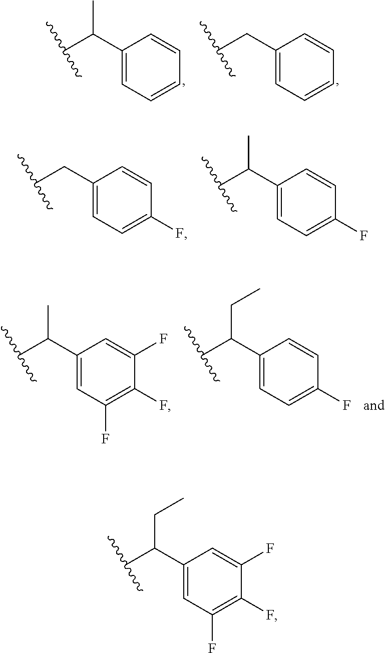

and
wherein the R⁹-R¹⁰— moiety is:

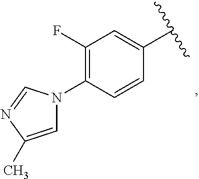

and
G is selected from the group consisting of —NH—, and a direct bond, and W is —S(O)₂—.

In another embodiment of this invention: (a) R¹ is a methyl or ethyl group substituted with one phenyl, or (b) R¹ is an methyl or alkyl group substituted with one phenyl, and said phenyl is substituted with one or two F, and (c) R¹⁰ is phenyl substituted with one —OR¹⁵ group, wherein R¹⁵ is methyl, and (d) R⁹ is selected from the group consisting of imidazolyl and imidazolyl substituted with one methyl group, and (e) G is selected from the group consisting of —NH—, and a direct bond, and (f) W is —C(═NR¹⁴)—.

In another embodiment of this invention R¹ is selected from the group consisting of:

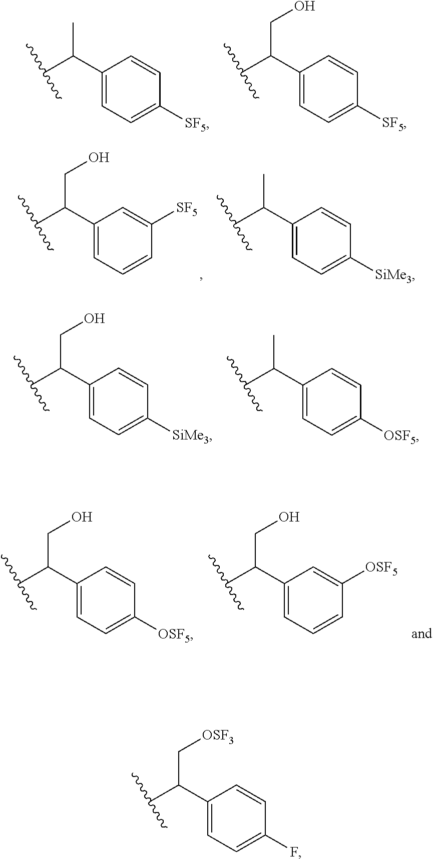

and
wherein the R⁹-R¹⁰— moiety is:

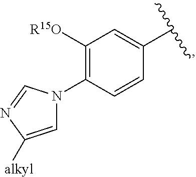

and
G is selected from the group consisting of —NH—, and a direct bond, and W is —C(═NR¹⁴)—.

In another embodiment of this invention R¹ is selected from the group consisting of:

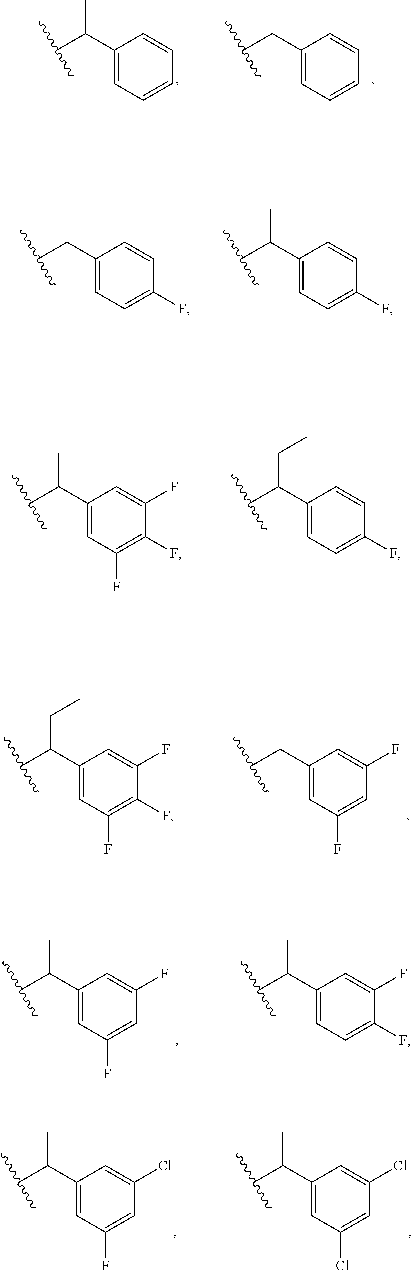

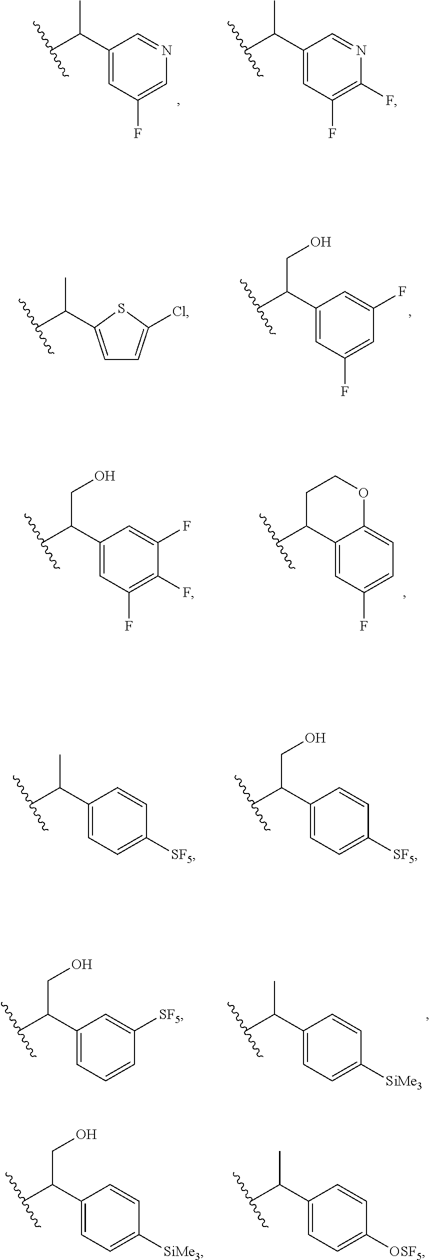

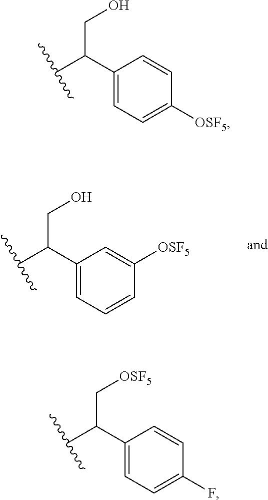

and
wherein the R⁹-R¹⁰— moiety is:

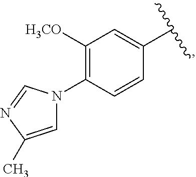

and
G is selected from the group consisting of —NH—, and a direct bond, and W is —C(═NR¹⁴)—.

In another embodiment of this invention R¹ is selected from the group consisting of:

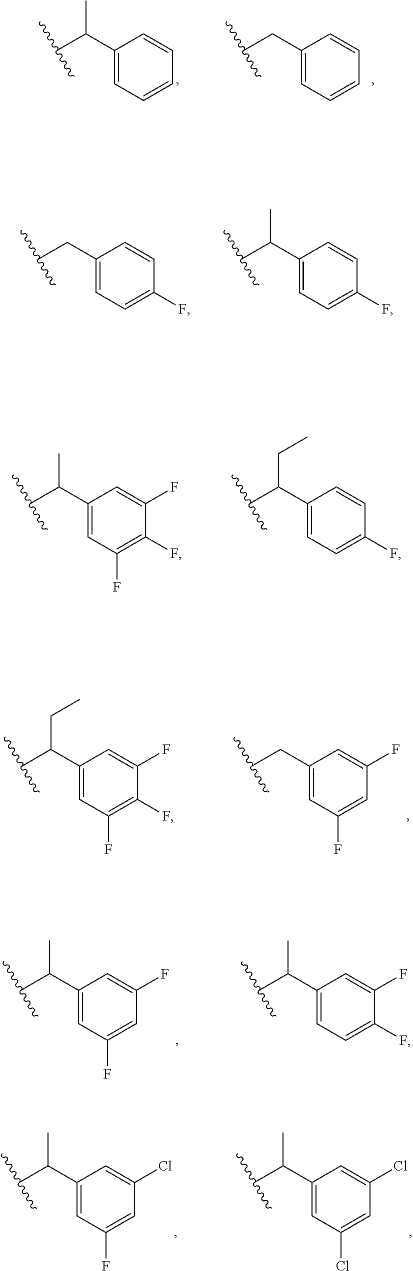

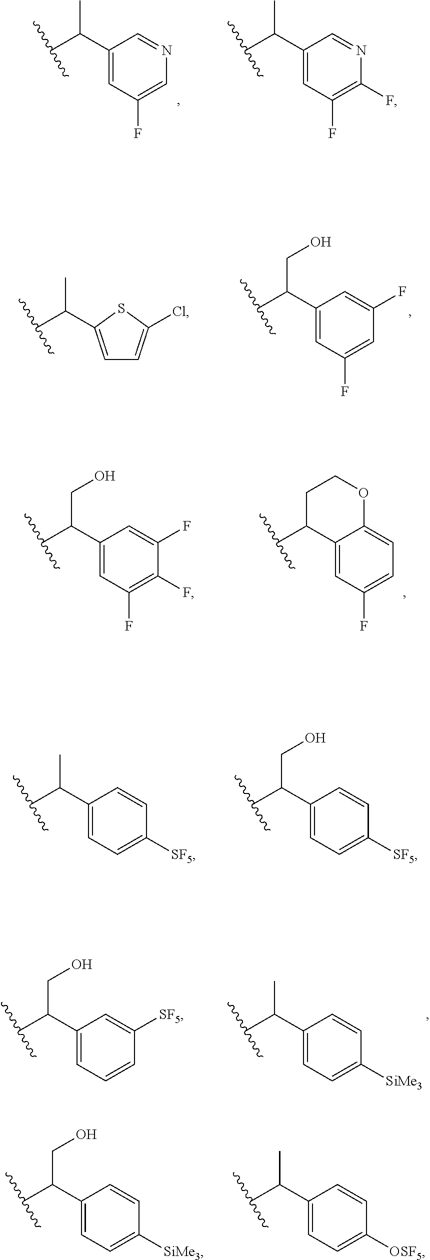

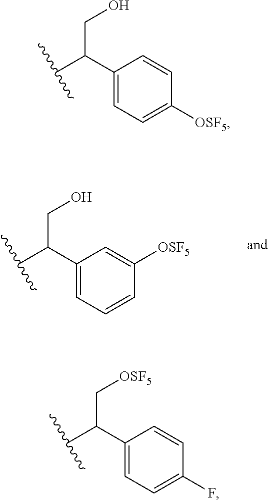

and
wherein the R⁹-R¹⁰— moiety is:

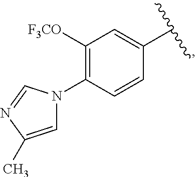

and
G is selected from the group consisting of —NH—, and a direct bond, and W is —C(═NR¹⁴)—.

In another embodiment of this invention R¹ is selected from the group consisting of:

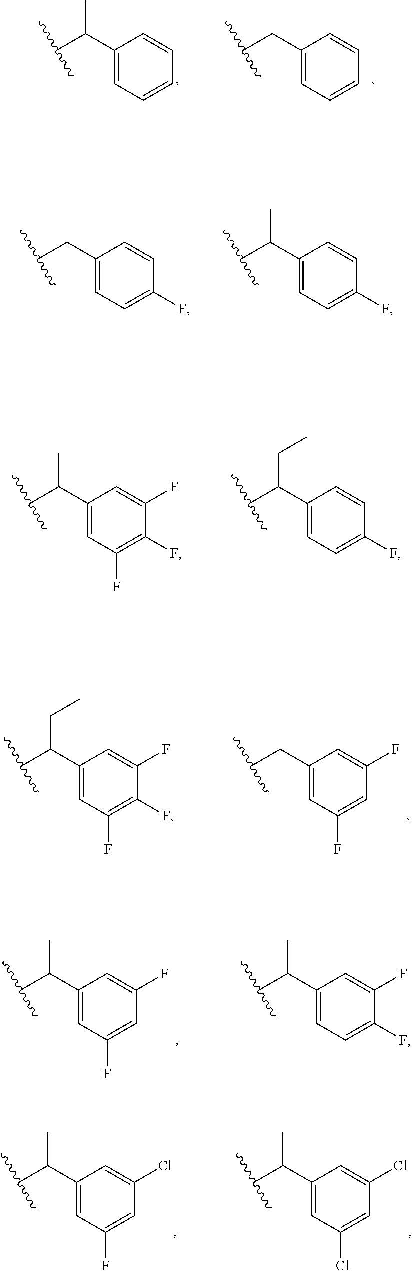

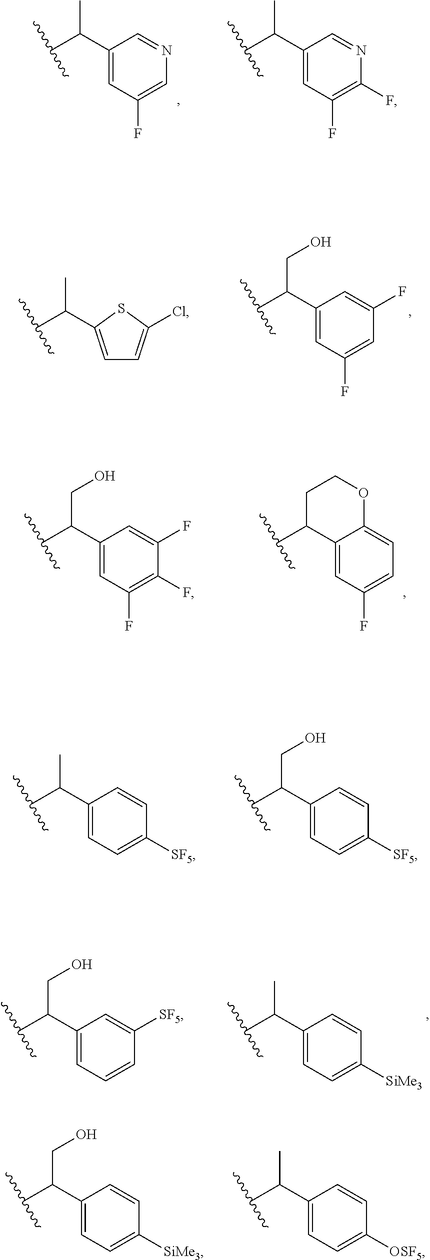

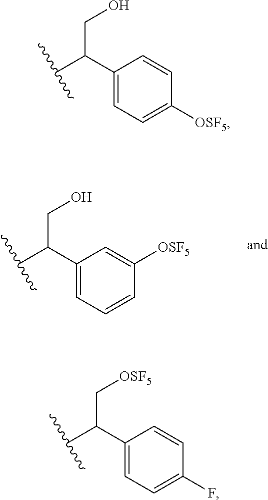

and
wherein the R⁹-R¹⁰— moiety is:

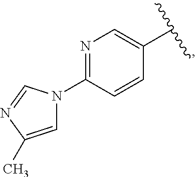

and
G is selected from the group consisting of —NH—, and a direct bond, and W is —C(═NR¹⁴)—.

In another embodiment of this invention R¹ is selected from the group consisting of:

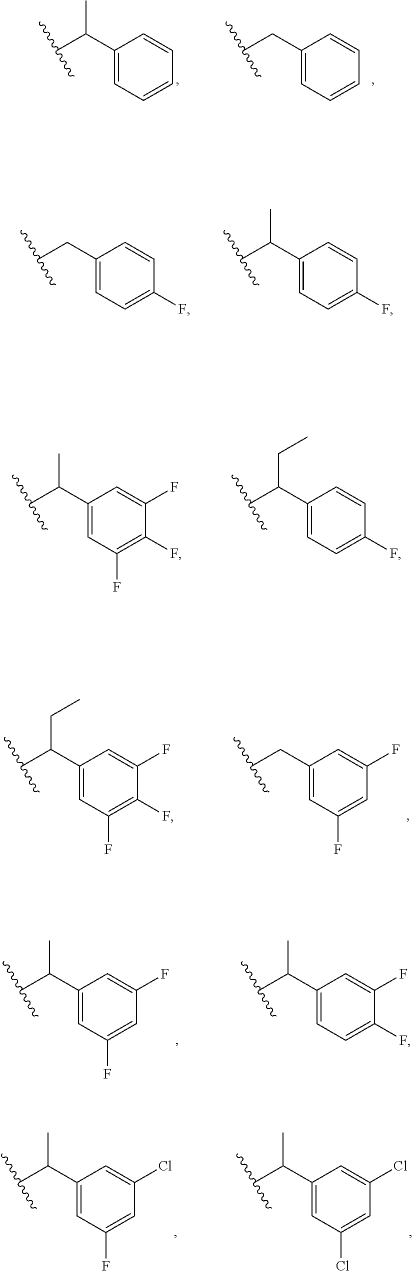

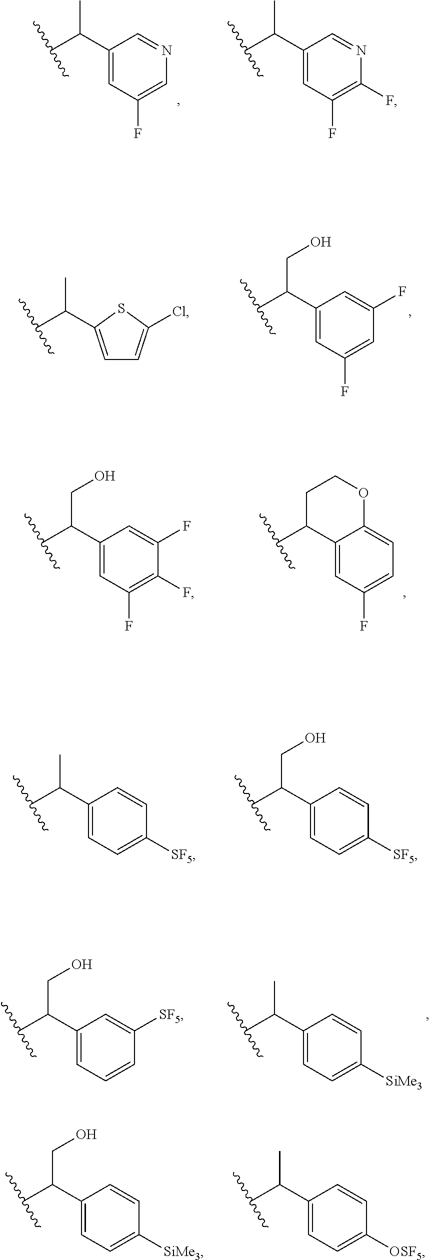

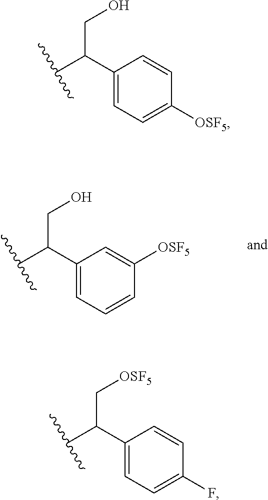

and
wherein the R⁹-R¹⁰— moiety is:

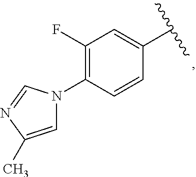

and
G is selected from the group consisting of —NH—, and a direct bond, and W is —C(═NR¹⁴)—.

In another embodiment of this invention R¹ is selected from the group consisting of:

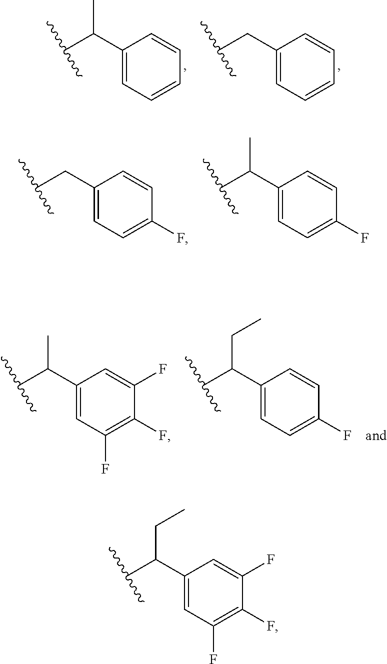

and
wherein the R⁹-R¹⁰— moiety is:

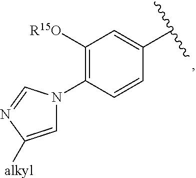

and
G is selected from the group consisting of —NH—, and a direct bond, and W is —C(═NR¹⁴)—.

In another embodiment of this invention R¹ is selected from the group consisting of:

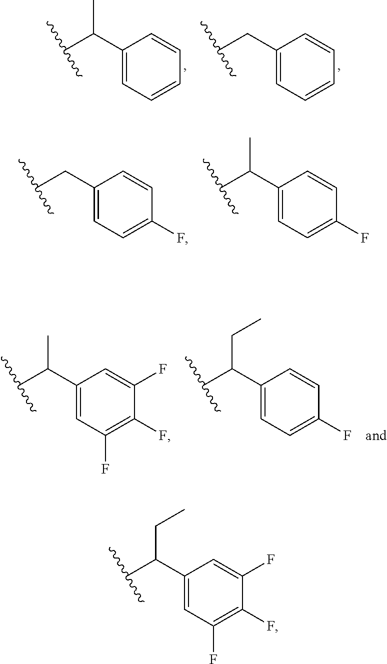

and
wherein the R⁹-R¹⁰— moiety is:

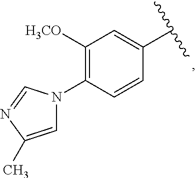

and
G is selected from the group consisting of —NH—, and a direct bond, and W is —C(═NR¹⁴)—.

In another embodiment of this invention R¹ is selected from the group consisting of:

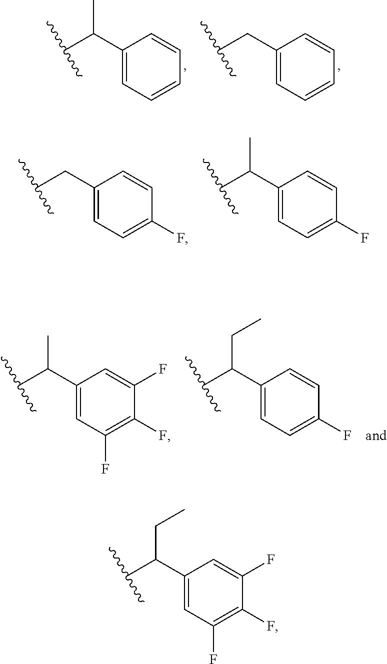

and
wherein the R⁹-R¹⁰— moiety is:

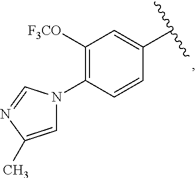

and
G is selected from the group consisting of —NH—, and a direct bond, and W is —C(═NR¹⁴)—.

In another embodiment of this invention R¹ is selected from the group consisting of:

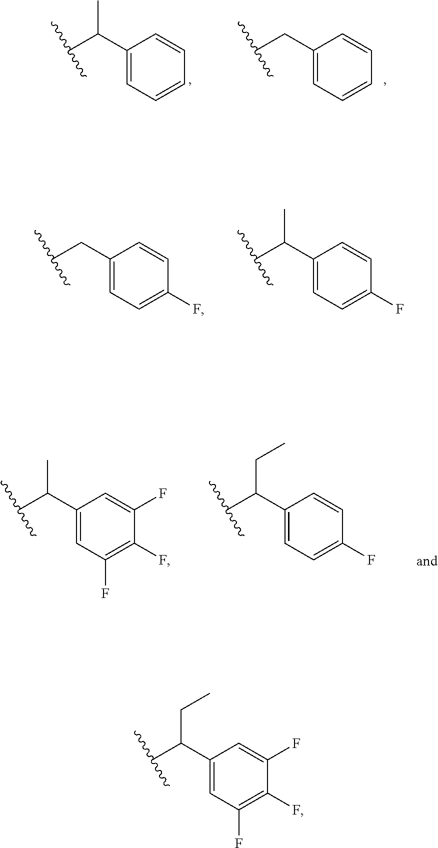

and
wherein the R⁹-R¹⁰— moiety is:

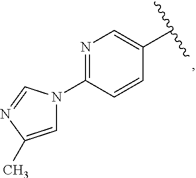

and
G is selected from the group consisting of —NH—, and a direct bond, and W is —C(═NR¹⁴)—.

In another embodiment of this invention R¹ is selected from the group consisting of:

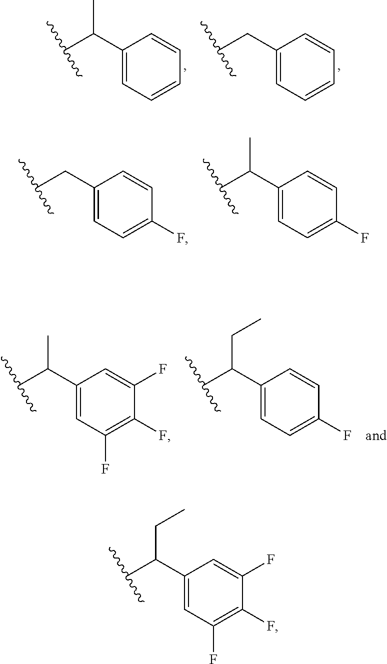

and
wherein the R⁹-R¹⁰— moiety is:

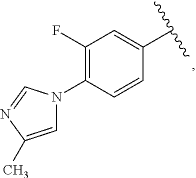

and
G is selected from the group consisting of —NH—, and a direct bond, and W is —C(═NR¹⁴)—.

Other embodiments of this invention are directed to compounds of formula (I) wherein R¹ is selected from the group consisting of: benzofusedcycloalkyl (i.e., fused benzocycloalkyl), fused benzoheterocycloalkyl, fused heteroarylcycloalkyl, fused heteroarylheterocycloalkyl, and wherein said R¹ groups are optionally substituted with 1-5 independently selected R²¹ groups. In one example, the R²¹ groups are halo (e.g., F).

Examples of the fused ring R¹ groups include, but are not limited to:

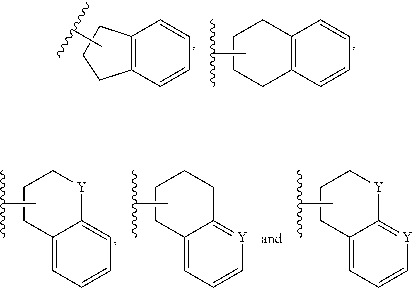

wherein each Y is independently selected from the group consisting of: —O—, —NR¹⁴— and —C(R²¹)_{q}—, wherein q is as defined above (i.e., 0, 1 or 2 and each R²¹ is independently selected), and wherein R¹⁴ and R²¹ are as defined for formula (I). Examples of these R¹ groups include, for example:

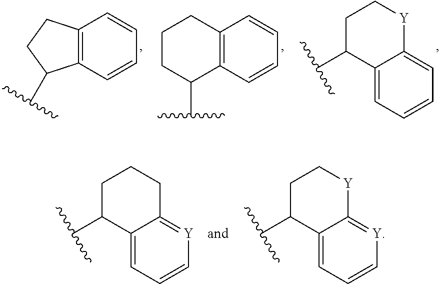

Compounds of formula (I) also include compounds wherein R¹ is an alkyl group (e.g., ethyl) substituted with one R²¹ group. Examples of said R¹ groups include alkyl (e.g., methyl or ethyl) substituted with the R²¹ moiety aryl (e.g., phenyl or naphthyl). Examples of said R¹ groups also include alkyl (e.g., methyl or ethyl) substituted with the R²¹ moiety aryl (e.g., phenyl or naphthyl), which in turn is substituted with one or more (e.g., one or two) independently selected R²² groups (e.g., R²² is halo, such as, for example, F).

Examples of the substituted R¹ alkyl groups include, but are not limited to:

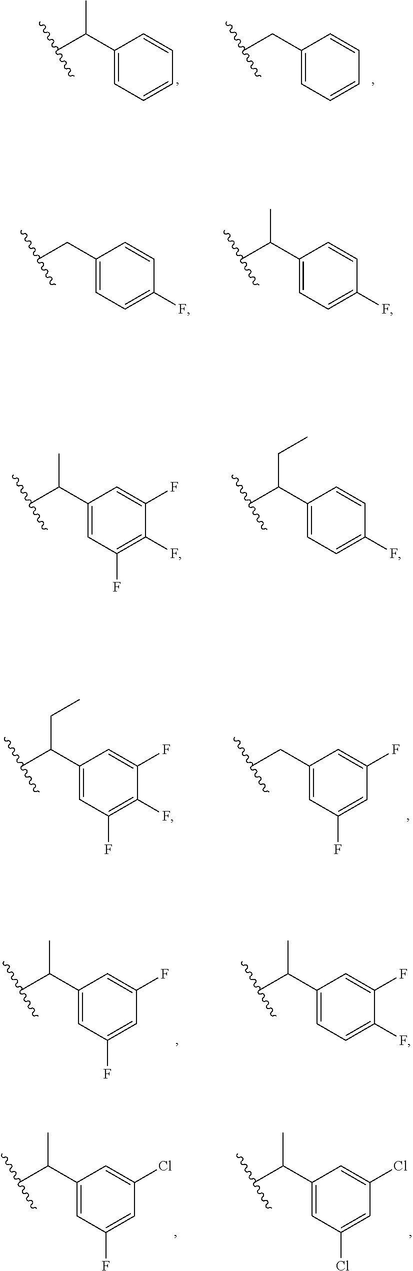

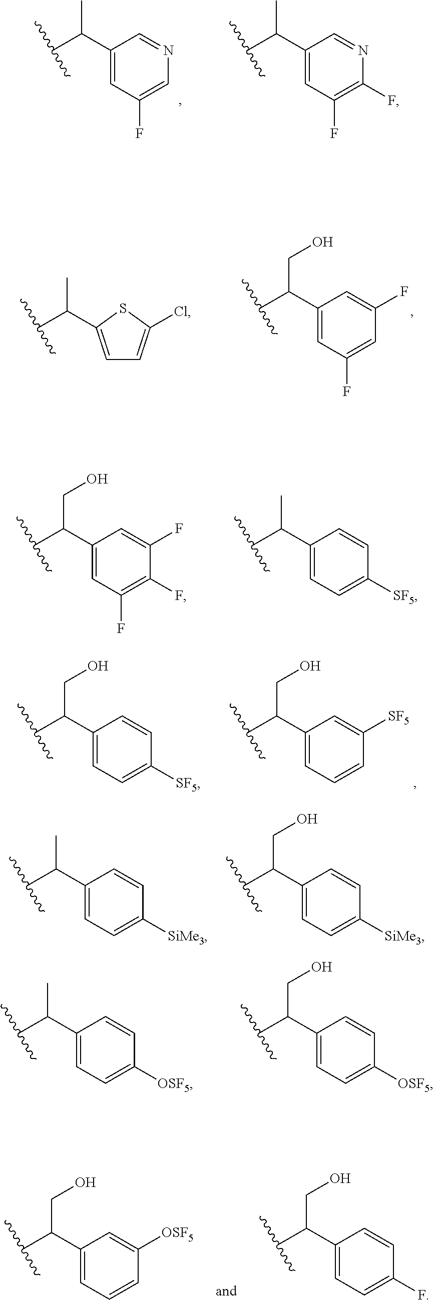

Examples of the substituted R¹ alkyl groups include, but are not limited to:

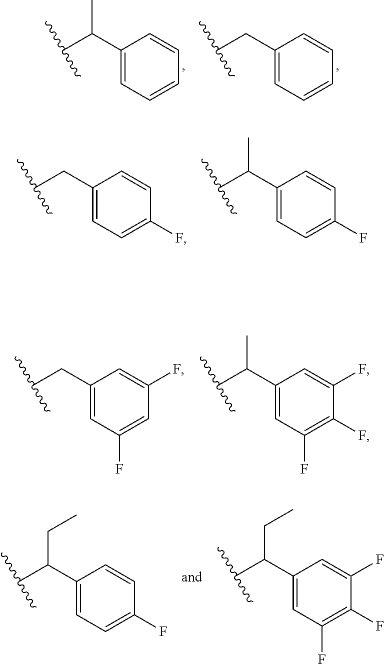

Examples of the substituted R¹ alkyl groups include, but are not limited to:

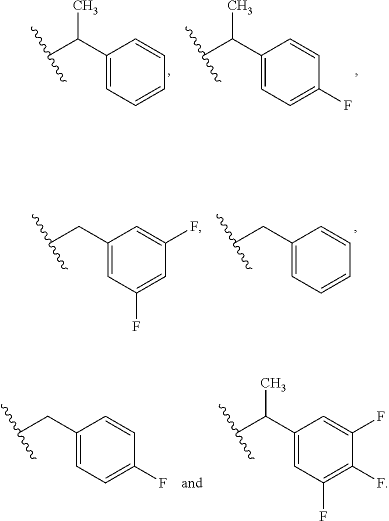

Other embodiments of this invention are directed to compounds of formula (I) wherein R¹ is a cycloalkyl group (e.g., cyclopropyl or cyclobutyl) substituted with one R²¹ group (e.g., aryl, such as, for example, phenyl), or a cycloalkyl group (e.g., cyclopentyl or cyclohexyl) substituted with one R²¹ group (e.g., aryl, such as, for example, phenyl) which in turn is substituted with one or more (e.g., one or two) independently selected R²² groups (e.g., halo, such as, for example, F). In one example the R²¹ group is bound to the same carbon of the R¹ group that binds the R¹ group to the rest of the molecule.

Examples of the cycloalkyl R¹ groups include, but are not limited to:

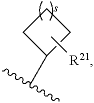

such as, for example,

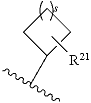

wherein s is 0 (i.e., the ring is cyclopropyl), or 1 (i.e., the ring is cyclobutyl). Examples of these R¹ groups include, but are not limited to:

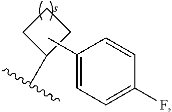

such as, for example,

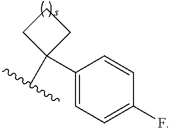

wherein s is 0 (i.e., the ring is cyclopropyl), or 1 (i.e., the ring is cyclobutyl).

Other embodiments of this invention are directed to compounds of formula (I) wherein R¹ is

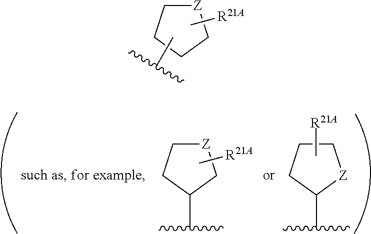

wherein Z is selected from the group consisting of: (1) —O—, (2) —NR¹⁴—, (3) —C(R²¹)_{q}— wherein q is 0, 1 or 2, and each R²¹ is independently selected, (4) —C(R²¹)_{q}—C(R²¹)_{q}— wherein each q is independently 0, 1 or 2 and each R²¹ is indepenendently selected, (5) —(C(R²¹)_{q})_{q}—O—(C(R²¹)_{q})_{q}— wherein each q is independently 0, 1 or 2, and each R²¹ is independently selected, and (6) —(C(R²¹)_{q})_{q}—N(R¹⁴)—(C(R²¹)_{q})_{q}— wherein each q is independently 0, 1 or 2, and each R²¹ is independently selected. R^{21A} is defined the same as R²¹ for formula (I). Examples of R^{21A} include, but are not limited to, aryl (e.g., phenyl) and aryl (e.g., phenyl) substituted with one or more (e.g., one or two, or one) independently selected R²² groups (e.g., halo, such as, for example, F). Examples of this R¹ include, but are not limited to:

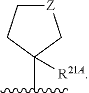

Thus, examples of this R¹ group include, but are not limited to:

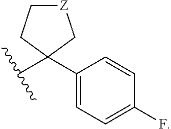

Examples of R¹ also include, but are not limited to:

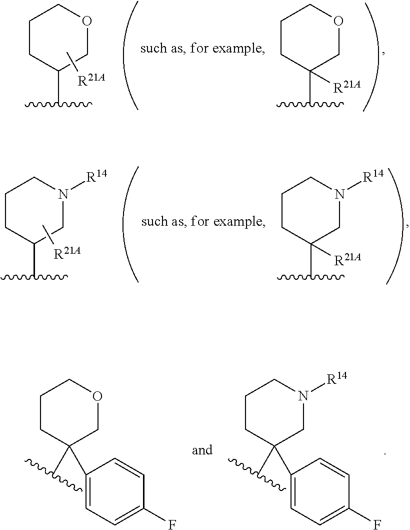

Examples of the R¹ group

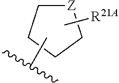

also include, but are not limited to:

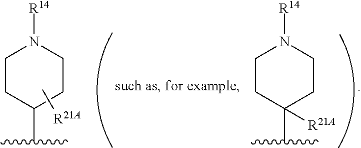

Examples of the R¹ group

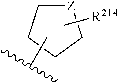

also include, but are not limited to:

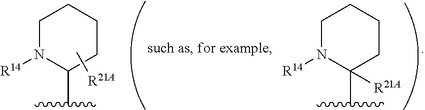

Examples of the R¹ group

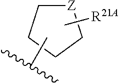

also include, but are not limited to:

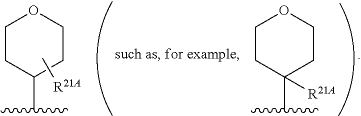

Examples of the R¹ group

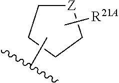

also include, but are not limited to:

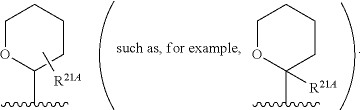

Other embodiments of this invention are directed to compounds of formula (I) wherein R¹⁰ is aryl (e.g., phenyl) or aryl (e.g., phenyl) substituted with one or more (e.g., one or two, or one) R²¹ groups (e.g., —OR¹⁵, wherein, for example, R¹⁵ is alkyl, such as, for example, methyl), and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

Thus, examples of the

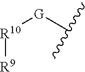

moiety of the compounds of this invention include, but are not limited to:

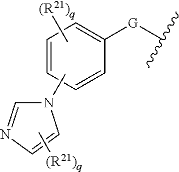

wherein q is 0, 1 or 2, such as, for example,

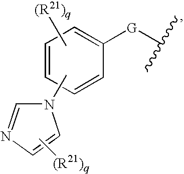

such as, for example,

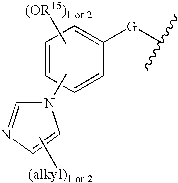

wherein R¹⁵ is alkyl (e.g., methyl), such as, for example,

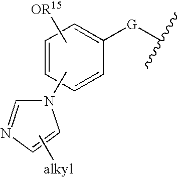

wherein R¹⁵ is alkyl (e.g., methyl), such as, for example,

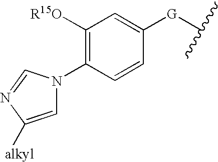

wherein R¹⁵ is alkyl (e.g., methyl), such as, for example,

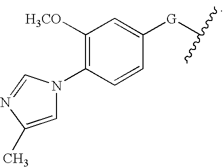

Other embodiments of this invention are directed to the compounds of formula (I) wherein R¹⁰ is heteroaryl or heteroaryl substituted with one or more R²¹ groups, and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

In another embodiment of the compounds of formula (I) R¹⁰ is aryl substituted with one R²¹ group, wherein said R²¹ group is —OR¹⁵. In one example, R¹⁵ is alkyl. In another example R¹⁵ is methyl.

In another embodiment of the compounds of formula (I) R¹⁰ is phenyl substituted with one R²¹ group, wherein said R²¹ group is —OR¹⁵. In one example, R¹⁵ is alkyl. In another example R¹⁵ is methyl.

In another embodiment of the compounds of formula (I) R¹⁰ is heteroaryl.

In another embodiment of the compounds of formula (I) R⁹ is heteroaryl.

In another embodiment of the compounds of formula (I) R⁹ is heteroaryl substituted with one or more (e.g., one) independently selected R²¹ groups.

In another embodiment of the compounds of formula (I) R⁹ is heteroaryl substituted with one or more (e.g., one) independently selected R²¹ groups, wherein each R²¹ group is the same or different alkyl group (e.g., methyl).

In another embodiment of the compounds of formula (I) R⁹ is heteroaryl substituted with one R²¹ group.

In another embodiment of the compounds of formula (I) R⁹ is heteroaryl substituted with one R²¹ group, wherein R²¹ is an alkyl group (e.g., methyl).

In another embodiment of the compounds of formula (I) R⁹ is imidazolyl.

In another embodiment of the compounds of formula (I) R⁹ is imidazolyl substituted with one or more (e.g., one) independently selected R²¹ groups.

In another embodiment of the compounds of formula (I) R⁹ is imidazolyl substituted with one or more (e.g., one) independently selected R²¹ groups, wherein each R²¹ group is the same or different alkyl group (e.g., methyl).

In another embodiment of the compounds of formula (I) R⁹ is imidazolyl substituted with one R²¹ group.

In another embodiment of the compounds of formula (I) R⁹ is imidazolyl substituted with one R²¹ group, wherein R²¹ is an alkyl group (e.g., methyl).

In another embodiment of the compounds of formula (I) R⁹ is heteroaryl, optionally substituted with one or more R²¹ groups, and R¹⁰ is aryl optionally substituted with one or more (e.g., one) R²¹ groups.

In another embodiment of the compounds of formula (I) R⁹ is heteroaryl, optionally substituted with one R²¹ group, and R¹⁰ is aryl optionally substituted with one R²¹ group.

In another embodiment of the compounds of formula (I) R⁹ is heteroaryl, optionally substituted with one or more R²¹ groups, and R¹⁰ is phenyl optionally substituted with one or more (e.g., one) R²¹ groups.

In another embodiment of the compounds of formula (I) R⁹ is heteroaryl, optionally substituted with one R²¹ group, and R¹⁰ is phenyl optionally substituted with one R²¹ group.

In another embodiment of the compounds of formula (I) R⁹ is imidazolyl, optionally substituted with one or more R²¹ groups, and R¹⁰ is aryl optionally substituted with one or more (e.g., one) R²¹ groups.

In another embodiment of the compounds of formula (I) R⁹ is imidazolyl, optionally substituted with one R²¹ group, and R¹⁰ is aryl optionally substituted with one R²¹ group.

In another embodiment of the compounds of formula (I) R⁹ is imidazolyl, optionally substituted with one or more R²¹ groups, and R¹⁰ is phenyl optionally substituted with one or more (e.g., one) R²¹ groups.

In another embodiment of the compounds of formula (I) R⁹ is imidazolyl, optionally substituted with one R²¹ group, and R¹⁰ is phenyl optionally substituted with one R²¹ group.

In another embodiment of the compounds of formula (I) R⁹ is heteroaryl, optionally substituted with one or more R²¹ groups, R¹⁰ is aryl optionally substituted with one or more (e.g., one) R²¹ groups, W is —C(O)—. In one example the R²¹ groups for R⁹ are independently selected from alkyl. In another example of this embodiment the R²¹ groups for R¹⁰ are independently selected from —OR¹⁵ (wherein, for example, R¹⁵ is alkyl, such as, for example, methyl). In one example of this embodiment R⁹ is substituted with one R²¹ group. In another example of this embodiment R¹⁰ is substituted with one R²¹ group. In another example of this embodiment R⁹ is substituted with one R²¹ group, and R¹⁰ is substituted with one R²¹ group, each R²¹ being independently selected. In another example of this embodiment the R⁹ is substituted with one R²¹ group and said R²¹ group is alkyl (e.g., methyl), and R¹⁰ is substituted with one R²¹ group and this R²¹ group is —OR¹⁵ (wherein R¹⁵ is, for example, alkyl, such as, for example, methyl).

In another embodiment of this invention R⁹ is selected from the group consisting of:

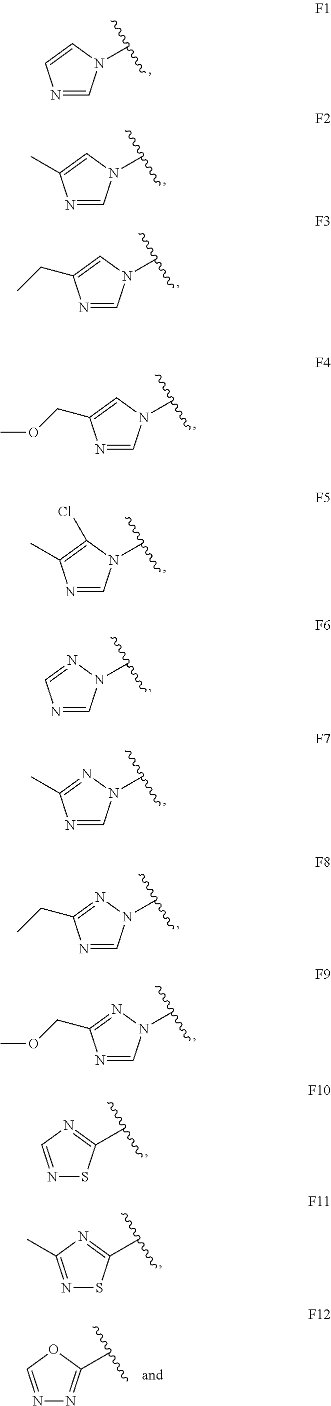

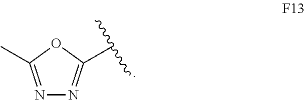

In another embodiment of this invention R⁹ is F1. In another embodiment of this invention R⁹ is F2. In another embodiment of this invention R⁹ is F3. In another embodiment of this invention R⁹ is F4. In another embodiment of this invention R⁹ is. F5. In another embodiment of this invention R⁹ is F6. In another embodiment of this invention R⁹ is F7. In another embodiment of this invention R⁹ is F8. In another embodiment of this invention R⁹ is F9. In another embodiment of this invention R⁹ is F10. In another embodiment of this invention R⁹ is F11. In another embodiment of this invention R⁹ is F12.

Other embodiments of the compounds of formula (I) are directed to any one of the above embodiments wherein R⁹ is:

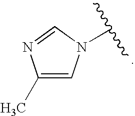

Other embodiments of the compounds of formula (I) are directed to any one of the above embodiments wherein R¹⁰ is:

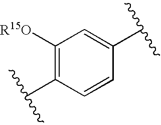

(wherein the —OR¹⁵ is ortho to the carbon to which R⁹ is bound to, i.e., the R⁹-R¹⁰— moiety is:

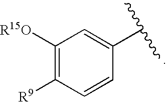

Other embodiments for the compounds of formula (I) are directed to any one of the above embodiments wherein R¹⁰ is:

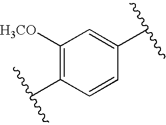

(wherein the —OCH₃ is ortho to the carbon to which R⁹ is bound to, i.e., the R⁹-R¹⁰— moiety is:

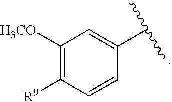

In another embodiment of the compounds of formula (I) R¹ is benzofusedcycloalkyl.

In another embodiment of the compounds of formula (I) R¹ is:

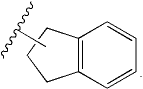

In another embodiment of the compounds of formula (I) R¹ is:

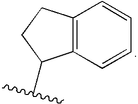

In another embodiment of the compounds of formula (I) R¹ is:

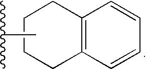

In another embodiment of the compounds of formula (I) R¹ is:

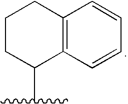

In another embodiment of the compounds of formula (I) R¹ is alkyl substituted with one R²¹ group.

In another embodiment of the compounds of formula (I) R¹ is alkyl substituted with one R²¹ group, and said alkyl is

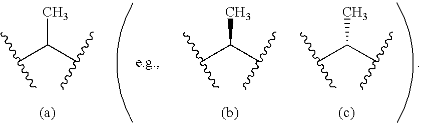

In another embodiment of the compounds of formula (I) R¹ is alkyl (e.g., (a), (b) or (c) described above) substituted with one R²¹ group wherein said R²¹ group is aryl.

In another embodiment of the compounds of formula (I) R¹ is alkyl (e.g., (a), (b) or (c) described above) substituted with one R²¹ group wherein said R²¹ group is phenyl.

In another embodiment of the compounds of formula (I) R¹ is alkyl (e.g., (a), (b) or (c) described above) substituted with one R²¹ group wherein said R²¹ group is naphthyl.

In another embodiment of the compounds of formula (I) R¹ is alkyl substituted with one R²¹ group, and said R²¹ group is substituted with two independently selected R²² groups.

In another embodiment of the compounds of formula (I) R¹ is alkyl substituted with one R²¹ group, and said R²¹ group is substituted with one R²² group.

In another embodiment of the compounds of formula (I) R¹ is alkyl substituted with one R²¹ group, wherein said alkyl group is (a) (e.g., (b) or (c)), as described above, and said R²¹ group is substituted with two independently selected R²² groups.

In another embodiment of the compounds of formula (I) R¹ is alkyl substituted with one R²¹ group, wherein said alkyl group is (a) (e.g., (b) or (c)), as described above, and said R²¹ group is substituted with one R²² group.

In another embodiment of the compounds of formula (I) R¹ is alkyl substituted with one R²¹ group, wherein said R²¹ group is aryl, and said R²¹ group is substituted with two independently selected R²² groups.

In another embodiment of the compounds of formula (I) R¹ is alkyl substituted with one R²¹ group, wherein said R²¹ group is aryl, and said R²¹ group is substituted with one R²² group.

In another embodiment of the compounds of formula (I) R¹ is alkyl substituted with one R²¹ group, wherein said R²¹ group is aryl, said alkyl group is (a) (e.g., (b) or (c)), as described above, and said R²¹ group is substituted with two independently selected R²² groups.

In another embodiment of the compounds of formula (I) R¹ is alkyl substituted with one R²¹ group, wherein said R²¹ group is aryl, wherein said alkyl group is (a) (e.g., (b) or (c)), as described above, and said R²¹ group is substituted with one R²² group.

In another embodiment of the compounds of formula (I) R¹ is alkyl substituted with one R²¹ group, wherein said R²¹ group is aryl, said R²¹ group is substituted with two independently selected R²² groups, and each R²² is halo.

In another embodiment of the compounds of formula (I) R¹ is alkyl substituted with one R²¹ group, wherein said R²¹ group is aryl, and said R²¹ group is substituted with one R²² group, and said R²² is halo.

In another embodiment of the compounds of formula (I) R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is halo.

In another embodiment of the compounds of formula (I) R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is halo.

In another embodiment of the compounds of formula (I) R¹ is alkyl substituted with one R²¹ group, wherein said R²¹ group is aryl, said R²¹ group is substituted with two independently selected R²² groups, and each R²² is F.

In another embodiment of the compounds of formula (I) R¹ is alkyl substituted with one R²¹ group, wherein said R²¹ group is aryl, and said R²¹ group is substituted with one R²² group, and said R²² is F.

In another embodiment of the compounds of formula (I) R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is F.

In another embodiment of the compounds of formula (I) R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is F.

In another embodiment of the compounds of formula (I) R¹ is:

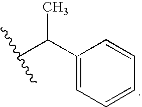

In another embodiment of the compounds of formula (I) R¹ is:

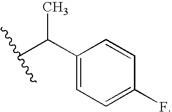

In another embodiment of the compounds of formula (I) R¹ is:

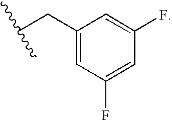

In another embodiment of the compounds of formula (I) R¹ is:

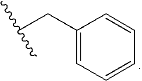

In another embodiment of the compounds of formula (I) R¹ is:

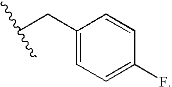

In another embodiment of the compounds of formula (I) R¹ is:

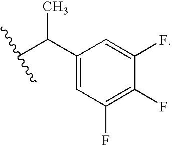

In another embodiment R¹ is

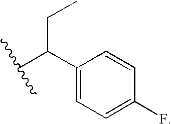

In another embodiment R¹ is

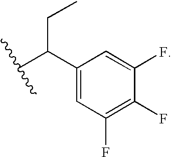

In another embodiment R¹ is

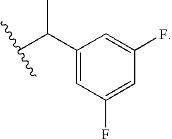

In another embodiment R¹ is

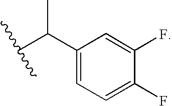

In another embodiment R¹ is

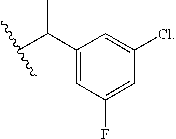

In another embodiment R¹ is

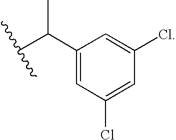

In another embodiment R¹ is

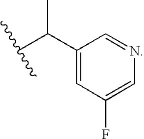

In another embodiment R¹ is

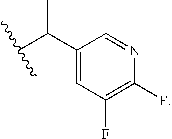

In another embodiment R¹ is

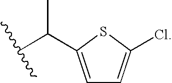

In another embodiment R¹ is

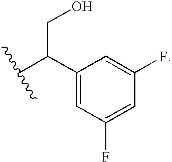

In another embodiment R¹ is

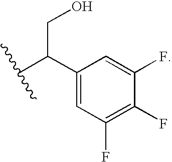

In another embodiment R¹ is

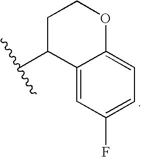

In another embodiment R¹ is

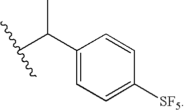

In another embodiment R¹ is

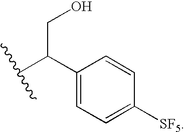

In another embodiment R¹ is

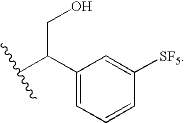

In another embodiment R¹ is

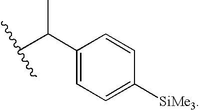

In another embodiment R¹ is

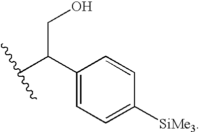

In another embodiment R¹ is

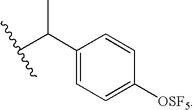

In another embodiment R¹ is

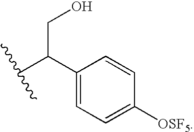

In another embodiment R¹ is

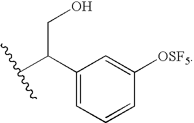

In another embodiment R¹ is

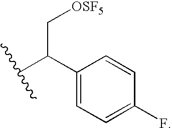

In another embodiment of this invention the compound of formula (I) is a compound of the formula:

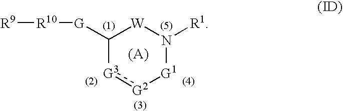

In another embodiment of this invention the compound of formula (I) is a compound of the formula:

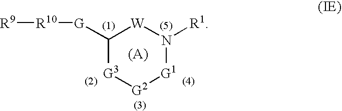

In another embodiment of this invention the compound of formula (I) is a compound of the formula:

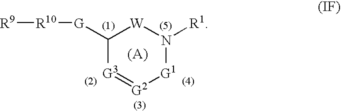

In another embodiment of this invention the compound of formula (I) is a compound of the formula:

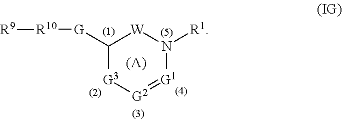

In another embodiment of this invention the compound of formula (I) is a compound of the formula:

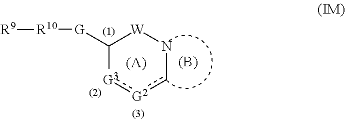

wherein (B) is an optional 4 to 6 membered ring (including the atoms common to Rings (A) and (B)), said Ring (B) optionally comprises 1 to 3 additional heteroatoms selected from the group consisting of —NR²—, —O—, —S—, —S(O)—, and —S(O)₂, and wherein said fused ring (B) optionally comprises 1 to 3 double bonds (and in one example, Ring (A) is a five membered ring and said fused ring (B) is a 6 membered ring (including the atoms common to both rings), and said fused ring additionally comprises a N atom double bonded to G¹, and G¹ is carbon).

In another embodiment of this invention the compound of formula (I) is a compound of the formula:

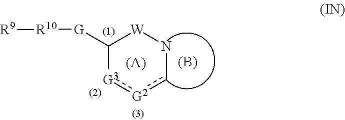

wherein (B) is a 4 to 6 membered ring (including the atoms common to Rings (A) and (B)), said Ring (B) optionally comprises 1 to 3 additional heteroatoms selected from the group consisting of —NR²—, —O—, —S—, —S(O)—, and —S(O)₂, and wherein said fused ring (B) optionally comprises 1 to 3 double bonds (and in one example, Ring (A) is a five membered ring and said fused ring (B) is a 6 membered ring (including the atoms common to both rings), and said fused ring additionally comprises a N atom double bonded to G¹, and G¹ is carbon).

In another embodiment of this invention the compound of formula (I) is a compound of the formula:

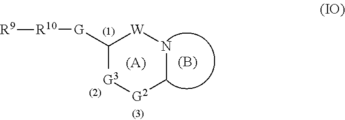

wherein (B) is a 4 to 6 membered ring (including the atoms common to Rings (A) and (B)), said Ring (B) optionally comprises 1 to 3 additional heteroatoms selected from the group consisting of —NR²—, —O—, —S—, —S(O)—, and —S(O)₂, and wherein said fused ring (B) optionally comprises 1 to 3 double bonds (and in one example, Ring (A) is a five membered ring and said fused ring (B) is a 6 membered ring (including the atoms common to both rings), and said fused ring additionally comprises a N atom double bonded to G¹, and G¹ is carbon).

In another embodiment of this invention the compound of formula (I) is a compound of the formula:

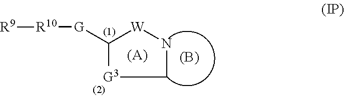

wherein (B) is a 4 to 6 membered ring (including the atoms common to Rings (A) and (B)), said Ring (B) optionally comprises 1 to 3 additional heteroatoms selected from the group consisting of —NR²—, —O—, —S—, —S(O)—, and —S(O)₂, and wherein said fused ring (B) optionally comprises 1 to 3 double bonds (and in one example, Ring (A) is a five membered ring and said fused ring (B) is a 6 membered ring (including the atoms common to both rings), and said fused ring additionally comprises a N atom double bonded to G¹, and G¹ is carbon).

In another embodiment of this invention the compound of formula (I) is a compound of the formula:

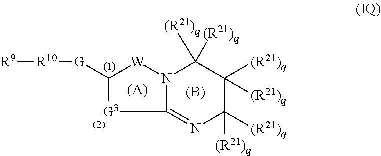

wherein each q is independently 0 or 1, and each R²¹ is independently selected.

In another embodiment of this invention the compound of formula (I) is a compound of the formula:

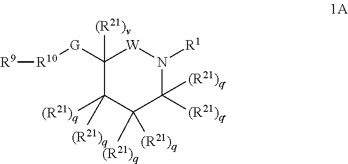

In another embodiment of this invention the compound of formula (I) is a compound of the formula:

In another embodiment of this invention the compound of formula (I) is a compound of the formula:

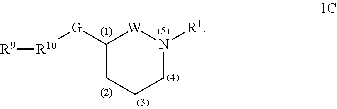

In another embodiment of this invention the compound of formula (I) is a compound of the formula:

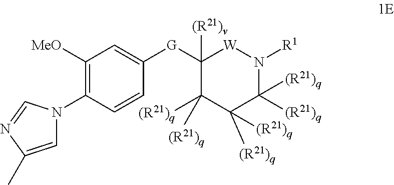

wherein each q is independently 0 or 1, and each R²¹ is independently selected.

In another embodiment of this invention the compound of formula (I) is a compound of the formula:

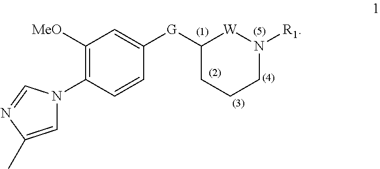

In another embodiment of this invention the compound of formula (I) is a compound of the formula:

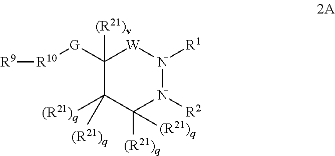

wherein each q is independently 0 or 1, and each R²¹ is independently selected. wherein each q is independently 0 or 1, and each R²¹ is independently selected.

In another embodiment of this invention the compound of formula (I) is a compound of the formula:

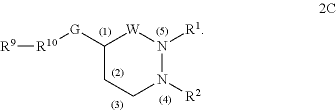

In another embodiment of this invention the compound of formula (I) is a compound of the formula:

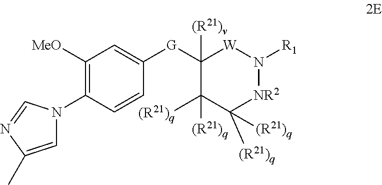

wherein each q is independently 0 or 1, and each R²¹ is independently selected.

In another embodiment of this invention the compound of formula (I) is a compound of the formula:

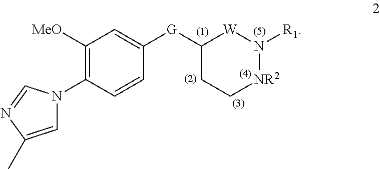

In another embodiment of this invention the compound of formula (I) is a compound of the formula:

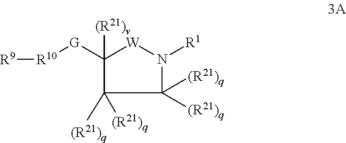

wherein each q is independently 0 or 1, and each R²¹ is independently selected.

In another embodiment of this invention the compound of formula (I) is a compound of the formula:

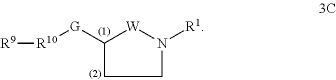

In another embodiment of this invention the compound of formula (I) is a compound of the formula:

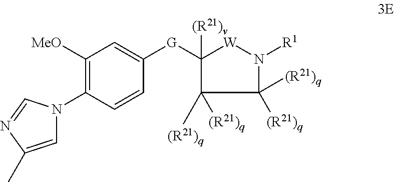

wherein each q is independently 0 or 1, and each R²¹ is independently selected.

In another embodiment of this invention the compound of formula (I) is a compound of the formula:

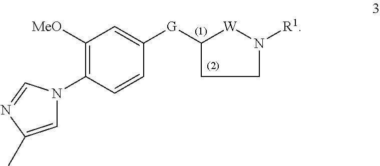

In another embodiment of this invention the compound of formula (I) is a compound of the formula:

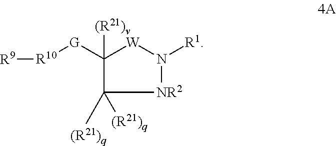

wherein each q is independently 0 or 1, and each R²¹ is independently selected.

In another embodiment of this invention the compound of formula (I) is a compound of the formula:

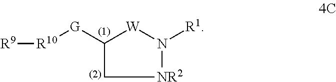

In another embodiment of this invention the compound of formula (I) is a compound of the formula:

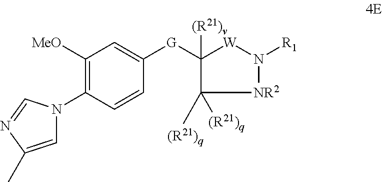

wherein each q is independently 0 or 1, and each R²¹ is independently selected.

In another embodiment of this invention the compound of formula (I) is a compound of the formula:

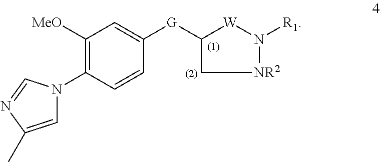

In another embodiment of this invention the compound of formula (I) is a compound of the formula:

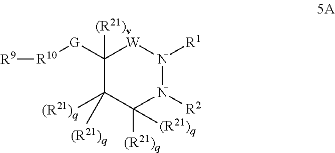

wherein each q is independently 0 or 1, and each R²¹ is independently selected.

In another embodiment of this invention the compound of formula (I) is a compound of the formula:

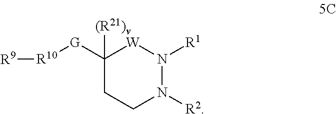

In another embodiment of this invention the compound of formula (I) is a compound of the formula:

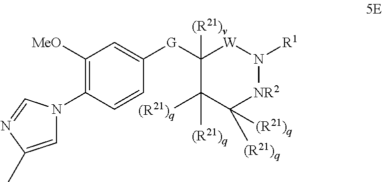

wherein each q is independently 0 or 1, and each R²¹ is independently selected.

In another embodiment of this invention the compound of formula (I) is a compound of the formula:

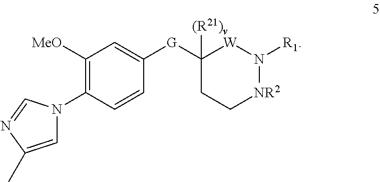

In another embodiment of this invention the compound of formula (I) is a compound of the formula:

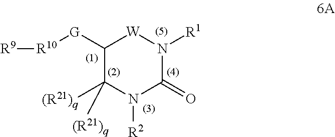

wherein each q is independently 0 or 1, and each R²¹ is independently selected.

In another embodiment of this invention the compound of formula (I) is a compound of the formula:

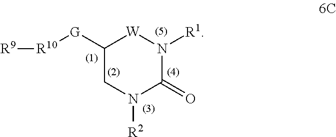

In another embodiment of this invention the compound of formula (I) is a compound of the formula:

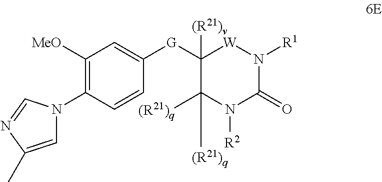

wherein each q is independently 0 or 1, and each R²¹ is independently selected.

In another embodiment of this invention the compound of formula (I) is a compound of the formula:

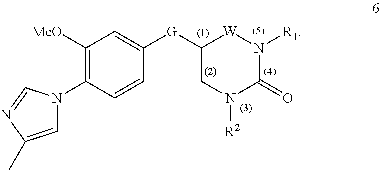

In another embodiment of this invention the compound of formula (I) is a compound of the formula:

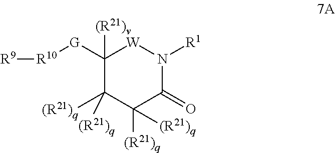

wherein each q is independently 0 or 1, and each R²¹ is independently selected.

In another embodiment of this invention the compound of formula (I) is a compound of the formula:

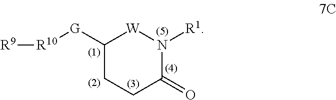

In another embodiment of this invention the compound of formula (I) is a compound of the formula:

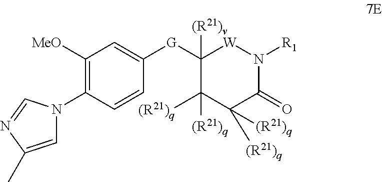

wherein each q is independently 0 or 1, and each R²¹ is independently selected.

In another embodiment of this invention the compound of formula (I) is a compound of the formula:

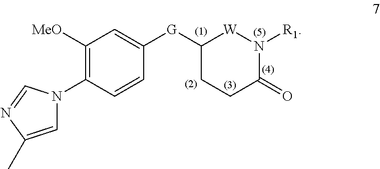

In another embodiment of this invention the compound of formula (I) is a compound of the formula:

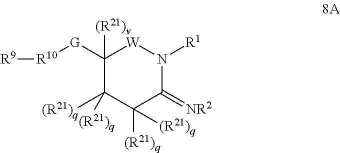

wherein each q is independently 0 or 1, and each R²¹ is independently selected.

In another embodiment of this invention the compound of formula (I) is a compound of the formula:

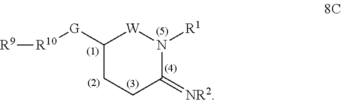

In another embodiment of this invention the compound of formula (I) is a compound of the formula:

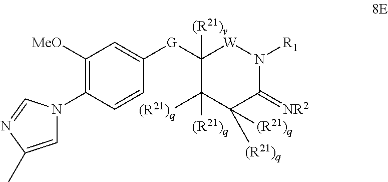

wherein each q is independently 0 or 1, and each R²¹ is independently selected.

In another embodiment of this invention the compound of formula (I) is a compound of the formula:

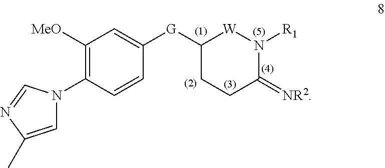

In another embodiment of this invention the compound of formula (I) is a compound of the formula:

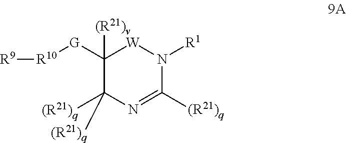

wherein each q is independently 0 or 1, and each R²¹ is independently selected.

In another embodiment of this invention the compound of formula (I) is a compound of the formula:

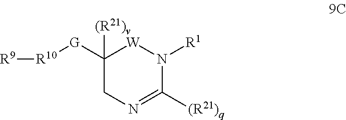

wherein each q is independently 0 or 1, and each R²¹ is independently selected.

In another embodiment of this invention the compound of formula (I) is a compound of the formula:

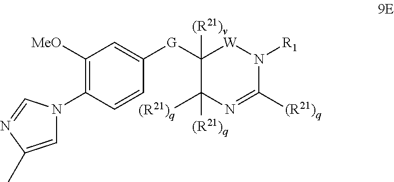

wherein each q is independently 0 or 1, and each R²¹ is independently selected.

In another embodiment of this invention the compound of formula (I) is a compound of the formula:

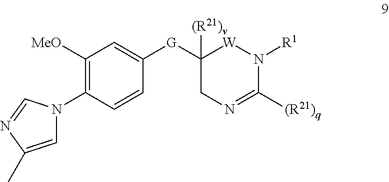

wherein each q is independently 0 or 1, and each R²¹ is independently selected.

In another embodiment of this invention the compound of formula (I) is a compound of the formula:

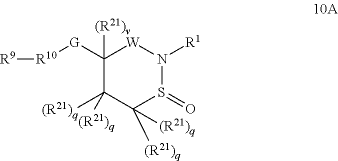

wherein each q is independently 0 or 1, and each R²¹ is independently selected.

In another embodiment of this invention the compound of formula (I) is a compound of the formula:

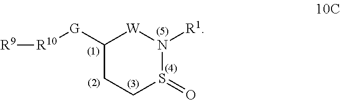

In another embodiment of this invention the compound of formula (I) is a compound of the formula:

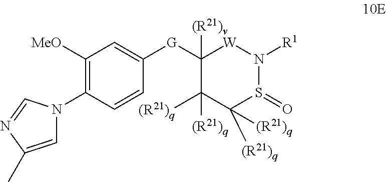

wherein each q is independently 0 or 1, and each R²¹ is independently selected.

In another embodiment of this invention the compound of formula (I) is a compound of the formula:

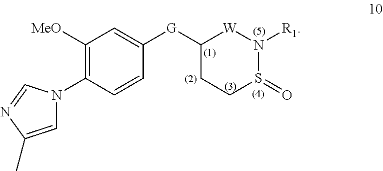

In another embodiment of this invention the compound of formula (I) is a compound of the formula:

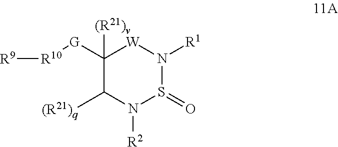

wherein each q is independently 0 or 1, and each R²¹ is independently selected.

In another embodiment of this invention the compound of formula (I) is a compound of the formula:

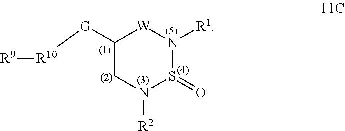

In another embodiment of this invention the compound of formula (I) is a compound of the formula:

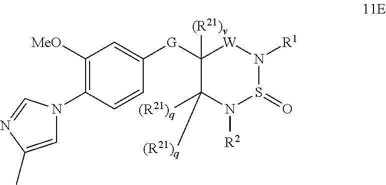

wherein each q is independently 0 or 1, and each R²¹ is independently selected.

In another embodiment of this invention the compound of formula (I) is a compound of the formula:

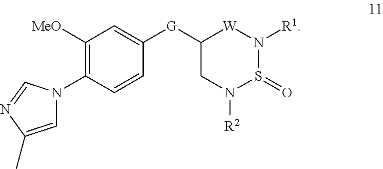

In another embodiment of this invention the compound of formula (I) is a compound of the formula:

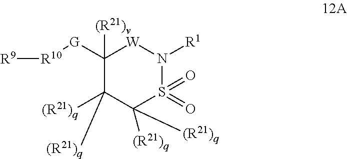

wherein each q is independently 0 or 1, and each R²¹ is independently selected.

In another embodiment of this invention the compound of formula (I) is a compound of the formula:

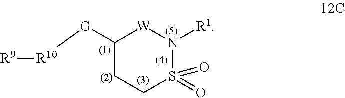

In another embodiment of this invention the compound of formula (I) is a compound of the formula:

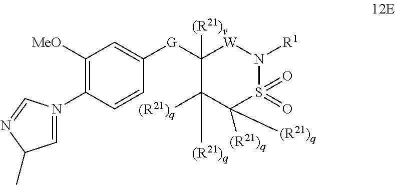

wherein each q is independently 0 or 1, and each R²¹ is independently selected.

In another embodiment of this invention the compound of formula (I) is a compound of the formula:

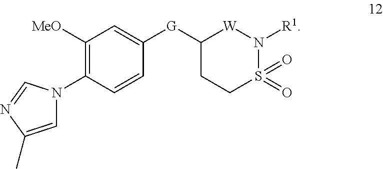

In another embodiment of this invention the compound of formula (I) is a compound of the formula:

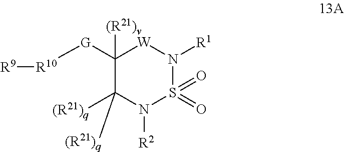

wherein each q is independently 0 or 1, and each R²¹ is independently selected.

In another embodiment of this invention the compound of formula (I) is a compound of the formula:

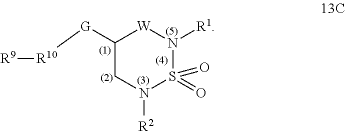

In another embodiment of this invention the compound of formula (I) is a compound of the formula:

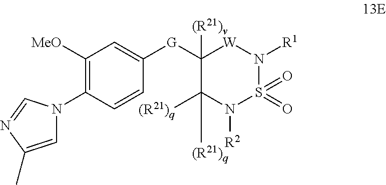

wherein each q is independently 0 or 1, and each R²¹ is independently selected.

In another embodiment of this invention the compound of formula (I) is a compound of the formula:

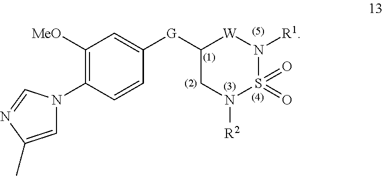

In another embodiment of this invention the compound of formula (I) is a compound of the formula:

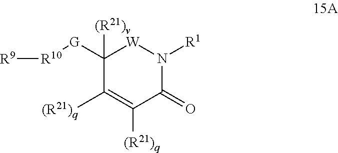

wherein each q is independently 0 or 1, and each R²¹ is independently selected.

In another embodiment of this invention the compound of formula (I) is a compound of the formula:

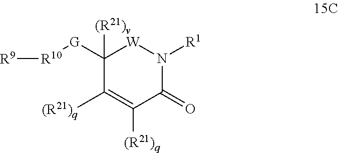

wherein each q is independently 0 or 1, and each R²¹ is independently selected.

In another embodiment of this invention the compound of formula (I) is a compound of the formula:

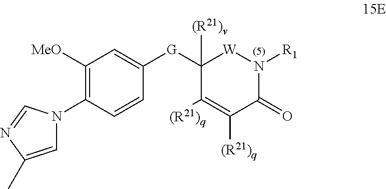

wherein each q is independently 0 or 1, and each R²¹ is independently selected.

In another embodiment of this invention the compound of formula (I) is a compound of the formula:

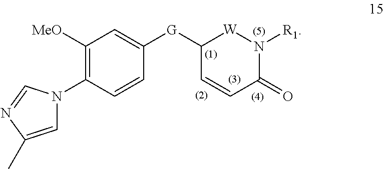

In another embodiment of this invention the compound of formula (I) is a compound of the formula:

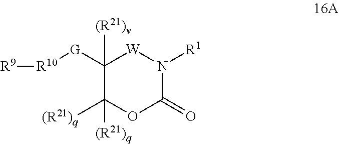

wherein each q is independently 0 or 1, and each R²¹ is independently selected.

In another embodiment of this invention the compound of formula (I) is a compound of the formula:

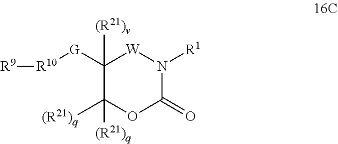

wherein each q is independently 0 or 1, and each R²¹ is independently selected.

In another embodiment of this invention the compound of formula (I) is a compound of the formula:

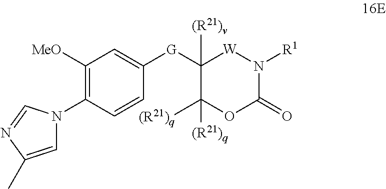

wherein each q is independently 0 or 1, and each R²¹ is independently selected.

In another embodiment of this invention the compound of formula (I) is a compound of the formula:

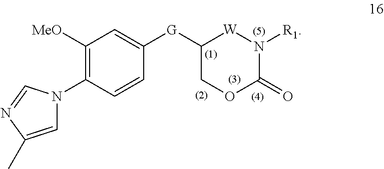

In another embodiment of this invention the compound of formula (I) is a compound of the formula:

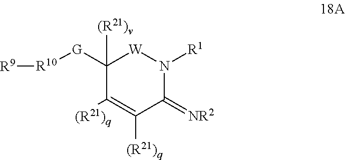

wherein each q is independently 0 or 1, and each R²¹ is independently selected.

In another embodiment of this invention the compound of formula (I) is a compound of the formula:

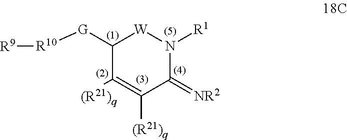

wherein each q is independently 0 or 1, and each R²¹ is independently selected.

In another embodiment of this invention the compound of formula (I) is a compound of the formula:

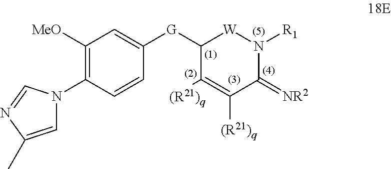

wherein each q is independently 0 or 1, and each R²¹ is independently selected.

In another embodiment of this invention the compound of formula (I) is a compound of the formula:

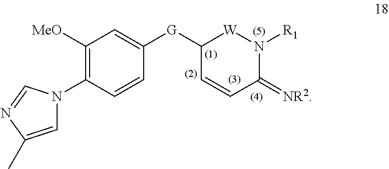

In another embodiment of this invention the compound of formula (I) is a compound of the formula:

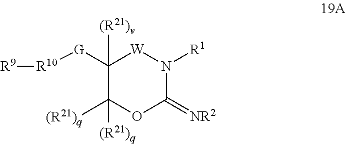

wherein each q is independently 0 or 1, and each R²¹ is independently selected.

In another embodiment of this invention the compound of formula (I) is a compound of the formula:

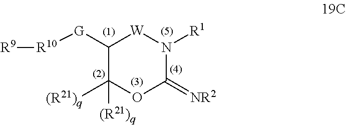

wherein each q is independently 0 or 1, and each R²¹ is independently selected.

In another embodiment of this invention the compound of formula (I) is a compound of the formula:

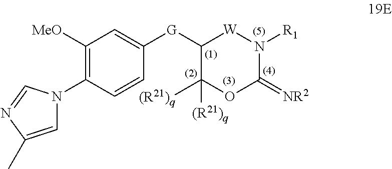

wherein each q is independently 0 or 1, and each R²¹ is independently selected.

In another embodiment of this invention the compound of formula (I) is a compound of the formula:

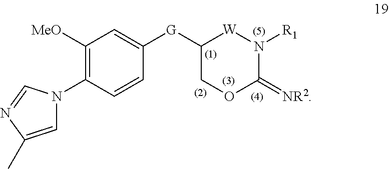

In another embodiment of this invention the compound of formula (I) is a compound of the formula:

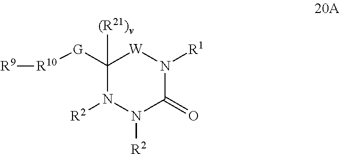

wherein each R² is independently selected.

In another embodiment of this invention the compound of formula (I) is a compound of the formula:

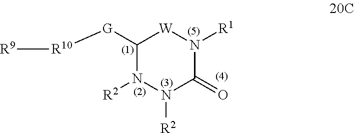

wherein each R² is independently selected.

In another embodiment of this invention the compound of formula (I) is a compound of the formula:

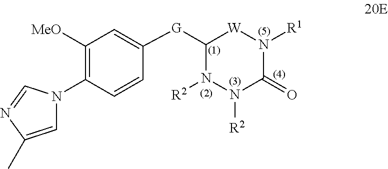

wherein each R² is independently selected.

In another embodiment of this invention the compound of formula (I) is a compound of the formula:

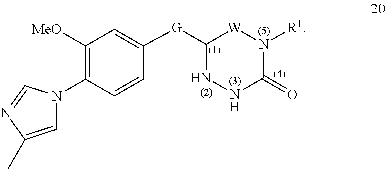

In another embodiment of this invention the compound of formula (I) is a compound of the formula:

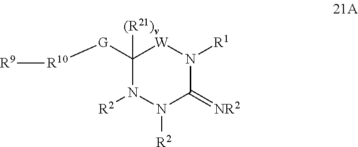

wherein each R² is independently selected.

In another embodiment of this invention the compound of formula (I) is a compound of the formula:

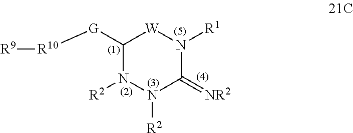

wherein each R² is independently selected.

In another embodiment of this invention the compound of formula (I) is a compound of the formula:

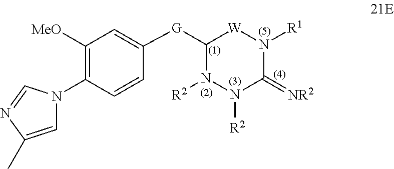

wherein each R² is independently selected.

In another embodiment of this invention the compound of formula (I) is a compound of the formula:

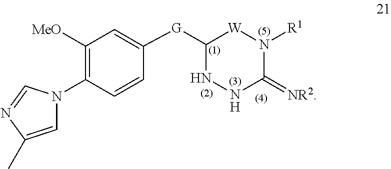

In another embodiment of this invention the compound of formula (I) is a compound of the formula:

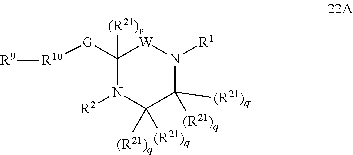

wherein each q is independently 0 or 1, and each R²¹ is independently selected.

In another embodiment of this invention the compound of formula (I) is a compound of the formula:

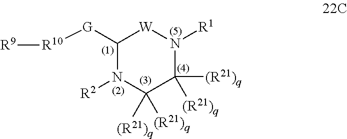

wherein each q is independently 0 or 1, and each R²¹ is independently selected.

In another embodiment of this invention the compound of formula (I) is a compound of the formula:

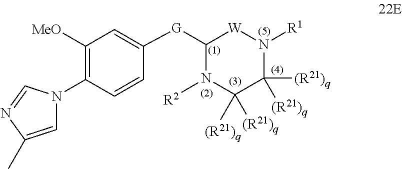

wherein each q is independently 0 or 1, and each R²¹ is independently selected.

In another embodiment of this invention the compound of formula (I) is a compound of the formula:

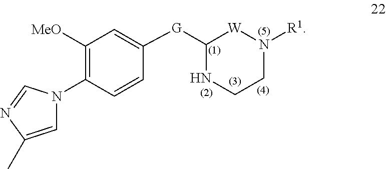

In another embodiment of this invention the compound of formula (I) is a compound of the formula:

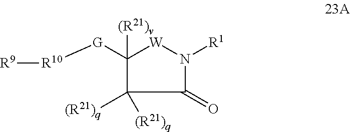

wherein each q is independently 0 or 1, and each R²¹ is independently selected.

In another embodiment of this invention the compound of formula (I) is a compound of the formula:

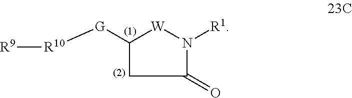

In another embodiment of this invention the compound of formula (I) is a compound of the formula:

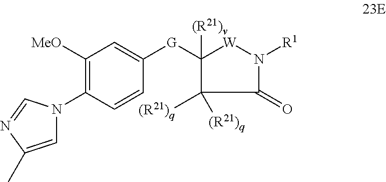

wherein each q is independently 0 or 1, and each R²¹ is independently selected.

In another embodiment of this invention the compound of formula (I) is a compound of the formula:

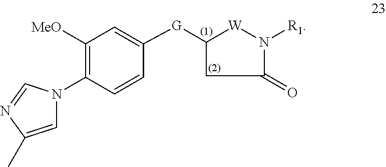

In another embodiment of this invention the compound of formula (I) is a compound of the formula:

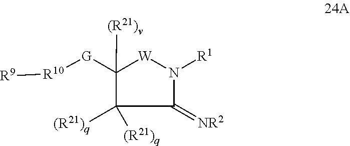

wherein each q is independently 0 or 1, and each R²¹ is independently selected.

In another embodiment of this invention the compound of formula (I) is a compound of the formula:

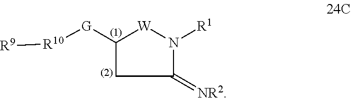

In another embodiment of this invention the compound of formula (I) is a compound of the formula:

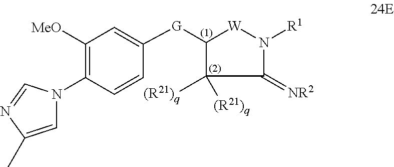

wherein each q is independently 0 or 1, and each R²¹ is independently selected.

In another embodiment of this invention the compound of formula (I) is a compound of the formula:

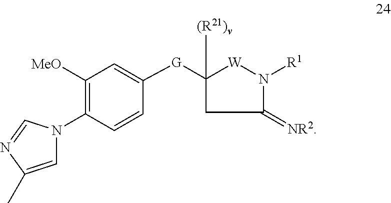

In another embodiment of this invention the compound of formula (I) is a compound of the formula:

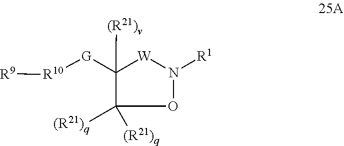

wherein each q is independently 0 or 1, and each R²¹ is independently selected.

In another embodiment of this invention the compound of formula (I) is a compound of the formula:

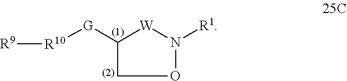

In another embodiment of this invention the compound of formula (I) is a compound of the formula:

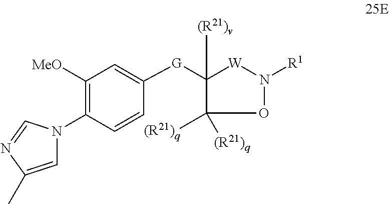

wherein each q is independently 0 or 1, and each R²¹ is independently selected.

In another embodiment of this invention the compound of formula (I) is a compound of the formula:

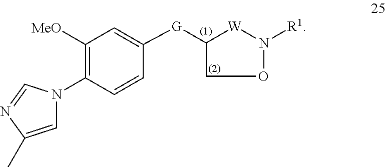

In another embodiment of this invention the compound of formula (I) is a compound of the formula:

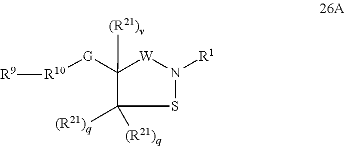

wherein each q is independently 0 or 1, and each R²¹ is independently selected.

In another embodiment of this invention the compound of formula (I) is a compound of the formula:

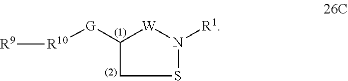

In another embodiment of this invention the compound of formula (I) is a compound of the formula:

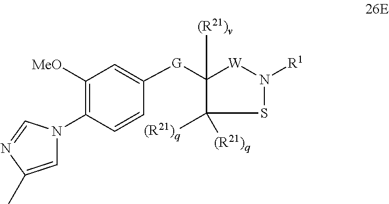

wherein each q is independently 0 or 1, and each R²¹ is independently selected.

In another embodiment of this invention the compound of formula (I) is a compound of the formula:

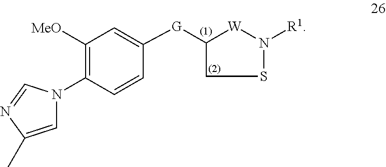

In another embodiment of this invention the compound of formula (I) is a compound of the formula:

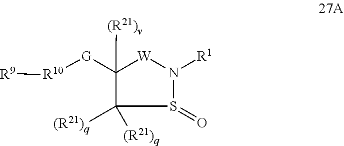

wherein each q is independently 0 or 1, and each R²¹ is independently selected.

In another embodiment of this invention the compound of formula (I) is a compound of the formula:

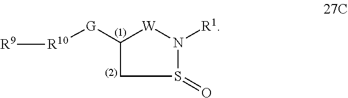

In another embodiment of this invention the compound of formula (I) is a compound of the formula:

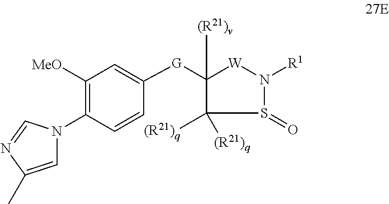

wherein each q is independently 0 or 1, and each R²¹ is independently selected.

In another embodiment of this invention the compound of formula (I) is a compound of the formula:

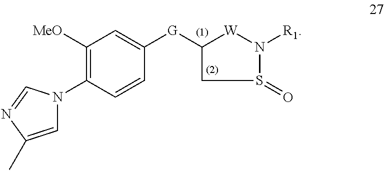

In another embodiment of this invention the compound of formula (I) is a compound of the formula:

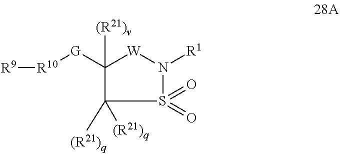

wherein each q is independently 0 or 1, and each R²¹ is independently selected.

In another embodiment of this invention the compound of formula (I) is a compound of the formula:

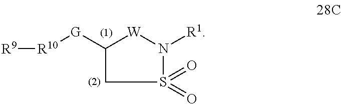

In another embodiment of this invention the compound of formula (I) is a compound of the formula:

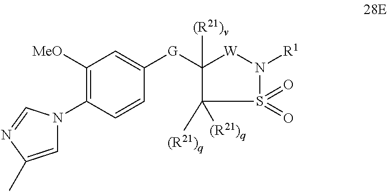

wherein each q is independently 0 or 1, and each R²¹ is independently selected.

In another embodiment of this invention the compound of formula (I) is a compound of the formula:

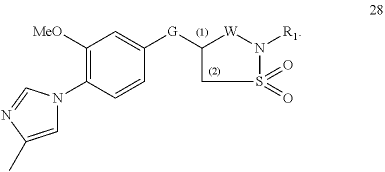

In another embodiment of this invention the compound of formula (I) is a compound of the formula:

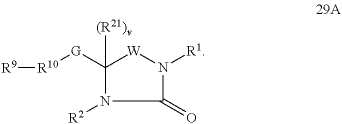

In another embodiment of this invention the compound of formula (I) is a compound of the formula:

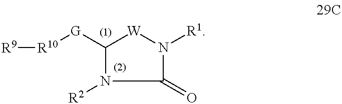

In another embodiment of this invention the compound of formula (I) is a compound of the formula:

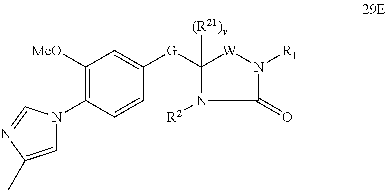

In another embodiment of this invention the compound of formula (I) is a compound of the formula:

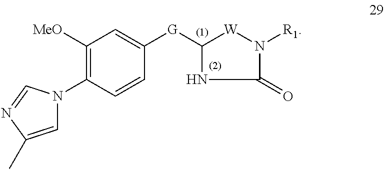

In another embodiment of this invention the compound of formula (I) is a compound of the formula:

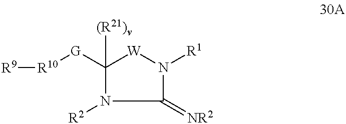

wherein each R² is independently selected.

In another embodiment of this invention the compound of formula (I) is a compound of the formula:

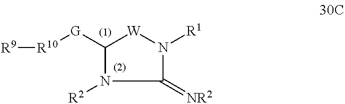

wherein each R² is independently selected.

In another embodiment of this invention the compound of formula (I) is a compound of the formula:

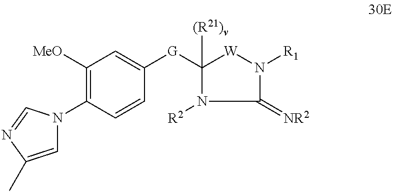

wherein each R² is independently selected.

In another embodiment of this invention the compound of formula (I) is a compound of the formula:

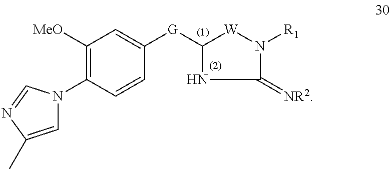

In another embodiment of this invention the compound of formula (I) is a compound of the formula:

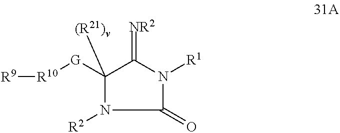

wherein each R² is independently selected.

In another embodiment of this invention the compound of formula (I) is a compound of the formula:

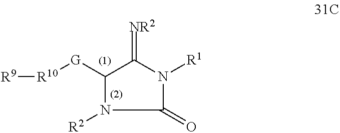

wherein each R² is independently selected.

In another embodiment of this invention the compound of formula (I) is a compound of the formula:

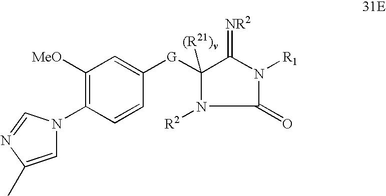

wherein each R² is independently selected.

In another embodiment of this invention the compound of formula (I) is a compound of the formula:

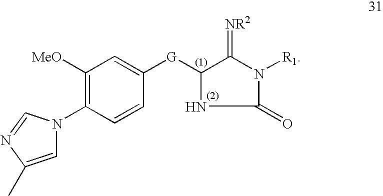

In another embodiment of this invention the compound of formula (I) is a compound of the formula:

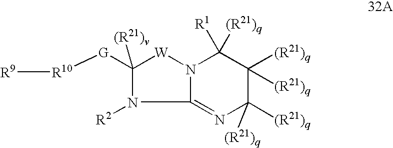

wherein each q is independently 0 or 1, and each R²¹ is independently selected.

In another embodiment of this invention the compound of formula (I) is a compound of the formula:

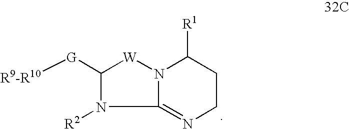

In another embodiment of this invention the compound of formula (I) is a compound of the formula:

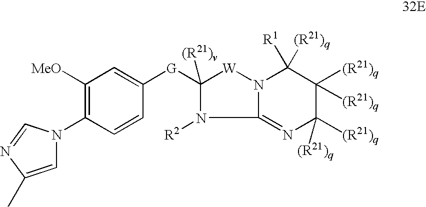

wherein each q is independently 0 or 1, and each R²¹ is independently selected.

In another embodiment of this invention the compound of formula (I) is a compound of the formula:

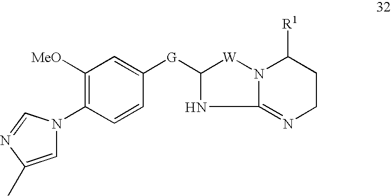

wherein R¹ is selected from the group consisting of: alkyl, alkenyl, alkynyl, cycloalkyl, cycloalkylalkyl-, heterocyclyl (e.g., heterocycloalkyl), cycloalkenyl, arylalkyl-, alkylaryl-, aryl (e.g., phenyl), heteroaryl (e.g., pyridyl), heterocyclenyl (i.e., heterocycloalkenyl), fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-), fused benzocycloalkylalkyl- (i.e., benzofusedcycloalkylalkyl-), fused benzoheterocycloalkylalkyl- (i.e., benzofusedheterocycloalkylalkyl-), fused heteroarylcycloalkylalkyl- (i.e., heteroarylfusedcycloalkylalkyl-), fused heteroarylheterocycloalkylalkyl- (i.e., heteroarylfusedheterocycloalkylalkyl-), fused cycloalkylarylalkyl- (i.e., cycloalkyfusedlarylalkyl-), fused heterocycloalkylarylalkyl- (i.e., heterocycloalkylfusedarylalkyl-), fused cycloalkylheteroarylalkyl- (i.e., cycloalkylfusedheteroarylalkyl-), fused heterocycloalkylheteroarylalkyl- (i.e., heterocycloalkylfusedheteroarylalkyl-), and wherein each of said: alkyl, alkenyl, alkynyl, cycloalkyl, heterocyclyl, cycloalkenyl, aryl, heteroaryl, heterocyclenyl, fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R¹ groups is optionally substituted with 1-5 independently selected R²¹ groups.

In another embodiment of this invention the compound of formula (I) is a compound of the formula:

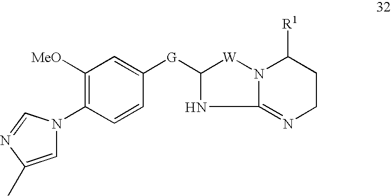

wherein R¹ is selected from the group consisting of: alkyl, alkenyl, alkynyl, cycloalkyl, cycloalkylalkyl-, heterocyclyl (e.g., heterocycloalkyl), cycloalkenyl, arylalkyl-, alkylaryl-, aryl (e.g., phenyl), heteroaryl (e.g., pyridyl), heterocyclenyl (i.e., heterocycloalkenyl), fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl (i.e., cycloalkyfusedlaryl-), fused heterocycloalkylaryl- (i.e., heterocycloalkylfusedaryl-), fused cycloalkylheteroaryl- (i.e., cycloalkylfusedheteroaryl-), fused heterocycloalkylheteroaryl- (i.e., heterocycloalkylfusedheteroaryl-), fused benzocycloalkylalkyl- (i.e., benzofusedcycloalkylalkyl-), fused benzoheterocycloalkylalkyl- (i.e., benzofusedheterocycloalkylalkyl-), fused heteroarylcycloalkylalkyl- (i.e., heteroarylfusedcycloalkylalkyl-), fused heteroarylheterocycloalkylalkyl- (i.e., heteroarylfusedheterocycloalkylalkyl-), fused cycloalkylarylalkyl- (i.e., cycloalkyfusedlarylalkyl-), fused heterocycloalkylarylalkyl- (i.e., heterocycloalkylfusedarylalkyl-), fused cycloalkylheteroarylalkyl- (i.e., cycloalkylfusedheteroarylalkyl-), fused heterocycloalkylheteroarylalkyl- (i.e., heterocycloalkylfusedheteroarylalkyl-), and wherein each of said: alkyl, alkenyl, alkynyl, cycloalkyl, heterocyclyl, cycloalkenyl, aryl, heteroaryl, heterocyclenyl, fused benzocycloalkyl (i.e., benzofusedcycloalkyl), fused benzoheterocycloalkyl (i.e., benzofusedhetero-cycloalkyl), fused heteroarylcycloalkyl (i.e., heteroarylfusedcycloalkyl), fused heteroarylheterocycloalkyl (i.e., heteroarylfusedheterocycloalkyl), fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R¹ groups is optionally substituted with 1-5 independently selected R²¹ groups, provided that provided that no R²¹ group is —NH₂.

In another embodiment of this invention the compound of formula (I) is a compound of the formula:

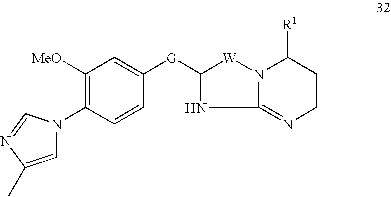

wherein R¹ is selected from the group consisting of: alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzoheterocycloalkyl (i.e., benzofusedhetero-cycloalkyl), fused heteroarylheterocycloalkyl heteroarylfusedheterocycloalkyl), heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylheterocycloalkyl, heteroaryl-, heteroarylalkyl-, heterocyclyl-, heterocyclenyl and heterocyclyalkyl-R¹ groups is optionally substituted with 1-5 independently selected R²¹ groups.

In another embodiment of this invention the compound of formula (I) is a compound of the formula:

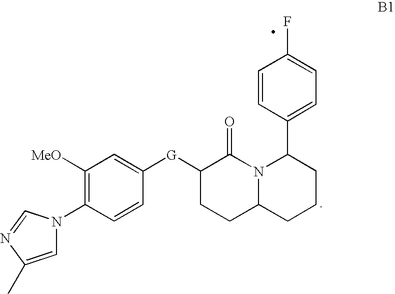

In another embodiment of this invention the compound of formula (I) is a compound of the formula:

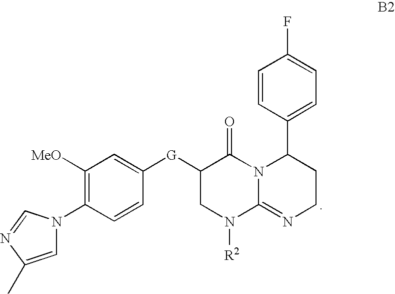

In another embodiment of this invention the compound of formula (I) is a compound of the formula:

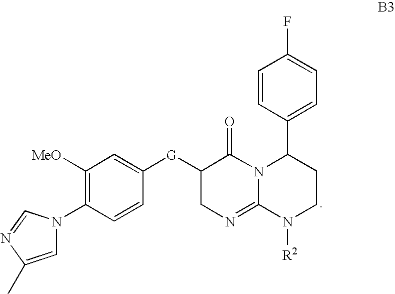

In another embodiment of this invention the compound of formula (I) is a compound of the formula:

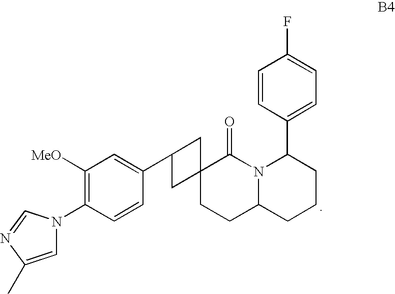

In another embodiment of this invention the compound of formula (I) is a compound of the formula:

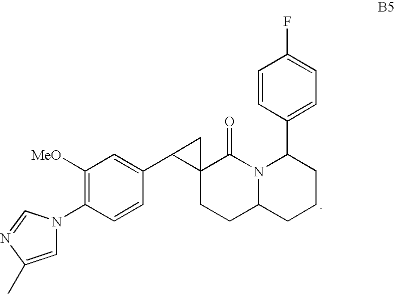

In another embodiment of this invention the compound of formula (I) is a compound of the formula:

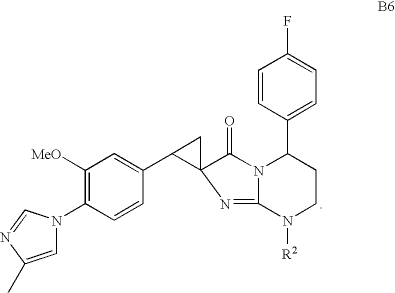

In another embodiment of this invention the compound of formula (I) is a compound of the formula:

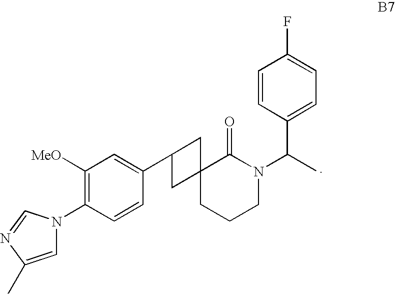

In another embodiment of this invention the compound of formula (I) is a compound of the formula:

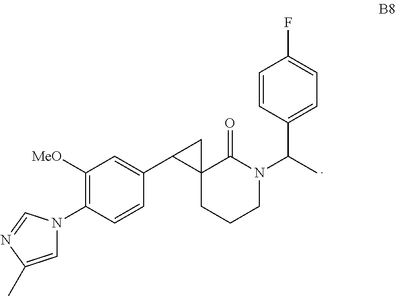

In another embodiment of this invention the compound of formula (I) is a compound of the formula:

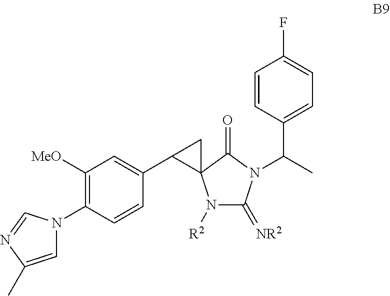

wherein each R² is independently selected.

In another embodiment of this invention the compound of formula (I) is a compound of the formula:

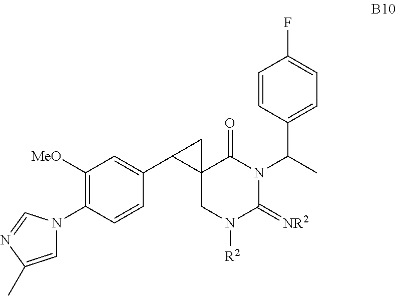

wherein each R² is independently selected.

Examples of R²¹ groups include —OR¹⁵ wherein, for example, R¹⁵ is alkyl (such as methyl or ethyl), or R¹⁵ is cycloalkylalkyl (such as, for example, —CH₂-cyclopropyl), or R¹⁵ is -alkyl-(R¹⁸)ₙ (wherein, for example, said R¹⁸ is —OR²⁰, and said R²⁰ is alkyl, and wherein examples of said -alkyl-(R¹⁸), moiety is —(CH₂)₂OCH₃).

Examples of R²¹ also include —C(O)OR¹⁵ wherein, for example, R¹⁵ is alkyl, such as, for example, methyl).

Examples of R²¹ also include —C(O)NR¹⁵R¹⁶, wherein, for example, one of R¹⁵ or R¹⁶ is H, and the other is selected from the group consisting of: (R¹⁸)ₙ-arylalkyl-, (R¹⁸)ₙ-alkyl-, and cycloalkyl. In one example of this —C(O)NR¹⁵R¹⁶ moiety the R¹⁸ is —OR²⁰, n is 1, R²⁰ is alkyl, said cycloalkyl is cyclobutyl, and said arylalkyl- is benzyl.

Examples of R²¹ also include halo (e.g., Br, Cl or F).

Examples of R²¹ also include arylalkyl, such as, for example, benzyl.

Another embodiment of this invention is directed to a compound of formula (I).

Another embodiment of this invention is directed to a pharmaceutically acceptable salt of a compound of formula (I).

Another embodiment of this invention is directed to a pharmaceutically acceptable ester of a compound of formula (I).

Another embodiment of this invention is directed to a solvate of a compound of formula (I).

Another embodiment of this invention is directed to a compound of formula (I) in isolated form.

Another embodment of this invention is directed to a compound of formula (I) in pure form.

Another embodiment of this invention is directed to a compound of formula (I) selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutically acceptable salt of a compound of formula (I), said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutically acceptable ester of a compound of formula (I), said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a solvate of a compound of formula (I), said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a compound of formula (I) in isolated form, said compound of formula (I) being selected from the group consisting of: (ID) to (IG), (IM) to (IQ), 1 to 32, 1A to 32A, 1C to 32C, 1E to 32E, B1 to B3; B6, B9 and B10.

Another embodment of this invention is directed to a compound of formula (I) in pure form, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a compound of formula (I) in pure and isolated form, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutical composition comprising an effective amount of one or more (e.g., one) compounds of formula (I) and a pharmaceutically acceptable carrier. Another embodiment is directed to a pharmaceutical composition comprising an effective amount of one or more (e.g., one) compounds of formula (I) and a pharmaceutically acceptable carrier, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutical composition comprising an effective amount of a pharmaceutically acceptable salt of one or more (e.g., one) compounds of formula (I) and a pharmaceutically acceptable carrier. Another embodiment is directed to a pharmaceutical composition comprising an effective amount of a pharmaceutically acceptable salt of one or more (e.g., one) compounds of formula (I) and a pharmaceutically acceptable carrier, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutical composition comprising an effective amount of a pharmaceutically acceptable ester of one or more (e.g., one) compounds of formula (I) and a pharmaceutically acceptable carrier. Another embodiment is directed to a pharmaceutical composition comprising an effective amount of a pharmaceutically acceptable ester of one or more (e.g., one) compounds of formula (I) and a pharmaceutically acceptable carrier, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutical composition comprising an effective amount of a solvate of one or more (e.g., one) compounds of formula (I) and a pharmaceutically acceptable carrier. Another embodiment is directed to a pharmaceutical composition comprising an effective amount of a solvate of one or more (e.g., one) compounds of formula (I) and a pharmaceutically acceptable carrier, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutical composition comprising an effective amount of one or more (e.g., one) compounds of formula (I), or a pharmaceutically acceptable salt, ester or solvate thereof, and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising an effective amount of one or more (e.g., one) compounds of formula (I), and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutical composition comprising a therapeutically effective amount of at least one compound of Formula (I), or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors. In another embodiment the compound of formula (I) is selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutical composition comprising an effective amount of one or more (e.g., one) compounds of formula (I), and effective amount of one or more BACE inhibitors, and a pharmaceutically acceptable carrier.

Another embodiment of this invention is directed to a pharmaceutical composition comprising an effective amount of one or more (e.g., one) compounds of formula (I), and effective amount of one or more BACE inhibitors, and a pharmaceutically acceptable carrier, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a pharmaceutical composition comprising an effective amount of one or more (e.g., one) compounds selected from the group consisting of: (ID) to (IG), (IM) to (IQ), 1 to 32, 1A to 32A, 1C to 32C, 1E to 32E, B1 to B3, B6, B9 and B10, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds selected from the group consisting of: (ID) to (IG), (IM) to (IQ), 1 to 32, 1A to 32A, 1C to 32C, 1E to 32E, B1 to B3, B6, B9 and B10, and effective amount of one or more muscarinic antagonists (e.g., m₁ agonists or m₂ antagonists), and a pharmaceutically acceptable carrier.

Another embodiment of this invention is directed to a pharmaceutical composition comprising an effective amount of one or more (e.g., one) compounds of formula (I), and effective amount of Exelon (rivastigmine), and a pharmaceutically acceptable carrier.

Another embodiment of this invention is directed to a pharmaceutical composition comprising an effective amount of one or more (e.g., one) compounds of formula (I), and effective amount of Cognex (tacrine), and a pharmaceutically acceptable carrier.

Another embodiment of this invention is directed to a pharmaceutical composition comprising an effective amount of one or more (e.g., one) compounds of formula (I), and effective amount of a Tau kinase inhibitor, and a pharmaceutically acceptable carrier.

Another embodiment of this invention is directed to a pharmaceutical composition comprising an effective amount of one or more (e.g., one) compounds of formula (I), and effective amount of one or more Tau kinase inhibitor (e.g., GSK3beta inhibitor, cdk5 inhibitor, ERK inhibitor), and a pharmaceutically acceptable carrier.

Another embodiment of this invention is directed to a pharmaceutical composition comprising an effective amount of one or more (e.g., one) compounds of formula (I), and effective amount of one anti-Abeta vaccine (active immunization), and a pharmaceutically acceptable carrier.

Another embodiment of this invention is directed to a pharmaceutical composition comprising an effective amount of one or more (e.g., one) compounds of formula (I), and effective amount of one or more APP ligands, and a pharmaceutically acceptable carrier.

Another embodiment of this invention is directed to a pharmaceutical composition comprising an effective amount of one or more (e.g., one) compounds of formula (I), and effective amount of one or more agents that upregulate insulin degrading enzyme and/or neprilysin, and a pharmaceutically acceptable carrier.

Another embodiment of this invention is directed to a pharmaceutical composition comprising an effective amount of one or more (e.g., one) compounds of formula (I), and effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe), and a pharmaceutically acceptable carrier.

Another embodiment of this invention is directed to a pharmaceutical composition comprising an effective amount of one or more (e.g., one) compounds of formula (I), and effective amount of one or more fibrates (for example, clofibrate, Clofibride, Etofibrate, Aluminium Clofibrate), and a pharmaceutically acceptable carrier

Another embodiment of this invention is directed to a pharmaceutical composition comprising an effective amount of one or more (e.g., one) compounds of formula (I), and effective amount of one or more LXR agonists, and a pharmaceutically acceptable carrier.

Another embodiment of this invention is directed to a pharmaceutical composition comprising an effective amount of one or more (e.g., one) compounds of formula (I), and effective amount of one or more LRP mimics, and a pharmaceutically acceptable carrier.

Another embodiment of this invention is directed to a pharmaceutical composition comprising an effective amount of one or more (e.g., one) compounds of formula (I), and effective amount of one or more 5-HT6 receptor antagonists, and a pharmaceutically acceptable carrier.

Another embodiment of this invention is directed to a pharmaceutical composition comprising an effective amount of one or more (e.g., one) compounds of formula (I), and effective amount of one or more nicotinic receptor agonists, and a pharmaceutically acceptable carrier.

Another embodiment of this invention is directed to a pharmaceutical composition comprising an effective amount of one or more (e.g., one) compounds of formula (I), and effective amount of one or more H3 receptor antagonists, and a pharmaceutically acceptable carrier.

Another embodiment of this invention is directed to a pharmaceutical composition comprising an effective amount of one or more (e.g., one) compounds of formula (I), and effective amount of one or more histone deacetylase inhibitors, and a pharmaceutically acceptable carrier.

Another embodiment of this invention is directed to a pharmaceutical composition comprising an effective amount of one or more (e.g., one) compounds of formula (I), and effective amount of one or more hsp90 inhibitors, and a pharmaceutically acceptable carrier.

Another embodiment of this invention is directed to a pharmaceutical composition comprising an effective amount of one or more (e.g., one) compounds of formula (I), and effective amount of one or more m1 muscarinic receptor agonists, and a pharmaceutically acceptable carrier.

Another embodiment of this invention is directed to combinations, i.e., a pharmaceutical composition, comprising a pharmaceutically acceptable carrier, an effective (i.e., therapeutically effective) amount of one or more compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

Another embodiment of this invention is directed to a pharmaceutical composition comprising an effective amount of one or more (e.g., one) compounds of formula (I), and effective amount of one or more 5-HT6 receptor antagonists mGluR1 or mGluR5 positive allosteric modulators or agonists, and a pharmaceutically acceptable carrier.

Another embodiment of this invention is directed to a pharmaceutical composition comprising an effective amount of one or more (e.g., one) compounds of formula (I), and effective amount of one or more one mGluR2/3 antagonists, and a pharmaceutically acceptable carrier.

Another embodiment of this invention is directed to a pharmaceutical composition comprising an effective amount of one or more (e.g., one) compounds of formula (I), and effective amount of one or more anti-inflammatory agents that can reduce neuroinflammation, and a pharmaceutically acceptable carrier.

Another embodiment of this invention is directed to a pharmaceutical composition comprising an effective amount of one or more (e.g., one) compounds of formula (I), and effective amount of one or more Prostaglandin EP2 receptor antagonists, and a pharmaceutically acceptable carrier.

Another embodiment of this invention is directed to a pharmaceutical composition comprising an effective amount of one or more (e.g., one) compounds of formula (I), and effective amount of one or more PAI-1 inhibitors, and a pharmaceutically acceptable carrier.

Another embodiment of this invention is directed to a pharmaceutical composition comprising an effective amount of one or more (e.g., one) compounds of formula (I), and effective amount of one or more agents that can induce Abeta efflux such as gelsolin, and a pharmaceutically acceptable carrier.

The compounds of formula (I) can be useful as gamma secretase modulators and can be useful in the treatment and prevention of diseases such as, for example, central nervous system disorders (such as Alzheimers disease and Downs Syndrome), mild cognitive impairment, glaucoma, cerebral amyloid angiopathy, stroke, dementia, microgliosis, brain inflammation, and olfactory function loss.

The compounds of formulas (ID) to (IG), (IM) to (IQ), 1 to 32, 1A to 32A, 1C to 32C, 1E to 32E, B1 to B3, B6, B9 and B10 can be useful as gamma secretase modulators and can be useful in the treatment and prevention of diseases such as, for example, central nervous system disorders such as Alzheimers disease and Downs Syndrome.

The compounds of formulas (ID) to (IG), (IM) to (IQ), 1 to 32, 1A to 32A, 1C to 32C, 1E to 32E, B1 to B3, B6, B9 and B10 can be useful as gamma secretase modulators and can be useful in the treatment and prevention of diseases such as, for example, mild cognitive impairment, glaucoma, cerebral amyloid angiopathy, stroke, dementia, microgliosis, brain inflammation, and olfactory function loss.

Another embodiment of this invention is directed to a method of treating a central nervous system disorder comprising administering a therapeutically effective amount of at least one compound of Formula (I) to a patient in need of such treatment.

Another embodiment of this invention is directed to a method of treating a central nervous system disorder comprising administering a therapeutically effective amount of a pharmaceutical composition comprising a therapeutically effective amount of at least one compound of Formula (I), or a pharmaceutically acceptable salt, solvate, or ester thereof, and at least one pharmaceutically acceptable carrier.

Another embodiment of this invention is directed to a method of treating a central nervous system disorder comprising administering a therapeutically effective amount of a pharmaceutical composition comprising a therapeutically effective amount of at least one compound of Formula (I), or a pharmaceutically acceptable salt, solvate, or ester thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

Thus, another embodiment of this invention is directed to a method for modulating (including inhibiting, antagonizing and the like) gamma-secretase comprising administering an effective (i.e., therapeutically effective) amount of one or more (e.g., one) compounds of formula (I) to a patient in need of such treatment.

Another embodiment of this invention is directed to a method for modulating (including inhibiting, antagonizing and the like) gamma-secretase, comprising administering an effective (i.e., therapeutically effective) amount of a compound of formula (I) to a patient in need of treatment.

Thus, another embodiment of this invention is directed to a method for modulating (including inhibiting, antagonizing and the like) gamma-secretase comprising administering an effective (i.e., therapeutically effective) amount of one or more (e.g., one) compounds of formula (I) to a patient in need of such treatment, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a method for modulating (including inhibiting, antagonizing and the like) gamma-secretase, comprising administering an effective (i.e., therapeutically effective) amount of a compound of formula (I) to a patient in need of treatment, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a method of treating one or more neurodegenerative diseases, comprising administering an effective (i.e., therapeutically effective) amount of one or more (e.g., one) compounds of formula (I) to a patient in need of treatment.

Another embodiment of this invention is directed to a method of treating one or more neurodegenerative diseases, comprising administering an effective (i.e., therapeutically effective) amount of a compound of formula (I) to a patient in need of treatment.

Another embodiment of this invention is directed to a method of treating one or more neurodegenerative diseases, comprising administering an effective (i.e., therapeutically effective) amount of one or more (e.g., one) compounds of formula (I) to a patient in need of treatment, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a method of treating one or more neurodegenerative diseases, comprising administering an effective (i.e., therapeutically effective) amount of a compound of formula (I) to a patient in need of treatment, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of one or more (e.g., one) compounds of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of one or more (e.g., one) compounds of formula (I) to a patient in need of treatment, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment, said compound of formula (I) being selected from the group consisting of: (ID) to (IG), (IM) to (IQ), 1 to 32, 1A to 32A, 10 to 32C, 1E to 32E, B1 to B3, B6, B9 and B10.

Another embodiment of this invention is directed to a method of treating Alzheimer's disease, comprising administering an effective (i.e., therapeutically effective) amount of one or more (e.g., one) compounds of formula (I) to a patient in need of treatment.

Another embodiment of this invention is directed to a method of treating Alzheimer's disease, comprising administering an effective (i.e., therapeutically effective) amount of a compound of formula (I) to a patient in need of treatment.

Another embodiment of this invention is directed to a method of treating Alzheimer's disease, comprising administering an effective (i.e., therapeutically effective) amount of one or more (e.g., one) compounds of formula (I) to a patient in need of treatment, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a method of treating Alzheimer's disease, comprising administering an effective (i.e., therapeutically effective) amount of a compound of formula (I) to a patient in need of treatment, said compound of formula (I) being selected from the group consisting of: (ID) to (IG), (IM) to (IQ), 1 to 32, 1A to 32A, 1C to 32C, 1E to 32E, B1 to B3, B6, B9 and B10.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of one or more (e.g., one) compounds of formula (I) to a patient in need of treatment.

Another embodiment of this invention is directed to a method of treating mild cognitive impairment, glaucoma, cerebral amyloid angiopathy, stroke, dementia, microgliosis, brain inflammation, or olfactory function loss, comprising administering an effective (i.e., therapeutically effective) amount of a compound of formula (I) to a patient in need of treatment.

Another embodiment of this invention is directed to a method of treating mild cognitive impairment, comprising administering an effective amount of one or more (e.g., one) compounds of formula (I) to a patient in need of treatment.

Another embodiment of this invention is directed to a method of treating glaucoma, comprising administering an effective amount of one or more (e.g., one) compounds of formula (I) to a patient in need of treatment.

Another embodiment of this invention is directed to a method of treating cerebral amyloid angiopathy, comprising administering an effective amount of one or more (e.g., one) compounds of formula (I) to a patient in need of treatment.

Another embodiment of this invention is directed to a method of treating stroke, comprising administering an effective amount of one or more (e.g., one) compounds of formula (I) to a patient in need of treatment.

Another embodiment of this invention is directed to a method of treating dementia, comprising administering an effective amount of one or more (e.g., one) compounds of formula (I) to a patient in need of treatment.

Another embodiment of this invention is directed to a method of treating microgliosis, comprising administering an effective amount of one or more (e.g., one) compounds of formula (I) to a patient in need of treatment.

Another embodiment of this invention is directed to a method of treating brain inflammation, comprising administering an effective amount of one or more (e.g., one) compounds of formula (I) to a patient in need of treatment.

Another embodiment of this invention is directed to a method of treating olfactory function loss, comprising administering an effective amount of one or more (e.g., one) compounds of formula (I) to a patient in need of treatment.

Another embodiment of this invention is directed to a method of treating Downs syndrome, comprising administering an effective amount of one or more (e.g., one) compounds of formula (I) to a patient in need of treatment.

Another embodiment of this invention is directed to a method of treating Downs syndrome, comprising administering an effective amount of a compound of formula (I) to a patient in need of treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of an effective amount of one or more (e.g. one) compounds of formula (I) and the administration of an effective amount of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

Thus, other embodiments of this invention are directed to any one of the methods of treatment, or methods of inhibiting, described herein, wherein an effective amount of the compound of formula (I) is used in combination with an effective amount of one or more other pharmaceutically active ingredients (e.g., drugs). The other pharmaceutically active ingredients (i.e., drugs) are selected from the group consisting of: BACE inhibitors (beta secretase inhibitors); muscarinic antagonists (e.g., m₁ agonists or m₂ antagonists); cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors (e.g., GSK3beta inhibitors, cdk5 inhibitors, or ERK inhibitors); anti-Abeta vaccine; APP ligands; agents that upregulate insulin cholesterol lowering agents (for example, statins such as Atorvastatin, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LRP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

Another embodiment of this invention is directed to combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (ID) to (IG), (IM) to (IQ), 1 to 32, 1A to 32A, 1C to 32C, 1E to 32E, B1 to B3, B6, B9 or B10, and the administration of one or more (e.g., one) other pharmaceutical active ingredients (e.g., drugs). The compounds of formula (ID) to (IG), (IM) to (IQ), 1 to 32, 1A to 32A, 1C to 32C, 1E to 32E, B1 to B3, B6, B9 or B10, and the other drugs can be administered separately (i.e., each is in its own separate dosage form), or the compounds of formula (ID) to (IG), (IM) to (IQ), 1 to 32, 1A to 32A, 1C to 32C, 1E to 32E, B1 to B3, B6, B9 or B10 can be combined with the other drugs in the same dosage form.

Thus, other embodiments of this invention are directed to any one of the methods of treatment, or methods of inhibiting, described herein, wherein the compounds of formulas (ID) to (IG), (IM) to (IQ), 1 to 32, 1A to 32A, 1C to 32C, 1E to 32E, B1 to B3, B6, B9 or B10 are used in combination with an effective amount of one or more other pharmaceutically active ingredients selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe).

Another embodiment of this invention is directed to a method of treating Alzheimer's disease, comprising administering an effective amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective amount of a compound of formula (I), in combination with an effective amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of one or more (e.g., one) compounds of formulas (ID) to (IG), (IM) to (IQ), 1 to 32, 1A to 32A, 1C to 32C, 1E to 32E, B1 to B3, B6, B9 or B10, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of a compound of formula (ID) to (IG), (IM) to (IQ), 1 to 32, 1A to 32A, 1C to 32C, 1E to 32E, B1 to B3, B6, B9 or B10, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of one or more (e.g., one) compounds of formulas (ID) to (IG), (IM) to (IQ), 1 to 32, 1A to 32A, 1C to 32C, 1E to 32E, B1 to B3, B6, B9 or B10, in combination with an effective (i.e., therapeutically effective) amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective (i.e., therapeutically effective) amount of one or more (e.g., one) compounds of formulas (ID) to (IG), (IM) to (IQ), 1 to 32, 1A to 32A, 1C to 32C, 1E to 32E, B1 to B3, B6, B9 and B10, in combination with an effective (i.e., therapeutically effective) amount of one or more BACE inhibitors.

Another embodiment of this invention is directed to a method of treating Alzheimer's disease, comprising administering an effective amount of one or more compounds of formula (I), in combination with an effective amount of Exelon (rivastigmine).

Another embodiment of this invention is directed to a method of treating Alzheimer's disease, comprising administering an effective amount of one or more compounds of formula (I), in combination with an effective amount of Cognex (tacrine).

Another embodiment of this invention is directed to a method of treating Alzheimer's disease, comprising administering an effective amount of one or more compounds of formula (I), in combination with an effective amount of a Tau kinase inhibitor.

Another embodiment of this invention is directed to a method of treating Alzheimer's disease, comprising administering an effective amount of one or more compounds of formula (I), in combination with an effective amount of one or more Tau kinase inhibitor (e.g., GSK3beta inhibitor, cdk5 inhibitor, ERK inhibitor).

This invention also provides a method of treating Alzheimer's disease, comprising administering an effective amount of one or more compounds of formula (I), in combination with an effective amount of one anti-Abeta vaccination (active immunization).

Another embodiment of this invention is directed to a method of treating Alzheimer's disease, comprising administering an effective amount of one or more compounds of formula (I), in combination with an effective amount of one or more APP ligands.

Another embodiment of this invention is directed to a method of treating Alzheimer's disease, comprising administering an effective amount of one or more compounds of formula (I), in combination with an effective amount of one or more agents that upregulate insulin degrading enzyme and/or neprilysin.

Another embodiment of this invention is directed to a method of treating Alzheimer's disease, comprising administering an effective amount of one or more compounds of formula (I), in combination with an effective amount of one or more cholesterol lowering agents (for example, statins such as Atorvastatin, Fluvastatin, Lovastatin, Mevastatin, Pitavastatin, Pravastatin, Rosuvastatin, Simvastatin, and cholesterol absorption inhibitor such as Ezetimibe).

This invention also provides a method of treating Alzheimer's disease, comprising administering an effective amount of one or more compounds of formula (I), in combination with an effective amount of one or more fibrates (for example, clofibrate, Clofibride, Etofibrate, Aluminium Clofibrate).

Another embodiment of this invention is directed to a method of treating Alzheimer's disease, comprising administering an effective amount of one or more compounds of formula (I), in combination with an effective amount of one or more LXR agonists.

Another embodiment of this invention is directed to a method of treating Alzheimer's disease, comprising administering an effective amount of one or more compounds of formula (I), in combination with an effective amount of one or more LRP mimics.

Another embodiment of this invention is directed to a method of treating Alzheimer's disease, comprising administering an effective amount of one or more compounds of formula (I), in combination with an effective amount of one or more 5-HT6 receptor antagonists.

Another embodiment of this invention is directed to a method of treating Alzheimer's disease, comprising administering an effective amount of one or more compounds of formula (I), in combination with an effective amount of one or more nicotinic receptor agonists.

Another embodiment of this invention is directed to a method of treating Alzheimer's disease, comprising administering an effective amount of one or more compounds of formula (I), in combination with an effective amount of one or more H3 receptor antagonists.

This invention also provides a method of treating Alzheimer's disease, comprising administering an effective amount of one or more compounds of formula (I), in combination with an effective amount of one or more histone deacetylase inhibitors.

Another embodiment of this invention is directed to a method of treating Alzheimer's disease, comprising administering an effective amount of one or more compounds of formula (I), in combination with an effective amount of one or more hsp90 inhibitors.

Another embodiment of this invention is directed to a method of treating Alzheimer's disease, comprising administering an effective amount of one or more compounds of formula (I), in combination with an effective amount of one or more m1 muscarinic receptor agonists.

Another embodiment of this invention is directed to a method of treating Alzheimer's disease, comprising administering an effective amount of one or more compounds of formula (I), in combination with an effective amount of one or more 5-HT6 receptor antagonists mGluR1 or mGluR5 positive allosteric modulators or agonists

Another embodiment of this invention is directed to a method of treating Alzheimer's disease, comprising administering an effective amount of one or more compounds of formula (I), in combination with an effective amount of one or more mGluR2/3 antagonists.

Another embodiment of this invention is directed to a method of treating Alzheimer's disease, comprising administering an effective amount of one or more compounds of formula (I), in combination with an effective amount of one or more anti-inflammatory agents that can reduce neuroinflammation.

Another embodiment of this invention is directed to a method of treating Alzheimer's disease, comprising administering an effective amount of one or more compounds of formula (I), in combination with an effective amount of one or more Prostaglandin EP2 receptor antagonists.

Another embodiment of this invention is directed to a method of treating Alzheimer's disease, comprising administering an effective amount of one or more compounds of formula (I), in combination with an effective amount of one or more PAI-1 inhibitors.

Another embodiment of this invention is directed to a method of treating Alzheimer's disease, comprising administering an effective amount of one or more compounds of formula (I), in combination with an effective amount of one or more agents that can induce Abeta efflux such as gelsolin.

Another embodiment of this invention is directed to a method of treating Downs syndrome, comprising administering an effective (i.e., therapeutically effective) amount of one or more (e.g., one) compounds of formula (ID) to (IG), (IM) to (IQ), 1 to 32, 1A to 32A, 10 to 32C, 1E to 32E, B1 to B3, B6, B9 or B10 to a patient in need of treatment.

Another embodiment of this invention is directed to a method of treating Downs syndrome, comprising administering an effective (i.e., therapeutically effective) amount of a compound of formula (ID) to (IG), (IM) to (IQ), 1 to 32, 1A to 32A, 1C to 32C, 1E to 32E, B1 to B3, B6, B9 or B10 to a patient in need of treatment.

Another embodiment of this invention is directed to a method of treating Downs syndrome, comprising administering an effective (i.e., therapeutically effective) amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective (i.e., therapeutically effective) amount of one or more (e.g., one) compounds of formula (ID) to (IG), (IM) to (IQ), 1 to 32, 1A to 32A, 1C to 32C, 1E to 32E, B1 to B3, B6, B9 or B10, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective (i.e., therapeutically effective) amount of a compound of formula (ID) to (IG), (IM) to (IQ), 1 to 32, 1A to 32A, 1C to 32C, 1E to 32E, B1 to B3, B6, B9 or B10, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to combinations (i.e., pharmaceutical compositions) comprising an effective (i.e., therapeutically effective) amount of one or more (e.g., one) compounds of formulas (ID) to (IG), (IM) to (IQ), 1 to 32, 1A to 32A, 1C to 32C, 1E to 32E, B1 to B3, B6, B9 or B10, in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors. The pharmaceutical compositions also comprise a pharmaceutically acceptable carrier.

In other embodiments of the methods described above the compound of formula (I) is selected from the group consisting of: A1 to A107, B4, B5, B7, and B8. In other embodiments of the pharmaceutical compositions described above the compound of formula (I) is selected from the group consisting of: A1 to A105. In other embodiments of the pharmaceutical compositions described above the compound of formula (I) is selected from the group consisting of: A3 to A16, and A86 to A89. In other embodiments of the pharmaceutical compositions described above the compound of formula (I) is selected from the group consisting of: A106 and A107. In other embodiments of the methods described above a compound selected from the group consisting of A7, A8, A9 and A11 is used instead of a compound of formula (I). In other embodiments of the pharmaceutical compositions described above a compound selected from the group consisting of A7, A8, A9 and A11 is used instead of a compound of formula (I).

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase, or (e) treat mild cognitive impairment, or (f) treat glaucoma, or (g) treat cerebral amyloid angiopathy, or (h) treat stroke, or (i) treat dementia, or (j) treat microgliosis, or (k) treat brain inflammation, or (l) treat olfactory function loss.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

This invention also provides a kit comprising, in separate containers, in a single package, pharmaceutical compositions for use in combination, wherein one container comprises an effective amount of a compound selected from the group consisting of the compounds of formulas (ID) to (IG), (IM) to (IQ), 1 to 32, 1A to 32A, 1C to 32C, 1E to 32E, B1 to 83, B6, B9 or B10 in a pharmaceutically acceptable carrier, and another container (i.e., a second container) comprises an effective amount of another pharmaceutically active ingredient (as described above), the combined quantities of the compounds of formulas (ID) to (IG), (IM) to (IQ)), 1 to 32, 1A to 32A, 1C to 32C, 1E to 32E, B1 to B3, B6, B9 or B10 and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any of the above embodiments wherein one or more (e.g., one) compounds selected from the group consisting of A1, A2, B4, B5, B7 or B8 are used instead of the compounds of formulas (ID) to (IG), (IM) to (IQ), 1 to 32, 1A to 32A, 1C to 32C, 1E to 32E, B1 to B3, B6, B9 or B10.

Other embodiments of this invention are directed to any one of the above embodiments directed to pharmaceutical compositions, methods, and kits wherein the compound of formula (I) used is selected from the group consisting of: A1 to A6, A10, A12 to A107, B4, B5, B7, and B8. Other embodiments of this invention are directed to any one of the above embodiments directed to pharmaceutical compositions, methods, and kits wherein the compound of formula (I) used is selected from the group consisting of: A1 to A6, A10, A12 to A105. Other embodiments of this invention are directed to any one of the above embodiments directed to pharmaceutical compositions, methods, and kits wherein the compound of formula (I) used is selected from the group consisting of: A3 to A6, A10, A12 to A16, and A86 to A89. Other embodiments of this invention are directed to any one of the above embodiments directed to pharmaceutical compositions, methods, and kits wherein a compound of formula (I) used is selected from the group consisting of: A106 and A107. Other embodiments of this invention are directed to any one of the above embodiments directed to pharmaceutical compositions, methods, and kits wherein a compound selected from the group consisting of: A7, A8, A9 and A11 is used instead of a compound of formula (I).

Another embodiment of this invention is directed to a compound selected from the group consisting of: A1 to A6, A10, A12 to A107, B4, B5, B7, and B8.

Another embodiment of this invention is directed to a pharmaceutically acceptable salt of a compound selected from the group consisting of: A1 to A6, A10, A12 to A107, B4, B5, B7, and B8.

Another embodiment of this invention is directed to a pharmaceutically acceptable ester of a compound selected from the group consisting of: A6, A10, A12, A33-A48, A55-A61, A68-A73, A80-A85, A94-A97, and A102-A105.

Another embodiment of this invention is directed to a solvate of a compound selected from the group consisting of: A1 to A6, A10, A12 to A107, B4, B5, B7, and B8.

Another embodiment of this invention is directed to a compound selected from the group consisting of: A1 to A6, A10, A12 to A105.

Another embodiment of this invention is directed to a pharmaceutically acceptable salt of a compound selected from the group consisting of: A1 to A6, A10, A12 to 105.

Another embodiment of this invention is directed to a solvate of a compound selected from the group consisting of: A1 to A6, A10, A12 to A105.

Another embodiment of this invention is directed to a compound selected from the group consisting of: A3 to A6, A10, A12 to A16, and A86 to A89.

Another embodiment of this invention is directed to a pharmaceutically acceptable salt of a compound selected from the group consisting of: A3 to A6, A10, A12 to A16, and A86 to A89.

Another embodiment of this invention is directed to a solvate of a compound selected from the group consisting of: A3 to A6, A10, A12 to A16, and A86 to A89.

Another embodiment of this invention is directed to a compound selected from the group consisting of: A106 and A107.

Another embodiment of this invention is directed to a pharmaceutically acceptable salt of a compound selected from the group consisting of: A106 and A107.

Another embodiment of this invention is directed to a solvate of a compound selected from the group consisting of: A106 and A107.

Another embodiment of this invention is directed to a compound selected from the group consisting of: B4, B5, B7, and B8.

Another embodiment of this invention is directed to a pharmaceutically acceptable salt of a compound selected from the group consisting of: B4, B5, B7, and B8.

Another embodiment of this invention is directed to a solvate of a compound selected from the group consisting of: B4, B5, B7, and B8.

Another embodiment of this invention is directed to a compound selected from the group consisting of: A7, A8, A9 and A11.

Another embodiment of this invention is directed to a pharmaceutically acceptable salt of a compound selected from the group consisting of: A7, A8, A9 and A11.

Another embodiment of this invention is directed to a solvate of a compound selected from the group consisting of: A7, A8, A9 and A11.

Another embodiment of this invention is directed to compound A1.

Another embodiment of this invention is directed to compound A2.

Another embodiment of this invention is directed to compound A3.

Another embodiment of this invention is directed to compound A4.

Another embodiment of this invention is directed to compound A5.

Another embodiment of this invention is directed to compound A6.

Another embodiment of this invention is directed to compound A7.

Another embodiment of this invention is directed to compound A8.

Another embodiment of this invention is directed to compound A9.

Another embodiment of this invention is directed to compound A10.

Another embodiment of this invention is directed to compound A11.

Another embodiment of this invention is directed to compound A12.

Another embodiment of this invention is directed to compound A13.

Another embodiment of this invention is directed to compound A14.

Another embodiment of this invention is directed to compound A15.

Another embodiment of this invention is directed to compound A16.

Another embodiment of this invention is directed to compound A17.

Another embodiment of this invention is directed to compound A18.

Another embodiment of this invention is directed to compound A19.

Another embodiment of this invention is directed to compound A20.

Another embodiment of this invention is directed to compound A21.

Another embodiment of this invention is directed to compound A22.

Another embodiment of this invention is directed to compound A23.

Another embodiment of this invention is directed to compound A24.

Another embodiment of this invention is directed to compound A25.

Another embodiment of this invention is directed to compound A26.

Another embodiment of this invention is directed to compound A27.

Another embodiment of this invention is directed to compound A28.

Another embodiment of this invention is directed to compound A29.

Another embodiment of this invention is directed to compound A30.

Another embodiment of this invention is directed to compound A31.

Another embodiment of this invention is directed to compound A32.

Another embodiment of this invention is directed to compound A33.

Another embodiment of this invention is directed to compound A34.

Another embodiment of this invention is directed to compound A35.

Another embodiment of this invention is directed to compound A36.

Another embodiment of this invention is directed to compound A37.

Another embodiment of this invention is directed to compound A38.

Another embodiment of this invention is directed to compound A39.

Another embodiment of this invention is directed to compound A40.

Another embodiment of this invention is directed to compound A41.

Another embodiment of this invention is directed to compound A42.

Another embodiment of this invention is directed to compound A43.

Another embodiment of this invention is directed to compound A44.

Another embodiment of this invention is directed to compound A45.

Another embodiment of this invention is directed to compound A46.

Another embodiment of this invention is directed to compound A47.

Another embodiment of this invention is directed to compound A48.

Another embodiment of this invention is directed to compound A49.

Another embodiment of this invention is directed to compound A50.

Another embodiment of this invention is directed to compound A51.

Another embodiment of this invention is directed to compound A52.

Another embodiment of this invention is directed to compound A53.

Another embodiment of this invention is directed to compound A54.

Another embodiment of this invention is directed to compound A55.

Another embodiment of this invention is directed to compound A56.

Another embodiment of this invention is directed to compound A57.

Another embodiment of this invention is directed to compound A58.

Another embodiment of this invention is directed to compound A59.

Another embodiment of this invention is directed to compound A60.

Another embodiment of this invention is directed to compound A61.

Another embodiment of this invention is directed to compound A62.

Another embodiment of this invention is directed to compound A63.

Another embodiment of this invention is directed to compound A64.

Another embodiment of this invention is directed to compound A65.

Another embodiment of this invention is directed to compound A66.

Another embodiment of this invention is directed to compound A67.

Another embodiment of this invention is directed to compound A68.

Another embodiment of this invention is directed to compound A69.

Another embodiment of this invention is directed to compound A70.

Another embodiment of this invention is directed to compound A71.

Another embodiment of this invention is directed to compound A72.

Another embodiment of this invention is directed to compound A73.

Another embodiment of this invention is directed to compound A74.

Another embodiment of this invention is directed to compound A75.

Another embodiment of this invention is directed to compound A76.

Another embodiment of this invention is directed to compound A77.

Another embodiment of this invention is directed to compound A78.

Another embodiment of this invention is directed to compound A79.

Another embodiment of this invention is directed to compound A80.

Another embodiment of this invention is directed to compound A81.

Another embodiment of this invention is directed to compound A82.

Another embodiment of this invention is directed to compound A83.

Another embodiment of this invention is directed to compound A84.

Another embodiment of this invention is directed to compound A85.

Another embodiment of this invention is directed to compound A86.

Another embodiment of this invention is directed to compound A87.

Another embodiment of this invention is directed to compound A88.

Another embodiment of this invention is directed to compound A89.

Another embodiment of this invention is directed to compound A90.

Another embodiment of this invention is directed to compound A91.

Another embodiment of this invention is directed to compound A92.

Another embodiment of this invention is directed to compound A93.

Another embodiment of this invention is directed to compound A94.

Another embodiment of this invention is directed to compound A95.

Another embodiment of this invention is directed to compound A96.

Another embodiment of this invention is directed to compound A97.

Another embodiment of this invention is directed to compound A98.

Another embodiment of this invention is directed to compound A99.

Another embodiment of this invention is directed to compound A100.

Another embodiment of this invention is directed to compound A101.

Another embodiment of this invention is directed to compound A102.

Another embodiment of this invention is directed to compound A103.

Another embodiment of this invention is directed to compound A104.

Another embodiment of this invention is directed to compound A105.

Another embodiment of this invention is directed to compound A106.

Another embodiment of this invention is directed to compound A107.

Another embodiment of this invention is directed to compound B4.

Another embodiment of this invention is directed to compound B5.

Another embodiment of this invention is directed to compound B7.

Another embodiment of this invention is directed to compound B8.

Another embodiment of this invention is directed to a pharmaceutically acceptable salt of compound A1.

Another embodiment of this invention is directed to a pharmaceutically acceptable salt of compound A2.

Another embodiment of this invention is directed to a pharmaceutically acceptable salt of compound A3.

Another embodiment of this invention is directed to a pharmaceutically acceptable salt of compound A4.

Another embodiment of this invention is directed to a pharmaceutically acceptable salt of compound A5.

Another embodiment of this invention is directed to a pharmaceutically acceptable salt of compound A6.

Another embodiment of this invention is directed to a pharmaceutically acceptable salt of compound A7.

Another embodiment of this invention is directed to a pharmaceutically acceptable salt of compound A8.

Another embodiment of this invention is directed to a pharmaceutically acceptable salt of compound A9.

Another embodiment of this invention is directed to a pharmaceutically acceptable salt of compound A10.

Another embodiment of this invention is directed to a pharmaceutically acceptable salt of compound A11.

Another embodiment of this invention is directed to a pharmaceutically acceptable salt of compound A12.

Another embodiment of this invention is directed to a pharmaceutically acceptable salt of compound A13.

Another embodiment of this invention is directed to a pharmaceutically acceptable salt of compound A14.

Another embodiment of this invention is directed to a pharmaceutically acceptable salt of compound A15.

Another embodiment of this invention is directed to a pharmaceutically acceptable salt of compound A16.

Another embodiment of this invention is directed to a pharmaceutically acceptable salt of compound A17.

Another embodiment of this invention is directed to a pharmaceutically acceptable salt of compound A18.

Another embodiment of this invention is directed to a pharmaceutically acceptable salt of compound A19.

Another embodiment of this invention is directed to a pharmaceutically acceptable salt of compound A20.

Another embodiment of this invention is directed to a pharmaceutically acceptable salt of compound A21.

Another embodiment of this invention is directed to a pharmaceutically acceptable salt of compound A22.

Another embodiment of this invention is directed to a pharmaceutically acceptable salt of compound A23.

Another embodiment of this invention is directed to a pharmaceutically acceptable salt of compound A24.

Another embodiment of this invention is directed to a pharmaceutically acceptable salt of compound A25.

Another embodiment of this invention is directed to a pharmaceutically acceptable salt of compound A26.

Another embodiment of this invention is directed to a pharmaceutically acceptable salt of compound A27.

Another embodiment of this invention is directed to a pharmaceutically acceptable salt of compound A28.

Another embodiment of this invention is directed to a pharmaceutically acceptable salt of compound A29.

Another embodiment of this invention is directed to a pharmaceutically acceptable salt of compound A30.

Another embodiment of this invention is directed to a pharmaceutically acceptable salt of compound A31.

Another embodiment of this invention is directed to a pharmaceutically acceptable salt of compound A32.

Another embodiment of this invention is directed to a pharmaceutically acceptable salt of compound A33.

Another embodiment of this invention is directed to a pharmaceutically acceptable salt of compound A34.

Another embodiment of this invention is directed to a pharmaceutically acceptable salt of compound A35.

Another embodiment of this invention is directed to a pharmaceutically acceptable salt of compound A36.

Another embodiment of this invention is directed to a pharmaceutically acceptable salt of compound A37.

Another embodiment of this invention is directed to a pharmaceutically acceptable salt of compound A38.

Another embodiment of this invention is directed to a pharmaceutically acceptable salt of compound A39.

Another embodiment of this invention is directed to a pharmaceutically acceptable salt of compound A40.

Another embodiment of this invention is directed to a pharmaceutically acceptable salt of compound A41.

Another embodiment of this invention is directed to a pharmaceutically acceptable salt of Compound A42.

Another embodiment of this invention is directed to a pharmaceutically acceptable salt of compound A43.

Another embodiment of this invention is directed to a pharmaceutically acceptable salt of compound A44.

Another embodiment of this invention is directed to a pharmaceutically acceptable salt of compound A45.

Another embodiment of this invention is directed to a pharmaceutically acceptable salt of compound A46.

Another embodiment of this invention is directed to a pharmaceutically acceptable salt of compound A47.

Another embodiment of this invention is directed to a pharmaceutically acceptable salt of compound A48.

Another embodiment of this invention is directed to a pharmaceutically acceptable salt of compound A49.

Another embodiment of this invention is directed to a pharmaceutically acceptable salt of compound A50.

Another embodiment of this invention is directed to a pharmaceutically acceptable salt of compound A51.

Another embodiment of this invention is directed to a pharmaceutically acceptable salt of compound A52.

Another embodiment of this invention is directed to a pharmaceutically acceptable salt of compound A53.

Another embodiment of this invention is directed to a pharmaceutically acceptable salt of compound A54.

Another embodiment of this invention is directed to a pharmaceutically acceptable salt of compound A55.

Another embodiment of this invention is directed to a pharmaceutically acceptable salt of compound A56.

Another embodiment of this invention is directed to a pharmaceutically acceptable salt of compound A57.

Another embodiment of this invention is directed to a pharmaceutically acceptable salt of compound A58.

Another embodiment of this invention is directed to a pharmaceutically acceptable salt of compound A59.

Another embodiment of this invention is directed to a pharmaceutically acceptable salt of compound A60.

Another embodiment of this invention is directed to a pharmaceutically acceptable salt of compound A61.

Another embodiment of this invention is directed to a pharmaceutically acceptable salt of compound A62.

Another embodiment of this invention is directed to a pharmaceutically acceptable salt of compound A63.

Another embodiment of this invention is directed to a pharmaceutically acceptable salt of compound A64.

Another embodiment of this invention is directed to a pharmaceutically acceptable salt of compound A65.

Another embodiment of this invention is directed to a pharmaceutically acceptable salt of compound A66.

Another embodiment of this invention is directed to a pharmaceutically acceptable salt of compound A67.

Another embodiment of this invention is directed to a pharmaceutically acceptable salt of compound A68.

Another embodiment of this invention is directed to a pharmaceutically acceptable salt of compound A69.

Another embodiment of this invention is directed to a pharmaceutically acceptable salt of compound A70.

Another embodiment of this invention is directed to a pharmaceutically acceptable salt of compound A71.

Another embodiment of this invention is directed to a pharmaceutically acceptable salt of compound A72.

Another embodiment of this invention is directed to a pharmaceutically acceptable salt of compound A73.

Another embodiment of this invention is directed to a pharmaceutically acceptable salt of compound A74.

Another embodiment of this invention is directed to a pharmaceutically acceptable salt of compound A75.

Another embodiment of this invention is directed to a pharmaceutically acceptable salt of compound A76.

Another embodiment of this invention is directed to a pharmaceutically acceptable salt of compound A77.

Another embodiment of this invention is directed to a pharmaceutically acceptable salt of compound A78.

Another embodiment of this invention is directed to a pharmaceutically acceptable salt of compound A79.

Another embodiment of this invention is directed to a pharmaceutically acceptable salt of compound A80.

Another embodiment of this invention is directed to a pharmaceutically acceptable salt of compound A81.

Another embodiment of this invention is directed to a pharmaceutically acceptable salt of compound A82.

Another embodiment of this invention is directed to a pharmaceutically acceptable salt of compound A83.

Another embodiment of this invention is directed to a pharmaceutically acceptable salt of compound A84.

Another embodiment of this invention is directed to a pharmaceutically acceptable salt of compound A85.

Another embodiment of this invention is directed to a pharmaceutically acceptable salt of compound A86.

Another embodiment of this invention is directed to a pharmaceutically acceptable salt of compound A87.

Another embodiment of this invention is directed to a pharmaceutically acceptable salt of compound A88.

Another embodiment of this invention is directed to a pharmaceutically acceptable salt of compound A89.

Another embodiment of this invention is directed to a pharmaceutically acceptable salt of compound A90.

Another embodiment of this invention is directed to a pharmaceutically acceptable salt of compound A91.

Another embodiment of this invention is directed to a pharmaceutically acceptable salt of compound A92.

Another embodiment of this invention is directed to a pharmaceutically acceptable salt of compound A93.

Another embodiment of this invention is directed to a pharmaceutically acceptable salt of compound A94.

Another embodiment of this invention is directed to a pharmaceutically acceptable salt of compound A95.

Another embodiment of this invention is directed to a pharmaceutically acceptable salt of compound A96.

Another embodiment of this invention is directed to a pharmaceutically acceptable salt of compound A97.

Another embodiment of this invention is directed to a pharmaceutically acceptable salt of compound A98.

Another embodiment of this invention is directed to a pharmaceutically acceptable salt of compound A99.

Another embodiment of this invention is directed to a pharmaceutically acceptable salt of compound A100.

Another embodiment of this invention is directed to a pharmaceutically acceptable salt of compound A101.

Another embodiment of this invention is directed to a pharmaceutically acceptable salt of compound A102.

Another embodiment of this invention is directed to a pharmaceutically acceptable salt of compound A103.

Another embodiment of this invention is directed to a pharmaceutically acceptable salt of compound A104.

Another embodiment of this invention is directed to a pharmaceutically acceptable salt of compound A105.

Another embodiment of this invention is directed to a pharmaceutically acceptable salt of compound A106.

Another embodiment of this invention is directed to a pharmaceutically acceptable salt of compound A107.

Another embodiment of this invention is directed to a pharmaceutically acceptable salt of compound B4.

Another embodiment of this invention is directed to a pharmaceutically acceptable salt of compound B5.

Another embodiment of this invention is directed to a pharmaceutically acceptable salt of compound B7.

Another embodiment of this invention is directed to a pharmaceutically acceptable salt of compound B8.

Another embodiment of this invention is directed to a pharmaceutically acceptable ester of compound A6.

Another embodiment of this invention is directed to a pharmaceutically acceptable ester of compound A10.

Another embodiment of this invention is directed to a pharmaceutically acceptable ester of compound A12.

Another embodiment of this invention is directed to a pharmaceutically acceptable ester of compound A33.

Another embodiment of this invention is directed to a pharmaceutically acceptable ester of compound A34.

Another embodiment of this invention is directed to a pharmaceutically acceptable ester of compound A35.

Another embodiment of this invention is directed to a pharmaceutically acceptable ester of compound A36.

Another embodiment of this invention is directed to a pharmaceutically acceptable ester of compound A37.

Another embodiment of this invention is directed to a pharmaceutically acceptable ester of compound A38.

Another embodiment of this invention is directed to a pharmaceutically acceptable ester of compound A39.

Another embodiment of this invention is directed to a pharmaceutically acceptable ester of compound A40.

Another embodiment of this invention is directed to a pharmaceutically acceptable ester of compound A41.

Another embodiment of this invention is directed to a pharmaceutically acceptable ester of compound A42.

Another embodiment of this invention is directed to a pharmaceutically acceptable ester of compound A43.

Another embodiment of this invention is directed to a pharmaceutically acceptable ester of compound A44.

Another embodiment of this invention is directed to a pharmaceutically acceptable ester of compound A45.

Another embodiment of this invention is directed to a pharmaceutically acceptable ester of compound A46.

Another embodiment of this invention is directed to a pharmaceutically acceptable ester of compound A47.

Another embodiment of this invention is directed to a pharmaceutically acceptable ester of compound A48.

Another embodiment of this invention is directed to a pharmaceutically acceptable ester of compound A55.

Another embodiment of this invention is directed to a pharmaceutically acceptable ester of compound A56.

Another embodiment of this invention is directed to a pharmaceutically acceptable ester of compound A57.

Another embodiment of this invention is directed to a pharmaceutically acceptable ester of compound A58.

Another embodiment of this invention is directed to a pharmaceutically acceptable ester of compound A59.

Another embodiment of this invention is directed to a pharmaceutically acceptable ester of compound A60.

Another embodiment of this invention is directed to a pharmaceutically acceptable ester of compound A61.

Another embodiment of this invention is directed to a pharmaceutically acceptable ester of compound A68.

Another embodiment of this invention is directed to a pharmaceutically acceptable ester of compound A69.

Another embodiment of this invention is directed to a pharmaceutically acceptable ester of compound A70.

Another embodiment of this invention is directed to a pharmaceutically acceptable ester of compound A71.

Another embodiment of this invention is directed to a pharmaceutically acceptable ester of compound A72.

Another embodiment of this invention is directed to a pharmaceutically acceptable ester of compound A73.

Another embodiment of this invention is directed to a pharmaceutically acceptable ester of compound A80.

Another embodiment of this invention is directed to a pharmaceutically acceptable ester of compound A81.

Another embodiment of this invention is directed to a pharmaceutically acceptable ester of compound A82.

Another embodiment of this invention is directed to a pharmaceutically acceptable ester of compound A83.

Another embodiment of this invention is directed to a pharmaceutically acceptable ester of compound A84.

Another embodiment of this invention is directed to a pharmaceutically acceptable ester of compound A85.

Another embodiment of this invention is directed to a pharmaceutically acceptable ester of compound A94.

Another embodiment of this invention is directed to a pharmaceutically acceptable ester of compound A95.

Another embodiment of this invention is directed to a pharmaceutically acceptable ester of compound A96.

Another embodiment of this invention is directed to a pharmaceutically acceptable ester of compound A97.

Another embodiment of this invention is directed to a pharmaceutically acceptable ester of compound A102.

Another embodiment of this invention is directed to a pharmaceutically acceptable ester of compound A103.

Another embodiment of this invention is directed to a pharmaceutically acceptable ester of compound A104.

Another embodiment of this invention is directed to a pharmaceutically acceptable ester of compound A105.

Another embodiment of this invention is directed to a solvate of compound A1.

Another embodiment of this invention is directed to a solvate of compound A2.

Another embodiment of this invention is directed to a solvate of compound A3.

Another embodiment of this invention is directed to a solvate of compound A4.

Another embodiment of this invention is directed to a solvate of compound A5.

Another embodiment of this invention is directed to a solvate of compound A6.

Another embodiment of this invention is directed to a solvate of compound A7.

Another embodiment of this invention is directed to a solvate of compound A8.

Another embodiment of this invention is directed to a solvate of compound A9.

Another embodiment of this invention is directed to a solvate of compound A10.

Another embodiment of this invention is directed to a solvate of compound A11.

Another embodiment of this invention is directed to a solvate of compound A12.

Another embodiment of this invention is directed to a solvate of compound A13.

Another embodiment of this invention is directed to a solvate of compound A14.

Another embodiment of this invention is directed to a solvate of compound A15.

Another embodiment of this invention is directed to a solvate of compound A16.

Another embodiment of this invention is directed to a solvate of compound A17.

Another embodiment of this invention is directed to a solvate of compound A18.

Another embodiment of this invention is directed to a solvate of compound A19.

Another embodiment of this invention is directed to a solvate of compound A20.

Another embodiment of this invention is directed to a solvate of compound A21.

Another embodiment of this invention is directed to a solvate of compound A22.

Another embodiment of this invention is directed to a solvate of compound A23.

Another embodiment of this invention is directed to a solvate of compound A24.

Another embodiment of this invention is directed to a solvate of compound A25.

Another embodiment of this invention is directed to a solvate of compound A26.

Another embodiment of this invention is directed to a solvate of compound A27.

Another embodiment of this invention is directed to a solvate of compound A28.

Another embodiment of this invention is directed to a solvate of compound A29.

Another embodiment of this invention is directed to a solvate of compound A30.

Another embodiment of this invention is directed to a solvate of compound A31.

Another embodiment of this invention is directed to a solvate of compound A32.

Another embodiment of this invention is directed to a solvate of compound A33.

Another embodiment of this invention is directed to a solvate of compound A34.

Another embodiment of this invention is directed to a solvate of compound A35.

Another embodiment of this invention is directed to a solvate of compound A36.

Another embodiment of this invention is directed to a solvate of compound A37.

Another embodiment of this invention is directed to a solvate of compound A38.

Another embodiment of this invention is directed to a solvate of compound A39.

Another embodiment of this invention is directed to a solvate of compound A40.

Another embodiment of this invention is directed to a solvate of compound A41.

Another embodiment of this invention is directed to a solvate of compound A42.

Another embodiment of this invention is directed to a solvate of compound A43.

Another embodiment of this invention is directed to a solvate of compound A44.

Another embodiment of this invention is directed to a solvate of compound A45.

Another embodiment of this invention is directed to a solvate of compound A46.

Another embodiment of this invention is directed to a solvate of compound A47.

Another embodiment of this invention is directed to a solvate of compound A48.

Another embodiment of this invention is directed to a solvate of compound A49.

Another embodiment of this invention is directed to a solvate of compound A50.

Another embodiment of this invention is directed to a solvate of compound A51.

Another embodiment of this invention is directed to a solvate of compound A52.

Another embodiment of this invention is directed to a solvate of compound A53.

Another embodiment of this invention is directed to a solvate of compound A54.

Another embodiment of this invention is directed to a solvate of compound A55.

Another embodiment of this invention is directed to a solvate of compound A56.

Another embodiment of this invention is directed to a solvate of compound A57.

Another embodiment of this invention is directed to a solvate of compound A58.

Another embodiment of this invention is directed to a solvate of compound A59.

Another embodiment of this invention is directed to a solvate of compound A60.

Another embodiment of this invention is directed to a solvate of compound A61.

Another embodiment of this invention is directed to a solvate of compound A62.

Another embodiment of this invention is directed to a solvate of compound A63.

Another embodiment of this invention is directed to a solvate of compound A64.

Another embodiment of this invention is directed to a solvate of compound A65.

Another embodiment of this invention is directed to a solvate of compound A66.

Another embodiment of this invention is directed to a solvate of compound A67.

Another embodiment of this invention is directed to a solvate of compound A68.

Another embodiment of this invention is directed to a solvate of compound A69.

Another embodiment of this invention is directed to a solvate of compound A70.

Another embodiment of this invention is directed to a solvate of compound A71.

Another embodiment of this invention is directed to a solvate of compound A72.

Another embodiment of this invention is directed to a solvate of compound A73.

Another embodiment of this invention is directed to a solvate of compound A74.

Another embodiment of this invention is directed to a solvate of compound A75.

Another embodiment of this invention is directed to a solvate of compound A76.

Another embodiment of this invention is directed to a solvate of compound A77.

Another embodiment of this invention is directed to a solvate of compound A78.

Another embodiment of this invention is directed to a solvate of compound A79.

Another embodiment of this invention is directed to a solvate of compound A80.

Another embodiment of this invention is directed to a solvate of compound A81.

Another embodiment of this invention is directed to a solvate of compound A82.

Another embodiment of this invention is directed to a solvate of compound A83.

Another embodiment of this invention is directed to a solvate of compound A84.

Another embodiment of this invention is directed to a solvate of compound A85.

Another embodiment of this invention is directed to a solvate of compound A86.

Another embodiment of this invention is directed to a solvate of compound A87.

Another embodiment of this invention is directed to a solvate of compound A88.

Another embodiment of this invention is directed to a solvate of compound A89.

Another embodiment of this invention is directed to a solvate of compound A90.

Another embodiment of this invention is directed to a solvate of compound A91.

Another embodiment of this invention is directed to a solvate of compound A92.

Another embodiment of this invention is directed to a solvate of compound A93.

Another embodiment of this invention is directed to a solvate of compound A94.

Another embodiment of this invention is directed to a solvate of compound A95.

Another embodiment of this invention is directed to a solvate of compound A96.

Another embodiment of this invention is directed to a solvate of compound A97.

Another embodiment of this invention is directed to a solvate of compound A98.

Another embodiment of this invention is directed to a solvate of compound A99.

Another embodiment of this invention is directed to a solvate of compound A100.

Another embodiment of this invention is directed to a solvate of compound A101.

Another embodiment of this invention is directed to a solvate of compound A102.

Another embodiment of this invention is directed to a solvate of compound A103.

Another embodiment of this invention is directed to a solvate of compound A104.

Another embodiment of this invention is directed to a solvate of compound A105.

Another embodiment of this invention is directed to a solvate of compound A106.

Another embodiment of this invention is directed to a solvate of compound A107.

Another embodiment of this invention is directed to a solvate of compound B4.

Another embodiment of this invention is directed to a solvate of compound B5.

Another embodiment of this invention is directed to a solvate of compound B7.

Another embodiment of this invention is directed to a solvate of compound B8.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ agonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated incorporated herein by reference thereto.

It is noted that the carbons of formula (I) and other formulas herein may be replaced with 1 to 3 silicon atoms so long as all valency requirements are satisfied.

As used above, and throughout this disclosure, the following terms, unless otherwise indicated, shall be understood to have the following meanings:

“Patient” includes both human and animals.

“Mammal” means humans and other mammalian animals.

“One or more” means that there is at least one and there can be more than one, and examples include 1, 2 or 3, or 1 and 2, or 1.

“At least one” means there is at least one and there can be more than one, and examples include 1, 2 or 3, or 1 and 2, or 1.

“Bn” means benzyl.

“BnBr” means benzyl bromide.

“DEAD” means diethyl azodicarboxylate.

“DPPA” diphenyl phosphoryl azide.

“EDCI” means N-(3-dimethylaminopropyl)-N′-ethylcarbodiimide.

“Et” means ethyl.

“i-pr” means isopropyl.

“Pr” means propyl.

“PMBO”: means para-methoxybenzyloxy.

“PMBOH” means para-methoxybenzyl alcohol.

“t-Bu” means tert-butyl.

“TBSCl” menas tert-butyl dimethyl silyl chloride

“Fused benzocycloalkyl ring” means a phenyl ring fused to a cycloalkyl ring (as cycloalkyl is defined below), such as, for example,

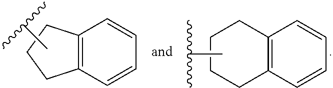

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain, “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

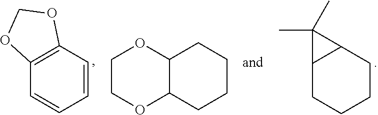

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” or “heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes rings wherein ═O replaces two available hydrogens on the same carbon atom on a ring system (i.e., heterocyclyl includes rings having a carbonyl in the ring). An example of such moiety is pyrrolidone:

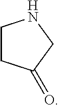

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein ═O replaces two available hydrogens on the same carbon atom on a ring system (i.e., heterocyclyl includes rings having a carbonyl in the ring). An example of such moiety is pyrrolidinone:

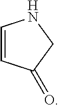

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

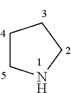

there is no —OH attached directly to carbons marked 2 and 5.

It should also be noted that tautomeric forms such as, for example, the moieties:

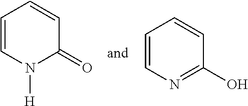

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences. And any one or more of these hydrogen atoms can be deuterium.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula (I) can form salts which are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula (I), and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl and ¹²³I, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. E.g., those labeled with positron-emitting isotopes like ¹¹C or ¹⁸F can be useful for application in Positron Emission Tomography (PET) and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half lives (T1/2>1 day), can generally be prepared by following procedures analogous to those disclosed in the Schemes and/or in the Examples herein below, by substituting an appropriate isotopically labeled reagent for a non-isotopically labeled reagent.

Polymorphic forms of the compounds of Formula (I), and of the salts, solvates, esters and prodrugs of the compounds of Formula (I), are intended to be included in the present invention.

The compounds according to the invention can have pharmacological properties; in particular, the compounds of Formula (I) can be modulators of gamma secretase (including inhibitors, antagonists and the like).

More specifically, the compounds of Formula (I) can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula (I), or a pharmaceutically acceptable salt, solvate, ester or prodrug of said compound to the mammal.

A preferred dosage is about 0.001 to 500 mg/kg of body weight/day of the compound of Formula (I). An especially preferred dosage is about 0.01 to 25 mg/kg of body weight/day of a compound of Formula (I), or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula (I), or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula (I), or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula (I), or a pharmaceutically acceptable salt, solvate, ester or prodrug of said compound and a pharmaceutically acceptable carrier, vehicle or diluent.

Yet another aspect of this invention is a kit comprising an amount of at least one compound of Formula (I), or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative schemes and examples which should not be construed to limit the scope of the disclosure. Alternative mechanistic pathways and analogous structures will be apparent to those skilled in the art.

Where NMR data are presented, 1H spectra were obtained on either a Varian VXR-200 (200 MHz, 1H), Varian Gemini-300 (300 MHz) or XL-400 (400 MHz), or Bruker 500 UltraShield (500 MHz) and are reported as ppm down field from Me4Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min—10% CH3CN, 5 min—95% CH3CN, 7 min—95% CH3CN, 7.5 min—10% CH3CN, 9 min—stop. The observed parent ion is given.”

The compounds of the invention can be prepared by the schemes and examples below.

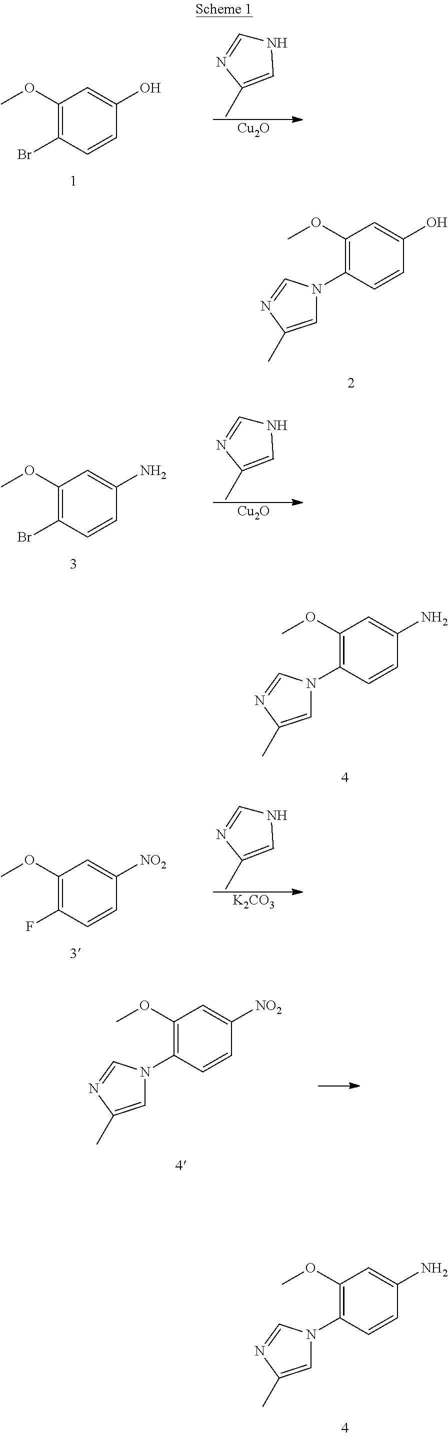

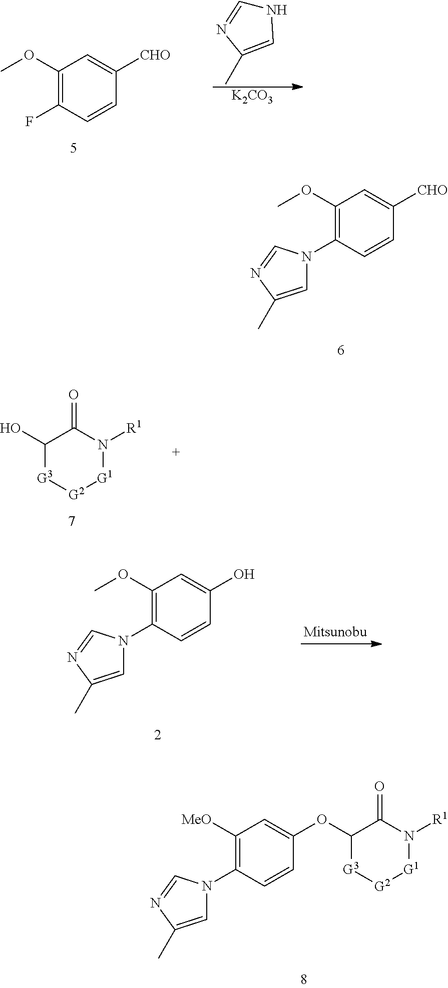

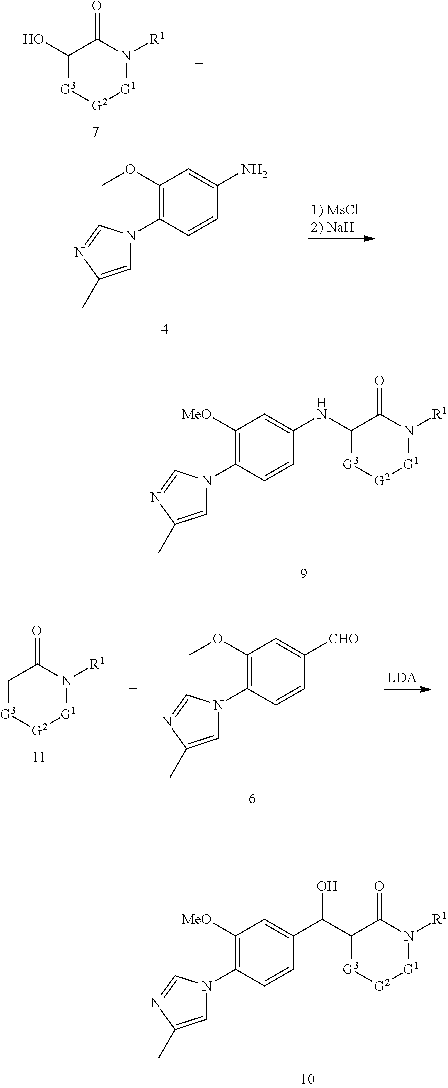

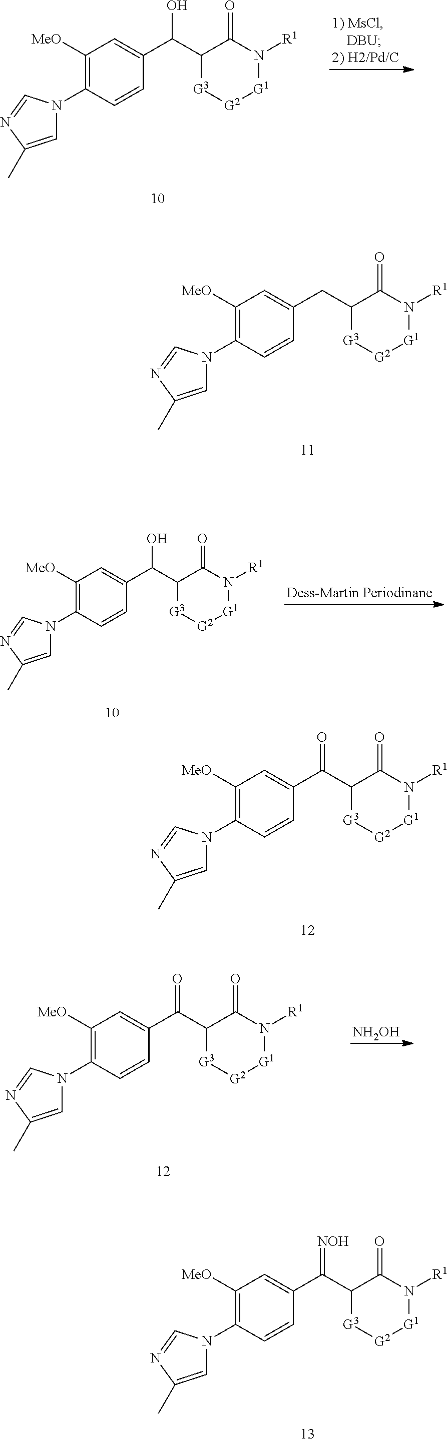

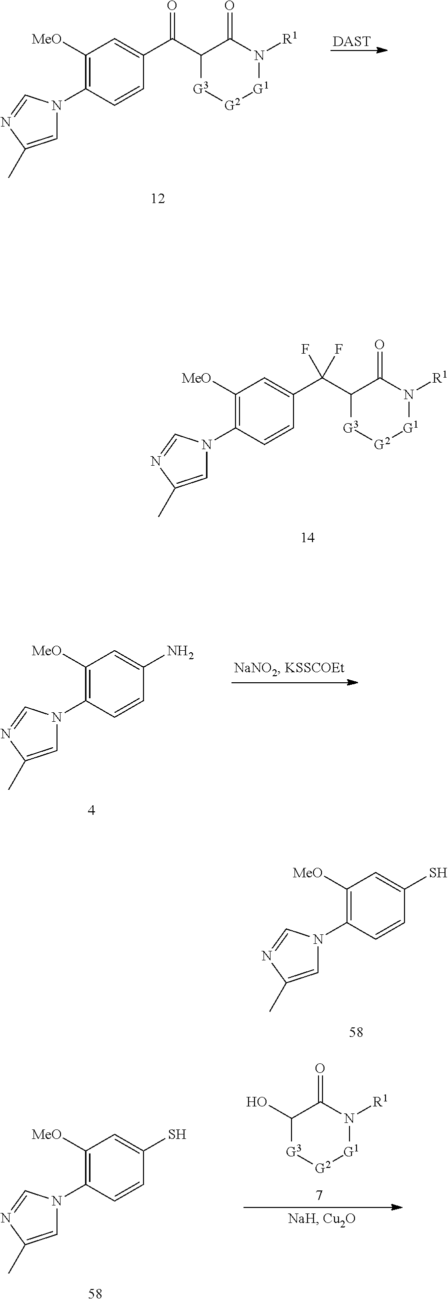

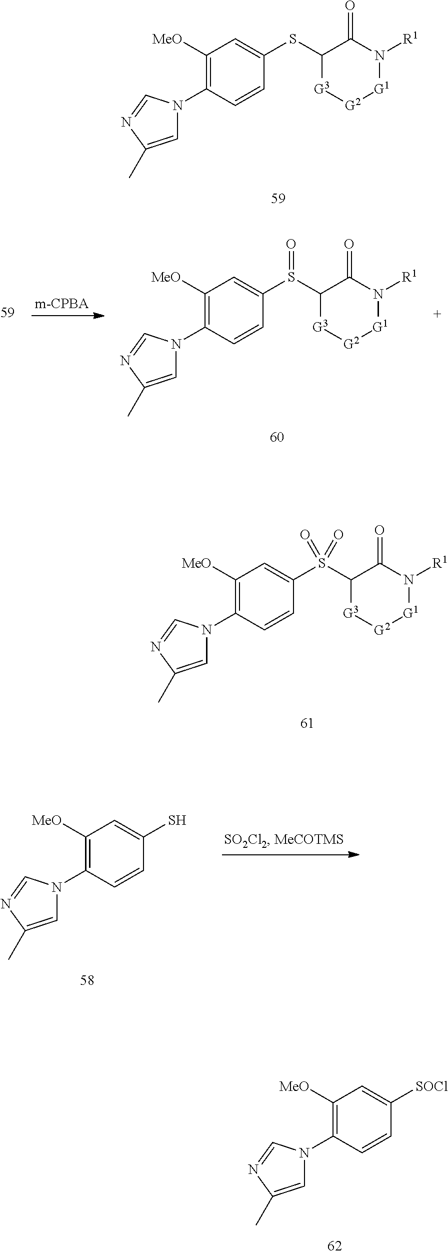

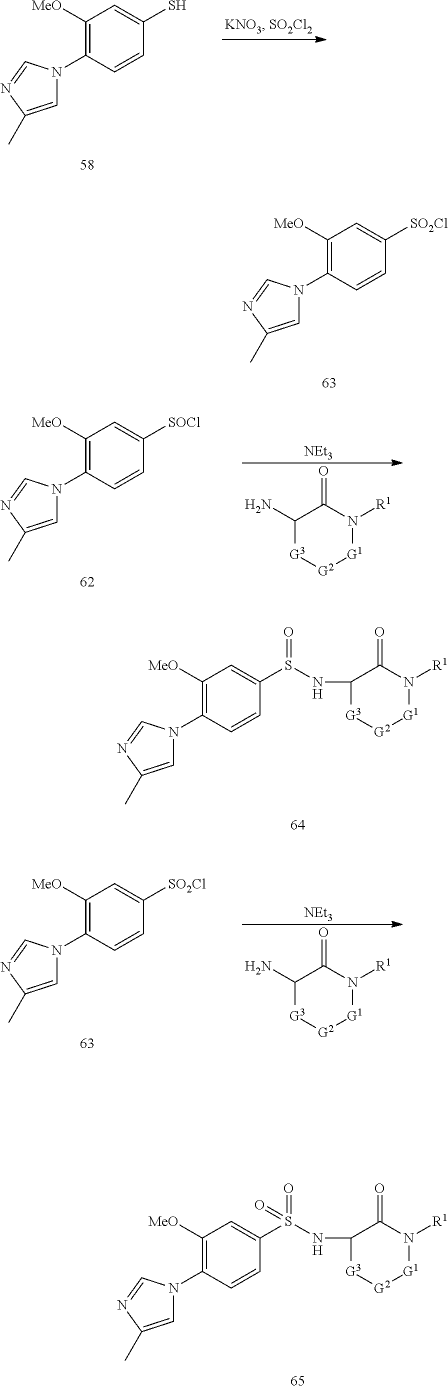

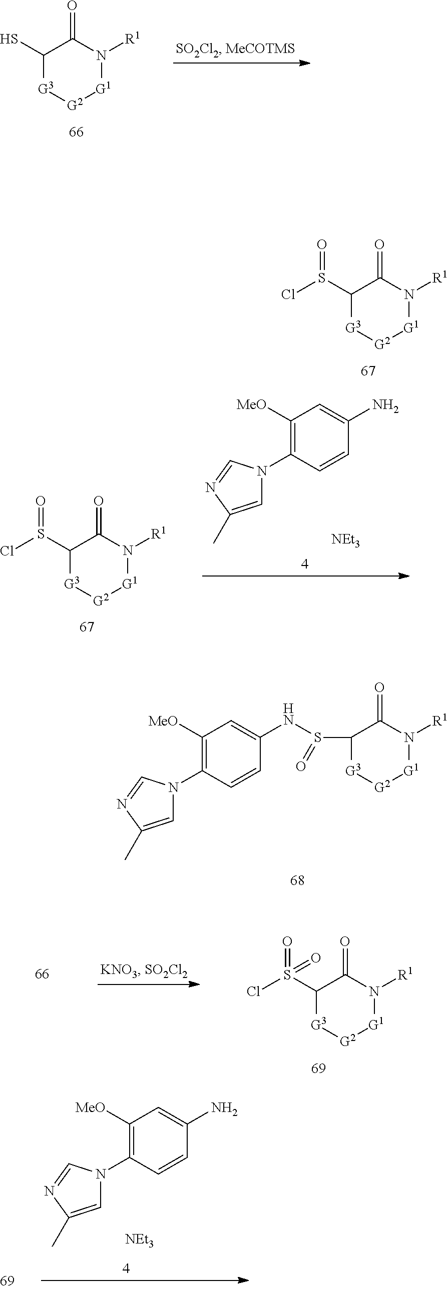

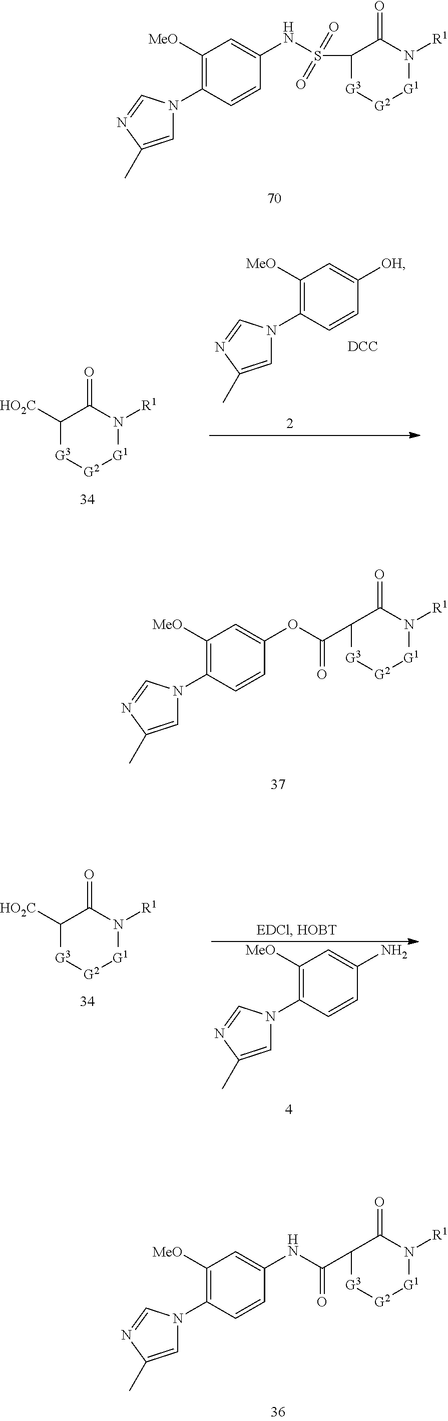

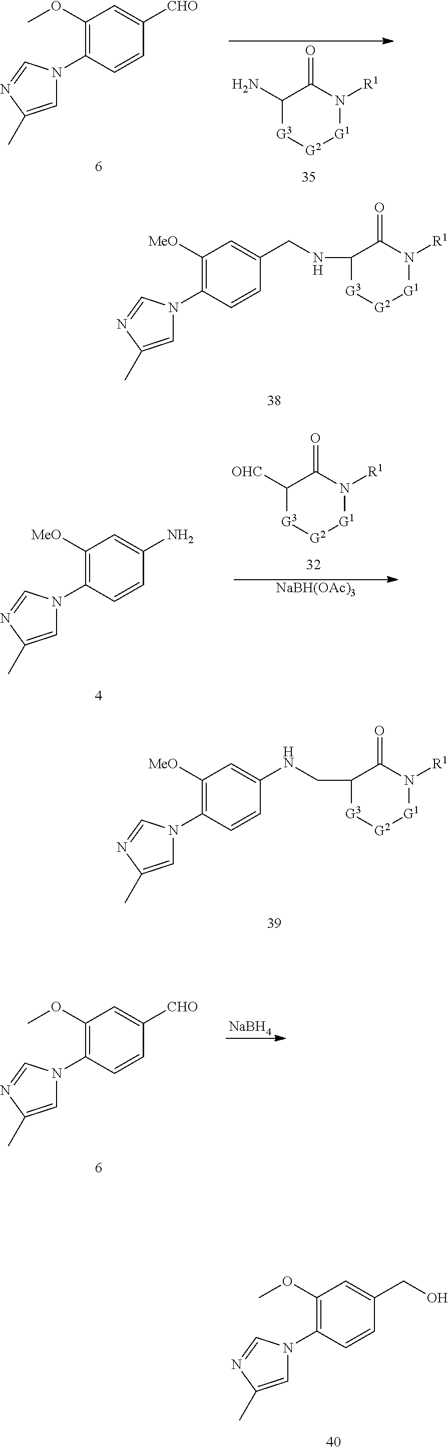

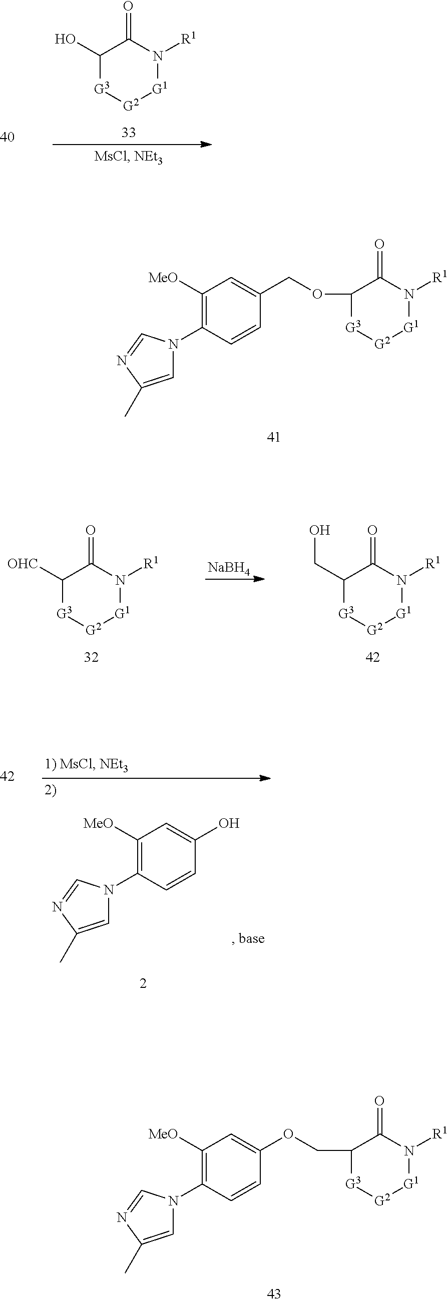

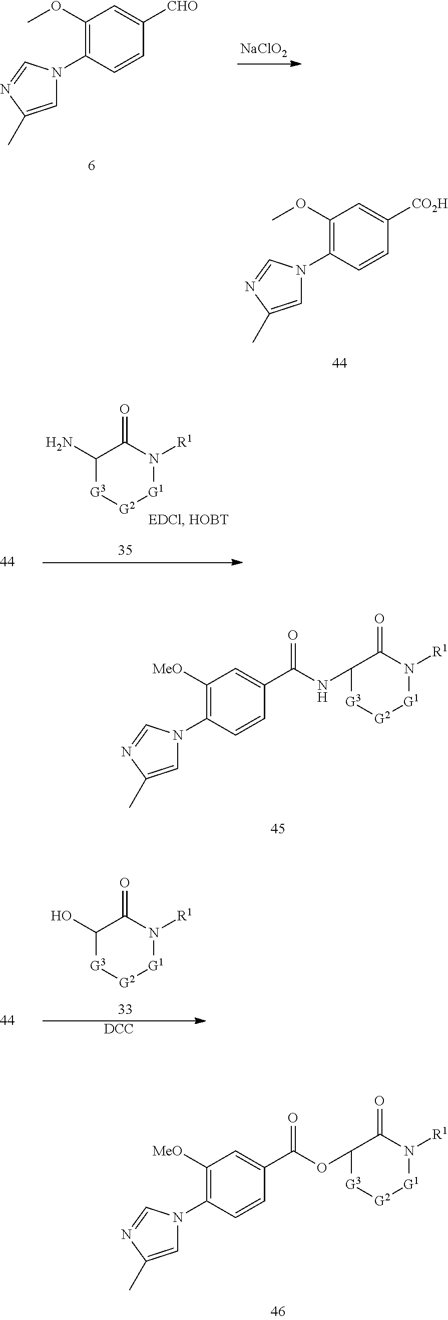

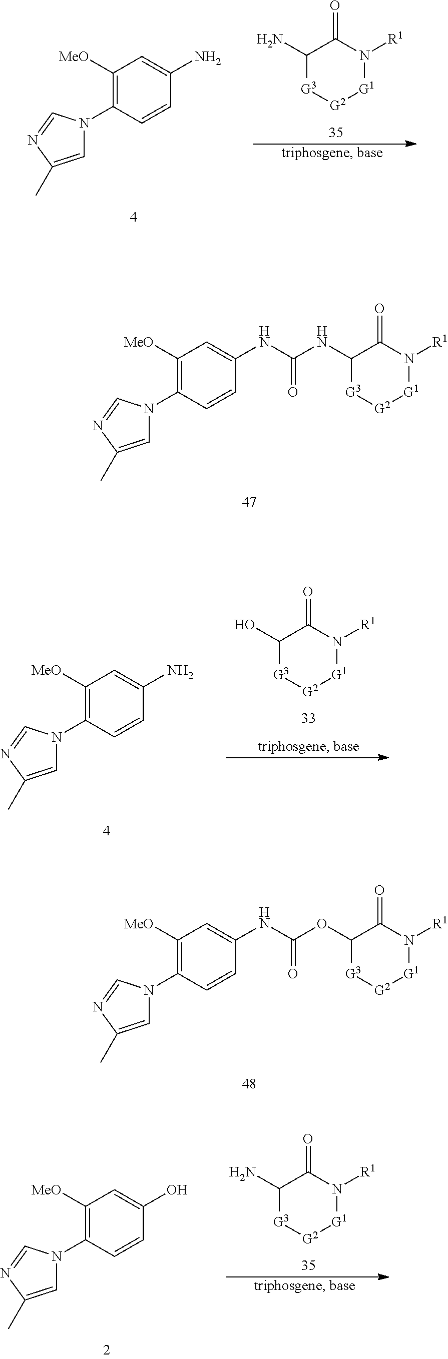

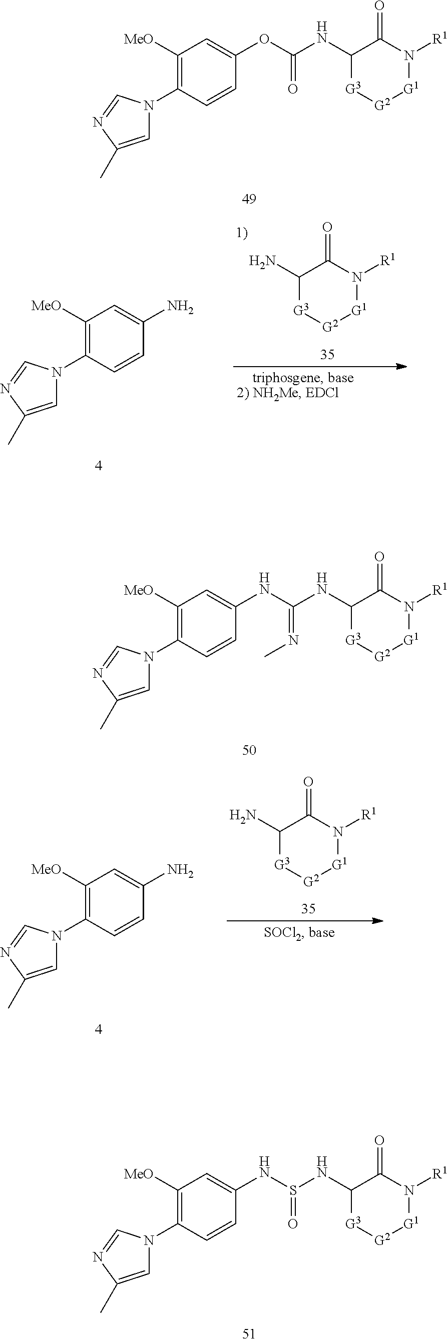

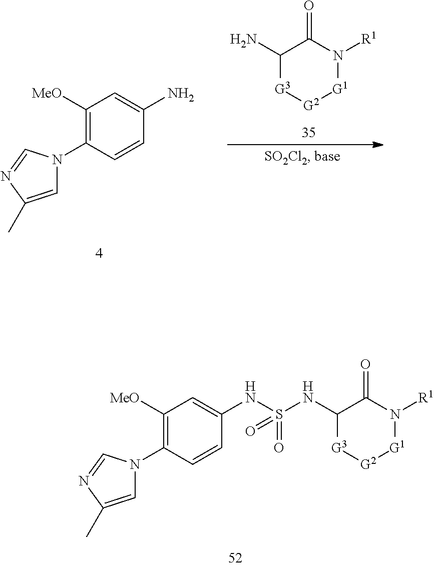

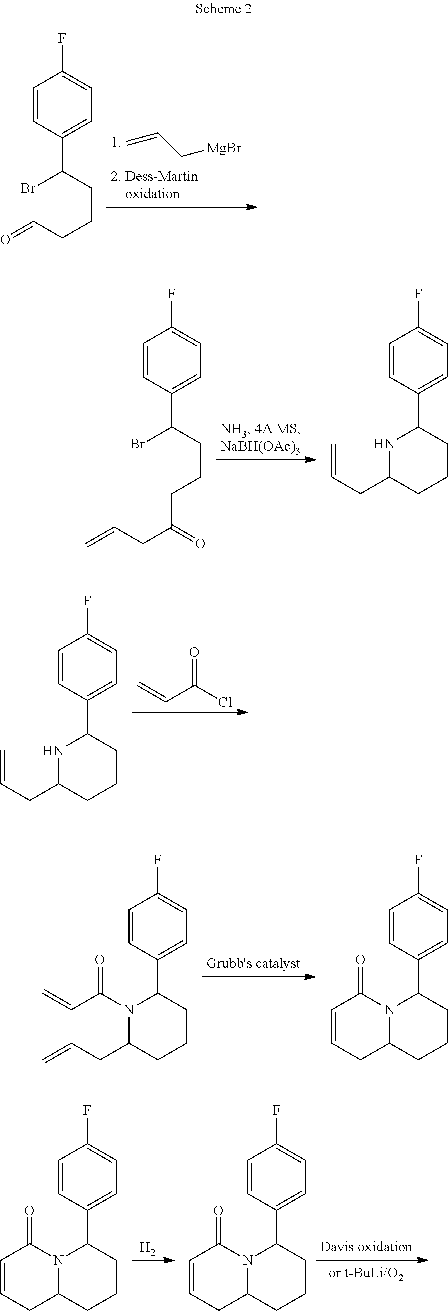

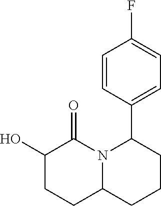

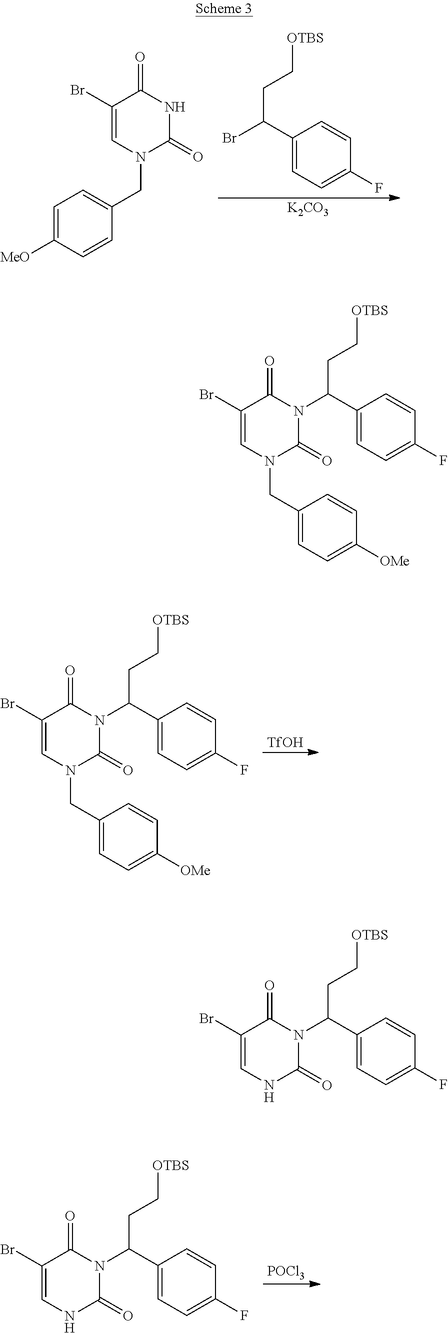

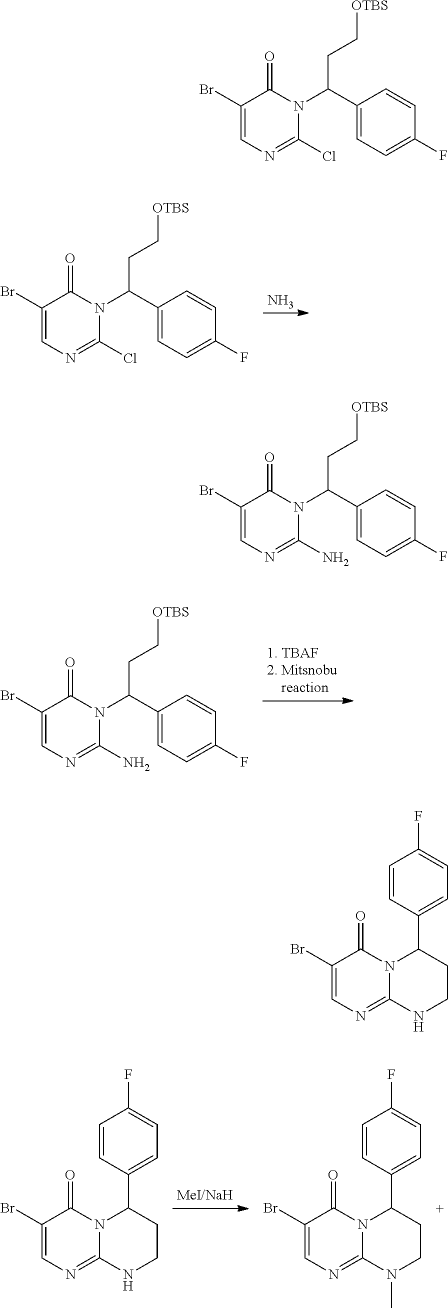

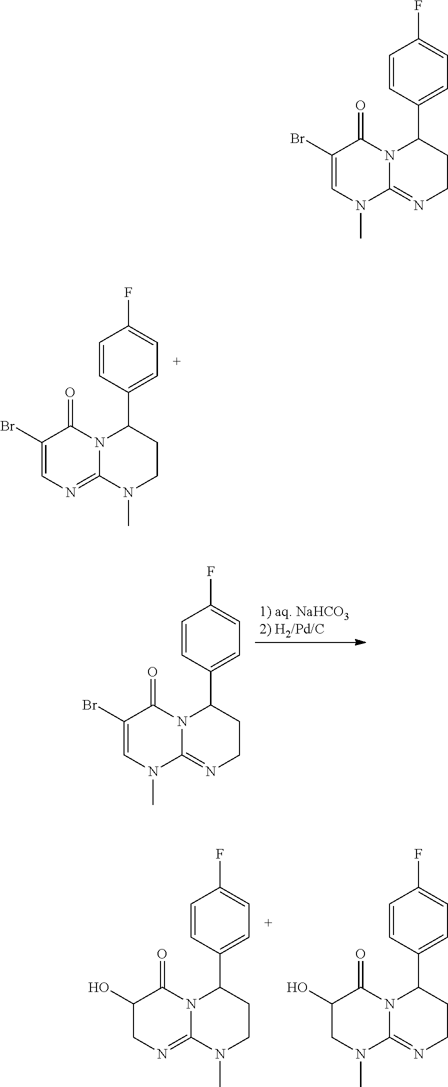

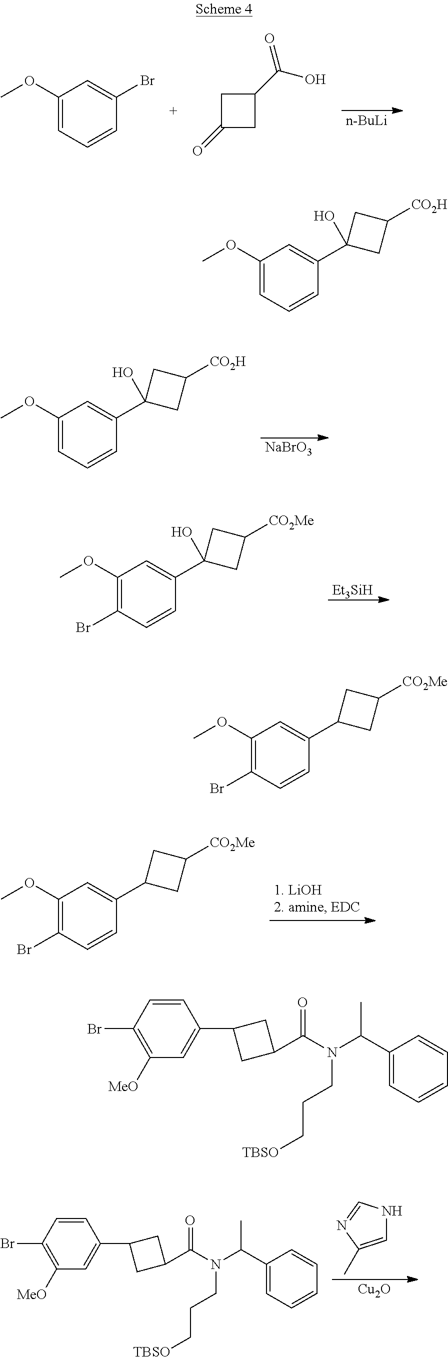

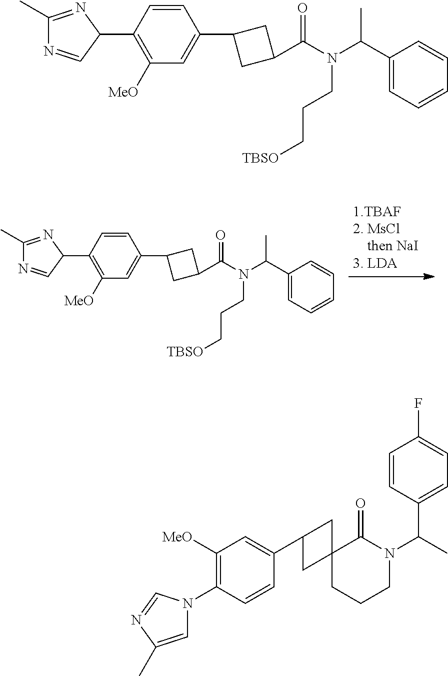

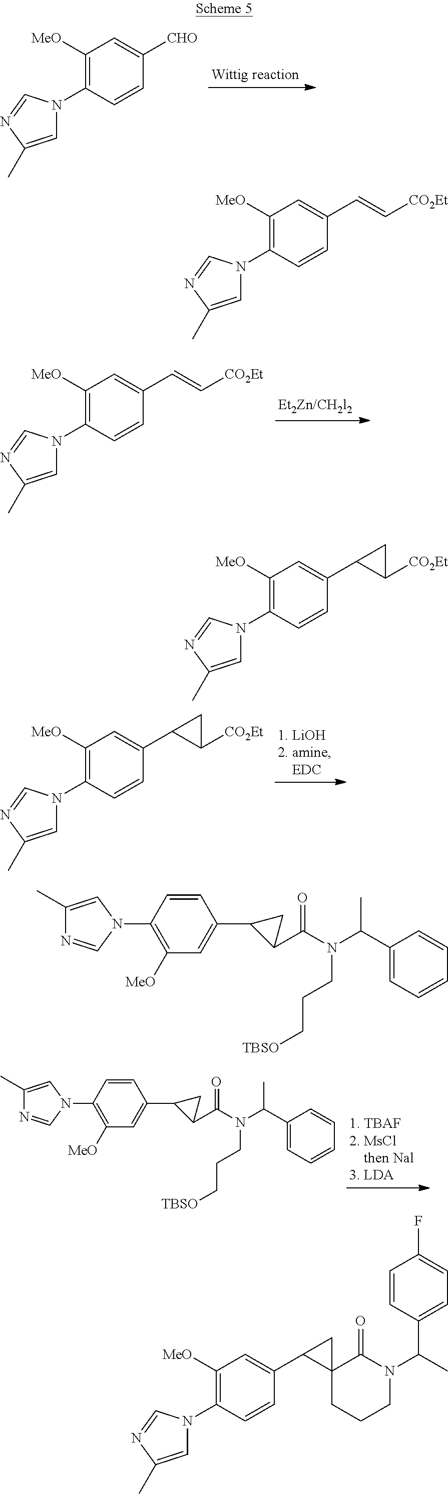

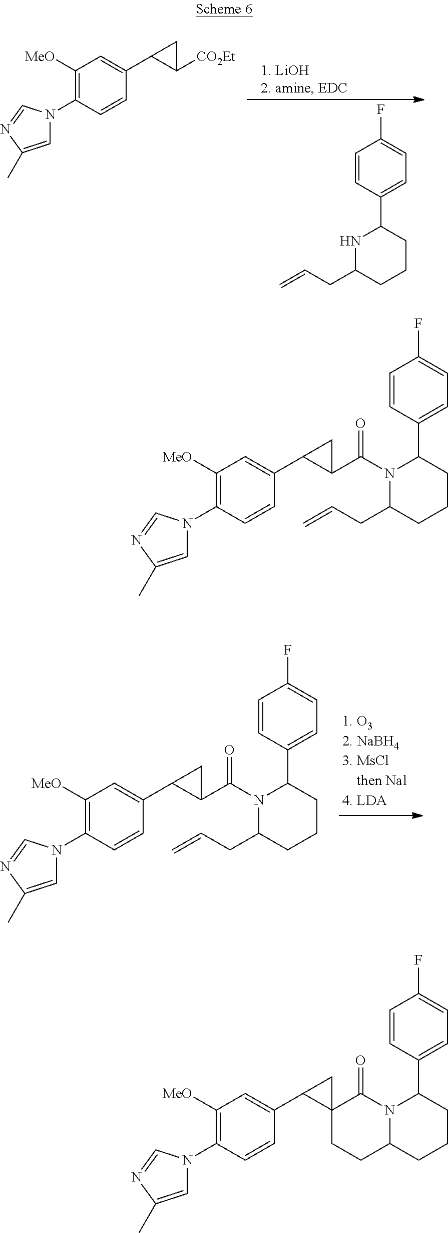

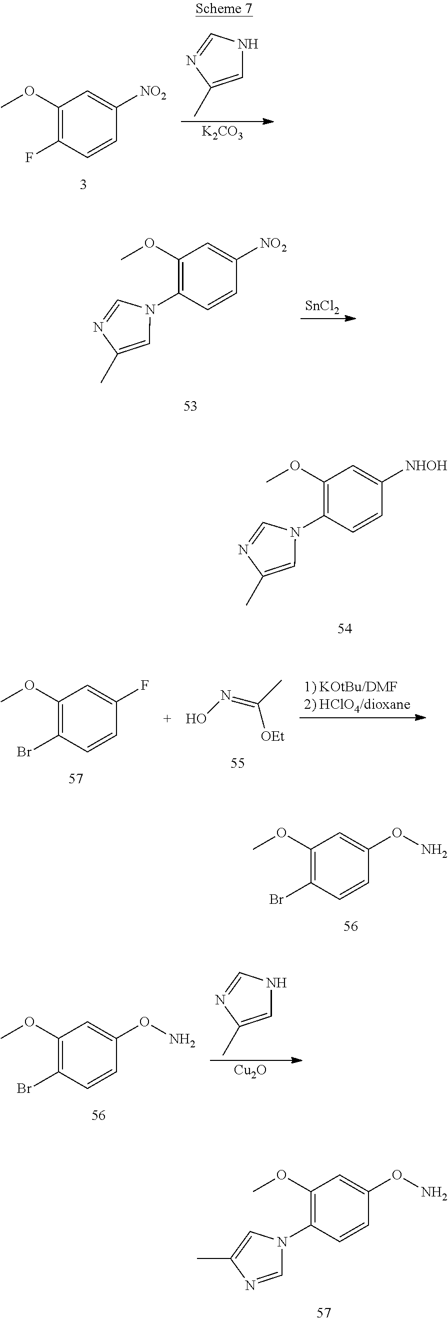

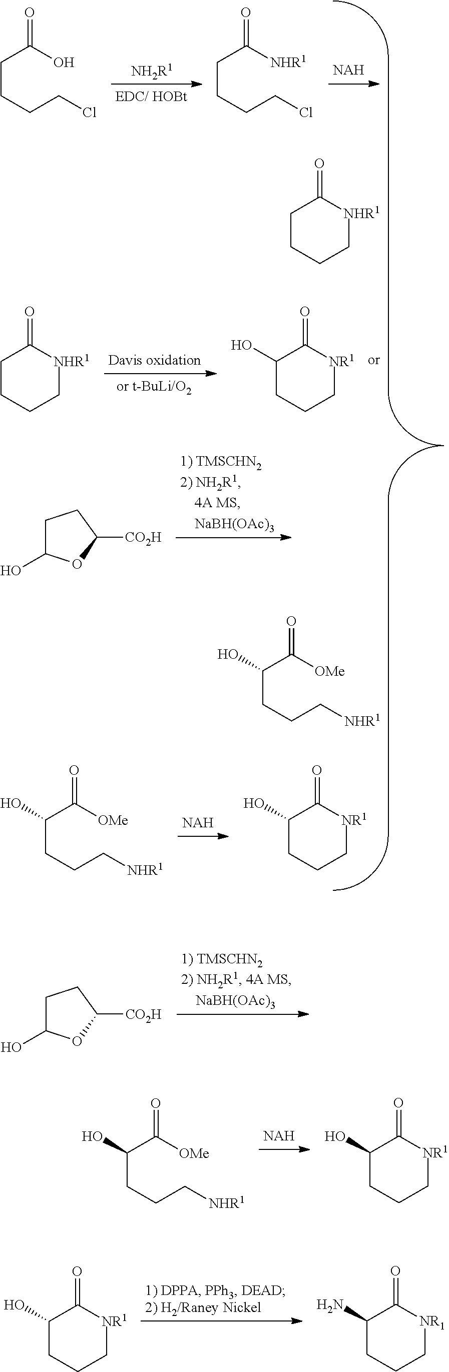

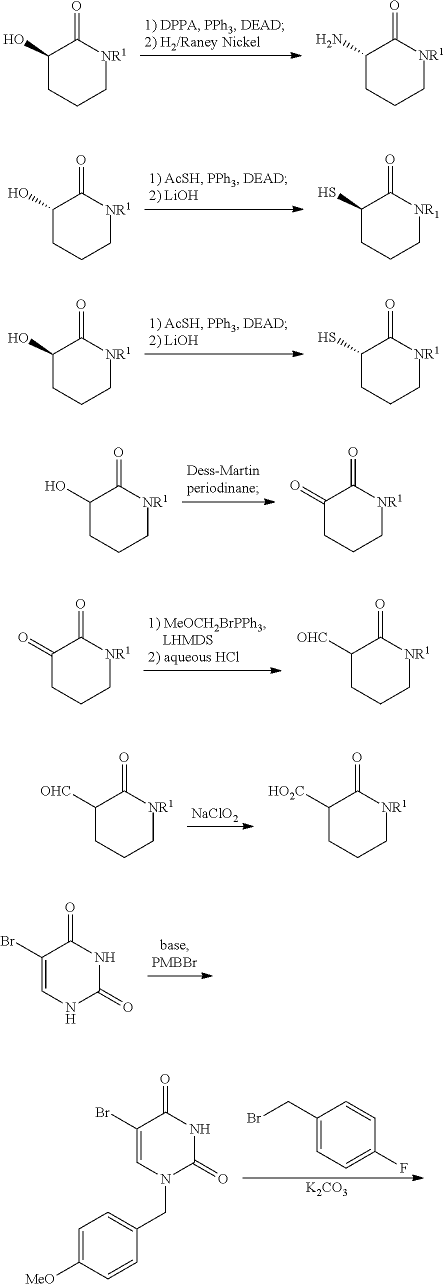

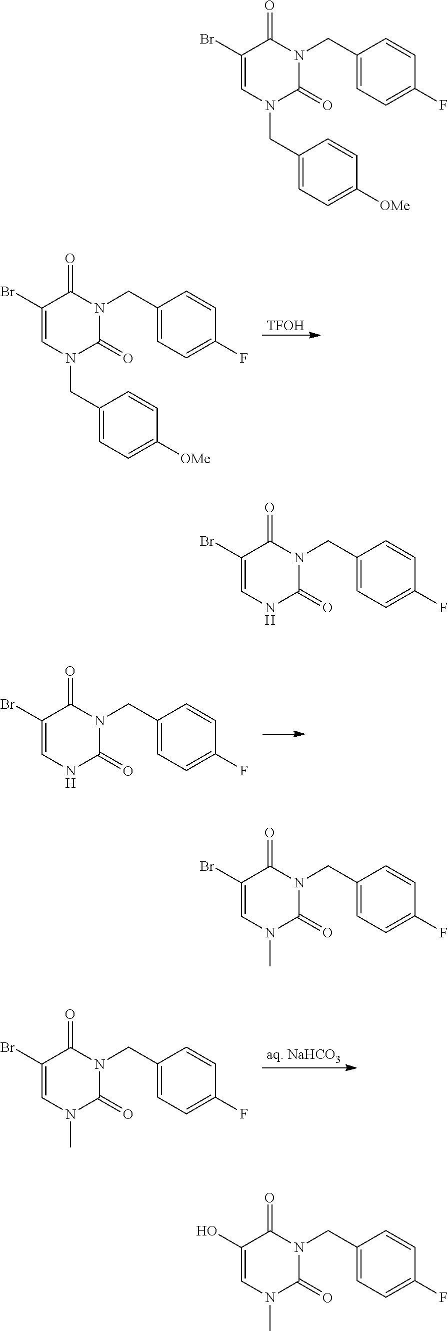

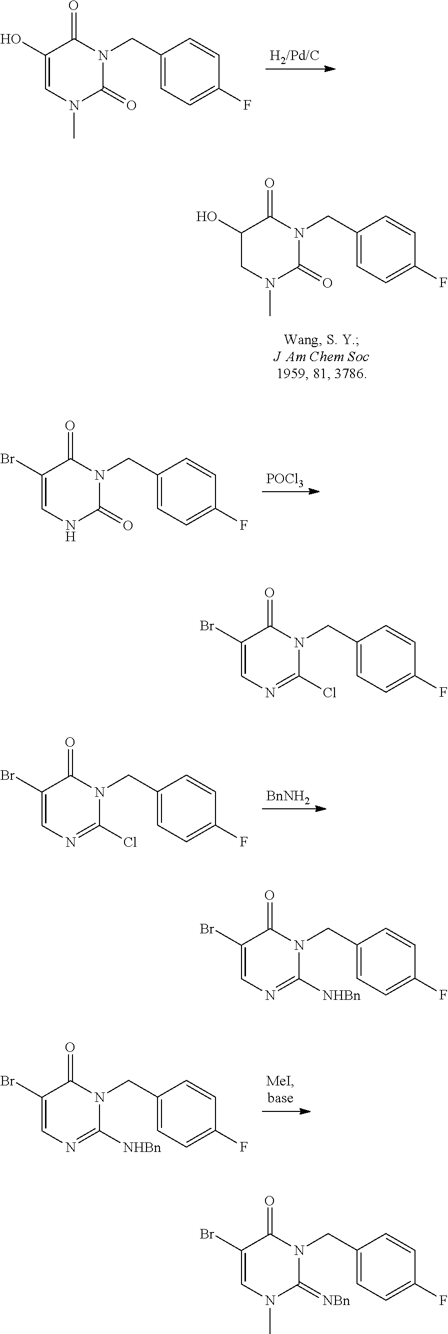

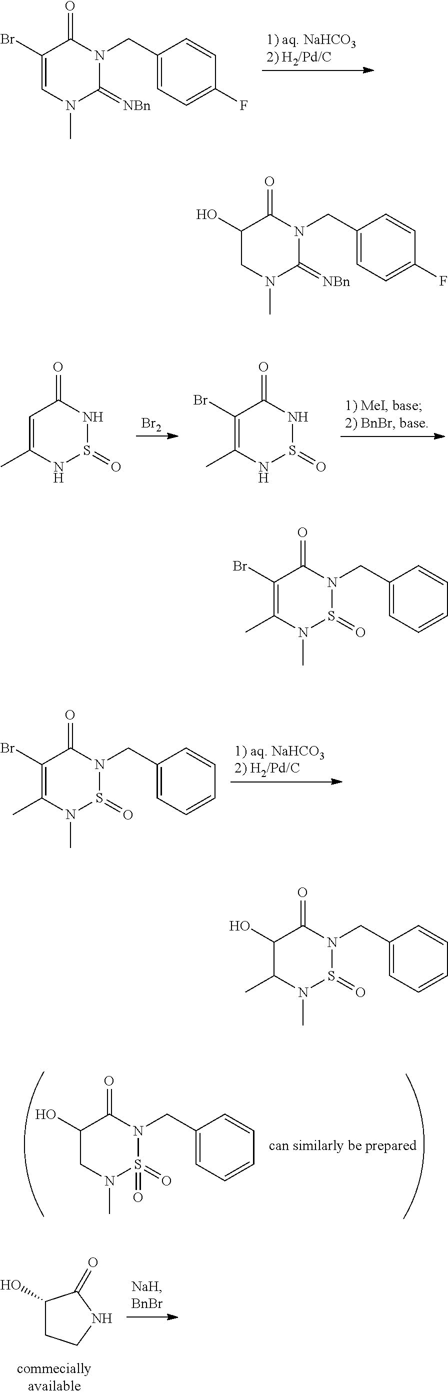

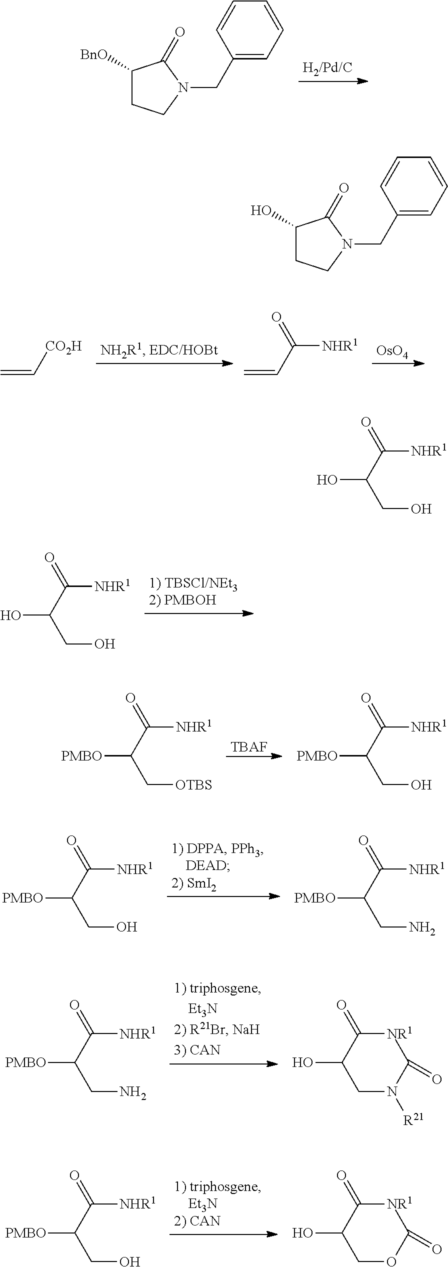

The following compounds can be prepared by the procedures described in the cited references, the disclosures of each reference being incorporated herein by reference thereto:

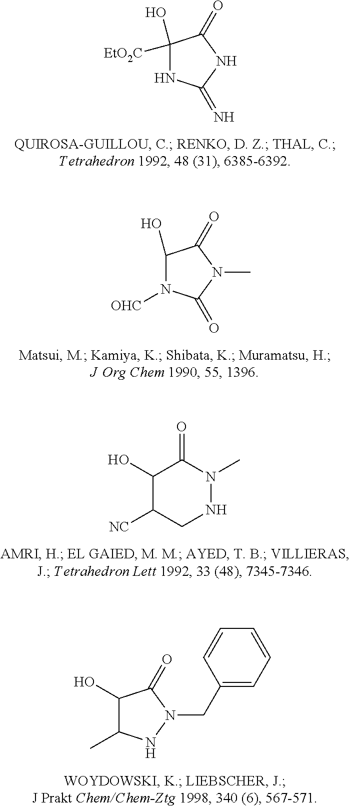

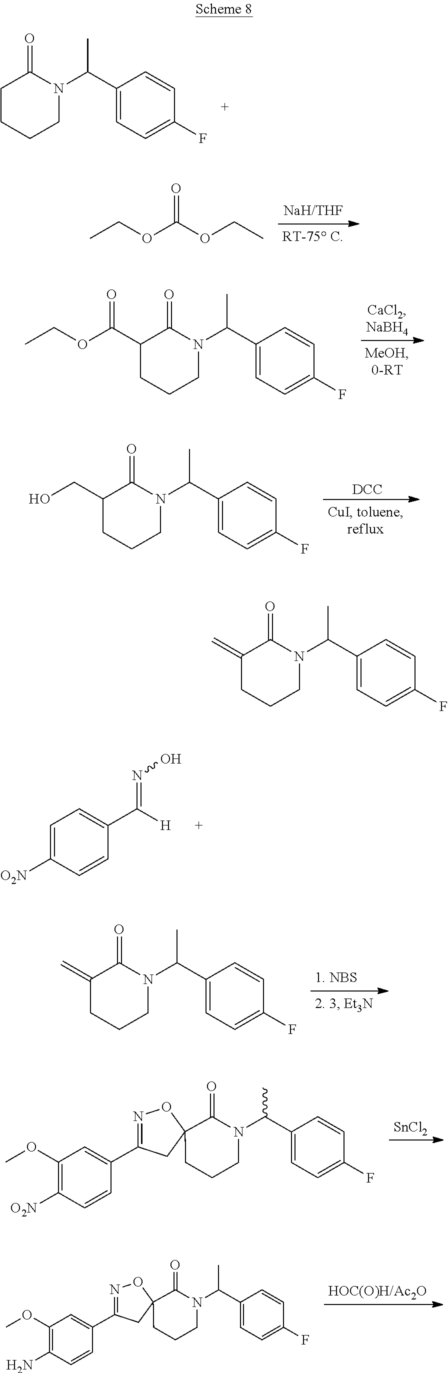

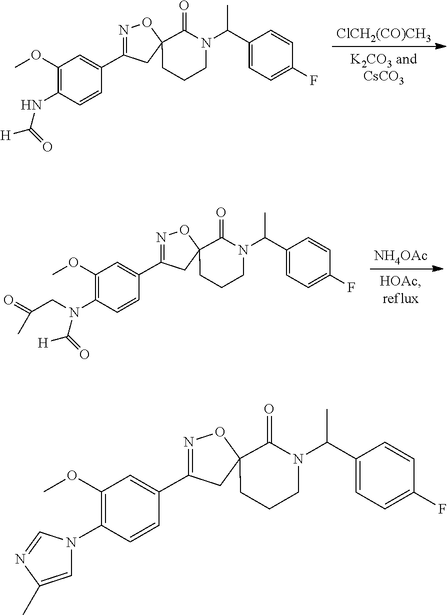

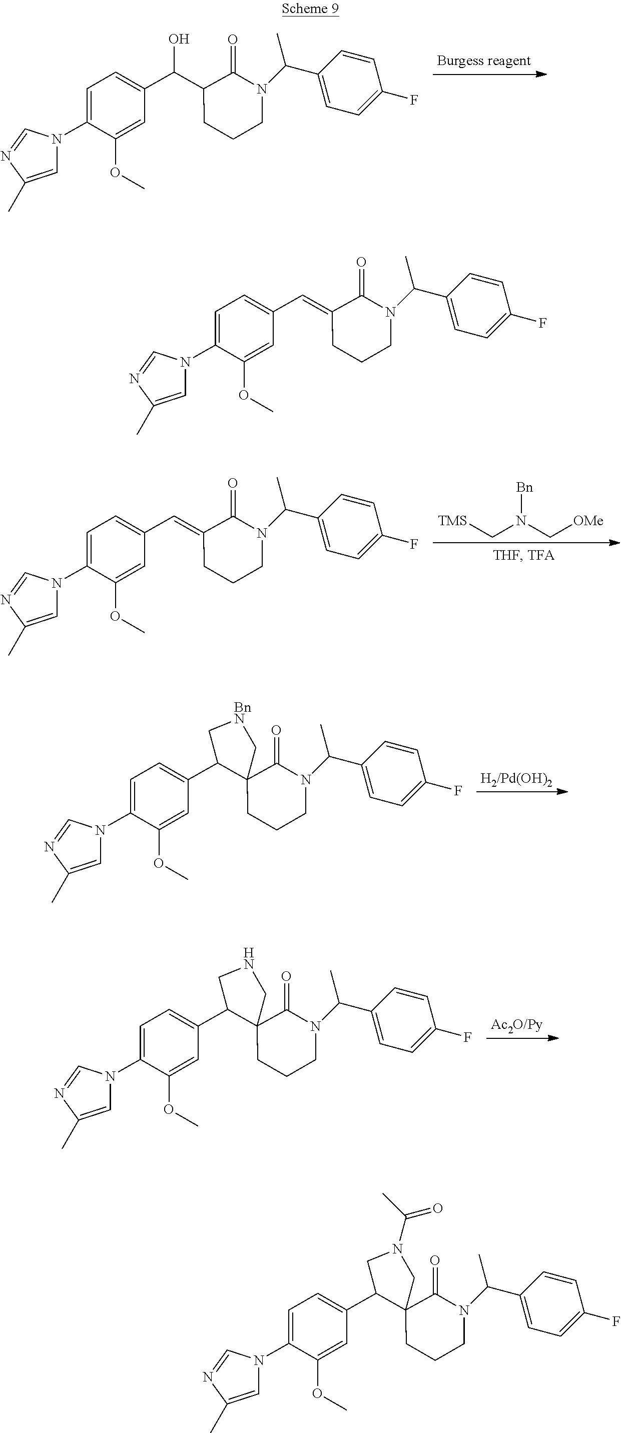

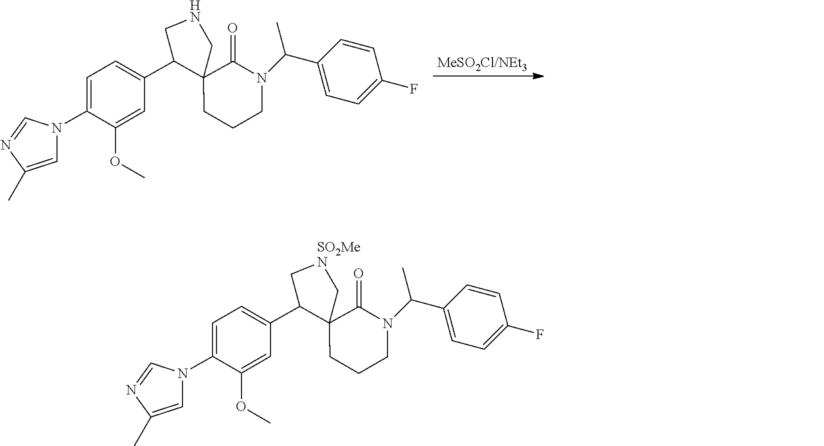

Compounds of the invention having —SF₅ and —Si(R¹⁵)₃ (e.g., —Si(CH₃)₃) groups can be prepared according to the scheme below and by techniques well known in the art. Those skilled in the art will appreciate that any carbon substitutable with a —CF₃ group can be substituted with a —SF₅ or a —Si(R¹⁵)₃ (e.g., —Si(CH₃)₃) group using techniques well known in the art.

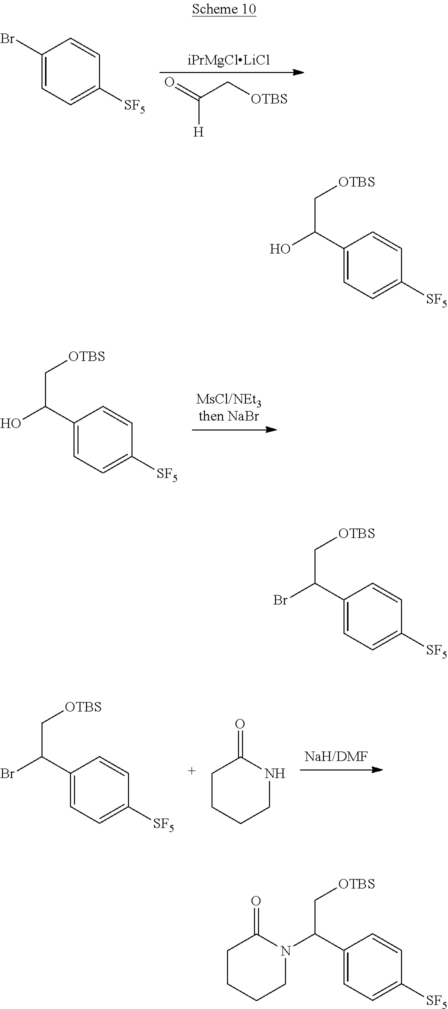

a-e can be prepared in a
similar manner:

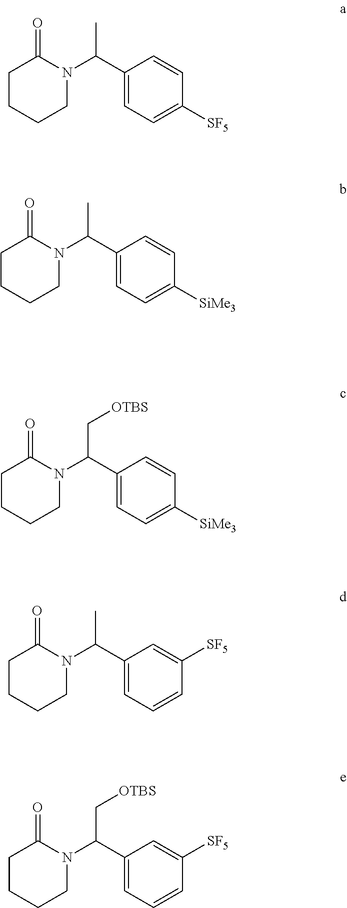

Compounds of the invention having —OSF₅ groups can be prepared according to the scheme below and by techniques well known in the art.

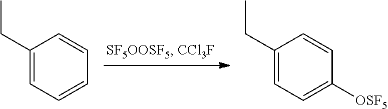

### Journal of the Chemical Society; 1962; 2107-2108

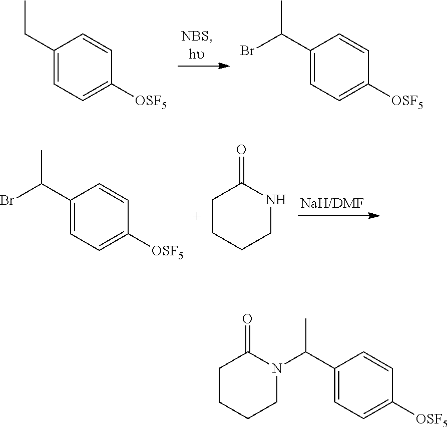

f-h can be prepared in a similar manner:

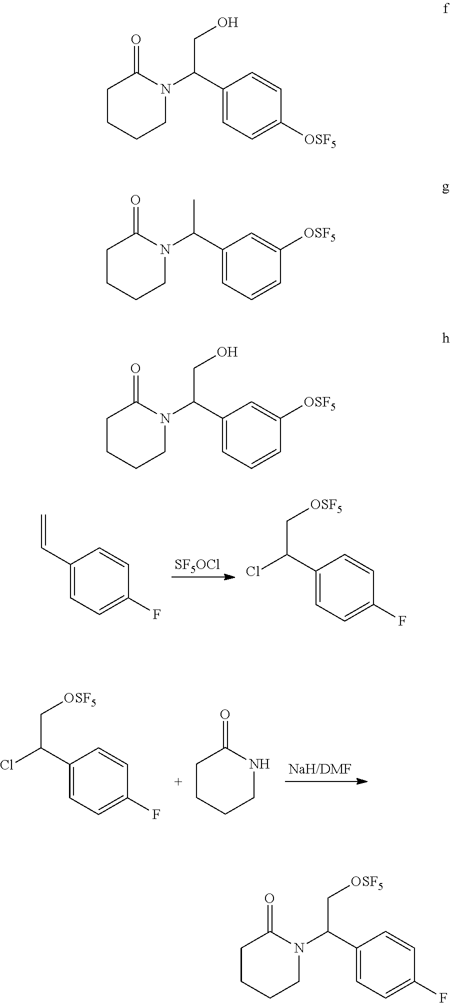

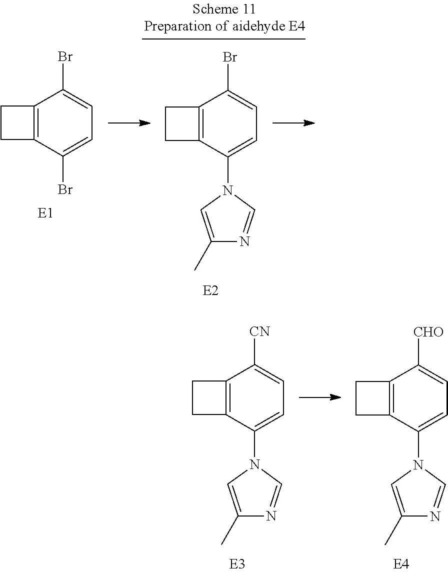

Compound E1 is obtained using a literature method by K. Walker, L., Markoski and J. Moore Synthesis, 1992, 1265.

### Step A:

To a solution of E1 (0.11 mmol) in dry 0.5 mL will be added 4-methyl imidazole (5 eq, 0.546 mmol, 44 mg), Cu₂O (0.4 equiv, 0.044 mmol, 6 mg), 4,7-dimethoxyl-1,8-phenanthracene (0.4 equiv, 0.044 mmol, 10 mg), Cs₂CO₃ (1.4 equiv, 0.154 mmol, 50 mg) and PEG (40 mg). The resulting solution will be degassed and heated at 110° C. for 40 h to give compound E1 after purification.

### Step B:

A procedure from P. Schirch and V. Bockclheide is adapted (J. Amer. Chem. Soc. 1981, 103, 6873). To a solution of E2 (1.5 g) will be added 5.0 eq of cuprous cyanide in 100 ml of N-methyl-2-pyrrolidinone. The mixture will be heated at 115° C. with stirring under nitrogen to give E3 after workup and purification.

### Step C

To a 140 mg of E3 in ether will be added 1 eq of DiBAL in hexane. After 1 h, 5 mL of MeOH will be added and the mixture will be poured into ice water followed by acidification with 10% HCl and extraction with ether. The organic layers will be combined and solvent evaporated to give a residue which will be chromatographed to give compound E4.

The following intermediates will be synthesized using method similar to that of E4:

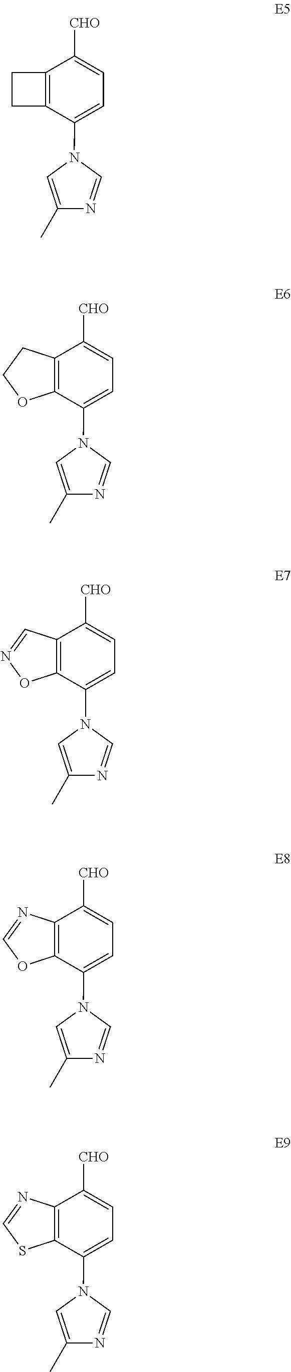

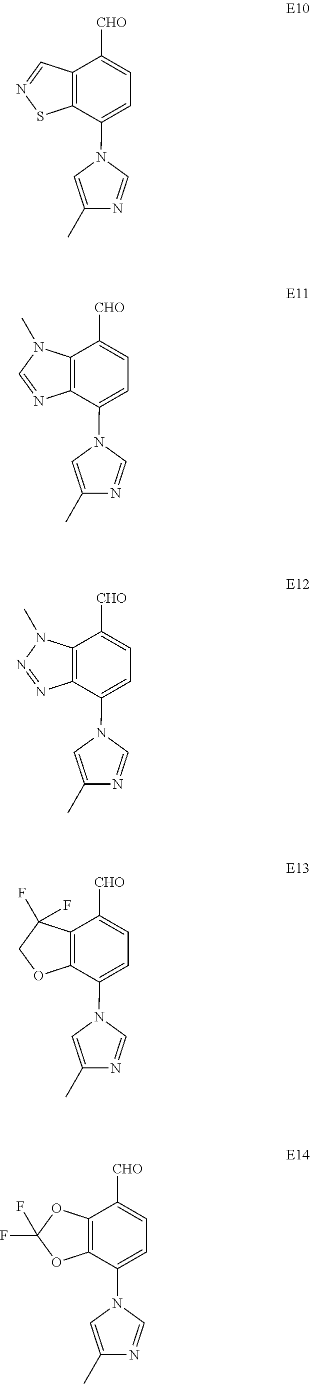

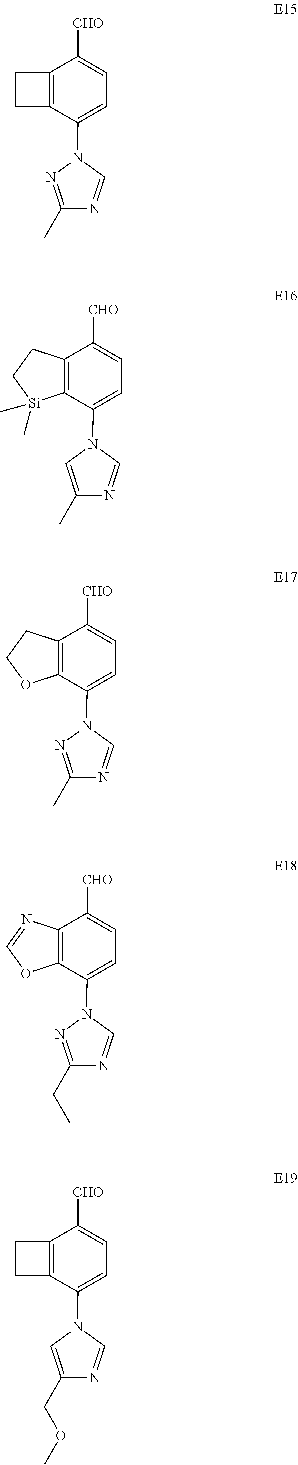

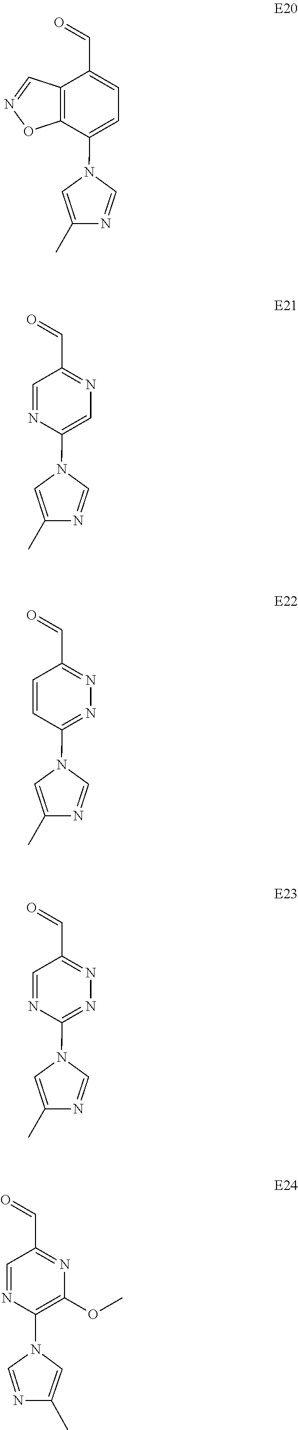

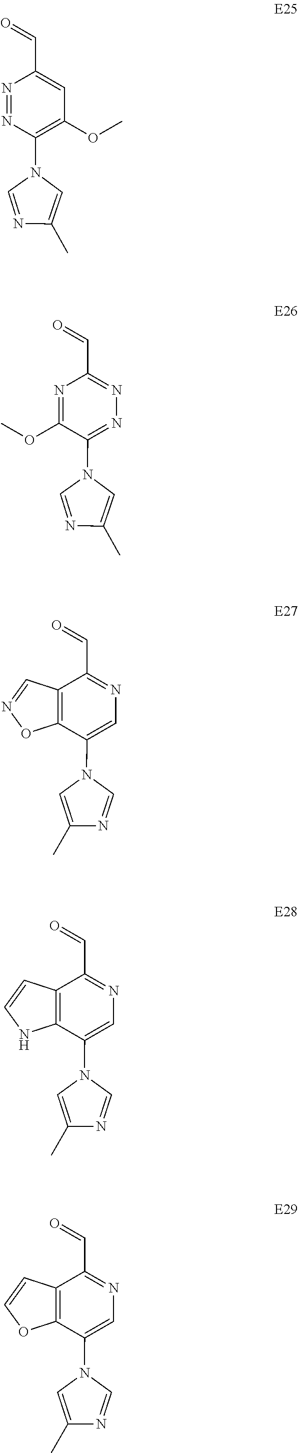

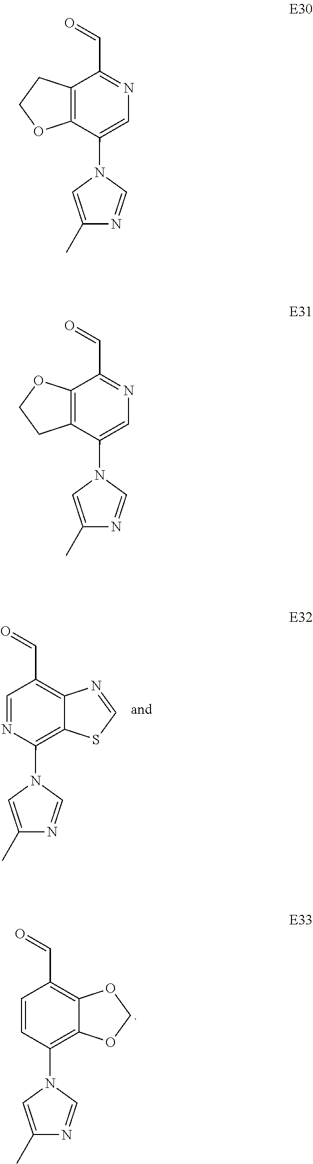

### Example 1

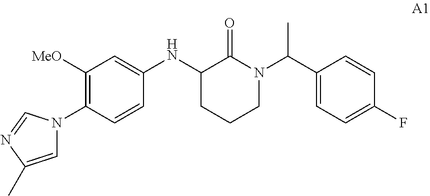

### Step A:

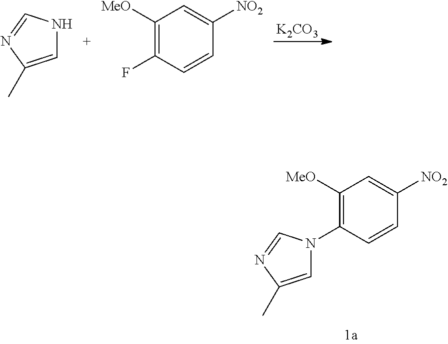

4-methylimidazole (2.0 mmol), 3-methoxy-4-fluoro-nitrobenzene (1.0 mmol) and K₂CO₃ (5 mmol) were stirred in CH₃CN (10 mL) at room temperature over night. The reaction mixture was filtered and concentrated under reduced pressure. The crude product was recrystallized with EtOAc to give desired product 1a.

### Step B:

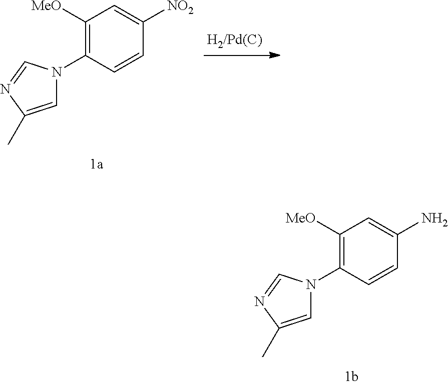

Compound 1a was hydrogenated with hydrogen balloon in the presence of Pd(C) as the catalyst (10 wt %) in MeOH over night. The mixture was filtered and concentrated under reduced pressure to give product 1b.

### Step C:

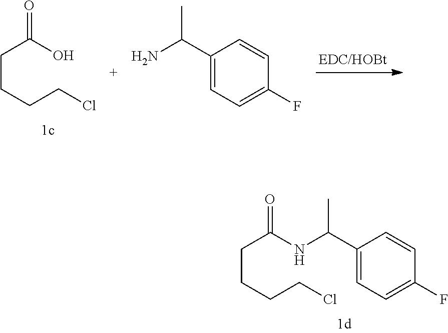

If the following steps were followed then 1d would be prepared from 1c. Stir Compound 1c (1.0 mmol), 1-(4-fluorophenyl)ethyl amine (1.0 mmol), EDCI (2.0 mmol), HOBt (2.0 mmol) and NEt₃ (3.0 mmol) in DMF (5 mL) at room temperature over night. Dilute the mixture with EtOAc (100 mL) and HCl solution (20 mL, 0.5 M). Wash the organic layer with water (3×), brine, dry over MgSO₄, and concentrate to give the crude product. Purify the crude residue by silica gel column chromatography eluting with EtOAc/hexanes to yield compound 1d.

### Step D:

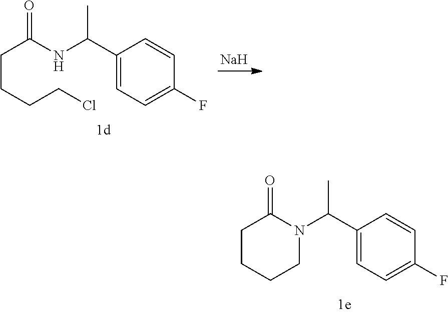

If the following steps were followed then 1e would be prepared from 1d. Add NaH (1.1 mmol) to a solution of compound 1d (1.0 mmol) in DMF (5 mL) at 0° C. Stir the mixture for 15 minutes before heating the mixture at 60° C. overnight. Cool the resulting reaction mixture and dilute it with EtOAc (100 mL) and HCl solution (20 mL, 0.5 M). Wash the organic layer with water (3×), brine, dried over MgSO₄, and concentrate to give the crude product. Purify the crude residue by silica gel column chromatography eluting with EtOAc/hexanes to yield compound 1e.

### Step E:

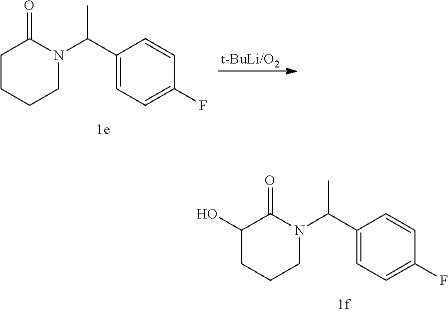

If the following steps were followed then 1f would be prepared from 1e. Treat a solution of compound 1e (0.5 mmol) in THF (4 mL) with t-BuLi (0.6 mmol, 1.0 M) at −78° C. Stir the mixture for 15 minutes before bubbling dry oxygen through the solution for 20 minutes. Dilute the resulting reaction mixture with EtOAc (50 mL) and HCl solution (10 mL, 0.5 M). Wash the organic layer with water, brine, dry over MgSO₄, and concentrate to give the crude product. Purify the crude residue by silica gel column chromatography eluting with EtOAc/hexanes to yield compound 1f.

### Step F:

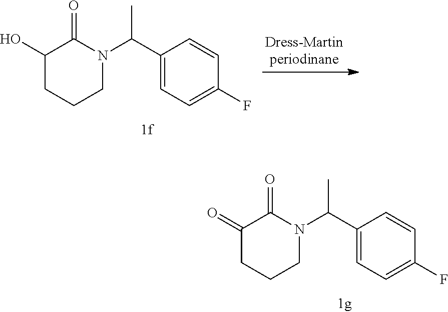

If the following steps were followed then 1g would be prepared from 1f. Treat a solution of compound 1f (0.25 mmol) in CH₂Cl₂ (3 mL) with Dess-Martin Periodinane (0.3 mmol) at room temperature. Stir the mixture for 1.5 hours before diluting it with EtOAc (50 mL) and Na₂S₂O₃ solution (10 mL, 0.5 M). Wash the organic layer with NaHCO₃ (3×), water, brine, dry over MgSO₄, and concentrate to give the crude product. Purify the crude residue by silica gel column chromatography eluting with EtOAc/hexanes to yield compound 1g.

### Step G:

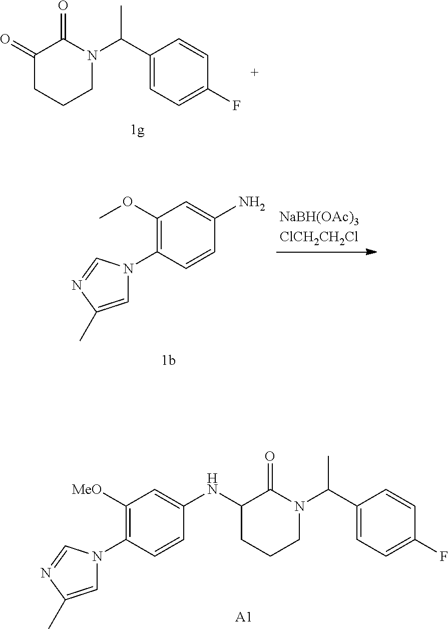

If the following steps were followed then A1 would be prepared from 1g and 1b. Treat a mixture of compound 1g (0.25 mmol), 1b (0.3 mmol) and 4 Å molecular sieves (0.2 g) in ClCH₂CH₂Cl (2 mL) with NaBH(OAc)₃ (0.75 mmol) at room temperature. Stir the reaction mixture at room temperature overnight before diluting it with EtOAc (50 mL) and NaHCO₃ solution (10 mL). Wash the organic layer with water, brine, dry over MgSO₄, and concentrate to give the crude product. Purify the crude residue by silica gel column chromatography eluting with EtOAc/hexanes to yield compound A1.

### Example 2

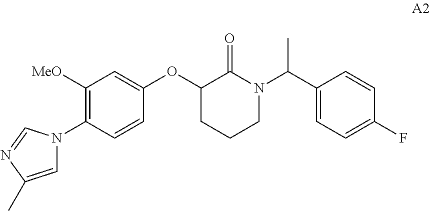

### Step A:

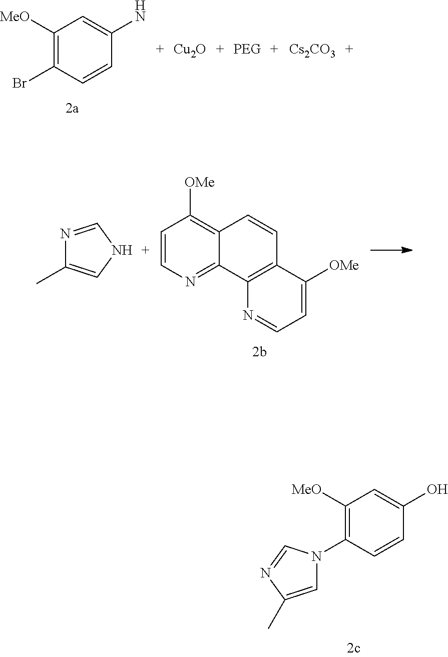

A mixture of compound 2a (2.03 g, 10 mmol), Cu₂O (0.288 g, 2 mmol), PEG (4.0), Cs₂CO₃ (9.77 g, 30 mmol), 4-methylimidazole (0.98 g, 12 mmol) and 2b (0.72 g, 3 mmol) in NMP (15 mL) was vacuum-nitrogen exchange degassed and stirred in a sealed tube at 120° C. for 48 hours. The mixture was cooled to room temperature and diluted with CH₂Cl₂ followed with addition of silica gel. The mixture was stirred for 20 minutes and filtered. The organic layer was washed with water (3×), brine, dried over MgSO₄, and concentrated to give the crude product. The crude residue was purified by column chromatography eluting with CH₂Cl₂/MeOH to yield compound 2c (0.2 g).

### Step B:

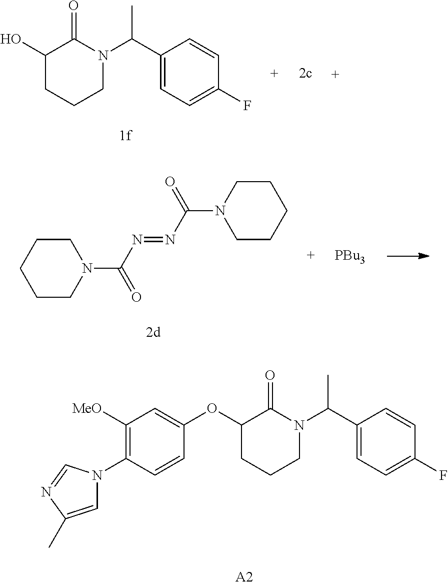

If the following steps were followed then A2 would be prepared from 1f, 2c, and 2d. Heat a mixture of compound 1f (0.22 mmol) (Example 1), 2c (0.26 mmol), reagent 2d (0.26 mmol) and PBu₃ (0.26 mmol) in THF (2.0 mL) at 50° C. overnight. Filter the mixture through a short pad of celite and wash with EtOAc. Concentrate the solvent to give the crude product. Purify the crude residue by silica gel column chromatography eluting with EtOAc/Hexane to yield compound A2.

### Example 3

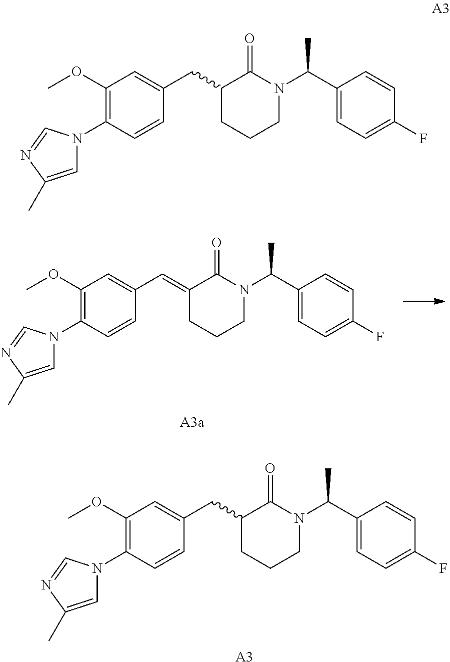

Into a Vial was added (S,E)-1-(1-(4-fluorophenyl)ethyl)-3-(3-methoxy-4-(4-methyl-1H-imidazol-1-yl)benzylidene)piperidin-2-one A3a (18 mg, 0.043 mmol), 20% Palladium Hydroxide on Carbon (2:8, Palladium hydroxide:carbon black, 8 mg), and Methanol (2 mL, 50 mmol) The reaction was degassed and stirred at room temperature under an atmosphere of Hydrogen for 16 hours. The reaction was filtered through a pad of silica and concentrated to yield as ˜2:1 mixture of diastereomers.

The diastereomers A4 and A5, in Table 1 below, were separated by SFC-AD semi-prep column to cleanly yield two diasteromers individually.

### Example 6

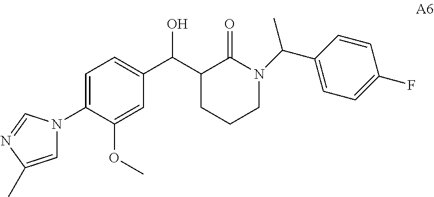

### Step A:

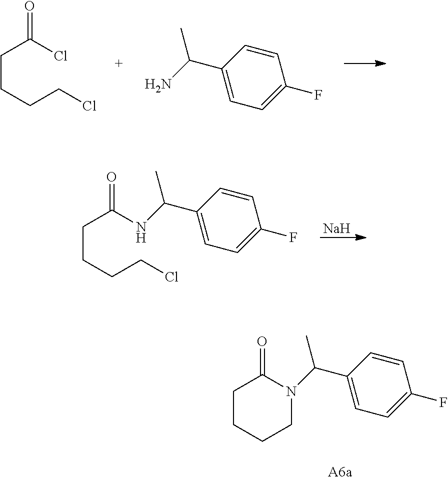

To a solution of 10.0 g (71.9 mmol) of 1-(4-fluorophenyl)ethanamine in 40 mL of DCM and 29 mL of pyridine, with ice cooling, was added dropwise a solution of 12.08 g (71.9 mmol) of 6-chlorohexanoyl chloride in 40 mL of DCM. The mixture was stirred overnight, washed with 2M HCl, and the organic phase was dried over MgSO₄. The solvent was evaporated, and 8.147 g of crude 5-chloro-N-(1-(4-fluorophenyl)ethyl)pentanamide (ES-MS, M+1) 258, was obtained. 18.09 g (70.39 mmol) of 5-chloro-N-(1-(4-fluorophenyl)ethyl)pentanamide was dissolved in 250 mL of THF, treated with 3.097 g (34.857 mmol) of 60% suspension of NaH in mineral oil, and refluxed for 5 h. The reaction mixture was cooled, quenched with water, and extracted with DCM. The organic phase was washed with water and brine, dried over Na₂SO₄, concentrated, and purified by chromatography on SiO₂ using a gradient of 0-70% of EtOAc and hexanes to furnish 13.8 g of the target 1-(1-(4-fluorophenyl)ethyl)piperidin-2-one A6a. (ES-MS, M+1) 258.

### Step B:

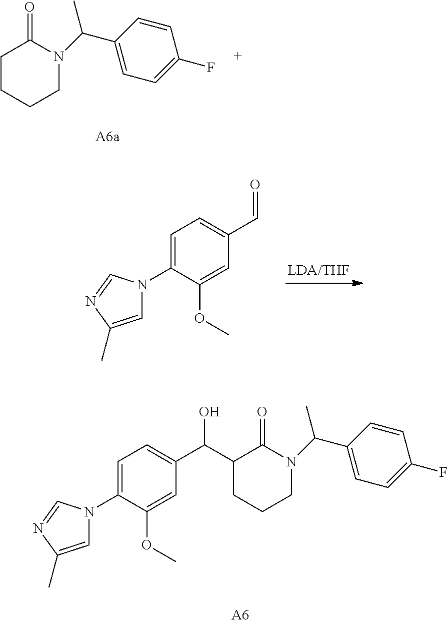

To a solution of 110 mg (0.497 mmol) of 1-(1-(4-fluorophenyl)ethyl)piperidin-2-one A6a in THF (2 ml) was added 1.24 mL of 2M LDA (in THF/Heptane, Acros) at −78° C. The reaction was stirred for 30 min at −78° C., then stirred for 30 min at −20° C., and was re-cooled to −78° C. Added 3-methoxy-4-(4-methyl-1H-imidazol-1-yl)benzaldehyde as solid and stirred for 30 min. The mixture was quenched with saturated aqueous NaHCO₃, extracted with EtOAc (2 times), and washed with brine (2×). The organic phase was dried (MgSO₄) and concentrated. The product was purified by a gradient reverse-phase HPLC (CH₃CN—H₂O with 0.1% formic acid) to furnish 110 mg of the product A6. (ES-LCMS, M+1) 438.2. Retention time: 2.94 min.

Compounds A10 and A12, in Table 1, were prepared in a similar procedure as that of A6.

### Example 7

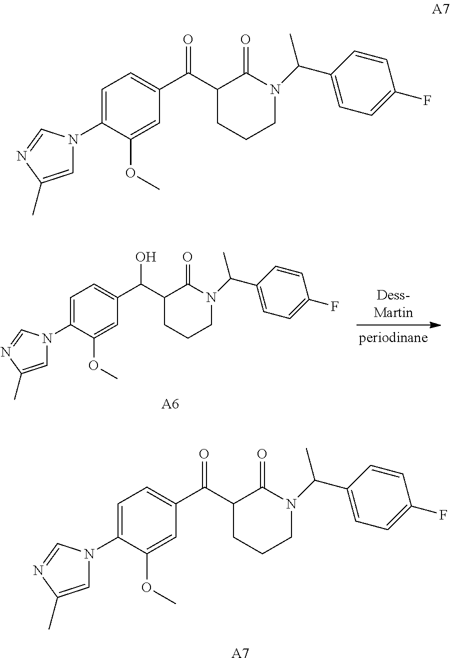

A solution on 590 mg (1.35 mmol) of A6 in 13 mL of DCM was treated with 573 mg (1.35 mmol) of Dess-Martin periodinane. The mixture was stirred for 3 h, quenced by addition of a mixture of 1.07 g (6.75 mmol) of sodium thiosulfate in 5 mL of water, followed by 5 mL of saturated aqueous NaHCO₃. The biphasic mixture was stirred for 3 h, the layers were separated, and the aqueous phase was extracted with DCM. Combined organic phases were dried over Na₂SO₄ and evaporated. The crude product was purified by a gradient reverse-phase HPLC (CH₃CN—H₂O with 0.1% formic acid) to furnish 330 mg of the product A7. (ES-LCMS, M+1) 436.2. Retention time: 3.06 min.

Compound A9, in Table 1, was prepared in a similar procedure as that of A7.

### Example 8

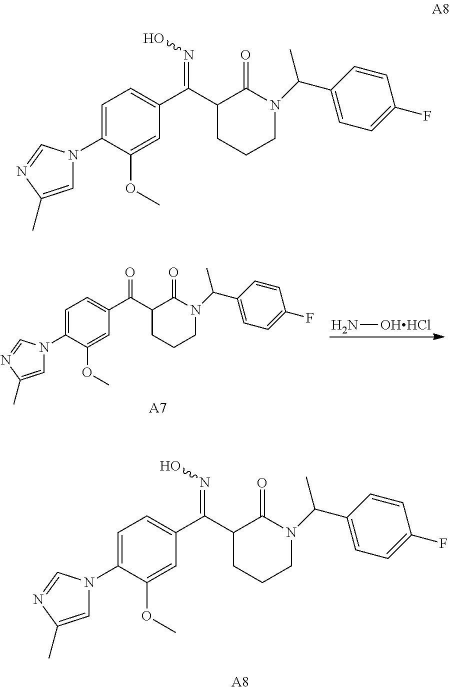

A mixture of 100 mg (0.23 mmol) of A7, 32 mg (0.46 mmol) of hydroxylamine hydrochloride, and 4 mL of methanol was heated at reflux for 2 h. The solvent was evaporated, and part of the material (ca 20 mg) was purified by a gradient reverse-phase HPLC (CH₃CN—H₂O with 0.1% formic acid) to furnish 2.0 mg of the product A8, existing as a mixture of 2 isomers. (ES-LCMS, M+1) 451.2. Retention time: 2.25 and 3.06 min.

Compound A11 was prepared in a similar procedure as that of A8.

### Example 13

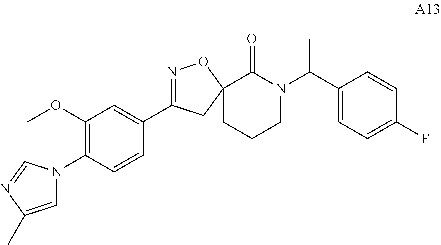

### Step A:

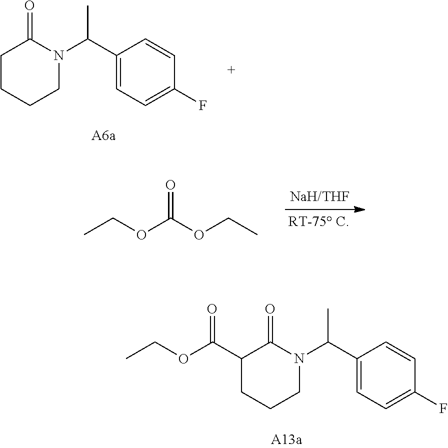

To 3.62 g (90.498 mmol) of 60% suspension of NaH in mineral oil was added THF (25 ml) followed by a mixture of 5.0 g (22.624 mmol) of 1-(1-(4-fluorophenyl)ethyl)piperidin-2-one A6a and 24.05 g (203.62 mmol) of diethyl carbonate in THF (20 ml) dropwise. Stirred for 9 h at 70° C. The reaction mixture was extracted with EtOAc, washed with water and brine. The organic phase was dried over Na₂SO₄ and concentrated to afford compound A13a (5.9 g). (ES-LCMS, M+1) 294. Retention time 2.14 min.

### Step B:

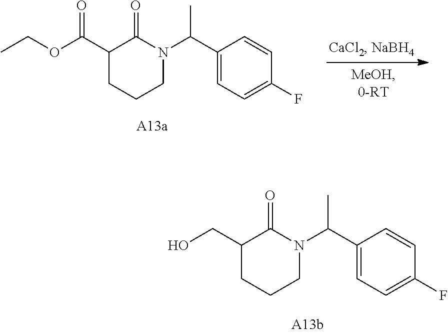

To a mixture of 6.986 g (23.832 mmol) of compound A13a and 4.31 g (28.598 mmol) of CaCl₂ in 40 mL of dry methanol was added in portions at 0° C. 1.081 g (28.598 mmol) of NaBH₄. The mixture was stirred at 0° C. for 1 h and was allowed to warm up to rt over a period of 5 h. The solids were filtered and washed with methanol. The filtrate was evaporated to form a solid, and it was extracted with DCM. The DCM phase was washed with water and brine and dried over Na₂SO₄, The solvent was evaporated, and the crude product was purified by SiO₂ chromatography using a gradient of 40-90% of EtOAc in hexanes to furnish 5.47 of alcohol A13b. (ES-MS, M+1) 252.

### Step C:

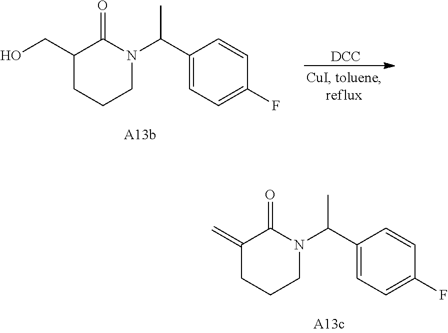

To a mixture of 5.068 g (20.181 mmol) of alcohol A13b and 5.205 g (25.22 mmol) of DCC in 30 mL of toluene at 105° C. was added 384 mg (1.018 mmol) of CuI. The reaction mixture was stirred for 1 h, cooled, treated with 10 mL of water, and stirred for an additional hour. The solids were filtered, the filtrate partitioned between DCM and water, the organic phase was washed with water and brine, dried over Na₂SO₄ and concentrated. The crude product was purified by SiO₂ chromatography using a gradient of 0-40% of EtOAc in hexanes to furnish 5.1 g of alkene A13c (ES-MS, M+1) 234.

### Step D:

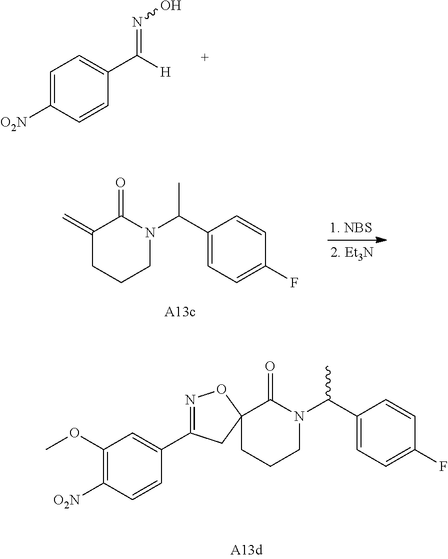

To a solution of 1.52 g (7.75 mmol) of oxime in 10 mL of DMF at 0° C. was added dropwise 1.433 g (8.050 mmol) of NBS in 5.0 mL of DMF. The mixture was stirred for 1 h at 0° C. A mixture of compound A13c (1.39 g, 5.963 mmol) and Et₃N (845 mg, 8.348 mmol) in 5.0 mL of DCM (pre-cooled to 0° C.) was added dropwise, and the addition funnel was rinsed with 1.0 mL of DMF. The reaction mixture was stirred for 4 h, and after the aqueous workup the diastereomeric mixture of compounds A13d and A13d′ was separated by chromatography on a 80 g SiO₂ column, at flow rate of 35 mL/min, using as the solvent a gradient of 0 to 50% of EtOAc in hexanes over 60 min followed by hold for 10 min, followed by another gradient of 50 to 60% of EtOAc in hexanes over 50 min. The first eluting diastereomer (ES-MS, M+1) 428, was designated as A13d (yield 600 mg), and the second eluting diastereomer was designated as A13d′ (yield 820 mg).

### Step E:

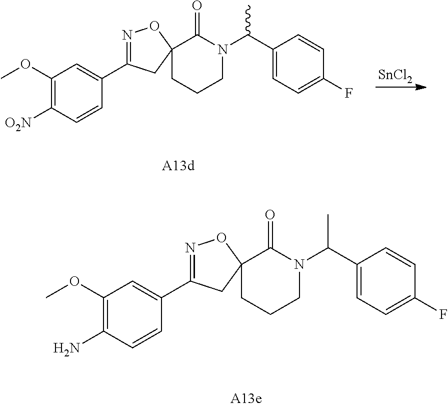

Stirred a mixture of 600 mg (1.405 mmol) of A13d and 2.131 g (11.24 mmol) of SnCl₂ in 5.0 mL of EtOH at reflux for 2 h. After the cooling, reaction mixture was diluted with DCM and treated with 30 mL of 1M NaOH. After 30 min of stirring, solids were filtered out, DCM layer was separated and concentrated, and the product was purified by SiO₂ chromatography using a gradient of 0-10% of methanol in DCM as the solvent to furnish 300 mg of A13e (ES-MS, M+1) 398.

### Step F:

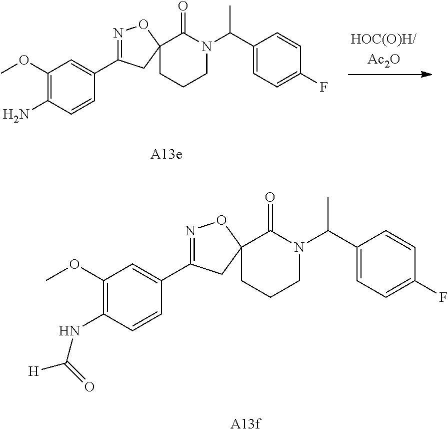

To 0.41 mL of anhydrous formic acid was added dropwise 308 mg (3.021 mmol) of acetic anhydride, with cooling of the reaction flask using tap water. Stirred the mixture for 1 h and added a solution of 300 mg (0.755 mmol) of A13e in 5.0 mL of THF. The mixture was stirred for 1 h, partitioned between water and EtOAc, the organic phase was washed with brine, dried and concentrated. The product was purified by SiO₂ chromatography using 5% of methanol in DCM as the solvent to furnish 321 mg of A131 (ES-MS, M+1) 426.

### Step G:

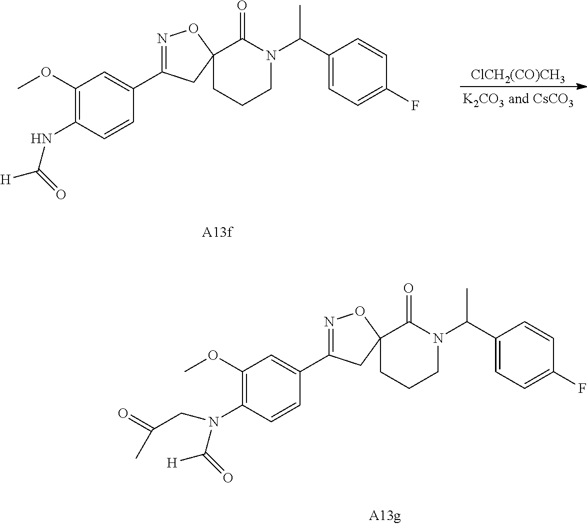

To a mixture of 320 mg (0.753 mmol) of A13f and 229 mg (1.656 mmol) of K₂CO₃ in 4.0 mL of acetone was added dropwise 84 mg of chloroacetone, and the mixture was stirred for 4 h at rt. Additional 139 mg (1.51 mmol) of chloroacetone and 363 mg (1.88 mmol) of CsCO₃ was added to the reaction mixture, and stirring was continued overnight at the reflux temperature. The reaction mixture was cooled, partitioned between water and EtOAc, the organic phase was washed with brine, dried over MgSO₄, concentrated, and the crude product was purified by SiO₂ chromatography using a gradient of 50% of EtOAc in hexanes as the solvent to furnish 315 mg of A13g (ES-MS, M+1) 482.

### Step H:

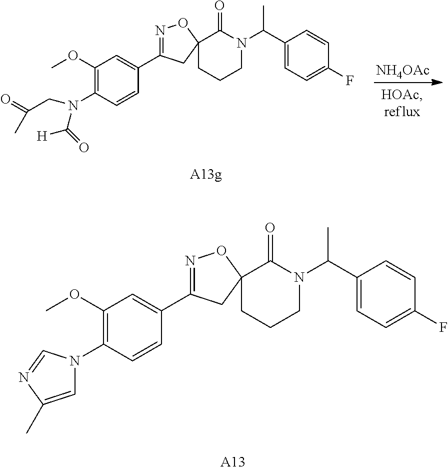

Heated a mixture of 315 mg (0.655 mmol) of A13g, 757 mg (9.82 mmol) of ammonium acetate and 4.0 mL of acetic acid at 95° C. for 19 h. The reaction mixture was cooled, poured into ice water, neutralized with ammonia, and extracted with EtOAc. The organic phase was washed with brine, concentrated, and the product was purified by SiO₂ chromatography using a gradient of 0-5% of MeOH in DCM as the solvent to furnish 170 mg of A13. (ES-LCMS, M+1) 463.2. Retention time: 2.01 min.

### Synthesis of Compound A14 (Table 1)

Compound A14 was prepared from A13d′ in a similar manner as the preparation of A13 from A13d (ES-LCMS, M+1) 463.2. Retention time: 2.02 min.

Compounds A49-A61, in Table 1, will be prepared using a similar sequence as in the preparation A13.

### Example 15

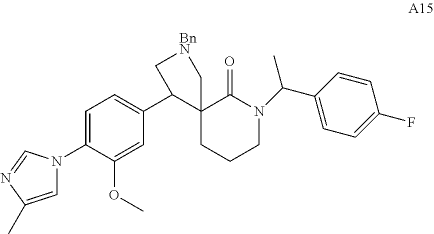

### Step A:

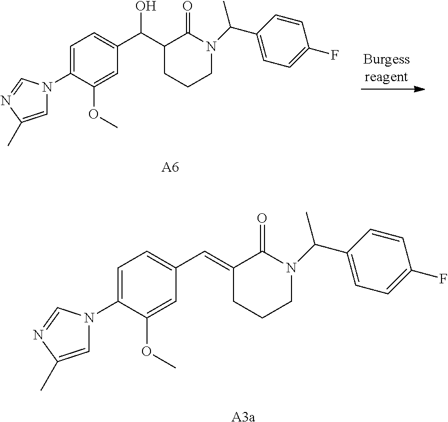

Compound A6 (1 mmol) was treated with Burgess reagent (2.0 mmol) in THF (5.0 mL). The mixture was stirred at reflux for 6 hours before it was cooled. Solvent was removed and crude was purified with silica gel column chromatography to give product A3a.

### Step B:

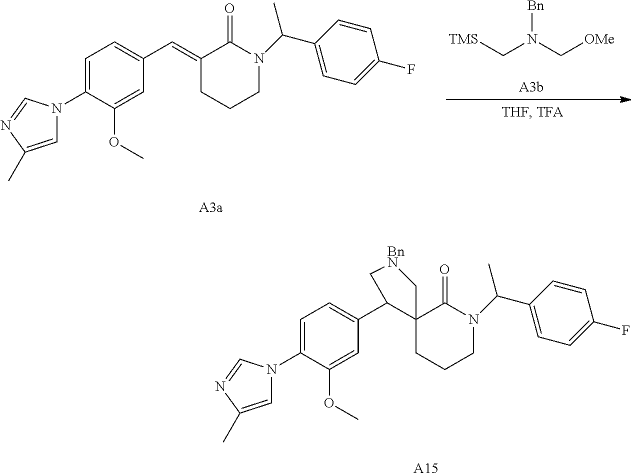

Compound A3a (1 mmol) was treated with A3b (3.0 mmol) and TFA (4.0 mmol) in THF (5.0 mL). The mixture was stirred at reflux for 24 hours before it was cooled. The mixture was diluted with EtOAc and 1N NaOH (5 mL). Aqueous phase was extracted with EtOAc. The combined organic phases were washed with water, brine, and dried (Na₂SO₄). Solvent was collected with filtration and removed under reduced pressure. The crude was purified with silica gel column chromatography to give product A15.

Compounds A16, in Table 1, was prepared using the same sequence as A15.

### Example 17

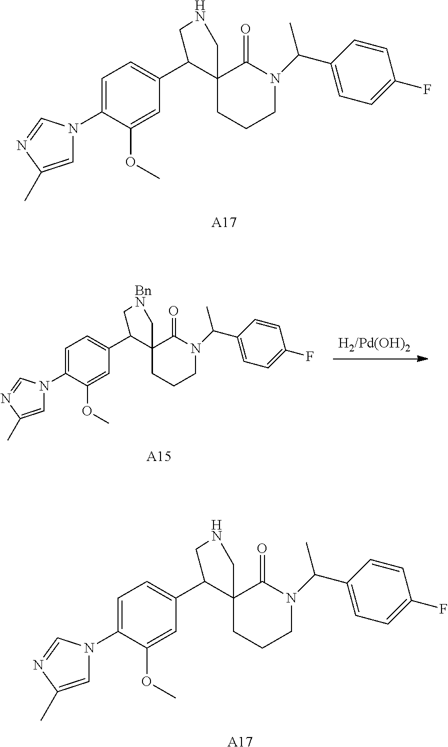

Compound A15 (1 mmol) will be hydrogenated in the presence of Pd(OH)₂/C. Solvent will be removed after filtration and crude will be purified with silica gel column chromatography to give product A17.

Compounds A18, A25-A26, A33-A34, and A41-A42, in Table 1, will be prepared using a similar sequence as A17.

### Example 19

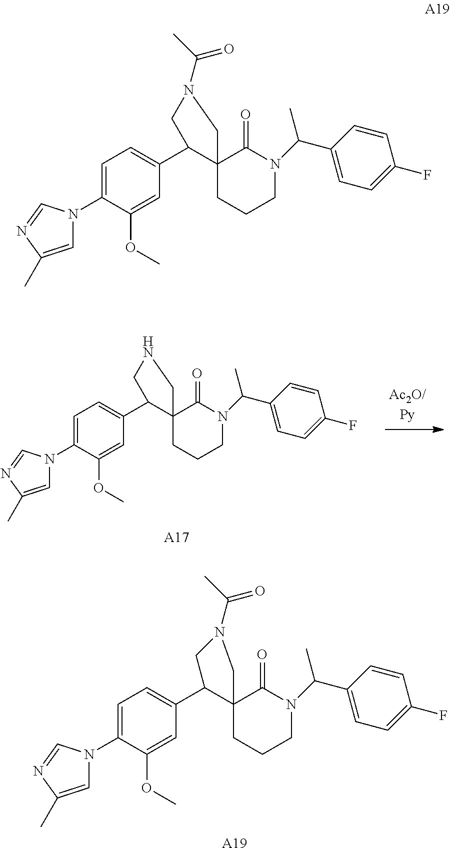

Compound A17 (1 mmol) will be treated with Ac₂O (2.0 eq) and py (3 mL) at room temperature overnight. Solvent will be removed and the crude will be purified with Gilson reverse phase HPLC to give product A19.

Compounds A20, A23-A24, A27-A28, A31-A32, A35-A36, A39-A40, A43-A44, A47-A48, in Table 1, will be prepared using a similar sequence as A19.

### Example 21

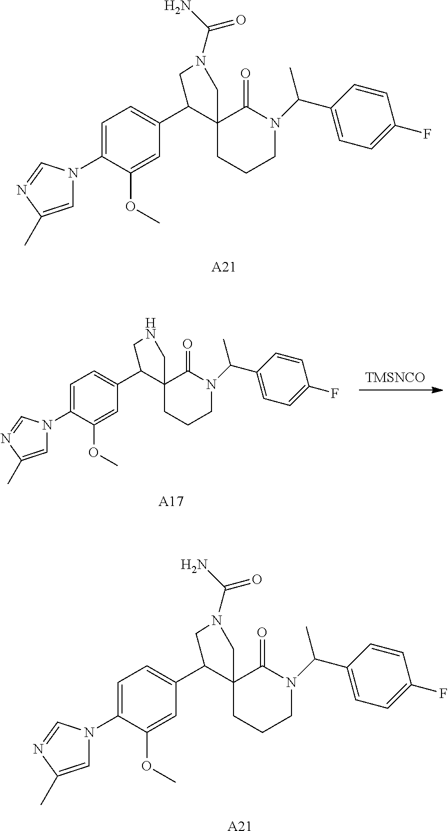

Compound A17 (1 mmol) will be treated with TMSNCO (1.5 mmol) and MeOH (3 mL) at room temperature overnight. Solvent will be removed and the crude will be purified with Gilson reverse phase HPLC to give product A21.

Compounds A22, A29-A30, A37-A38, and A45-A46, in Table 1, will be prepared using a similar sequence as A21.

Compounds A62-A73, in Table 1, will be prepared using a similar sequence as A1.

Compounds A74-A85, in Table 1, will be prepared using a similar sequence as A2.

TABLE 1

| Compound | Structure | LCMS |
|---|---|---|
| A3 | 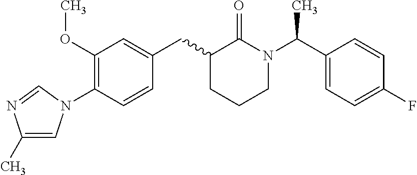 | 422.2 |
| A4 | 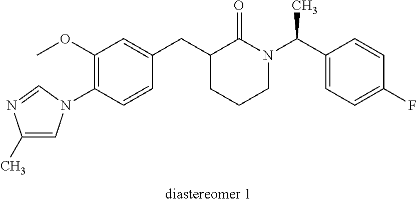 | 422.2 |
| A5 | 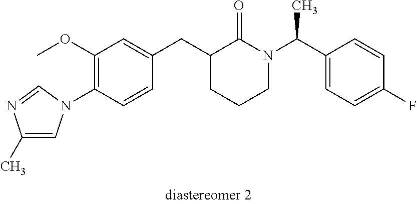 | 422.2 |
| A6 | 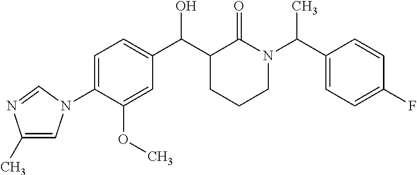 | 438.2 |
| A7 | 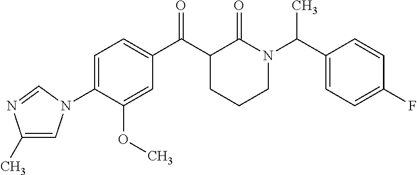 | 436.2 |
| A8 | 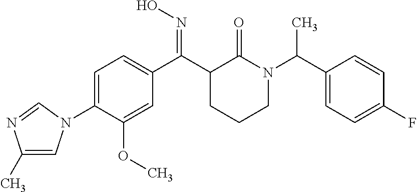 | 451.2 |
| A9 | 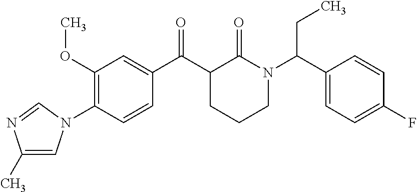 | 450.2 |
| A10 | 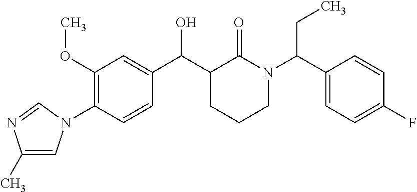 | 452.2 |
| A11 | 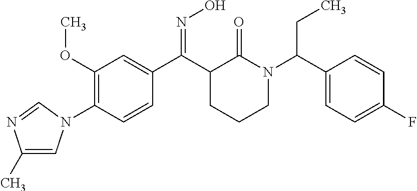 | 465.3 |
| A12 | 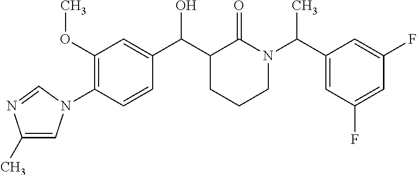 | 456.3 |
| A13 | 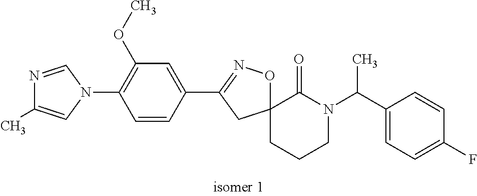 | 463.2 |
| A14 | 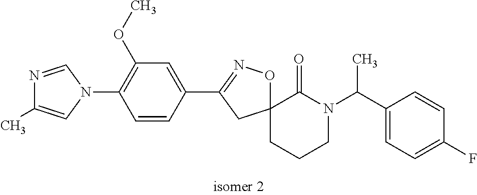 | 463.2 |
| A15 | 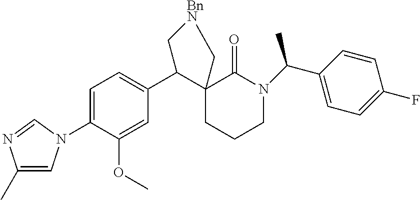 | 553.3 |
| A16 | 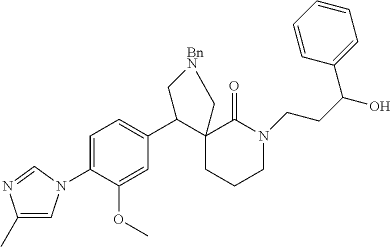 | 565.3 |
| A17 | 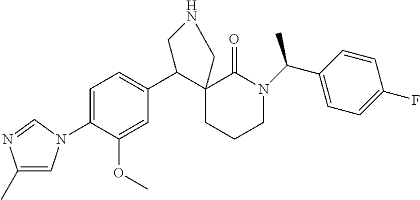 | — |
| A18 | 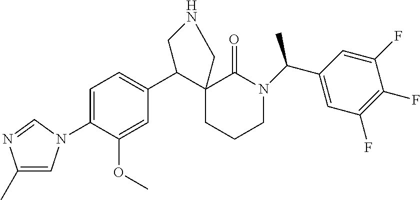 | — |
| A19 | 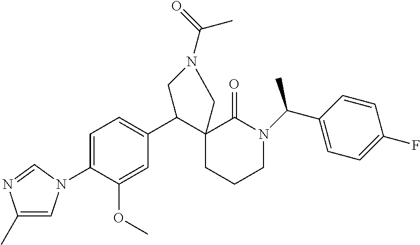 | — |
| A20 | 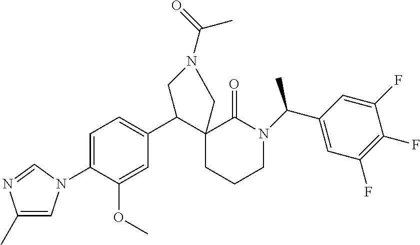 | — |
| A21 | 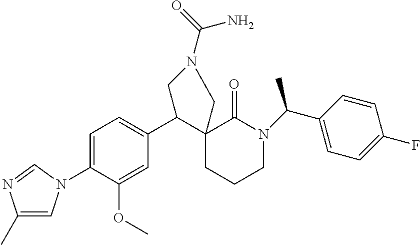 | — |
| A22 | 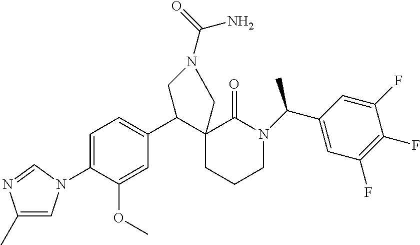 | — |
| A23 | 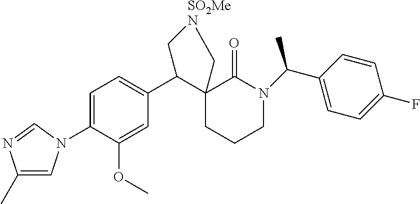 | — |
| A24 | 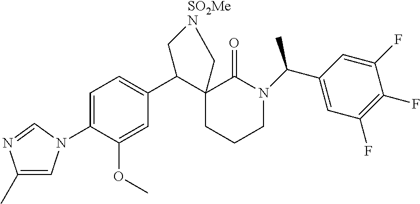 | — |
| A25 | 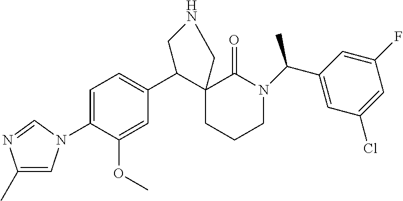 | — |
| A26 | 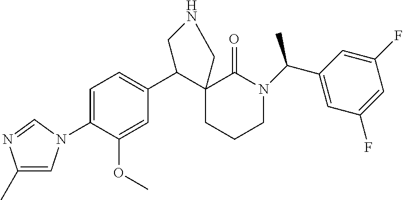 | — |
| A27 | 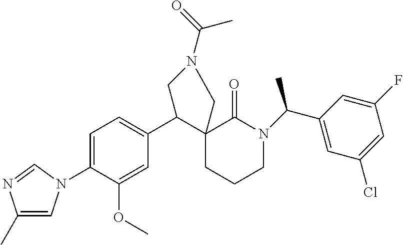 | — |
| A28 | 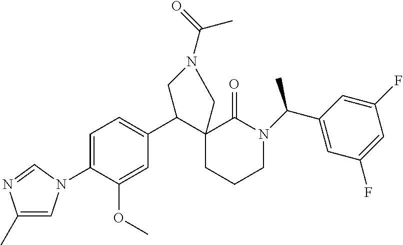 | — |
| A29 | 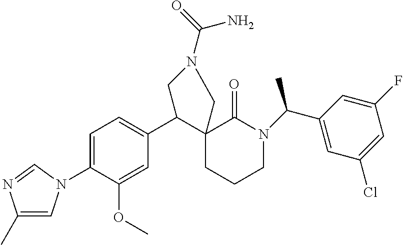 | — |
| A30 | 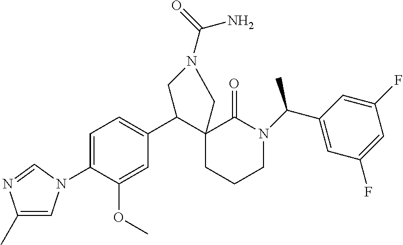 | — |
| A31 | 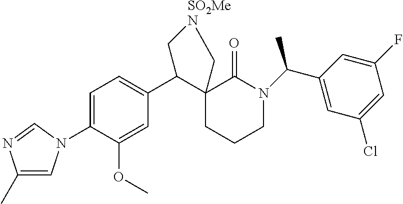 | — |
| A32 | 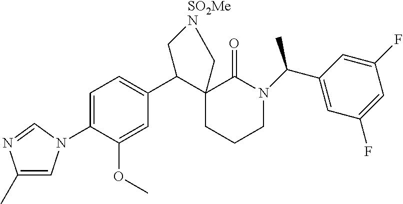 | — |
| A33 | 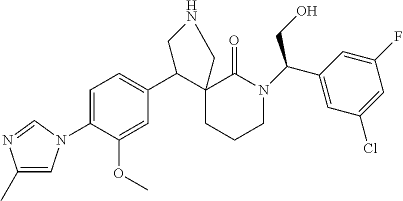 | — |
| A34 | 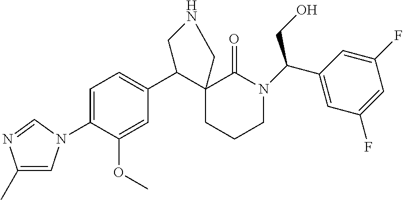 | — |
| A35 | 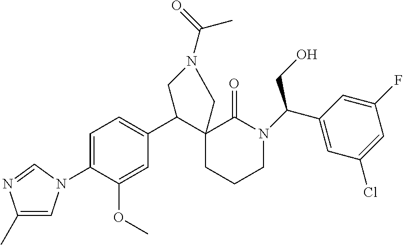 | — |
| A36 | 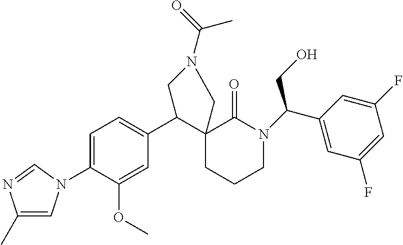 | — |
| A37 | 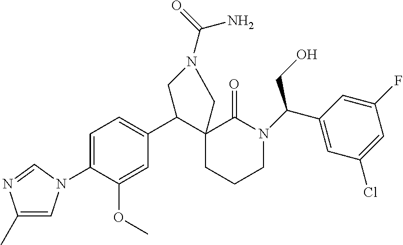 | — |
| A38 | 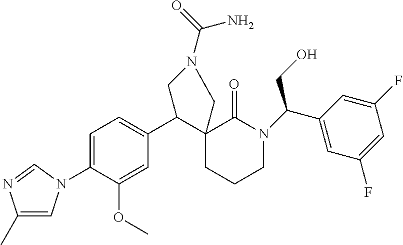 | — |
| A39 | 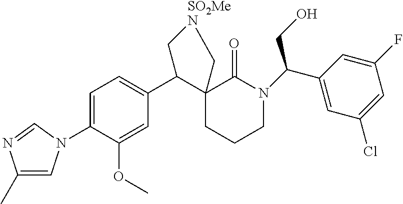 | — |
| A40 | 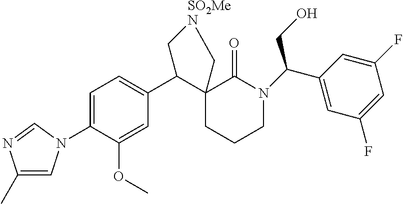 | — |
| A41 | 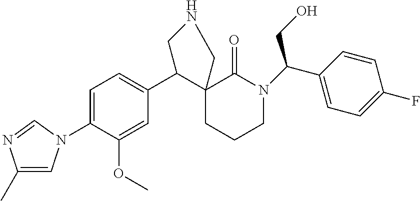 | — |
| A42 | 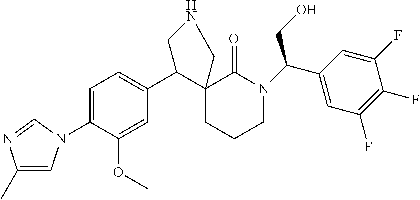 | — |
| A43 | 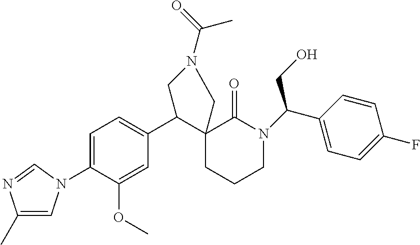 | — |
| A44 | 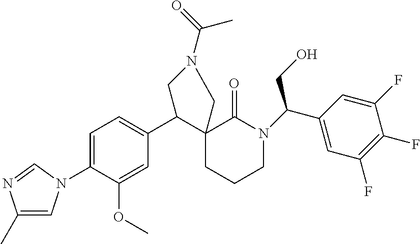 | — |
| A45 | 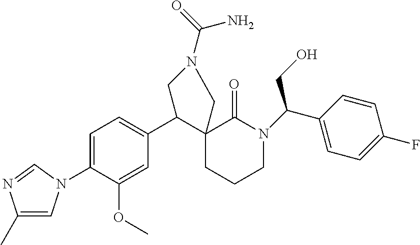 | — |
| A46 | 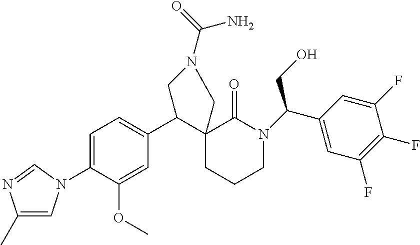 | — |
| A47 | 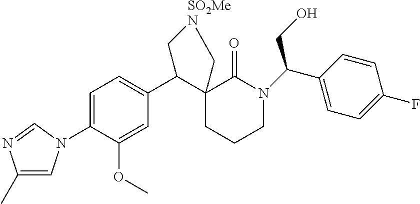 | — |
| A48 | 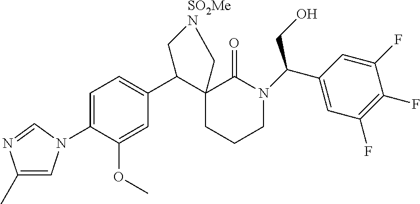 | — |
| A49 | 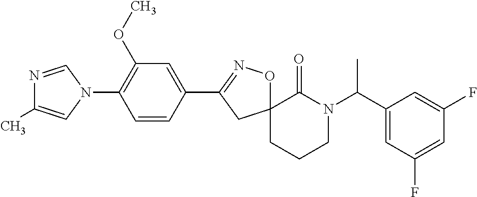 | — |
| A50 | 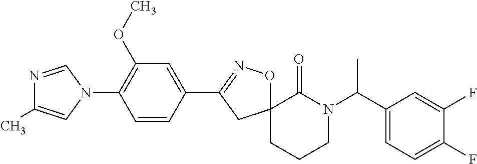 | — |
| A51 | 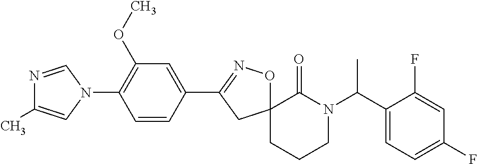 | — |
| A52 | 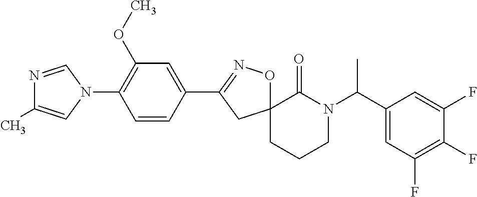 | — |
| A53 | 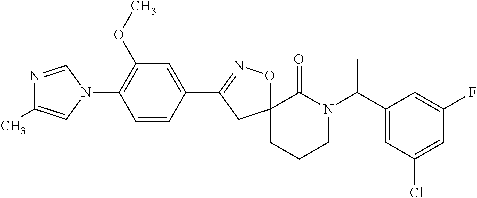 | — |
| A54 | 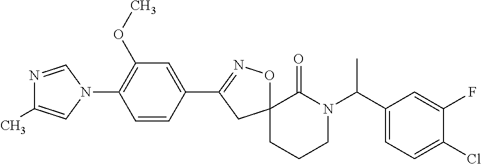 | — |
| A55 | 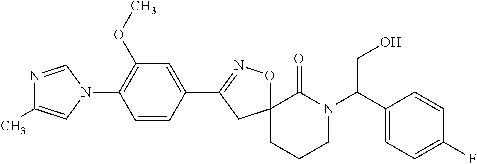 | — |
| A56 | 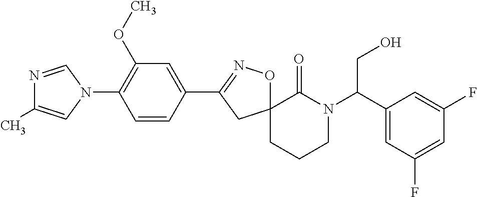 | — |
| A57 | 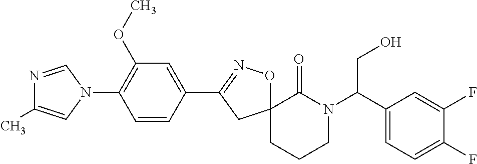 | — |
| A58 | 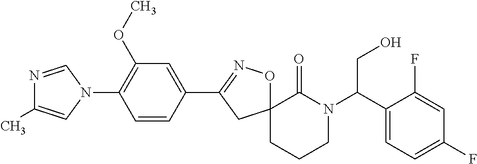 | — |
| A59 | 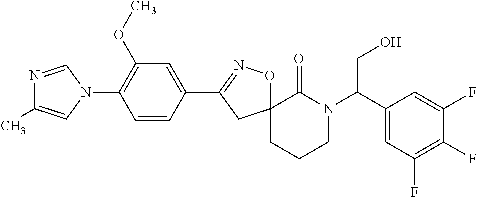 | — |
| A60 | 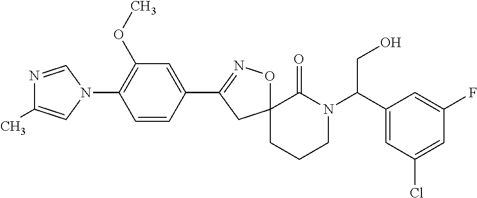 | — |
| A61 | 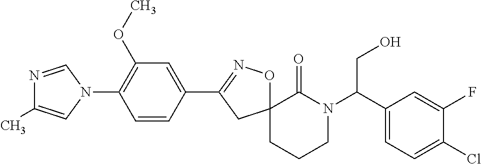 | — |
| A62 | 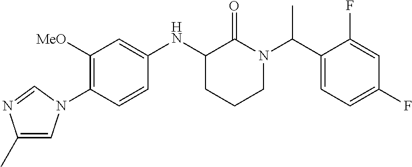 | — |
| A63 | 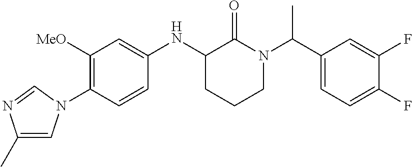 | — |
| A64 | 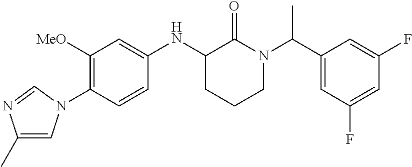 | — |
| A65 | 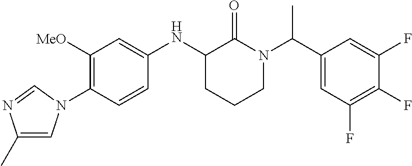 | — |
| A66 | 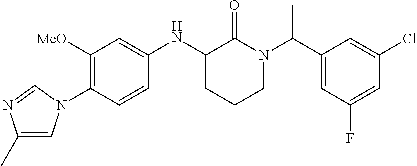 | — |
| A67 | 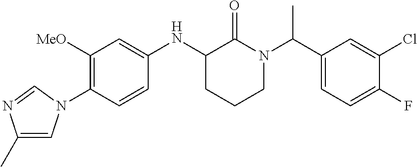 | — |
| A68 | 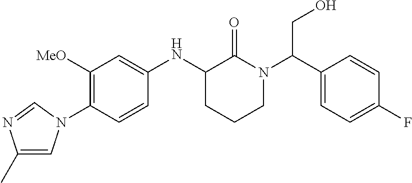 | — |
| A69 | 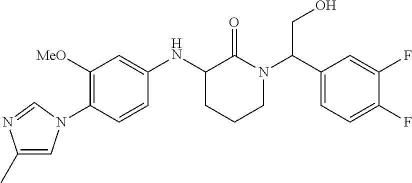 | — |
| A70 | 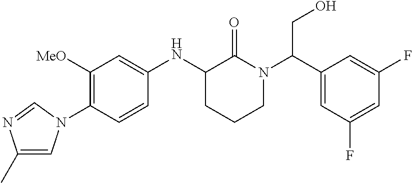 | — |
| A71 | 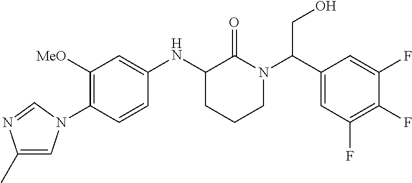 | — |
| A72 | 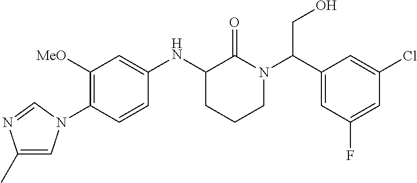 | — |
| A73 | 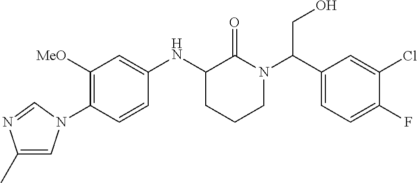 | — |
| A74 | 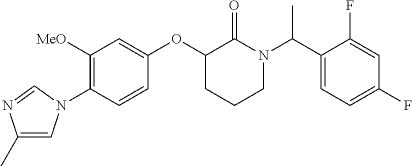 | — |
| A75 | 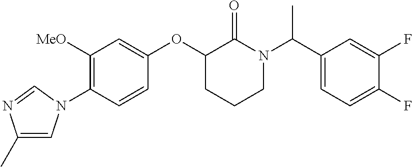 | — |
| A76 | 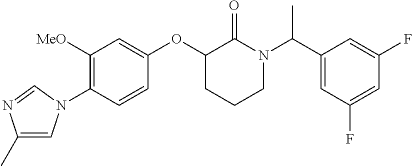 | — |
| A77 | 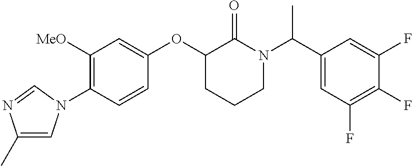 | — |
| A78 | 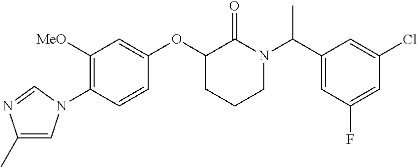 | — |
| A79 | 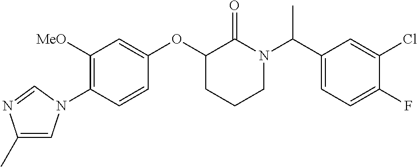 | — |
| A80 | 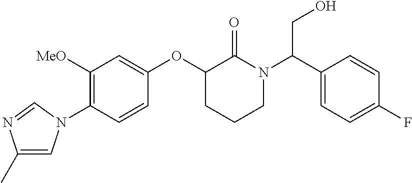 | — |
| A81 | 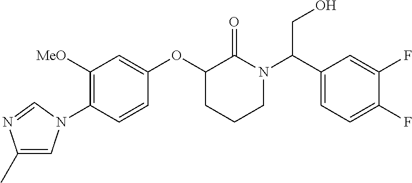 | — |
| A82 | 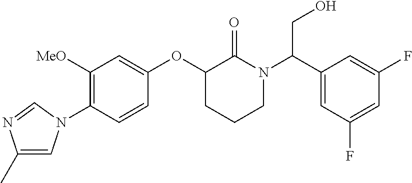 | — |
| A83 | 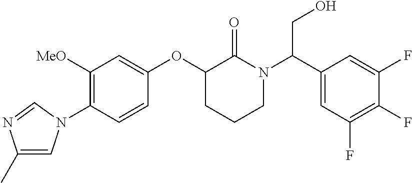 | — |
| A84 | 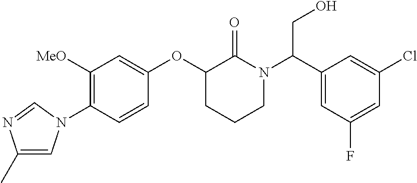 | — |
| A85 | 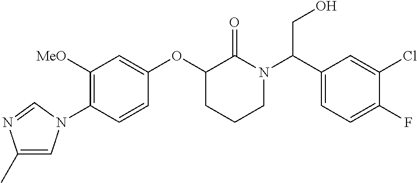 | — |

### Example 22

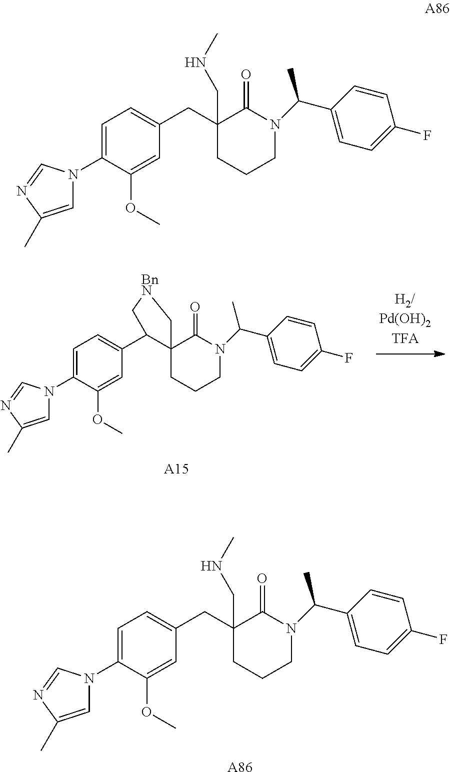

Compound A15 (1 mmol) was hydrogenated in the presence of Pd(OH)₂/C and TFA in MeOH at 60 psi. Solvent was removed after filtration and crude was purified with silica gel column chromatography to give product A86.

Compound A87, in Table 2, was prepared using a similar sequence as A86.

### Example 23

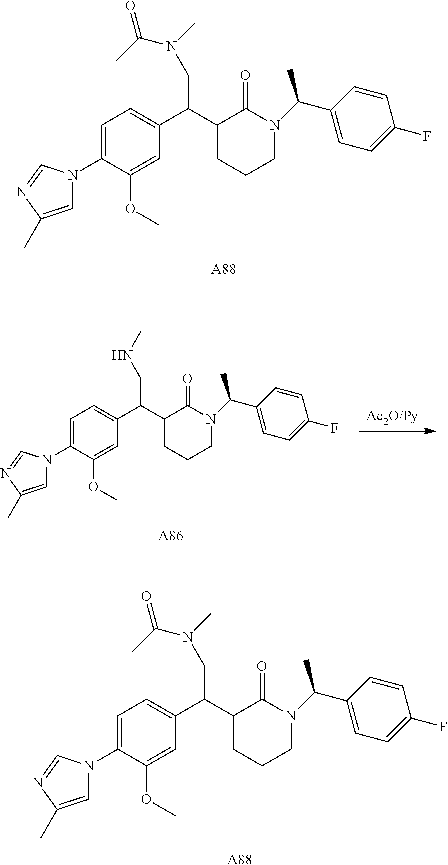

Compound A86 (1 mmol) was treated with AcCl (2.0 eq) and NEt₃ (4 eq) in CH₂Cl₂ (5 mL) at room temperature overnight. Solvent was removed and the crude was purified with preparative thin layer chromatography to give product A88.

Compound A89, in Table 2, was prepared using a similar sequence as A88.

### Example 24

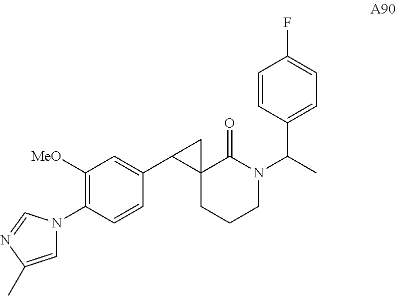

### Step A:

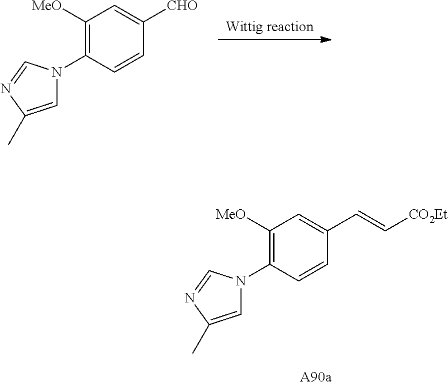

(EtO)₂P(O)CH₂CO₂Et (1.2 mmol) will be treated with NaH (1.2 mmol) in THF (5.0 mL) for 0.5 hour. 3-methoxy-4-(4-methyl-1H-imidazol-1-yl)benzaldehyde (1 mmol) will be added. The mixture will be stirred for 2 hours before it will be quenched with addition of NH₄Cl aqueous solution. Aqueous layer will extracted with EtOAc. Organic phase will be washed with water, brine and dried over MgSO₄. Solvent will be concentrated to give the crude product. The crude residue will be purified by silica gel column chromatography to yield compound A90a.

### Step B:

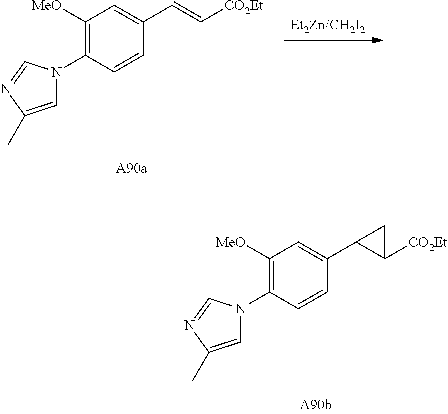

CH₂Cl₂ (3 mmol) will be added to Et₂Zn (3 mmol) in CH₂Cl₂ (6 mL) at 0° C. The mixture will be stirred for 15 minutes before A90a (1 mmol) will be introduced. The mixture will be stirred overnight before it will be quenched with addition of NH₄Cl aqueous solution. Aqueous layer will extracted with EtOAc. Organic phase will be washed with water, brine and dried over MgSO₄. Solvent will be concentrated to give the crude product. The crude residue will be purified by silica gel column chromatography to yield compound A90b.

### Step C:

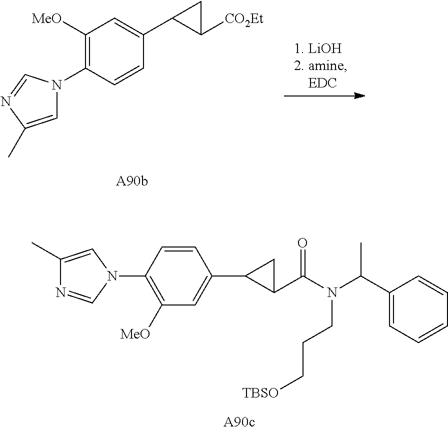

Compound A90b will be hydrolyzed with LiOH in MeOH/THF to give acid. The acid (1 mmol) will be treated with EDCI (2.0 mmol), HOBt (2.0 mmol) and secondary amine (1.2 mmol) in the presence of NEt₃ (3.0 mmol) in CH₂Cl₂ (8 mL). The mixture will be stirred overnight before it will be quenched with addition of NH₄Cl aqueous solution. Aqueous layer will extracted with EtOAc. Organic phase will be washed with water, brine and dried over MgSO₄. Solvent will be concentrated to give the crude product. The crude residue will be purified by silica gel column chromatography to yield compound A90c.

### Step D:

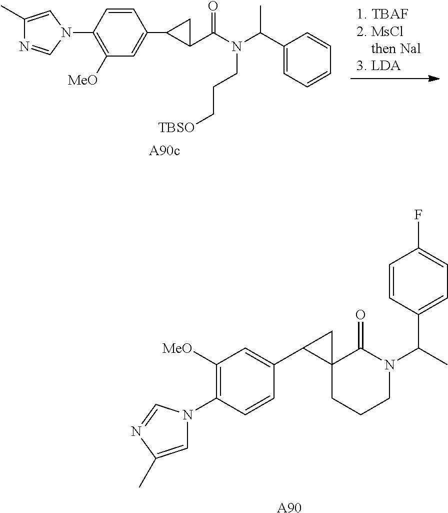

Compound A90c (1 mmol) will be treated with TBAF (2.0 eq) in THF (5 mL) at room temperature for 2 hours. Solvent was removed and the crude will be purified with silica gel chromatography to give product alcohol. The primary alcohol (1 mmol) will be treated with MsCl (2.0 eq) and NEt₃ (4 eq) in CH₂Cl₂ (5 mL) at room temperature for 1 hour. The crude will be diluted with CH₂Cl₂ and washed with water and dried over MgSO₄. Solvent will be removed and the crude will be dissolved in THF and treated with LDA (1.2 mmol) at −78° C. The reaction will be quenched with addition of NH₄Cl aqueous solution. Aqueous layer will extracted with EtOAc. Organic phase will be washed with water, brine and dried over MgSO₄. Solvent will be concentrated to give the crude product. The crude residue will be purified by silica gel column chromatography to yield compound A90.

Compounds A91-97 will be prepared using a similar sequence as A90.

### Example 25

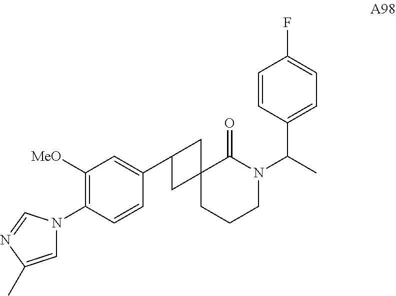

### Step A:

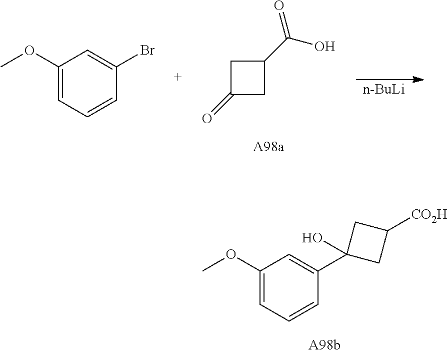

3-methoxy bromobenzene (2.0 mmol) in THF (6.0 mL) was treated with n-BuLi (2.0 mmol) at 0° C. for 1 hour before the mixture was transferred to a solution of A98a (1.0 mmol) in THF (5 mL). The mixture was stirred for 2 hours before it was quenched with addition of NH₄Cl aqueous solution. Aqueous layer was exacted with EtOAc. Organic phase was washed with water, brine and dried over MgSO₄. Solvent was concentrated to give the crude product. The crude residue was purified by silica gel column chromatography to yield compound A98b.

### Step B:

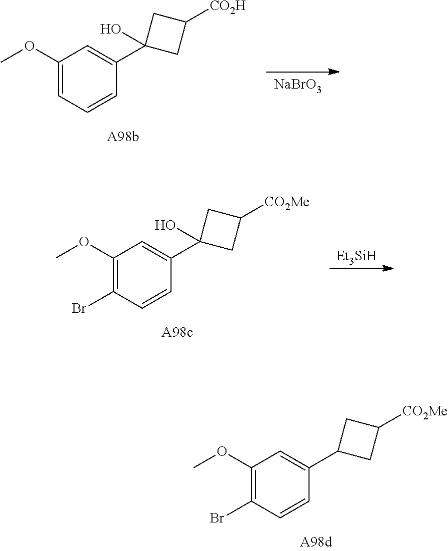

Compound A98b was converted to A98c with the treatment of NaBrO₃. Compound A98c was treated with Et₃SiH in THF to give product A98d.

### Step C:

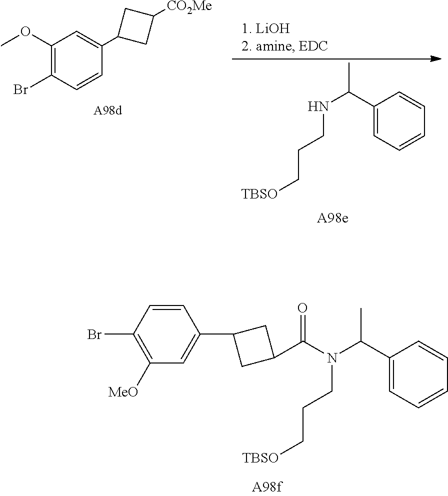

Compound A98d was hydrolyzed with LiOH in MeOH/THF to give acid. The acid (1 mmol) will be treated with EDCI (2.0 mmol), HOBt (2.0 mmol) and secondary amine A98e (1.2 mmol) in the presence of NEt₃ (3.0 mmol) in CH₂Cl₂ (8 mL). The mixture will be stirred overnight before it will be quenched with addition of NH₄Cl aqueous solution. Aqueous layer will be extracted with EtOAc. Organic phase will be washed with water, brine and dried over MgSO₄. Solvent will be concentrated to give the crude product. The crude residue will be purified by silica gel column chromatography to yield compound A98f.

### Step D:

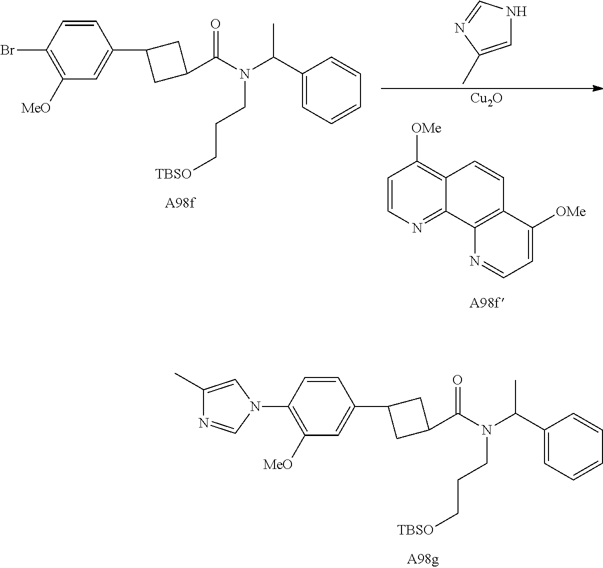

A mixture of compound A98f (1 mmol), Cu₂O (0.2 mmol), PEG (0.4 g), Cs₂CO₃ (3.0 mmol), 4-methylimidazole (1.2 mmol) and A98f′ (0.3 mmol) in NMP (2 mL) will be vacuum-nitrogen exchange degassed and stirred in a sealed tube at 120° C. for 48 hours. The mixture will be cooled to room temperature and diluted with CH₂Cl₂ followed with addition of silica gel. The mixture will be stirred for 20 minutes and filtered. The organic layer will be washed with water (3×), brine, dried over MgSO₄, and concentrated to give the crude product. The crude residue will be purified by column chromatography eluting with CH₂Cl₂/MeOH to yield compound A98g.

### Step E:

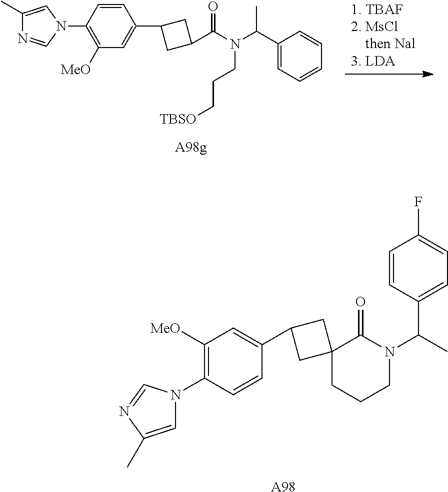

Compound A98g (1 mmol) will be treated with TBAF (2.0 eq) in THF (5 mL) at room temperature for 2 hours. Solvent will be removed and the crude will be purified with silica gel chromatography to give product alcohol. The primary alcohol (1 mmol) will be treated with MsCl (2.0 eq) and NEt₃ (4 eq) in CH₂Cl₂ (5 mL) at room temperature for 1 hour. The crude will be diluted with CH₂Cl₂ and washed with water and dried over MgSO₄. Solvent will be removed and the crude will be dissolved in THF and treated with LDA (1.2 mmol) at −78° C. The reaction will be quenched with addition of NH₄Cl aqueous solution. Aqueous layer will extracted with EtOAc. Organic phase will be washed with water, brine and dried over MgSO₄. Solvent will be concentrated to give the crude product. The crude residue will be purified by silica gel column chromatography to yield compound A98.

Compounds A99-105, in Table 2, will be prepared using a similar sequence as A98.

TABLE 2

| Compound | Structure | LCMS |
|---|---|---|
| A86 | 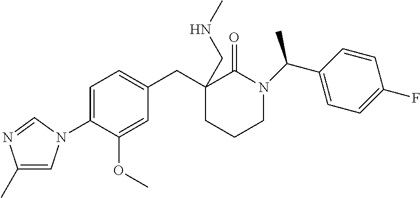 | 465.3 |
| A87 | 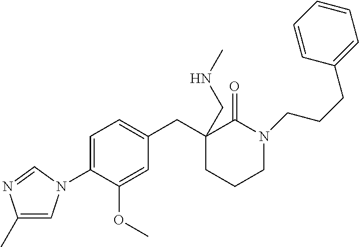 | 461.3 |
| A88 | 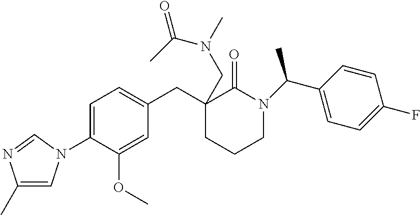 | 507.3 |
| A89 | 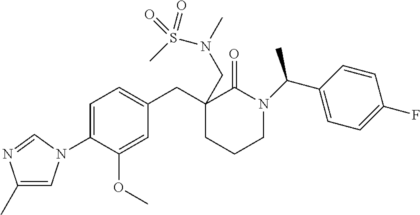 | 543.3 |
| A90 | 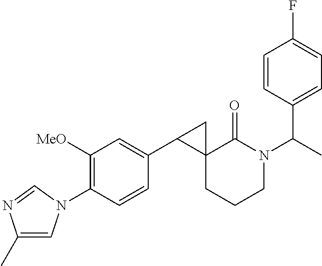 | |
| A91 | 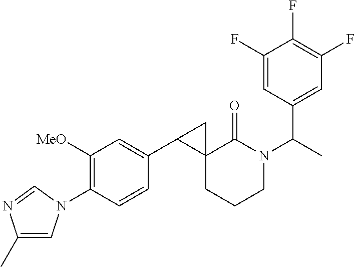 | |
| A92 | 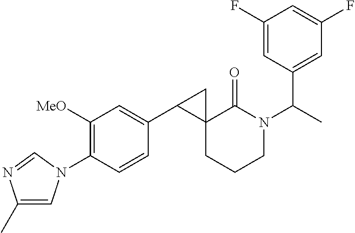 | |
| A93 | 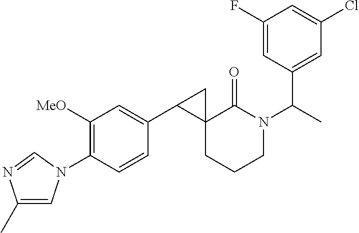 | |
| A94 | 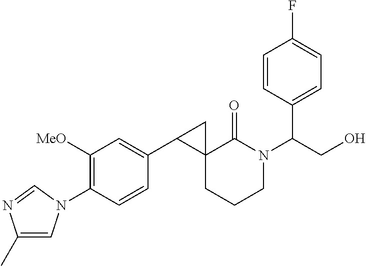 | |
| A95 | 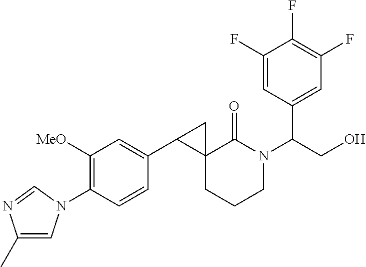 | |
| A96 | 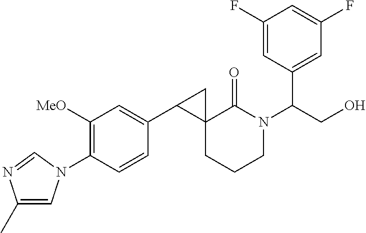 | |
| A97 | 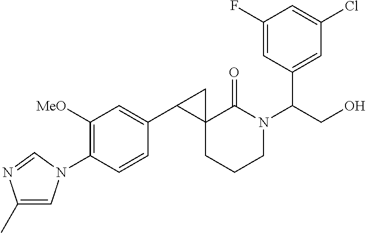 | |
| A98 | 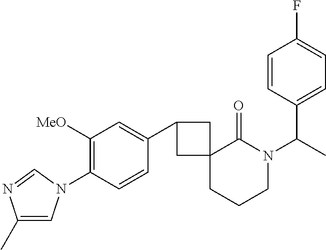 | |
| A99 | 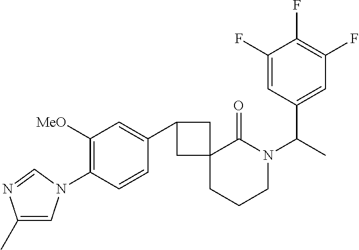 | |
| A100 | 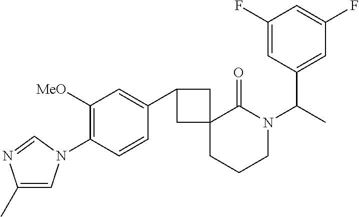 | |
| A101 | 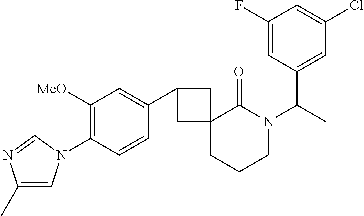 | |
| A102 | 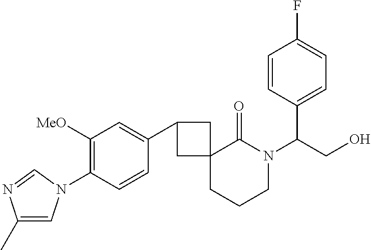 | |
| A103 | 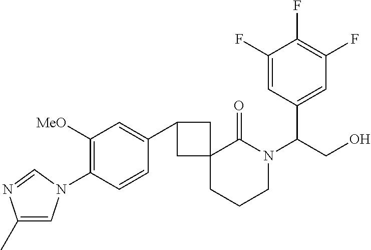 | |
| A104 | 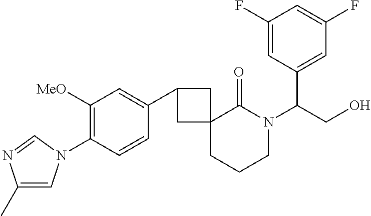 | |
| A105 | 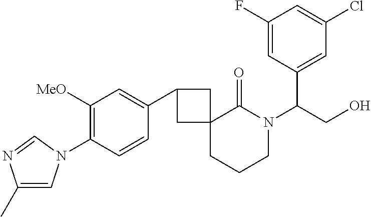 | |

### Example 106

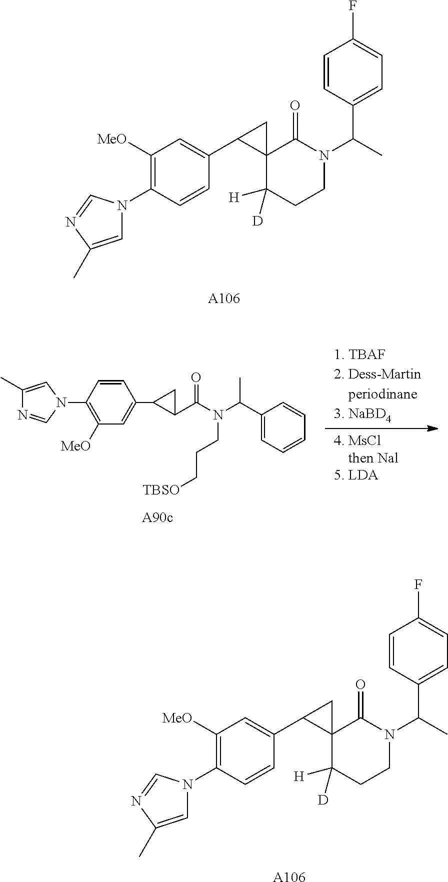

Compound A90c (1 mmol) will be treated with TBAF (2.0 eq) in THF (5 mL) at room temperature for 2 hours. Solvent was removed and the crude will be purified with silica gel chromatography to give product alcohol. The primary alcohol (1 mmol) will be treated with Dess-Martin Periodinane (1.5 mmol) in CH₂Cl₂ at room temperature. The crude will be diluted with CH₂Cl₂ and washed with Na₂S₂O₃ aqueous solution, NaHCO₃ aqueous solution, water, brine and dried over MgSO₄. Solvent will be removed and the crude aldehyde will be dissolved in THF and treated with NaBD₄ (1.5 mmol). The reaction mixture will be diluted with NH₄Cl and EtOAc. The organic phase will be washed with water and dried over MgSO₄. Solvent will be removed and the crude will be treated with MsCl (2.0 eq) and NEt₃ (4 eq) in CH₂Cl₂ (5 mL) at room temperature for 1 hour. The crude will be diluted with CH₂Cl₂ and washed with water and dried over MgSO₄. Solvent will be removed and the crude will be dissolved in THF and treated with LDA (1.2 mmol) at −78° C. The reaction will be quenched with addition of NH₄Cl aqueous solution. Aqueous layer will extracted with EtOAc. Organic phase will be washed with water, brine and dried over MgSO₄. Solvent will be concentrated to give the crude product. The crude residue will be purified by silica gel column chromatography to yield compound A106.

### Example 27

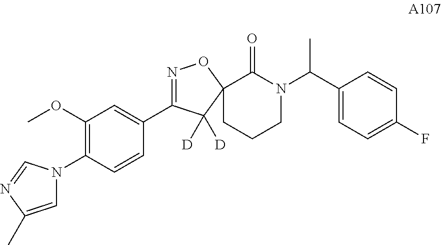

### Step A:

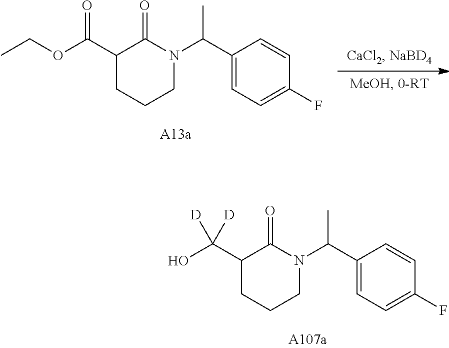

To a mixture of 6.986 g (23.832 mmol) of compound A13a and 4.31 g (28.598 mmol) of CaCl₂ in 40 mL of dry methanol will be added in portions at 0° C. 1.081 g (28.598 mmol) of NaBD₄. The mixture will be stirred at 0° C. for 1 h and will be allowed to warm up to rt over a period of 5 h. The solids will be filtered and washed with methanol. The filtrate will be evaporated to form a solid, and it will be extracted with DCM. The DCM phase will be washed with water and brine and dried over Na₂SO₄, The solvent will be evaporated, and the crude product will be purified by SiO₂ chromatography using a gradient of 40-90% of EtOAc in hexanes to give alcohol A107a.

A107a will be transformed to the desired product A107 following a similar procedure for the preparation of compound A13.

### Assay:

Secretase Reaction and Aβ Analysis in Whole Cells: HEK293 cells overexpressing APP with Swedish and London mutations were treated with the specified compounds for 5 hour at 37° C. in 100 ml of DMEM medium containing 10% fetal bovine serum. At the end of the incubation, total Aβ, Aβ40 and Aβ42 were measured using electrochemiluminescence (ECL) based sandwich immunoassays. Total Aβ was determined using a pair of antibodies TAG-WO2 and biotin-4G8, Aβ40 was identified with antibody pairs TAG-G2-10 and biotin-4G8, while Aβ42 was identified with TAG-G2-11 and biotin-4G8. The ECL signal was measured using Sector Imager 2400 (Meso Scale Discovery).

MS Analysis of Aβ Profile: Aβ profile in conditioned media was determined using surface enhanced laser desorption/ionization (SELDI) mass spectrometry. Conditioned media was incubated with antibody WO2 coated PS20 ProteinChip array. Mass spectra of Aβ captured on the array were read on SELDI ProteinChip Reader (Bio-Rad) according to manufacture's instructions.

CSF Aβ Analysis: Aβ in rat CSF was determined using MSD technology as described above. Aβ40 was measured using antibody pair Tag-G2-10 and biotin-4G8, while Aβ42 was measured using Tag-anti Aβ42 (Meso Scale Discovery) and biotin-4G8. The ECL signal was measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra are acquired in the linear mode with an acceleration voltage of 20 kV. Each spectrum presented in this work represents an average of 256 laser shots. To prepare the sample-matrix solution, 1 μL of immunoprecipitated Aβ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

Compounds A3 to A14 had an Aβ42 IC₅₀ in the range of about 216 to about 5526 nM. Compounds A3 to A14 had an Aβ3 Total/Aβ42 in the range of about 3 to about 29.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1-42. (canceled)

43. A compound of the formula (I): 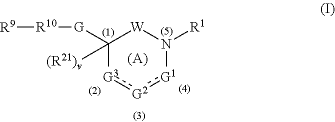
or a pharmaceutically acceptable salt thereof, wherein:
the numbers (1), (2), (3), (4), and (5) are reference numbers to identify positions of the Ring (A); G³ is at position (2), G² is at position (3), G¹ is at position (4) and the N is at position (5);
R¹, R⁹, R¹⁰, R²¹, v, G¹, G², G³, and W are each independently selected;
the dotted line (----) represents an optional bond between positions (2) and (3) or positions (3) and (4), that is when the optional bond is present between positions (2) and (3) the optional bond is absent between positions (3) and (4), and when the optional bond is present between positions (3) and (4) the optional bond is absent between positions (2) and (3);
d is 0 or 1;
m is 0 to 6;
n is 1 to 5;
p is 0 to 5;
q is 0, 1 or 2, and each q is independently selected;
r is 1 to 3;
t is 1 or 2
v is 0 or 1;
W is selected from the group consisting of: —C(O)—, —S(O)₂—, —S(O)—, and —C(═NR²)—;
G is selected from the group consisting of: a direct bond, —C(O)—, —(C═NR²)—, —(C═C(R⁶)₂)—, —CHR³—, —C(R⁴)₂—, —CF₂—, —N(R²)—, —O—, —S—, —S(O)ₜ, —CR⁴(OH)—, —CR⁴(OR⁴)—, —C═C—, alkynyl, —(CH₂)ᵣN(R²)—, —(CHR⁴)ᵣN(R²)—, —(C(R⁴)₂)ᵣN(R²)—, —N(R²)(CH₂)ᵣ—, —N(R²)(CHR⁴)ᵣ—, —N(R²)(C(R⁴)₂)ᵣ—, —(CH₂)ᵣ—O—, —(CHR⁴)ᵣ—O—, —(C(R⁴)₂)ᵣ—O—, —O—(CH₂)ᵣ—, —O—(CHR⁴)ᵣ—, —O—(C(R⁴)₂)ᵣ—, —(CH₂)ᵣ—O—C(O)—, —(CHR⁴)ᵣ—O—C(O)—, —(C(R⁴)₂)ᵣ—O—C(O)—, —C(O)—O—(CH₂)ᵣ—, —C(O)—O—(CHR⁴)ᵣ—, —C(O)—O—(C(R⁴)₂)ᵣ—, —C(O)NR⁵—, —O—C(O)—, —C(O)—O—, —O—C(O)—NR⁵—, —NR⁵C(O)—, —(CH₂)ᵣNR⁵—C(O)—, —(CHR⁴)ᵣNR⁵—C(O)—, —(C(R⁴)₂)ᵣNR⁵—C(O)—, —C(O)NR⁵(CH₂)ᵣ—, —C(O)NR⁵(CHR⁴)ᵣ—, —C(O)NR⁵(C(R⁴)₂)ᵣ—, —NR⁵S(O)ₜ—, —(CH₂)ᵣNR⁵S(O)ₜ—, —(CHR⁴)ᵣNR⁵S(O)ₜ—, —(C(R⁴)₂)ᵣNR⁵S(O)ₜ—, —S(O)ₜNR⁵—, —S(O)ₜNR⁵(CH₂)ᵣ—, —S(O)ₜNR⁵(CHR⁴)ᵣ—, —S(O)ₜNR⁵(C(R⁴)₂)ᵣ—, —NR⁵—C(O)—O—, —NR⁵—C(O)—NR⁵—, —NR⁵—S(O)ₜ—NR⁵—, —NR⁵—C(═NR²)—NR⁵—, —NR⁵—C(═NR²)—O—, —O—C(═NR²)—NR⁵—, —C(R⁴)═N—O—, —O—N═C(R⁴)—, —O—C(R⁴)═N—, —N═C(R⁴)—O—, —(CH₂)₂₋₃—, —(C(R⁴)₂)₂₋₃—, —(CHR⁴)₂₋₃—, cycloalkyl, and heterocycloalkyl (comprising 1 to 4 heteroatoms independently selected from the group consisting of: —O—, —NR²—, —S—, —S(O)—, and —S(O)₂);
G¹ is selected from the group consisting of: a direct bond, —O—, —C(R²¹)_{q}—, —N(R²)_{d}—, —C(O)—, —C(═NR²)—, —S—, —S(O)₂, and —S(O)—; and with the proviso that when the optional double bond between (3) and (4) is present then:
(a) q for the —C(R²¹)_{q}— group is 0 or 1 (and when 0 there is a H on the carbon), and
(b) d for the —N(R²)_{d}— group is 0 (and there is no H on the N due to the double bond between positions (3) and (4)); and
(c) G¹ is not —O—, —C(O)—, —C(═NR²)—, —S—, —S(O)₂, or S(O)—;
G² is selected from the group consisting of: a direct bond, —O—, —C(R²¹)_{q}—, —N(R²)_{d}—, —C(O)—, —C(═NR²)—, —S—, —S(O)₂, and —S(O)—; and with the proviso that when the optional double bond between (3) and (4) is present then:
(a) q for the —C(R²¹)_{q}— group is 0 or 1 (and when 0 there is a H on the carbon), and
(b) d for the —N(R²)_{d}— group is 0 (and there is no H on the N due to the double bond between positions (3) and (4)); and
(c) G² is not —O—, —C(O)—, —C(═NR²)—, —S—, —S(O)₂, or —S(O)—;
G³ is selected from the group consisting of: —C(R²¹)_{d}— wherein q is 0, 1 or 2, and when the optional bond between G² and G³ is present then q is 0 or 1 (and when q is 0 there is a H on the carbon), and —N(R²)_{d} wherein d is 0 or 1, and d is 0 when the optional bond between G² and G³ is present;
Optionally,
(a) G¹ and G² can be taken together to form a ring, wherein said ring is a 3 to 8 membered (including the atoms common to both rings) cycloalkyl, heterocycloalkyl, heteroaryl, aryl, cycloalkenyl, or heterocycloalkenyl ring, and wherein said ring is optionally substituted with 1 to 5 independently selected R²¹ substituents, and wherein said heterocycloalkyl, heteroaryl, and heterocycloalkenyl rings comprise 1 to 3 heteroatoms independently selected from the group consisting of: —O—, —S—, —S(O)—, —S(O)₂—, and —N(R²)—, or
(b) G² and G³ can be taken together to form a ring, wherein said ring is a 3 to 8 membered (including the atoms common to both rings) cycloalkyl, heterocycloalkyl, heteroaryl, aryl, cycloalkenyl, or heterocycloalkenyl ring, and wherein said ring is optionally substituted with 1 to 5 independently selected R²¹ substituents, and wherein said heterocycloalkyl, heteroaryl, and heterocycloalkenyl rings comprise 1 to 3 heteroatoms independently selected from the group consisting of: —O—, —S—, —S(O)—, —S(O)₂—, and —N(R²)—, or
(c) G and the Ring (A) carbon to which G is bound can be taken together to form a spiro ring, wherein said ring is a 3 to 8 membered (including the atom common to both rings) cycloalkyl, heterocycloalkyl, cycloalkenyl, or heterocycloalkenyl ring, and wherein said ring is optionally substituted with 1 to 5 independently selected R²¹ substituents, and wherein said heterocycloalkyl, and heterocycloalkenyl rings comprise 1 to 3 heteroatoms independently selected from the group consisting of: —O—, —S—, —S(O)—, —S(O)₂—, and —N(R²)—, or
(d) G and (R²¹)ᵥ can be taken together to form a spiro ring wherein said ring is a 3 to 8 membered (including the atom common to both rings) cycloalkyl, heterocycloalkyl, cycloalkenyl, or heterocycloalkenyl ring, and wherein said ring is optionally substituted with 1 to 5 independently selected R²¹ substituents, and wherein said heterocycloalkyl, and heterocycloalkenyl rings comprise 1 to 3 heteroatoms independently selected from the group consisting of: —O—, —S—, —S(O)—, —S(O)₂—, and —N(R²)—; and
when:
(a) G¹ and G² form a ring then:
(1) G¹ is selected from the group consisting of: (i) C and the optional bond between G¹ and G² is present, (ii) —C(R²¹)_{q}— wherein q is 1 and the optional bond between G¹ and G² is absent, (iii) —CH— and the optional bond between G¹ and G² is absent, (iv) N and the optional bond between G¹ and G² is absent, and (v) —C(═N) and the optional bond between G¹ and G² is absent; and
(2) G² is selected from the group consisting of: (i) C and the optional bond between G¹ and G² is present, (ii) C and the optional bond between G² and G³ is present, (iii) —C(R²¹)_{q}— wherein q is 1 and the optional bond between G¹ and G² is absent, and the optional bond between G² and G³ is absent, (iii) —CH— and the optional bond between G¹ and G² is absent, and the optional bond between G² and G³ is absent, and (iv) N and the optional bond between G¹ and G² is absent, and the optional bond between G² and G³ is absent; and wherein in one example, G² is —C(R²¹)_{q}—;
(b) G² and G³ form a ring then:
(1) G² is selected from the group consisting of: (i) C and the optional bond between G¹ and G² is present, (ii) C and the optional bond between G² and G³ is present, (iii) —C(R²¹)_{q}— wherein q is 1 and the optional bond between G¹ and G² is absent, and the optional bond between G² and G³ is absent, (iii) —CH— and the optional bond between G¹ and G² is absent, and the optional bond between G² and G³ is absent, and (iv) N and the optional bond between G¹ and G² is absent, and the optional bond between G² and G³ is absent; and wherein in one example, G² is —C(R²¹)_{q}—, and
(2) G³ is selected from the group consisting of: (i) C and the optional bond between G² and G³ is present, (ii) —C(R²¹)_{q}— wherein q is 1 and the optional bond between G² and G³ is absent, (iii) —CH— and the optional bond between G² and G³ is absent, and (iv) N and the optional bond between G² and G³ is absent; and wherein in one example, G³ is C; and
(c) G and the Ring (A) carbon to which G is bound form a spiro ring, then v is 0 for the R²¹ moiety at position 1, and there is no H bound to the carbon at position (1);
R¹ is selected from the group consisting of: alkyl, alkenyl, alkynyl, cycloalkyl, cycloalkylalkyl-, heterocyclyl, cycloalkenyl, arylalkyl-, alkylaryl-, aryl, heteroaryl, heterocyclenyl, fused benzocycloalkyl, fused benzoheterocycloalkyl, fused heteroarylcycloalkyl, fused heteroarylheterocycloalkyl, fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, fused heterocycloalkylheteroarylalkyl-, and wherein each of said: alkyl, alkenyl, alkynyl, cycloalkyl, heterocyclyl, cycloalkenyl, aryl, heteroaryl, heterocyclenyl, fused benzocycloalkyl, fused benzoheterocycloalkyl, fused heteroarylcycloalkyl, fused heteroarylheterocycloalkyl, fused cycloalkylaryl, fused heterocycloalkylaryl-, fused cycloalkylheteroaryl-, fused heterocycloalkylheteroaryl-, fused benzocycloalkylalkyl-, fused benzoheterocycloalkylalkyl-, fused heteroarylcycloalkylalkyl-, fused heteroarylheterocycloalkylalkyl-, fused cycloalkylarylalkyl-, fused heterocycloalkylarylalkyl-, fused cycloalkylheteroarylalkyl-, and fused heterocycloalkylheteroarylalkyl-R¹ groups is optionally substituted with 1-5 independently selected R²¹ groups; or R¹ taken together with the nitrogen to which it is bound, and taken together with G¹ form a 4 to 8 membered ring fused to Ring (A), wherein said fused ring optionally comprises 1 to 3 additional heteroatoms selected from the group consisting of —NR²—, —O—, —S—, —S(O)—, and —S(O)₂, and wherein said fused ring optionally comprises 1 to 3 double bonds, and wherein said fused ring is optionally substituted with 1 to 6 independently selected R²¹ groups, and wherein G¹ is selected from the group consisting of: (i) C and the optional bond between G¹ and G² is present, (ii) —C(R²¹)_{q}— wherein q is 1 and the optional bond between G¹ and G² is absent, (iii) —CH— and the optional bond between G¹ and G² is absent, (iv) N and the optional bond between G¹ and G² is absent, and (v) —C(═N) and the optional bond between G¹ and G² is absent,
R² is selected from the group consisting of: H, —OH, —O-alkyl, —O-(halo substituted alky), —NH(R⁴), —N(R⁴)₂, —NH₂, —S(R⁴), —S(O)R⁴, —S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstitued aryl, substitued aryl, unsubstitued heteroaryl, substitued heteroaryl, unsubstituted alkyl, substituted alkyl, unsubstitued arylalkyl-, substitued arylalkyl-, unsubstitued heteroarylalkyl-, substitued heteroarylalkyl-, unsubstitued alkenyl, substituted alkenyl, unsubstituted alkynyl, substituted alkynyl, unsubstitued cycloalkyl, and substituted cycloalkyl, wherein said substitued aryl, heteroaryl, alkyl, arylalkyl-, heteroarylalkyl-, alkenyl, alkynyl and cycloalkyl groups are substituted with 1 to 5 independently selected R²¹ groups;
R³ is selected from the group consisting of: H, —OH, halo, —O-alkyl, —O-(halo substituted alky), —NH(R⁴), —N(R⁴)₂, —NH₂, —S(R⁴), —S(O)R⁴, —S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
Each R⁴ is independently selected from the group consisting of: unsubstitued aryl, substitued aryl, unsubstitued heteroaryl, substitued heteroaryl, unsubstituted alkyl, substituted alkyl, unsubstitued arylalkyl-, substitued arylalkyl-, unsubstitued heteroarylalkyl-, substitued heteroarylalkyl-, unsubstitued alkenyl, substituted alkenyl, unsubstituted alkynyl, substituted alkynyl, unsubstitued cycloalkyl, and substituted cycloalkyl, wherein said substitued aryl, heteroaryl, alkyl, arylalkyl-, heteroarylalkyl-, alkenyl, alkynyl and cycloalkyl groups are substituted with 1 to 5 independently selected R²¹ groups;
Each R⁵ is independently selected from the group consisting of: H, unsubstitued alkyl, substituted alkyl, unsubstitued alkenyl, substituted alkenyl, unsubstitued alkynyl, substituted alkynyl, unsubstitued cycloalkyl, substituted cycloalkyl, unsubstituted aryl, substituted aryl, unsubstituted heteroaryl and substituted heteroaryl; wherein said substituted groups are substituted with one or more substituents independently selected from: R²;
each R⁶ is independently selected from the group consisting of aryl, heteroaryl, halo, —CF₃, —CN, —C(O)R²⁴, —C(O)OR²⁴, C(O)N)S(O)N(R²⁴)(R²⁵), —S(O)N(R²⁴)(R²⁵), —OR⁹, —S(O)₂N(R²⁴)(R²⁵), —C(═NOR²⁴)R²⁵, —P(O)(OR²⁴)(OR²⁵), —N(R²⁴)(R²⁵), —N(R²⁴)C(O)R²⁵, —N(R²⁴)S(O)R^{25A}, —N(R²⁴)S(O)₂R^{25A}, —N(R²⁴)S(O)₂N(R²⁵)(R²⁶), —N(R²⁴)S(O)N(R²⁵)(R²⁶), —N(R²⁴)C(O)N(R²⁵)(R²⁶), —N(R²⁴)C(O)OR²⁵, —S(O)R^{24A} and —S(O)₂R^{24A};
R⁹ is selected from the group consisting of: arylalkoxy-, heteroarylalkoxy-, arylalkylamino-, heteroarylalkylamino-, aryl-, arylalkyl-, cycloalkyl-, cycloalkenyl, cycloalkylalkyl-, heteroaryl-, heteroarylalkyl-, heterocyclyl-, heterocyclenyl-, and heterocyclyalkyl-, wherein each of said R⁹ arylalkoxy-, heteroarylalkoxy-, arylalkylamino-, heteroarylalkylamino-, aryl-, arylalkyl-, cycloalkyl-, cycloalkenyl, cycloalkylalkyl-, heteroaryl-, heteroarylalkyl-, heterocyclyl-, heterocyclenyl-, heterocyclyalkyl- and heterocyclyalkyl- is optionally substituted with 1-5 independently selected R²¹ groups;
R¹⁰ is selected from the group consisting of: aryl-, heteroaryl-, cycloalkyl-, cycloalkenyl, cycloalkylalkyl-, heterocyclyl-, heterocyclenyl-, heterocyclylalkyl-, heterocyclyalkenyl-, fused benzocycloalkyl-, fused benzoheterocycloalkyl-, fused heteroarylcycloalkyl-, fused heteroarylheterocycloalkyl-, fused cycloalkylaryl, fused heterocycloalkylaryl-, fused cycloalkylheteroaryl-, fused heterocycloalkylheteroaryl-, fused heteroarylheteroaryl-, fused heteroarylaryl-, fused arylheteroaryl-, fused arylaryl-, fused heterocycloalkenylaryl-, fused heterocycloalkenylheteroaryl-, 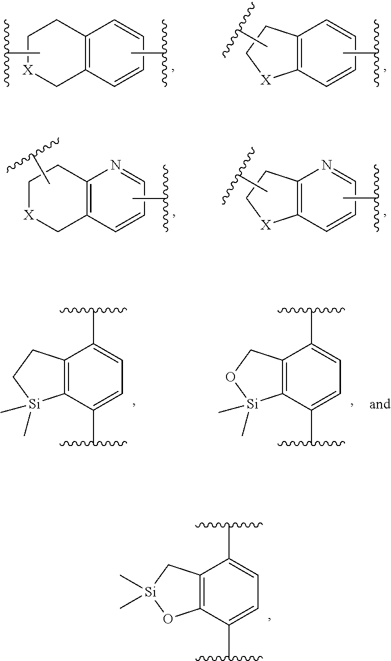
wherein X is selected from the group consisting of: O, —N(R¹⁴)— and —S—; and wherein each of said R¹⁰ moieties is optionally substituted with 1-5 independently selected R²¹ groups; or
R⁹ and R¹⁰ are linked together to form a fused tricyclic ring system wherein R⁹ and R¹⁰ are as defined above and the ring linking R⁹ and R¹⁰ is an alkyl ring, or a heteroalkyl ring, or an aryl ring, or a heteroaryl ring, or an alkenyl ring, or a heteroalkenyl ring;
R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶);
R^{15A} and R^{16A} are independently selected from the group consisting of alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;
R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl;
Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
two R¹⁸ moieties on adjacent carbons can be linked together to form a 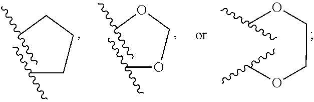
R¹⁹ is selected from the group consisting of: alkyl, cycloalkyl, aryl, arylalkyl and heteroarylalkyl;
R²⁰ is selected from the group consisting of: alkyl, cycloalkyl, aryl, halo substituted aryl, arylalkyl, heteroaryl and heteroarylalkyl;
each R²¹ is independently selected from the group consisting of: alkyl, alkenyl, alkynyl, cycloalkyl, cycloalkylalkyl, cycloalkenyl, heterocycloalkyl, ═O, ═N—R², heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SR¹⁵, —P(O)(CH₃)₂, —SO(═NR¹⁵)R¹⁶—, —SF₅, —OSF₅, —Si(R^{15A})₃ wherein each R^{15A} is independently selected —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R^{15A}, ═NOR¹⁵, —N₃, —NO₂, —S(O)₂R^{15A}, —O—N═C(R⁴)₂ (wherein each R⁴ is independently selected), and —O—N═C(R⁴)₂ wherein R⁴ is taken together with the carbon atom to which they are bound to form a 5 to 10 membered ring, said ring optionally containing 1 to 3 heteroatoms selected from the group consisting of —O—, —S—, —S(O)—, —S(O)₂—, and —NR²—; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups;
Each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —SF₅, —OSF₅, —Si(R^{15A})₃, —S(O)N(R¹⁵)(R¹⁶), —S(O)₂N(R¹⁵)(R¹⁶), —C(═NOR¹⁵)R¹⁶, —P(O)(OR¹⁵)(O R¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R^{16A}, —N(R¹⁵)S(O)₂R^{16A}, —CH₂—N(R¹⁵)S(O)₂R^{16A}, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R^{15A} and —S(O)₂R^{15A};
Each R^{24A} and R^{25A} is independently selected from the group consisting of alkyl, cycloalkyl, cycloalkylalkyl, heterocycloalkyl, heterocycloalkylalkyl, aryl, arylalkyl, heteroaryl, heteroarylalkyl, arylcycloalkyl, (R^{27A})ₙ-alkyl, (R^{27A})ₙ-cycloalkyl, (R^{27A})ₙ-cycloalkylalkyl, (R^{27A})ₙ-heterocycloalkyl, (R^{27A})ₙ-heterocycloalkylalkyl, (R^{27A})ₙ-aryl, (R^{27A})ₙ-arylalkyl, (R^{27A})ₙ-heteroaryl and (R^{27A})ₙ-heteroarylalkyl;
Each R²⁴, R²⁵ and R²⁶ is independently selected from the group consisting of H, alkyl, cycloalkyl, cycloalkylalkyl, heterocycloalkyl, heterocycloalkylalkyl, aryl, arylalkyl, heteroaryl, heteroarylalkyl, arylcycloalkyl, (R^{27A})ₙ-alkyl, (R^{27A})ₙ-cycloalkyl, (R^{27A})ₙ-cycloalkylalkyl, (R^{27A})ₙ-heterocycloalkyl, (R^{27A})ₙ-heterocycloalkylalkyl, (R^{27A})ₙ-aryl, (R^{27A})ₙ-arylalkyl, (R^{27A})ₙ-heteroaryl and (R^{27A})ₙ-heteroarylalkyl;
Each R^{27A} is independently selected from the group consisting of alkyl, aryl, arylalkyl, —NO₂, halo, —CF₃, —CN, alkyl-CN, —C(O)R²⁸, —C(O)OH, —C(O)OR²⁸, —C(O)NH R²⁹, —C(O)N(alkyl)₂, —C(O)N(alkyl)(aryl), —C(O)N(alkyl)(heteroaryl), —SR²⁸, —S(O)₂R²⁹, —S(O)NH₂, —S(O)NH(alkyl), —S(O)N(alkyl)(alkyl), —S(O)NH(aryl), —S(O)₂NH₂, —S(O)₂NHR²⁸, —S(O)₂NH(aryl), —S(O)₂NH(heterocycloalkyl), —S(O)₂N(alkyl)₂, —S(O)₂N(alkyl)(aryl), —OH, —OR²⁹, —O-heterocycloalkyl, —O-cycloalkylalkyl, —O-heterocycloalkylalkyl, —NH₂, —NHR²⁹, —N(alkyl)₂, —N(arylalkyl)₂, —N(arylalkyl)(heteroarylalkyl), —NHC(O) R²⁹, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁹, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
R²⁸ is selected from the group consisting of: alkyl, cycloalkyl, arylalkyl and heteroarylalkyl; and
R²⁹ is selected from the group consisting of; alkyl, cycloalkyl, aryl, arylalkyl, heteroaryl or heteroarylalkyl; and
provided that:
(a) Ring A does not have two adjacent —O— atoms in the ring; and
(b) Ring A does not have two adjacent sulfur groups in the ring; and
(c) Ring A does not have an —O— atom adjacent to a sulfur group; and
(d) When G¹ is N, then G² is not —O—; and
(e) When G¹ is —O—, then G² is not N; and
(f) When G¹ is N, then G² is not —S—; and
(g) When G¹ is —S—, then G² is not N; and
(h) When G¹ is a direct bond, and G² is —O—, then G³ is not N; and
(i) When G² is a direct bond, and G¹ is —O—, then G³ is not N; and
(j) When G¹ is N, and G³ is N, then G² is not N; and
(k) When G² is N, and G³ is N, then G¹ is not N; and
(l) When G¹ is N, and G² is N, then G³ is not N; and
(m) When W is SO or S(O)₂ then G is not —C(O)—, —(C═NR²)—, —(C═C(R⁶)₂)—, —C(R⁴)₂—, —CF₂—, —CR⁴(OH)—, —CR⁴(OR⁴)—, or —CHR³—; and
(n) When W is —C(O)— then R¹ is not a fused benzocycloalkyl substituted with —NH₂, or a fused benzoheterocycloalkyl substituted with —NH₂, or a fused heteroarylcycloalkyl substituted with —NH₂, or a fused heteroarylheterocycloalkyl substituted with —NH₂; and
(o) When the optional bond between G² and G³ is present, then v is 1 for the moiety (R²¹)ᵥ; and
(p) When G is —C(O)—, —(C═NR²)—, —(C═C(R⁶)₂)—, or —C═C—, then v is 1 for the moiety (R²¹)ᵥ; and
(q) When G¹ is —C(═NR²)—, and G² is a direct bond, and G³ is —N(R²)_{d}—, then G is not —C(O)—, —(C═NR²)—, —(C═C(R⁶)₂)—, —CHR³—, —C(R⁴)₂—, —CF₂—, —CR⁴(OH)—, or —CR⁴(OR⁴)—; and
(r) When G² is —C(═NR²)—, and G¹ is direct bond, and G³ is —N(R²)_{d}—, then G is not —C(O)—, —(C═NR²)—, —(C═C(R⁶)₂)—, —CHR³—, —C(R⁴)₂—, —CF₂—, —CR⁴(OH)—, or —CR⁴(OR⁴)—; and
(s) When G¹ is a direct bond, and G² is —C(R²¹)_{q}—, and G³ is —N(R²)_{d}—, and the optional bond between G² and G³ is present, then G is not —C(O)—, —(C═NR²)—, —(C═C(R⁶)₂)—, —CHR³—, —C(R⁴)₂—, —CF₂—, —CR⁴(OH)—, or —CR⁴(OR⁴)—.

44. The compound of claim 43, wherein said R¹⁰ is selected from the group consisting of aryl and aryl substituted with one or more R²¹ groups, and said R⁹ group is selected from the group consisting of heteroaryl and heteroaryl substituted with one or more R²¹ groups, wherein each R²¹ is independently selected.

45. The compound of claim 43, wherein said R¹⁰ is phenyl substituted with one R²¹ group, and said R⁹ is imidazolyl substituted with one R²¹ group, wherein each R²¹ is independently selected.

46. The compound of claim 43, wherein the R⁹-R¹⁰— moiety is: 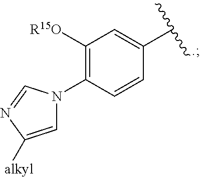
or
wherein the R⁹-R¹⁰— moiety is: 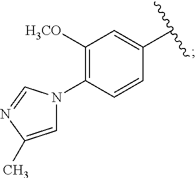
or
wherein the R⁹-R¹⁰— moiety is: 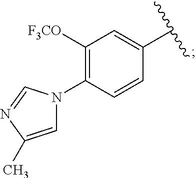
or
wherein the R⁹-R¹⁰— moiety is: 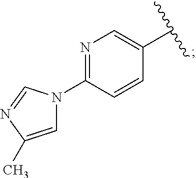
or
wherein the R⁹-R¹⁰— moiety is:

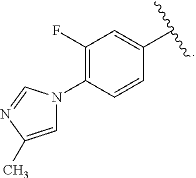

47. The compound of claim 42, wherein:
R¹ is an alkyl group substituted with one R²¹ group, and said R²¹ group is an aryl group; or
R¹ is an alkyl group substituted with one R²¹ group, and said R²¹ group is an aryl group, and said aryl is phenyl, and said alkyl group is methyl or ethyl; or
R¹ is an alkyl group substituted with one R²¹ group, and said R²¹ group is an aryl group, and said aryl group is substituted with one or more R²² groups; or
R¹ is an alkyl group substituted with one R²¹ group, and said R²¹ group is an aryl group, and said aryl group is substituted with one or more R²² groups wherein each R²² group is the same or different halo; or
R¹ is an alkyl group substituted with one R²¹ group, and said R²¹ group is an aryl group, and said aryl group is substituted with one or two R²² halo groups; or
R¹ is an alkyl group substituted with one R²¹ group, and said R²¹ group is an aryl group, and said aryl group is substituted with one or two R²² halo groups wherein the halo is F; or
R¹ is an alkyl group substituted with one R²¹ group, and said R²¹ group is an aryl group, and said aryl group is substituted with one or more R²² groups, and each R²² group is independently selected from the group consisting of; —SF₅, —OSF₅, —Si(R^{15A})₃; or
R¹ is an alkyl group substituted with one R²¹ group, and said R²¹ group is an aryl group, and said aryl group is substituted with one or two R²² groups, and each R²² group is independently selected from the group consisting of: —SF₅, —OSF₅, —Si(R^{15A})₃; or
R¹ is an alkyl group substituted with one R²¹ group, and said R²¹ group is an aryl group, and said aryl group is substituted with one R²² group, and said R²² group is selected from the group consisting of: —SF₅, —OSF₅, —Si(R^{15A})₃.

48. The compound of claim 43, wherein said R¹ is selected from the group consisting of:

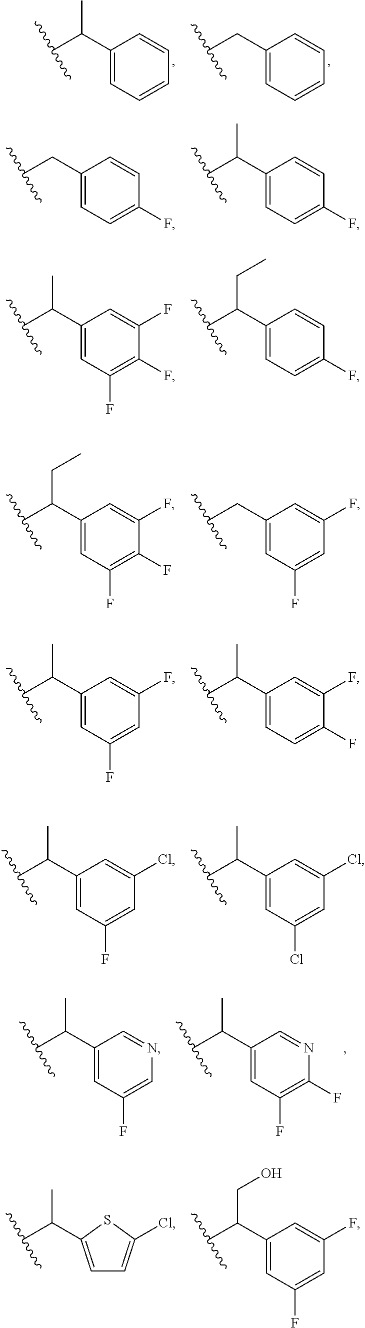

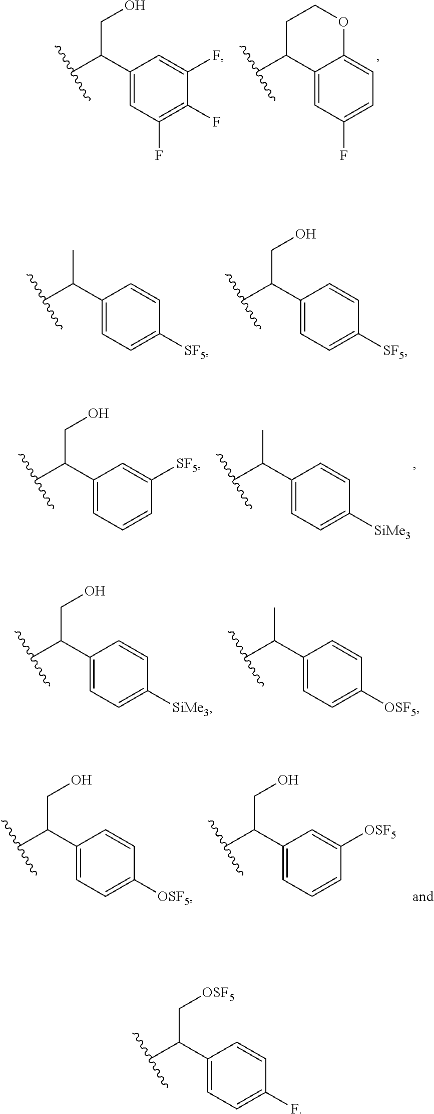

49. The compound of claim 43, wherein:
(1)
R¹ is an alkyl group substituted with one R²¹ group, or
R¹ is an alkyl group substituted with one R²¹ group, and said R²¹ group is substituted with one or more independently selected R²² groups, and
R¹⁰ is selected from the group consisting of aryl and aryl substituted with one or more independently selected R²¹ groups, and
R⁹ is selected from the group consisting of heteroaryl and heteroaryl substituted with one or more independently selected R²¹ groups; or
(2)
R¹ is an alkyl group substituted with one phenyl, or
R¹ is an alkyl group substituted with one phenyl, and said phenyl is substituted with one or more independently selected R²² groups, and
R¹⁰ is selected from the group consisting of phenyl and phenyl substituted with one or more independently selected R²¹ groups, and
R⁹ is selected from the group consisting of imidazolyl and imidazolyl substituted with one or more independently selected R²¹ groups; or
(3)
R¹ is a methyl or ethyl group substituted with one phenyl, or
R¹ is a methyl or ethyl group substituted with one phenyl, and said phenyl is substituted with one or more independently selected halos, and
R¹⁰ is selected from the group consisting of phenyl and phenyl substituted with one or more independently selected —OR¹⁵ groups, and
R⁹ is selected from the group consisting of imidazolyl and imidazolyl substituted with one or more independently selected alkyl groups groups; or
(4)
R¹ is a methyl or ethyl group substituted with one phenyl, or
R¹ is an methyl or ethyl group substituted with one phenyl, and said phenyl is substituted with one or two independently selected halos, and
R¹⁰ is selected from the group consisting of phenyl and phenyl substituted with one or two independently selected —OR¹⁵ groups, wherein R¹⁵ is alkyl, and
R⁹ is selected from the group consisting of imidazolyl and imidazolyl substituted with one or two independently selected alkyl groups groups; or
(5)
R¹ is a methyl or ethyl group substituted with one phenyl, or
R¹ is an methyl or ethyl group substituted with one phenyl, and said phenyl is substituted with one or two F, and
R¹⁰ is selected from the group consisting of phenyl and phenyl substituted with one or two independently selected —OR¹⁵ groups, wherein R¹⁵ is methyl, and
R⁹ is selected from the group consisting of imidazolyl and imidazolyl substituted with one or two independently selected methyl groups groups; or
(6)
R¹ is a methyl or ethyl group substituted with one phenyl, or
R¹ is an methyl or ethyl group substituted with one phenyl, and said phenyl is substituted with one or two F, and
R¹⁰ is phenyl substituted with one —OR¹⁵ group, wherein R¹⁵ is methyl, and
R⁹ is selected from the group consisting of imidazolyl and imidazolyl substituted with one methyl group; or
(7)
R¹ is a methyl or ethyl group substituted with one phenyl, or
R¹ is an methyl or ethyl group substituted with one phenyl, and said phenyl is substituted with one or two R²² groups independently selected from the group consisting of: —SF₅, —OSF₅, —Si(R^{15A})₃, and
R¹⁰ is phenyl substituted with one —OR¹⁵ group, wherein R¹⁵ is methyl, and
R⁹ is selected from the group consisting of imidazolyl and imidazolyl substituted with one methyl group; or
(8)
R¹ is selected from the group consisting of: 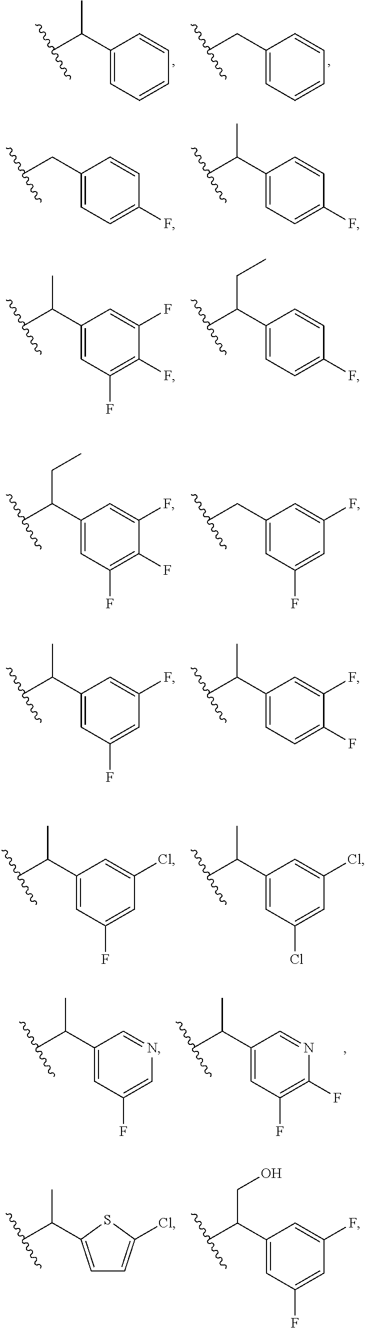 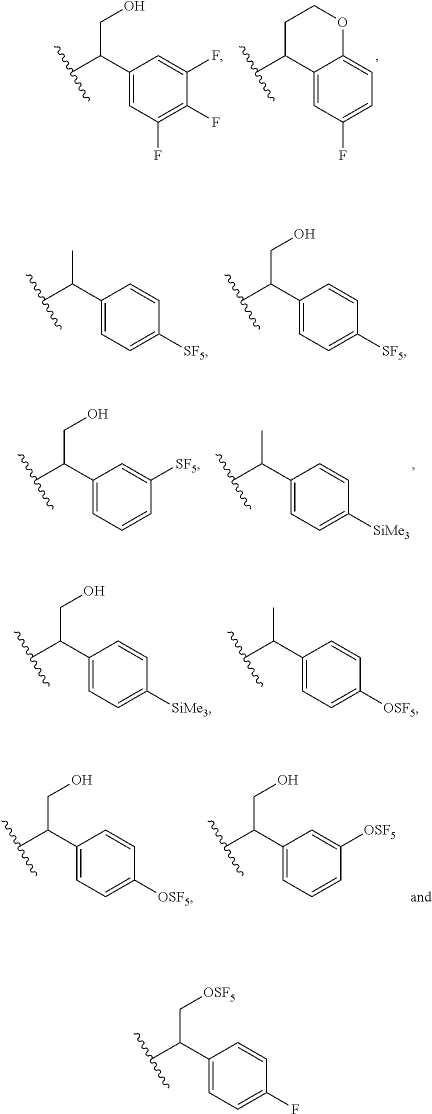
wherein the R⁹-R¹⁰— moiety is: 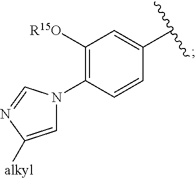
or
(9)
R¹ is selected from the group consisting of: 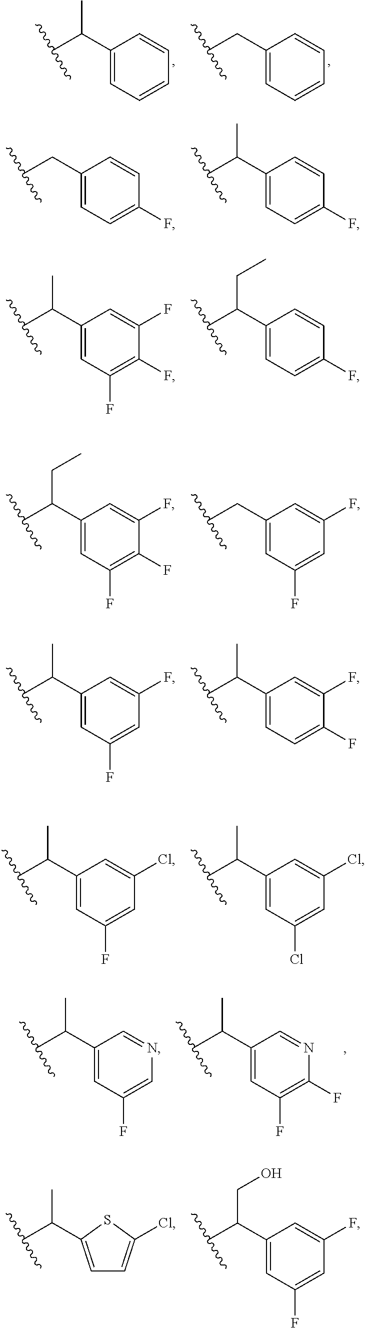 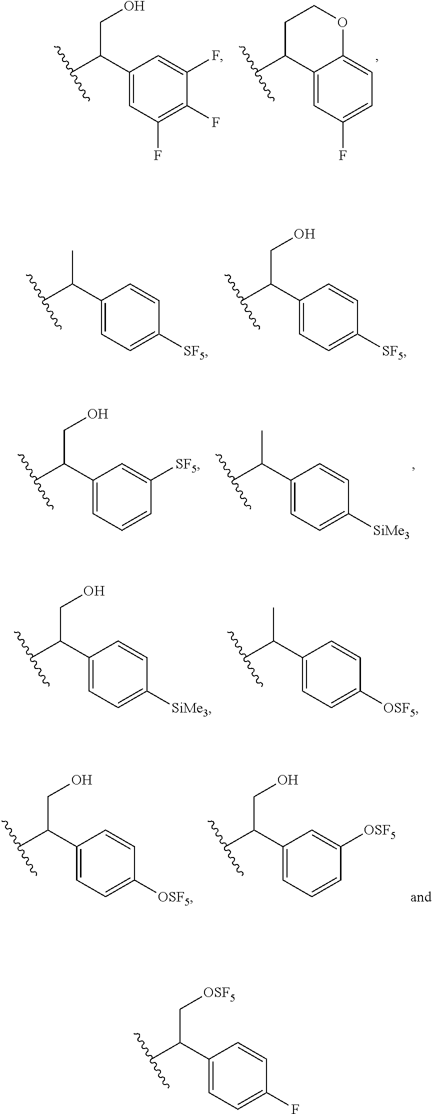
wherein the R⁹-R¹⁰— moiety is: 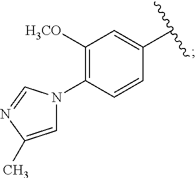
or
(10)
R¹ is selected from the group consisting of: 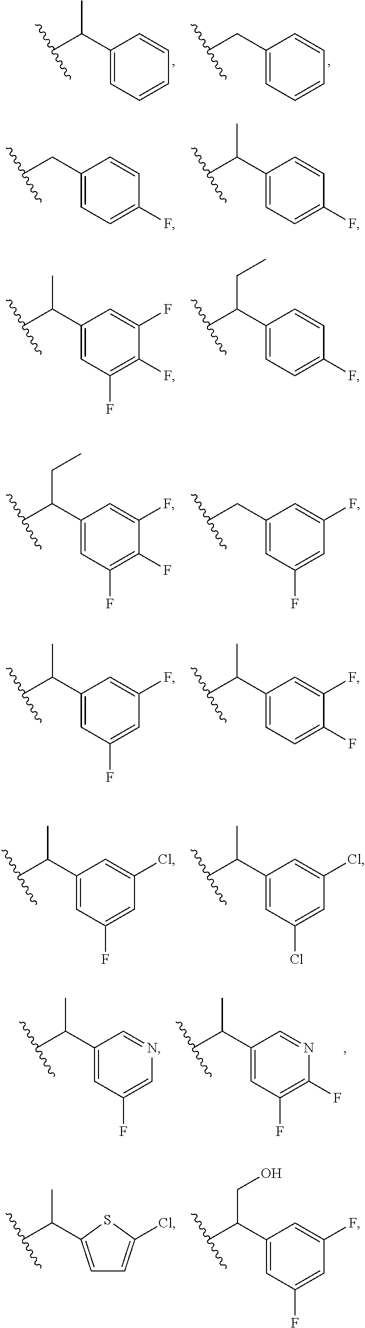 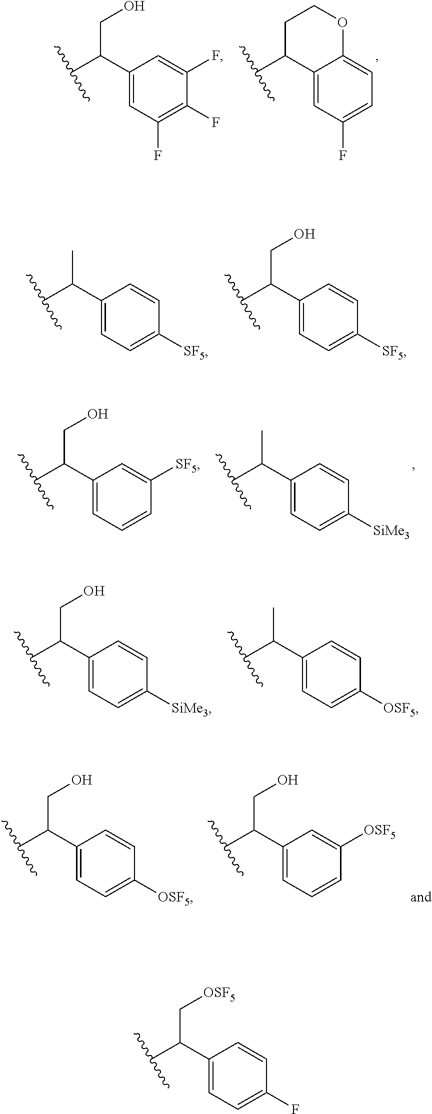
wherein the R⁹-R¹⁰— moiety is: 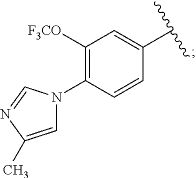
or
(11)
R¹ is selected from the group consisting of: 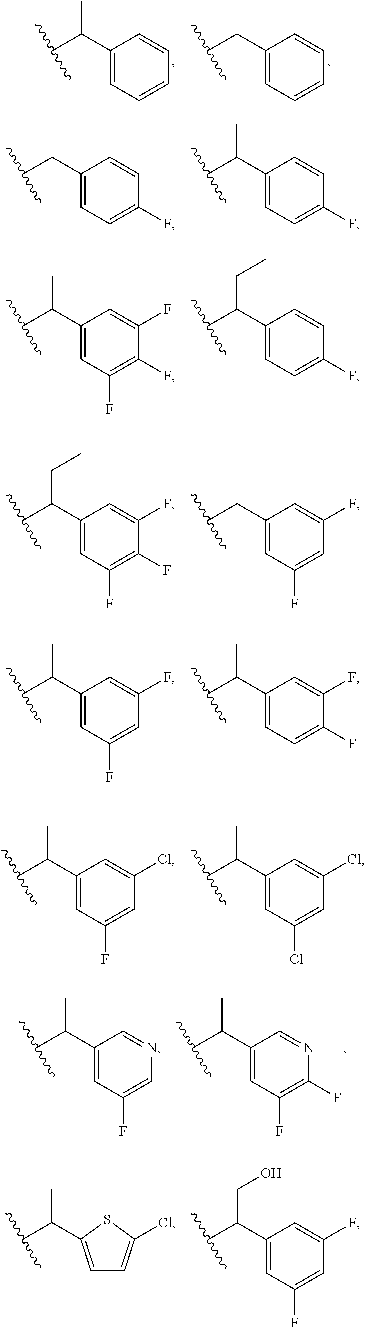 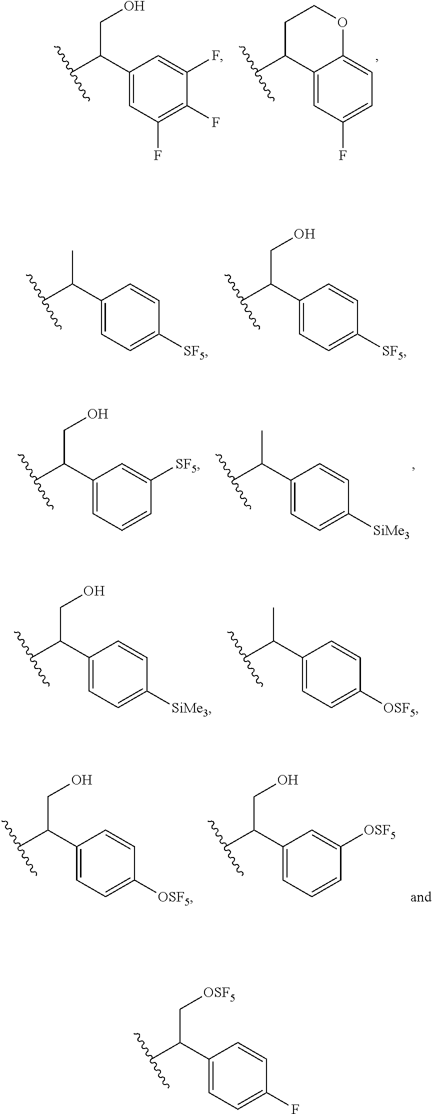
wherein the R⁹-R¹⁰— moiety is: 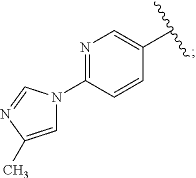
(12)
R¹ is selected from the group consisting of: 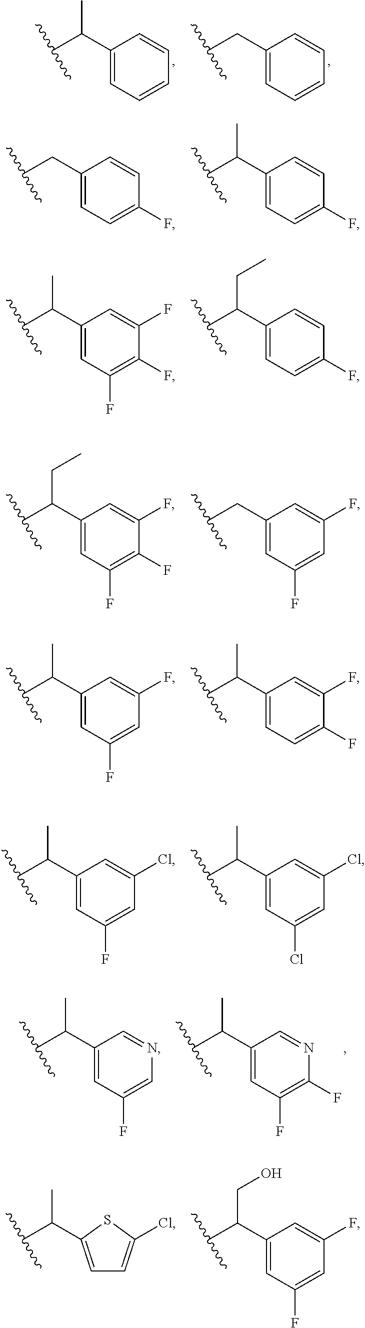 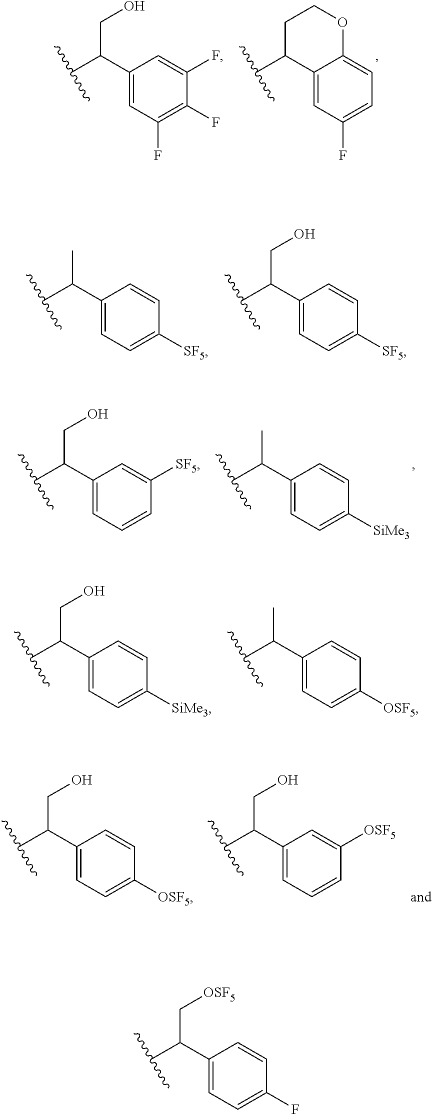
wherein the R⁹-R¹⁰— moiety is: 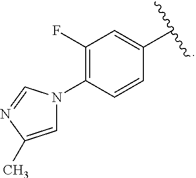
(13)
R¹ is selected from the group consisting of: 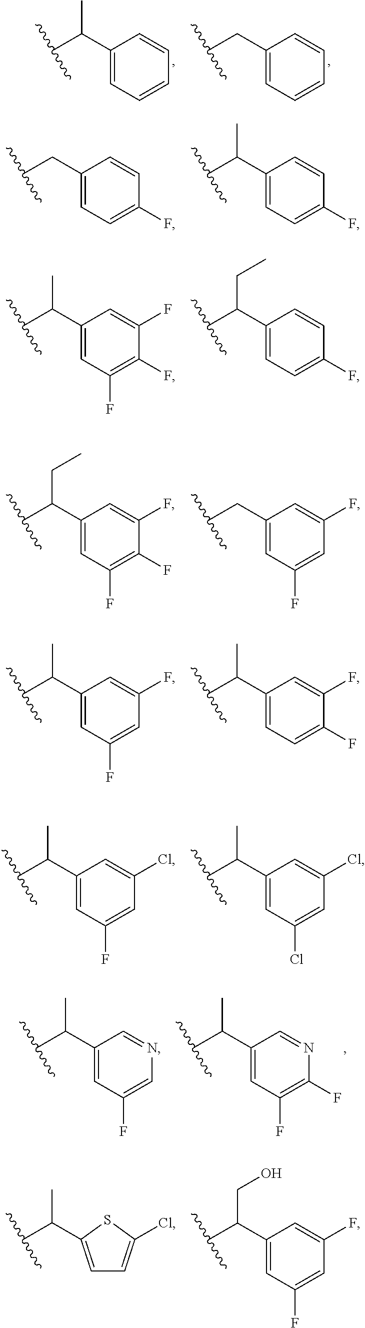 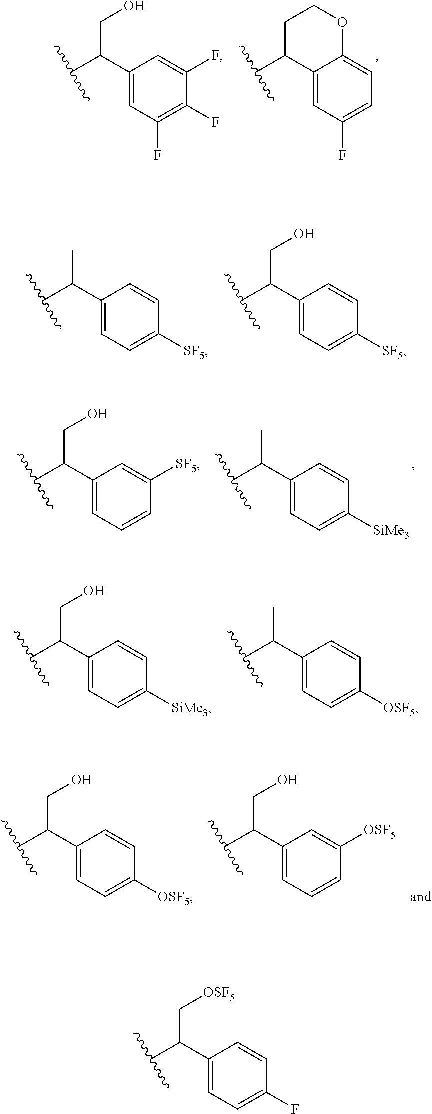
wherein the R⁹-R¹⁰— moiety is selected from the group consisting of:

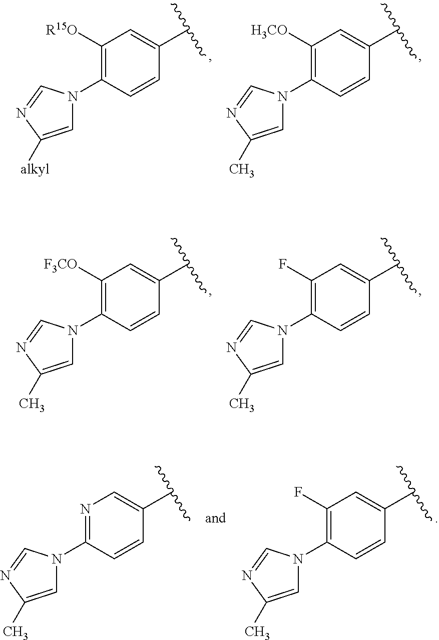

50. The compound of claim 49, wherein W is —C(O)—.

51. The compound of claim 50, wherein G is selected from the group consisting of —NH—, and a direct bond.

52. The compound of claim 43, wherein:
(1) R¹⁰ is an aryl substituted with 1-3 independently selected R²¹ groups; or
(2) R¹⁰ is phenyl substituted with 1-3 independently selected R²¹ groups; or
(3) R¹⁰ is phenyl substituted with 1-3 independently selected —OR¹⁵ groups; or
(4) R¹⁰ is phenyl substituted with two —OR¹⁵ groups, and one R¹⁵ is alkyl, and one R¹⁵ is aryl; or
(5) R¹⁰ is phenyl substituted with two —OR¹⁵ groups, and one R¹⁵ is methyl, and one R¹⁵ is phenyl.

53. The compound of claim 43 selected from the group consisting of:

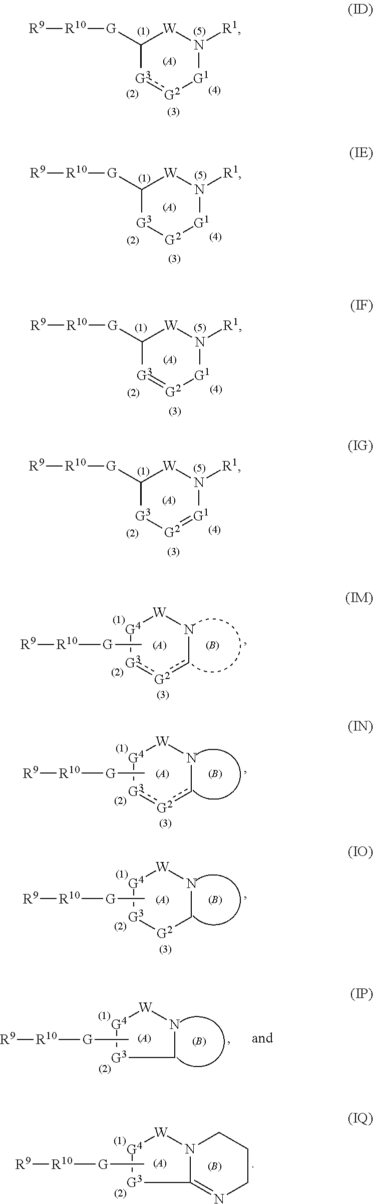

54. The compound of claim 43, wherein R¹ is: 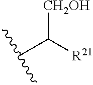
and R²¹ is unsubstituted aryl or aryl substituted with one or more independently selected R²² groups.

55. The compound of claim 43 selected from the group consisting of: compounds 1 to 32, 1A to 32A, 1C to 32C, 1E to 32E, B1 to B3, B4, B5, B6, B7, and B8, B9, B10, A1 to A6, A8, A9, A10, A11, and A12 to A107.

56. A pharmaceutical composition comprising:
(a) a therapeutically effective amount of at least one compound of claim 43, or a pharmaceutically acceptable salt thereof, and at least one pharmaceutically acceptable carrier; or
(b) a therapeutically effective amount of at least one compound of claim 43, or a pharmaceutically acceptable salt thereof, and at least one pharmaceutically acceptable carrier, and a therapeutically effective amount of one or more compounds selected from the group consisting of BACE inhibitors; muscarinic antagonists; cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon (rivastigmine); Cognex (tacrine); Tau kinase inhibitors; anti-Abeta vaccine; APP ligands; agents that upregulate insulin cholesterol lowering agents, Fluvastatin, Lovastatin, Mevastatin, Pitavastatin, Pravastatin, Rosuvastatin, Simvastatin); cholesterol absorption inhibitors (such as Ezetimibe); fibrates (such as, for example, for example, clofibrate, Clofibride, Etofibrate, and Aluminium Clofibrate); LXR agonists; LAP mimics; nicotinic receptor agonists; H3 receptor antagonists; histone deacetylase inhibitors; hsp90 inhibitors; m1 muscarinic receptor agonists; 5-HT6 receptor antagonists; mGluR1; mGluR5; positive allosteric modulators or agonists; mGluR2/3 antagonists; anti-inflammatory agents that can reduce neuroinflammation; Prostaglandin EP2 receptor antagonists; PAI-1 inhibitors; and agents that can induce Abeta efflux such as gelsolin.

57. At least one compound of claim 43, or a pharmaceutically acceptable salt thereof in combination with a BACE inhibitor.

58. A method for treating Alzheimer's Disease comprising administering to a patient in need thereof a therapeutically effective amount of a compound according to claim 1 or a pharmaceutically acceptable salt thereof.

59. The method according to claim 58, further comprising administering to the patient a compound selected from the group consisting of BACE inhibitors; muscarinic antagonists; cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Exelon; Cognex; Tau kinase inhibitors; anti-Abeta vaccine; APP ligands; agents that upregulate insulin cholesterol lowering agents; cholesterol absorption inhibitors; fibrates; LXR agonists; LRP mimics; nicotinic receptor agonists; H3 receptor antagonists; histone deacetylase inhibitors; hsp90 inhibitors; ml muscarinic receptor agonists; 5-HT6 receptor antagonists; mGluR1; mGluR5; positive allosteric modulators or agonists; mGluR2/3 antagonists; anti-inflammatory agents that can reduce neuroinflammation; prostaglandin EP2 receptor antagonists; PAI-1 inhibitors; and agents that can induce Abeta efflux such as gelsolin.

60. A method for inhibiting the deposition of amyloid protein in, on or around neurological tissue, the method comprising administering to a patient in need thereof an effective amount of a compound according to claim 43 or a pharmaceutically acceptable salt thereof.
